SEQUENCE LISTING (3 sequences)

SEQ ID NO: 1 - DNA, 17 bp, Artificial Sequence
  5B.2F Primer
atggacaggcgccctga

SEQ ID NO: 2 - DNA, 20 bp, Artificial Sequence
  5B.2R Primer
ttgatgggcagcttggtttc

SEQ ID NO: 3 - DNA, 17 bp, Artificial Sequence
  FAM labeled probe
cacgccatgcgctgcgg

(19) United States
(12) Patent Application Publication
(10) Pub. No.: US 2014/0378416 A1
(43) Pub. Date: Dec. 25, 2014

(54) SILYL-CONTAINING HETEROCYCLIC COMPOUNDS AND METHODS OF USE THEREOF FOR THE TREATMENT OF VIRAL DISEASES

(71) Applicants: Michael P. Dwyer, Scotch Plains, NJ (US); Kartik M. Keertikar, East Windsor, NJ (US); Qingbei Zeng, Edison, NJ (US); Robert D. Mazzola, JR., Stewartsville, NJ (US); Wensheng Yu, Edison, NJ (US); Haiqun Tang, Belle Mead, NJ (US); Seong Heon Kim, SLivingston, NJ (US); Ling Tong, Warren, NJ (US); Stuart B. Rosenblum, West Orange, NJ (US); Joseph A. Kozlowski, Princeton, NJ (US); Anilkumar Gopinadhan Nair, Edison, NJ (US)

(72) Inventors: Michael P. Dwyer, Scotch Plains, NJ (US); Kartik M. Keertikar, East Windsor, NJ (US); Qingbei Zeng, Edison, NJ (US); Robert D. Mazzola, JR., Stewartsville, NJ (US); Wensheng Yu, Edison, NJ (US); Haiqun Tang, Belle Mead, NJ (US); Seong Heon Kim, SLivingston, NJ (US); Ling Tong, Warren, NJ (US); Stuart B. Rosenblum, West Orange, NJ (US); Joseph A. Kozlowski, Princeton, NJ (US); Anilkumar Gopinadhan Nair, Edison, NJ (US)

(21) Appl. No.: 14/345,114

(22) PCT Filed: Sep. 11, 2012
(86) PCT No.: PCT/US12/54598

§ 371 Date: Sep. 9, 2014

Related U.S. Application Data
(60) Provisional application No. 61/534,583, filed on Sep. 14, 2011.

(51) Int. Cl. C07F 7/10; A61K 45/06; A61K 31/695

(52) U.S. Cl. CPC C07F 7/10; A61K 31/695; A61K 45/06; A61K 2300/00; A61K 2201/094
USPC 514/63; 548/110; 546/14

(57) ABSTRACT

The present invention relates to novel Silyl-Containing Heterocyclic Compounds of Formula (I): and pharmaceutically acceptable salts thereof, wherein A, B, C, D, E, F and L are as defined herein. The present invention also relates to compositions comprising at least one Silyl-Containing Heterocyclic Compound, and methods of using the Silyl-Containing Heterocyclic Compounds for treating or preventing HCV infection in a patient.

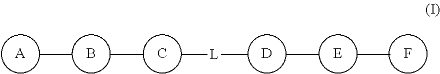

### FIELD OF THE INVENTION

The present invention relates to novel Silyl-Containing Heterocyclic Compounds, compositions comprising at least one Silyl-Containing Heterocyclic Compound, and methods of using the Silyl-Containing Heterocyclic Compounds for treating or preventing HCV infection in a patient.

### BACKGROUND OF THE INVENTION

Hepatitis C virus (HCV) is a major human pathogen. A substantial fraction of these HCV-infected individuals develop serious progressive liver disease, including cirrhosis and hepatocellular carcinoma, which are often fatal. HCV is a (+)-sense single-stranded enveloped RNA virus that has been implicated as the major causative agent in non-A, non-B hepatitis (NANBH), particularly in blood-associated NANBH (BB-NANBH) (see, International Publication No. WO 89/04669 and European Patent Publication No. EP 381 216). NANBH is to be distinguished from other types of viral-induced liver disease, such as hepatitis A virus (HAV), hepatitis B virus (HBV), delta hepatitis virus (HDV), cytomegalovirus (CMV) and Epstein-Barr virus (EBV), as well as from other forms of liver disease such as alcoholism and primary biliar cirrhosis.

It is well-established that persistent infection of HCV is related to chronic hepatitis, and as such, inhibition of HCV replication is a viable strategy for the prevention of hepatocellular carcinoma. Current therapies for HCV infection include α-interferon monotherapy and combination therapy comprising α-interferon and ribavirin. These therapies have been shown to be effective in some patients with chronic HCV infection, but suffer from poor efficacy and unfavorable side-effects and there are currently efforts directed to the discovery of HCV replication inhibitors that are useful for the treatment and prevention of HCV related disorders.

Current research efforts directed toward the treatment of HCV includes the use of antisense oligonucleotides, free bile acids (such as ursodeoxycholic acid and chenodeoxycholic acid) and conjugated bile acids (such as tauroursodeoxycholic acid). Phosphonoformic acid esters have also been proposed as potentially useful for the treatment of various viral infections, including HCV. Vaccine development, however, has been hampered by the high degree of viral strain heterogeneity and immune evasion and the lack of protection against reinfection, even with the same inoculum.

In light of these treatment hurdles, the development of small-molecule inhibitors directed against specific viral targets has become a major focus of anti-HCV research. The determination of crystal structures for NS3 protease, NS3 RNA helicase, NS5A, and NS5B polymerase, with and without bound ligands, has provided important structural insights useful for the rational design of specific inhibitors.

Recent attention has been focused toward the identification of inhibitors of HCV NS5A. HCV NS5A is a 447 amino acid phosphoprotein which lacks a defined enzymatic function. It runs as 56 kd and 58 kd bands on gels depending on phosphorylation state (Tanji, et al. J. Virol. 69:3980-3986 (1995)). HCV NS5A resides in replication complex and may be responsible for the switch from replication of RNA to production of infectious virus (Huang, Y, et al., Virology 364:1-9 (2007)).

Multicyclic HCV NS5A inhibitors have been reported. See U.S. Patent Publication Nos. US20080311075, US20080044379, US20080050336, US20080044380, US20090202483 and US2009020478. HCV NS5A inhibitors having fused tricyclic moieties are disclosed in International Patent Publication Nos. WO 10/065681, WO 10/065668, and WO 10/065674.

Other HCV NS5A inhibitors and their use for reducing viral load in HCV infected humans have been described in U.S. Patent Publication No. US20060276511.

### SUMMARY OF THE INVENTION

In one aspect, the present invention provides Compounds of Formula (I)

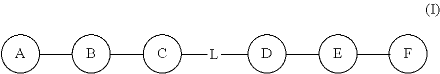

or a pharmaceutically acceptable salt thereof,
wherein:

A and F are each independently R¹² or

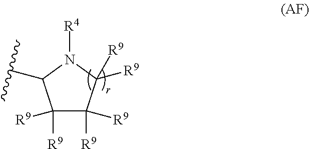

wherein each occurrence of (AF) can be independently and optionally fused to a benzene ring and wherein any two R⁹ groups that are attached to the same (AF) group, together with the ring carbon atom(s) to which they are attached, can join to form a 3 to 7-membered cycloalkyl group, such that when the group corresponding to variable D does not contains the group —Si(R¹¹)₂— as a ring member, then at least one of A and F is R¹²;

B and E are each independently imidazolyl or benzimidazolyl, wherein said imidazolyl group and said benzimidazolyl group can be optionally and independently substituted on a ring carbon atoms with R⁶;

C is selected from a bond, phenylene, naphthylene and 5 or 6-membered monocyclic heteroarylene, wherein said phenylene group, said naphthylene group and said 5 or 6-membered monocyclic heteroarylene group can be optionally and independently substituted on one or more ring carbon atoms with R¹⁰;

D is selected from phenylene, naphthylene, 5 or 6-membered monocyclic heteroarylene, 9 or 10-membered bicyclic heteroarylene and 13 to 14-membered tricyclic heteroarylene, wherein said 5-membered monocyclic heteroarylene group, said 9 or 10-membered bicyclic heteroarylene group and said 13 to 14-membered tricyclic heteroarylene group can be optionally and independently substituted on one or more ring carbon atoms with R¹⁰, and wherein said 9 or 10-membered bicyclic heteroarylene group and said 13 to 14-membered tricyclic heteroarylene group can optionally contain the group —Si(R¹¹)₂— as a ring member;

L is selected from a bond, C₁-C₃ alkylene, —CH═CH— and —C≡C—, such that when D is 13 to 14-membered tricyclic heteroarylene, then L is a bond;

each occurrence of R¹ is independently selected from C₁-C₆ alkyl, C₁-C₆ haloalkyl, 3- to 7-membered cycloalkyl, 4- to 7-membered heterocycloalkyl, aryl and 5 or 6-membered monocyclic heteroaryl, wherein said 3- to 7-membered cycloalkyl group, said 4- to 7-membered heterocycloalkyl group, said aryl group or said 5 or 6-membered monocyclic heteroaryl group can be optionally substituted with up to three groups, which can be the same or different, and are selected from C₁-C₆ alkyl, 3- to 7-membered cycloalkyl, 4- to 7-membered heterocycloalkyl, aryl, heteroaryl, halo, C₁-C₆ haloalkyl, —Si(R¹¹)₃, —CN, —OR², —N(R²)₂, —C(O)R³, —C(O)OR², —C(O)N(R²)₂, —NHC(O)R³, —NHC(O)NHR², —NHC(O)OR², —OC(O)R³, —SR² and —S(O)₂R³;

each occurrence of R² is independently selected from H, C₁-C₆ alkyl, C₁-C₆ haloalkyl, —C₁-C₆ alkylene-OC(O)(C₁-C₆ alkyl), C₁-C₆ hydroxyalkyl, 3 to 7-membered cycloalkyl, 4 to 7-membered heterocycloalkyl, aryl and 5 or 6-membered monocyclic heteroaryl wherein said 3- to 7-membered cycloalkyl group, said 4- to 7-membered heterocycloalkyl group, said aryl group or said 5 or 6-membered monocyclic heteroaryl group can be optionally and independently substituted with up to three groups, each independently selected from —OH, halo, C₁-C₆ alkyl, C₁-C₆ haloalkyl, —NH(C₁-C₆ alkyl) and —N(C₁-C₆ alkyl)₂;

each occurrence of R³ is independently selected from C₁-C₆ alkyl, C₁-C₆ haloalkyl, 3 to 7-membered cycloalkyl, 4 to 7-membered heterocycloalkyl, aryl, and 5 or 6-membered monocyclic heteroaryl;

each occurrence of R⁴ is independently selected from H, —C₁-C₆ alkyl, C₁-C₆ haloalkyl, —C(O)R¹, —C(O)OR¹, —C(O)C(R⁵)₂NHC(O)OR¹ and —C(O)—C(R⁵)₂—N(R¹)₂;

each occurrence of R⁵ is independently selected from H, C₁-C₆ alkyl, C₁-C₆ haloalkyl, -alkylene-O—(C₁-C₆ alkyl), C₁-C₆ silylalkyl, —(CH₂)ₙ-aryl, —(CH₂)ₙ-(3- to 7-membered cycloalkyl), —(CH₂)ₙ-(4- to 7-membered heterocycloalkyl group) and —(CH₂)ₙ-(5 or 6-membered monocyclic heteroaryl), wherein said 3- to 7-membered cycloalkyl group, said 4- to 7-membered heterocycloalkyl group, said aryl group or said 5 or 6-membered monocyclic heteroaryl group can be optionally and independently substituted with up to three R⁷ groups, or two R⁵ groups that are attached to the same carbon atom, together with the common carbon atom to which they are attached, can join to form a 3-6 membered cycloalkyl group;

each occurrence of R⁶ is independently selected from H, halo, C₁-C₆ alkyl and 3 to 7-membered cycloalkyl;

each occurrence of R⁷ is independently selected from H, C₁-C₆ alkyl, halo, —C₁-C₆ haloalkyl, C₁-C₆ hydroxyalkyl, —OH, —C(O)NH—(C₁-C₆ alkyl), —C(O)N(C₁-C₆ alkyl)₂, —O—(C₁-C₆ alkyl), —NH₂, —NH(C₁-C₆ alkyl), —N(C₁-C₆ alkyl)₂ and —NHC(O)—(C₁-C₆ alkyl) and —Si(R¹¹)₃;

each occurrence of R⁹ is independently selected from H, halo and C₁-C₆ alkyl;

each occurrence of R¹⁰ is independently selected from H, C₁-C₆ alkyl, C₁-C₆ haloalkyl, 3 to 7-membered cycloalkyl, 4 to 7-membered heterocycloalkyl, aryl, 5 or 6-membered monocyclic heteroaryl, halo, —CN, —OR³, —N(R²)₂, —C(O)R³, —C(O)OR², —C(O)N(R²)₂, —NHC(O)R³, —NHC(O)NHR², —NHC(O)OR², —OC(O)R³, —SR², —S(O)₂R³ and Si(R¹¹)₃, wherein any two R¹⁰ groups that are attached to the same ring, together with the ring carbon atom(s) to which they are attached, can optionally join to form a 5 to 7-membered cycloalkyl group or 4- to 7-membered heterocycloalkyl group;

each occurrence of R¹¹ is independently selected from C₁-C₆ alkyl, 3- to 7-membered cycloalkyl, 4- to 7-membered heterocycloalkyl, aryl, heteroaryl, C₁-C₆ haloalkyl, —CN and —OR², wherein two R¹¹ groups that are attached to the same silicon atom, together with the common silicon atom to which they are attached, can optionally join to form a 4- to 7-membered spirocyclic silicon-containing heterocycloalkyl ring;

each occurrence of R¹² is independently a monocyclic 5 to 7-membered silylheterocycloalkyl ring or a bicyclic 7 to 11-membered bicyclic silylheterocycloalkyl ring wherein said silylheterocycloalkyl rings contains as heteroatom ring members:
(i) one —Si(R¹¹)₂— group; and
(ii) one —N(R⁴)— group;

wherein an R¹² group can be optionally and independently substituted on one or more ring carbon atoms with R¹⁰;

each occurrence of n is independently 0, 1, 2 or 3; and

each occurrence of r is independently 0 or 1.

The Compounds of Formula (I) (also referred to herein as the “Silyl-Containing Heterocyclic Compounds”) and pharmaceutically acceptable salts thereof can be useful, for example, for inhibiting HCV viral replication or replicon activity, and for treating or preventing HCV infection in a patient. Without being bound by any specific theory, it is believed that the Silyl-Containing Heterocyclic Compounds inhibit HCV viral replication by inhibiting HCV NS5A.

Accordingly, the present invention provides methods for treating or preventing HCV infection in a patient, comprising administering to the patient an effective amount of at least one Silyl-Containing Heterocyclic Compound.

The details of the invention are set forth in the accompanying detailed description below.

Although any methods and materials similar to those described herein can be used in the practice or testing of the present invention, illustrative methods and materials are now described. Other embodiments, aspects and features of the present invention are either further described in or will be apparent from the ensuing description, examples and appended claims.

### DETAILED DESCRIPTION OF THE INVENTION

The present invention relates to novel Silyl-Containing Heterocyclic Compounds, compositions comprising at least one Silyl-Containing Heterocyclic Compound, and methods of using the Silyl-Containing Heterocyclic Compounds for treating or preventing HCV infection in a patient.

### DEFINITIONS AND ABBREVIATIONS

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc. . . .

As used herein, and throughout this disclosure, the following terms, unless otherwise indicated, shall be understood to have the following meanings:

A “patient” is a human or non-human mammal. In one embodiment, a patient is a human. In another embodiment, a patient is a chimpanzee.

The term “effective amount” as used herein, refers to an amount of Silyl-Containing Heterocyclic Compound and/or an additional therapeutic agent, or a composition thereof that is effective in producing the desired therapeutic, ameliorative, inhibitory or preventative effect when administered to a patient suffering from a viral infection or virus-related disorder. In the combination therapies of the present invention, an effective amount can refer to each individual agent or to the combination as a whole, wherein the amounts of all agents administered are together effective, but wherein the component agent of the combination may not be present individually in an effective amount.

The term “preventing,” as used herein with respect to an HCV viral infection or HCV-virus related disorder, refers to reducing the likelihood of HCV infection.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In different embodiments, an alkyl group contains from 1 to 6 carbon atoms (C₁-C₆ alkyl) or from about 1 to about 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkyl group is linear. In another embodiment, an alkyl group is branched. Unless otherwise indicated, an alkyl group is unsubstituted.

The term “alkenyl,” as used herein, refers to an aliphatic hydrocarbon group containing at least one carbon-carbon double bond and having one of its hydrogen atoms replaced with a bond. An alkenyl group may be straight or branched and contain from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. The term “C₂-C₆ alkenyl” refers to an alkenyl group having from 2 to 6 carbon atoms. Unless otherwise indicated, an alkenyl group is unsubstituted.

The term “alkynyl,” as used herein, refers to an aliphatic hydrocarbon group containing at least one carbon-carbon triple bond and having one of its hydrogen atoms replaced with a bond. An alkynyl group may be straight or branched and contain from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. The term “C₂-C₆ alkynyl” refers to an alkynyl group having from 2 to 6 carbon atoms. Unless otherwise indicated, an alkynyl group is unsubstituted.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and —CH₂CH(CH₃)CH₂—. In one embodiment, an alkylene group has from 1 to about 6 carbon atoms. In another embodiment, an alkylene group is branched. In another embodiment, an alkylene group is linear. In one embodiment, an alkylene group is —CH₂—. The term “C₁-C₆ alkylene” refers to an alkylene group having from 1 to 6 carbon atoms.

The term “aryl,” as used herein, refers to an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. In one embodiment, an aryl group can be optionally fused to a cycloalkyl or cycloalkanoyl group. Non-limiting examples of aryl groups include phenyl and naphthyl. In one embodiment, an aryl group is phenyl. Unless otherwise indicated, an aryl group is unsubstituted.

The term “arylene,” as used herein, refers to a bivalent group derived from an aryl group, as defined above, by removal of a hydrogen atom from a ring carbon of an aryl group. An arylene group can be derived from a monocyclic or multicyclic ring system comprising from about 6 to about 14 carbon atoms. In one embodiment, an arylene group contains from about 6 to about 10 carbon atoms. In another embodiment, an arylene group is a naphthylene group. In another embodiment, an arylene group is a phenylene group. An arylene group can be optionally substituted with one or more “ring system substituents” which may be the same or different, and are as defined herein below. An arylene group is divalent and either available bond on an arylene group can connect to either group flanking the arylene group. For example, the group “A-arylene-B,” wherein the arylene group is:

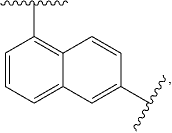 [SMILES: *c1ccc2c(*)cccc2c1]

is understood to represent both:

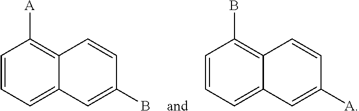

In one embodiment, an arylene group can be optionally fused to a cycloalkyl or cycloalkanoyl group. Non-limiting examples of arylene groups include phenylene and naphthalene. In one embodiment, an arylene group is unsubstituted. In another embodiment, an arylene group is:

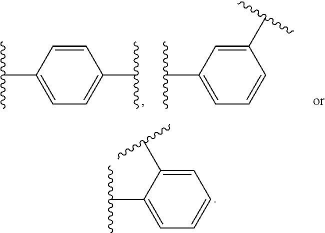

Unless otherwise indicated, an arylene group is unsubstituted.

The term “cycloalkyl,” as used herein, refers to a non-aromatic mono- or multicyclic ring system comprising from about 3 to about 10 ring carbon atoms. In one embodiment, a cycloalkyl contains from about 5 to about 10 ring carbon atoms. In another embodiment, a cycloalkyl contains from about 3 to about 7 ring atoms. In another embodiment, a cycloalkyl contains from about 5 to about 6 ring atoms. The term “cycloalkyl” also encompasses a cycloalkyl group, as defined above, which is fused to an aryl (e.g., benzene) or heteroaryl ring. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. In one embodiment, a cycloalkyl group is unsubstituted. The term “3 to 7-membered cycloalkyl” refers to a cycloalkyl group having from 3 to 7 ring carbon atoms. Unless otherwise indicated, a cycloalkyl group is unsubstituted. A ring carbon atom of a cycloalkyl group may be functionalized as a carbonyl group. An illustrative example of such a cycloalkyl group (also referred to herein as a “cycloalkanoyl” group) includes, but is not limited to, cyclobutanoyl:

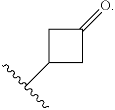 [SMILES: *C1CC(=O)C1]

The term “cycloalkenyl,” as used herein, refers to a non-aromatic mono- or multicyclic ring system comprising from about 4 to about 10 ring carbon atoms and containing at least one endocyclic double bond. In one embodiment, a cycloalkenyl contains from about 4 to about 7 ring carbon atoms. In another embodiment, a cycloalkenyl contains 5 or 6 ring atoms. Non-limiting examples of monocyclic cycloalkenyls include cyclopentenyl, cyclohexenyl, cyclohepta-1,3-dienyl, and the like. A cycloalkenyl group can be optionally substituted with one or more “ring system substituents” which may be the same or different, and are as defined herein below. A ring carbon atom of a cycloalkyl group may be functionalized as a carbonyl group. In one embodiment, a cycloalkenyl group is cyclopentenyl. In another embodiment, a cycloalkenyl group is cyclohexenyl. The term “4 to 7-membered cycloalkenyl” refers to a cycloalkenyl group having from 4 to 7 ring carbon atoms. Unless otherwise indicated, a cycloalkenyl group is unsubstituted.

The term “halo,” as used herein, means —F, —Cl, —Br or —I.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CF₃, —CH₂Cl and —CCl₃. The term “C₁-C₆ haloalkyl” refers to a haloalkyl group having from 1 to 6 carbon atoms.

The term “hydroxyalkyl,” as used herein, refers to an alkyl group as defined above, wherein one or more of the alkyl group's hydrogen atoms has been replaced with an —OH group. In one embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃. The term “C₁-C₆ hydroxyalkyl” refers to a hydroxyalkyl group having from 1 to 6 carbon atoms.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. In another embodiment, a heteroaryl group is bicyclic. A heteroaryl group can be optionally substituted by one or more “ring system substituents” which may be the same or different, and are as defined herein below. A heteroaryl group is joined via a ring carbon atom, and any nitrogen atom of a heteroaryl can be optionally oxidized to the corresponding N-oxide. The term “heteroaryl” encompasses any fused polycyclic ring system in which at least one of the fused rings is aromatic. The term “heteroaryl” also encompasses a heteroaryl group, as defined above, which is fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, oxadiazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, benzimidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like, and all isomeric forms thereof. The term “heteroaryl” also refers to partially saturated heteroaryl moieties such as, for example, tetrahydroisoquinolyl, tetrahydroquinolyl and the like. In one embodiment, a heteroaryl group is a 5-membered heteroaryl. In another embodiment, a heteroaryl group is a 6-membered heteroaryl. In another embodiment, a heteroaryl group comprises a 5- to 6-membered heteroaryl group fused to a benzene ring. Unless otherwise indicated, a heteroaryl group is unsubstituted.

The term “heteroarylene,” as used herein, refers to a bivalent group derived from an heteroaryl group, as defined above, by removal of a hydrogen atom from a ring carbon or ring heteroatom of a heteroaryl group. A heteroarylene group can be derived from a monocyclic or multicyclic ring system comprising about 5 to about 14 ring atoms, wherein from 1 to 4 of the ring atoms are each independently O, N or S and the remaining ring atoms are carbon atoms. A heteroarylene group can be optionally substituted by one or more “ring system substituents” which may be the same or different, and are as defined herein below. A heteroarylene group is joined via a ring carbon atom or by a nitrogen atom with an open valence, and any nitrogen atom of a heteroarylene can be optionally oxidized to the corresponding N-oxide. The term “heteroarylene” also encompasses a heteroarylene group, as defined above, which is fused to a benzene ring. Non-limiting examples of heteroarylenes include pyridylene, pyrazinylene, furanylene, thienylene, pyrimidinylene, pyridonylene (including those derived from N-substituted pyridonyls), isoxazolylene, isothiazolylene, oxazolylene, oxadiazolylene, thiazolylene, pyrazolylene, thiophenylene, furazanylene, pyrrolylene, triazolylene, 1,2,4-thiadiazolylene, pyrazinylene, pyridazinylene, quinoxalinylene, phthalazinylene, oxindolylene, imidazo[1,2-a]pyridinylene, imidazo[2,1-b]thiazolylene, benzofurazanylene, indolylene, azaindolylene, benzimidazolylene, benzothienylene, quinolinylene, imidazolylene, benzimidazolylene, thienopyridylene, quinazolinylene, thienopyrimidylene, pyrrolopyridylene, imidazopyridylene, isoquinolinylene, benzoazaindolylene, 1,2,4-triazinylene, benzothiazolylene and the like, and all isomeric forms thereof. The term “heteroarylene” also refers to partially saturated heteroarylene moieties such as, for example, tetrahydroisoquinolylene, tetrahydroquinolylene, and the like. A heteroarylene group is divalent and either available bond on a heteroarylene ring can connect to either group flanking the heteroarylene group. For example, the group “A-heteroarylene-B,” wherein the heteroarylene group is:

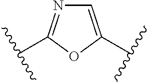 [SMILES: *c1cnc(*)o1]

is understood to represent both:

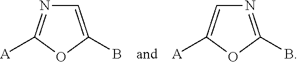

In one embodiment, a heteroarylene group is a monocyclic heteroarylene group or a bicyclic heteroarylene group. In another embodiment, a heteroarylene group is a monocyclic heteroarylene group. In another embodiment, a heteroarylene group is a bicyclic heteroarylene group. In still another embodiment, a heteroarylene group has from about 5 to about 10 ring atoms. In another embodiment, a heteroarylene group is monocyclic and has 5 or 6 ring atoms. In another embodiment, a heteroarylene group is bicyclic and has 9 or 10 ring atoms. In another embodiment, a heteroarylene group is tricyclic and has 13 or 14 ring atoms. In another embodiment, a heteroarylene group is a 5-membered monocyclic heteroarylene. In another embodiment, a heteroarylene group is a 6-membered monocyclic heteroarylene. In another embodiment, a bicyclic heteroarylene group comprises a 5 or 6-membered monocyclic heteroarylene group fused to a benzene ring. Unless otherwise indicated, a heteroarylene group is unsubstituted.

The term “heterocycloalkyl,” as used herein, refers to a non-aromatic saturated monocyclic or multicyclic ring system comprising 3 to about 11 ring atoms, wherein from 1 to 4 of the ring atoms are independently O, S, N or Si, and the remainder of the ring atoms are carbon atoms. A heterocycloalkyl group can be joined via a ring carbon, ring silicon atom or ring nitrogen atom. In one embodiment, a heterocycloalkyl group is monocyclic and has from about 3 to about 7 ring atoms. In another embodiment, a heterocycloalkyl group is monocyclic has from about 4 to about 7 ring atoms. In another embodiment, a heterocycloalkyl group is bicyclic and has from about 7 to about 11 ring atoms. In still another embodiment, a heterocycloalkyl group is monocyclic and has 5 or 6 ring atoms. In one embodiment, a heterocycloalkyl group is monocyclic. In another embodiment, a heterocycloalkyl group is bicyclic. There are no adjacent oxygen and/or sulfur atoms present in the ring system. Any —NH group in a heterocycloalkyl ring may exist protected such as, for example, as an —N(BOC), —N(Cbz), —N(Tos) group and the like; such protected heterocycloalkyl groups are considered part of this invention. The term “heterocycloalkyl” also encompasses a heterocycloalkyl group, as defined above, which is fused to an aryl (e.g., benzene) or heteroaryl ring. A heterocycloalkyl group can be optionally substituted by one or more “ring system substituents” which may be the same or different, and are as defined herein below. The nitrogen or sulfur atom of the heterocycloalkyl can be optionally oxidized to the corresponding N-oxide, S-oxide or S,S-dioxide. Non-limiting examples of monocyclic heterocycloalkyl rings include oxetanyl, piperidyl, pyrrolidinyl, piperazinyl, morpholinyl, thiomorpholinyl, thiazolidinyl, 1,4-dioxanyl, tetrahydrofuranyl, tetrahydrothiophenyl, delta-lactam, delta-lactone, silacyclopentane, silapyrrolidine and the like, and all isomers thereof. Non-limiting illustrative examples of a silyl-containing heterocycloalkyl group include:

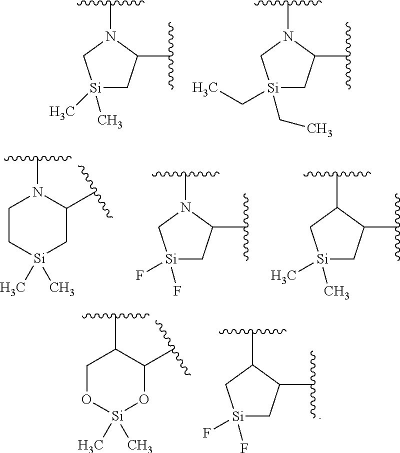

A ring carbon atom of a heterocycloalkyl group may be functionalized as a carbonyl group. An illustrative example of such a heterocycloalkyl group is:

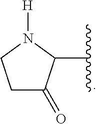 [SMILES: *C1NCCC1=O]

In one embodiment, a heterocycloalkyl group is a 5-membered monocyclic heterocycloalkyl. In another embodiment, a heterocycloalkyl group is a 6-membered monocyclic heterocycloalkyl. The term “3 to 7-membered monocyclic cycloalkyl” refers to a monocyclic heterocycloalkyl group having from 3 to 7 ring atoms. The term “4 to 7-membered monocyclic cycloalkyl” refers to a monocyclic heterocycloalkyl group having from 4 to 7 ring atoms. The term “7 to 11-membered bicyclic heterocycloalkyl” refers to a bicyclic heterocycloalkyl group having from 7 to 11 ring atoms. Unless otherwise indicated, an heterocycloalkyl group is unsubstituted.

The term “heterocycloalkenyl,” as used herein, refers to a heterocycloalkyl group, as defined above, wherein the heterocycloalkyl group contains from 4 to 10 ring atoms, and at least one endocyclic carbon-carbon or carbon-nitrogen double bond. A heterocycloalkenyl group can be joined via a ring carbon or ring nitrogen atom. In one embodiment, a heterocycloalkenyl group has from 4 to 7 ring atoms. In another embodiment, a heterocycloalkenyl group is monocyclic and has 5 or 6 ring atoms. In another embodiment, a heterocycloalkenyl group is bicyclic. A heterocycloalkenyl group can optionally substituted by one or more ring system substituents, wherein “ring system substituent” is as defined above. The nitrogen or sulfur atom of the heterocycloalkenyl can be optionally oxidized to the corresponding N-oxide, S-oxide or S,S-dioxide. Non-limiting examples of heterocycloalkenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluoro-substituted dihydrofuranyl, 7-oxabicyclo[2.2.1]heptenyl, dihydrothiophenyl, dihydrothiopyranyl, and the like and the like. A ring carbon atom of a heterocycloalkenyl group may be functionalized as a carbonyl group. In one embodiment, a heterocycloalkenyl group is a 5-membered heterocycloalkenyl. In another embodiment, a heterocycloalkenyl group is a 6-membered heterocycloalkenyl. The term “4 to 7-membered heterocycloalkenyl” refers to a heterocycloalkenyl group having from 4 to 7 ring atoms. Unless otherwise indicated, a heterocycloalkenyl group is unsubstituted.

The term “ring system substituent,” as used herein, refers to a substituent group attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, -alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, —Si(alkyl)₂, —Si(aryl)₂, —Si(heteroaryl)₂, —Si(alkyl)(aryl), —Si(alkyl)(cycloalkyl), —Si(alkyl)(heteroaryl), cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), —N(Y₁)(Y₂), -alkylene-N(Y₁)(Y₂), —C(O)N(Y₁)(Y₂) and —S(O)₂N(Y₁)(Y₂), wherein Y₁ and Y₂ can be the same or different and are independently selected from the group consisting of hydrogen, alkyl, aryl, cycloalkyl, and -alkylene-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

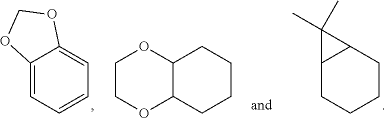

The term “silylalkyl,” as used herein, refers to an alkyl group as defined above, wherein one or more of the alkyl group's hydrogen atoms has been replaced with a —Si(R^{x})₃ group, wherein each occurrence of R^{x} is independently C₁-C₆ alkyl, phenyl or a 3- to 6-membered cycloalkyl group. In one embodiment, a silylalkyl group has from 1 to 6 carbon atoms. In another embodiment, a silyl alkyl group contains a —Si(CH₃)₃ moiety. Non-limiting examples of silylalkyl groups include

—CH₂—Si(CH₃)₃ and —CH₂CH₂—Si(CH₃)₃.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any substituent or variable (e.g., alkyl, R⁶, R^{a}, etc.) occurs more than one time in any constituent or in Formula (I), its definition on each occurrence is independent of its definition at every other occurrence, unless otherwise indicated.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide a Silyl-Containing Heterocyclic Compound or a pharmaceutically acceptable salt or solvate of the compound. The transformation may occur by various mechanisms (e.g., by metabolic or chemical processes), such as, for example, through hydrolysis in blood.

For example, if a Silyl-Containing Heterocyclic Compound or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a Silyl-Containing Heterocyclic Compound contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, —P(O)(OH)₂, —P(O)(O(C₁-C₆)alkyl)₂ or glycosyl (the radical resulting from the removal of a hydroxyl group of the hemiacetal form of a carbohydrate), and the like.

If a Silyl-Containing Heterocyclic Compound incorporates an amine functional group, a prodrug can be formed by the replacement of a hydrogen atom in the amine group with a group such as, for example, R-carbonyl-, RO-carbonyl-, NRR′-carbonyl-wherein R and R′ are each independently (C₁-C₁₀)alkyl, (C₃-C₇) cycloalkyl, benzyl, a natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl; carboxy (C₁-C₆)alkyl; amino(C₁-C₄)alkyl or mono-N- or di-N,N—(C₁-C₆)alkylaminoalkyl; —C(Y⁴)Y⁵ wherein Y⁴ is H or methyl and Y⁵ is mono-N- or di-N,N—(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (e.g., methyl, ethyl, n-propyl, isopropyl, t-butyl, sec-butyl or n-butyl), alkoxyalkyl (e.g., methoxymethyl), aralkyl (e.g., benzyl), aryloxyalkyl (for example, phenoxymethyl), aryl (e.g., phenyl optionally substituted with, for example, halogen, C₁₋₄alkyl, —O—(C₁₋₄alkyl) or amino); (2) sulfonate esters, such as alkyl- or aralkylsulfonyl (for example, methanesulfonyl); (3) amino acid esters (e.g., L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours. 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

The Silyl-Containing Heterocyclic Compounds can form salts which are also within the scope of this invention. Reference to a Silyl-Containing Heterocyclic Compound herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a Silyl-Containing Heterocyclic Compound contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Formula (I) may be formed, for example, by reacting a Silyl-Containing Heterocyclic Compound with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates) and the like. In one embodiment, a compound of formula (I) is present as its dihydrochloride salt. In another embodiment, a compound of formula (I) is present as its dimesylate salt. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures can be separated into their individual diastereomers on the basis of their physical chemical differences by methods well-known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the Silyl-Containing Heterocyclic Compounds may be atropisomers (e.g., substituted biaryls) and are considered as part of this invention. Enantiomers can also be directly separated using chiral chromatographic techniques.

It is also possible that the Silyl-Containing Heterocyclic Compounds may exist in different tautomeric forms, and all such forms are embraced within the scope of the invention. For example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. If a Silyl-Containing Heterocyclic Compound incorporates a double bond or a fused ring, both the cis- and trans-forms, as well as mixtures, are embraced within the scope of the invention.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

In the Compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched Compounds of Formula (I) can be prepared without undue experimentation by conventional techniques well known to those skilled in the art or by processes analogous to those described in the Schemes and Examples herein using appropriate isotopically-enriched reagents and/or intermediates. In one embodiment, a Compound of Formula (I) has one or more of its hydrogen atoms replaced with deuterium.

Polymorphic forms of the Silyl-Containing Heterocyclic Compounds, and of the salts, solvates, hydrates, esters and prodrugs of the Silyl-Containing Heterocyclic Compounds, are intended to be included in the present invention.

The following abbreviations are used below and have the following meanings: Ac is acyl; AcOH is acetic acid; BF₃.OEt₂ is boron trifluoride etherate; BOC or Boc is tert-butyloxycarbonyl; Boc₂O is Boc anhydride; Boc-Pro-OH is Boc protected proline; L-Boc-Val-OH is Boc protected L-valine; n-BuLi is n-butyllithium; dba is dibenzylideneacetone; DCM is dichloromethane; DIEA is diisopropylethylamine; DME is dimethoxyethane; DMF is N,N-dimethylformamide; dppf is diphenylphosphinoferrocene; DMSO is dimethylsulfoxide; EtOAc is ethyl acetate; Et₂O is diethyl ether; Et₃N is triethylamine; HATU is O-(7-azabenzotriazol-1-yl)-N,N,N′,N′-tetramethyluronium hexafluorophosphate; Hg(OAc)₂ is mercuric acetate; HPLC is high performance liquid chromatography; HRMS is high resolution mass spectrometry; KOAc is potassium acetate; Lawesson's Reagent is 2,4-Bis(4-methoxyphenyl)-1,3-dithiadiphosphetane-2,4-disulfide; LCMS is liquid chromatography/mass spectrometry; LRMS is low resolution mass spectrometry; mCPBA is m-chloroperbenzoic acid; MeOH is methanol; MTBE is tert-butylmethyl ether; NBS is N-bromosuccinimide; NH₄OAc is ammonium acetate; Pd(PPh₃)₄ is tetrakis(triphenylphosphine) palladium(0); PdCl₂(dppf)₂ is [1,1′-Bis(diphenylphosphino)ferrocene]dichloro palladium(II); PdCl₂(dppf)₂.CH₂Cl₂ is [1,1′-Bis(diphenylphosphino)ferrocene]dichloro palladium(II) complex with dichloromethane; pinacol₂B₂ is bis(pinacolato)diboron; PPTS is pyridinium p-toluene sulfonate; RPLC is reverse-phase liquid chromatography; SEM-Cl is 2-(trimethylsilyl)ethoxymethyl chloride; TBAF is tetrabutylammonium fluoride; TBAI is tetrabutylammonium iodide; TBDMSCl is tert-butyldimethylsilyl chloride; TFA is trifluoroacetic acid; THF is tetrahydrofuran; TLC is thin-layer chromatography; XPhos is 2-dicyclohexylphosphino-2′,4′,6′-triisopropylbiphenyl; and Z-Pro-OH is N-Benzyloxycarbonyl-L-proline.

### The Compounds of Formula (I)

The present invention provides Silyl-Containing Heterocyclic Compounds of Formula (I):

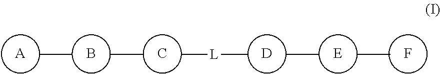

and pharmaceutically acceptable salts thereof, wherein A, B, C, D, E, F and L are defined above for the Compounds of Formula (I).

In one embodiment, for the Compounds of Formula (I), A and F are each independently selected from:

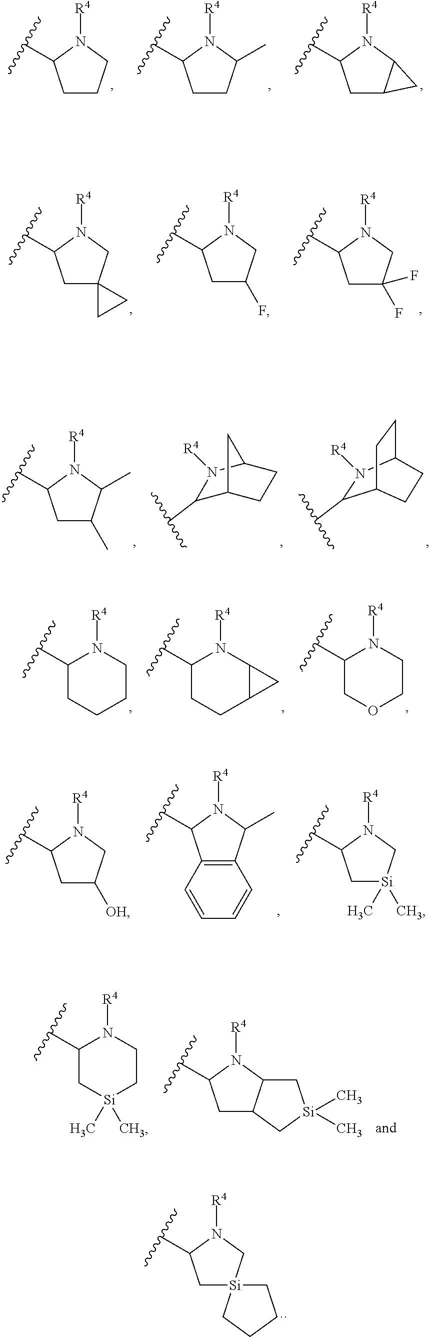

In another embodiment, for the Compounds of Formula (I), A and F are each independently selected from:

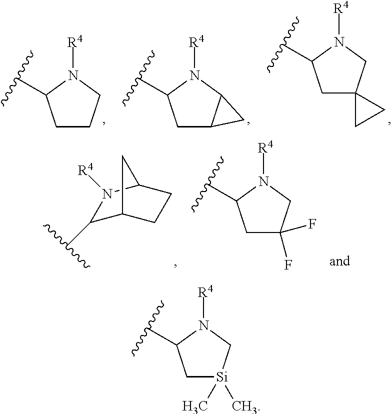

In one embodiment, for the Compounds of Formula (I), B and E are each independently:

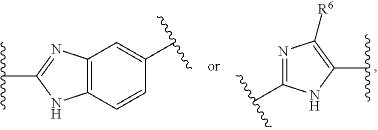

wherein each occurrence of R⁶ is independently H, F, Cl or cyclopropyl.

In another embodiment, for the Compounds of Formula (I), one of B and E is

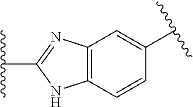 [SMILES: *c1ccc2[nH]c(*)nc2c1]

and the other of B and E is:

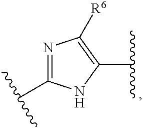

wherein R⁶ is H, F, Cl or cyclopropyl.

In one embodiment, for the Compounds of Formula (I), B and E are each independently:

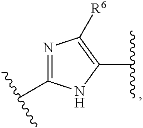

wherein each occurrence of R⁶ is independently H, F, Cl or cyclopropyl.

In one embodiment, for the Compounds of Formula (I), C is a bond or phenylene.

In another embodiment, for the Compounds of Formula (I), C is a bond.

In another embodiment, for the Compounds of Formula (I), C is a 5 or 6-membered monocyclic heteroarylene.

In still another embodiment, for the Compounds of Formula (I), C is a 9 or 10-membered bicyclic heteroarylene.

In another embodiment, for the Compounds of Formula (I), C is phenylene.

In another embodiment, for the Compounds of Formula (I), C is naphthylene.

In yet another embodiment, for the Compounds of Formula (I), C is

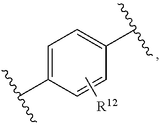

wherein R¹² is an optional ring substituent selected from F, —OCH₃, pyridyl, —OCH₂CH₂OH, —OCH₂CH₂OC(O)CH₃, cyclopropyl and thiophenyl

In yet another embodiment, for the Compounds of Formula (I), C is:

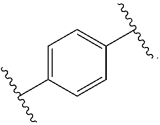 [SMILES: *c1ccc(*)cc1]

In one embodiment, for the Compounds of Formula (I), D is a 5 or 6-membered monocyclic heteroarylene.

In another embodiment, for the Compounds of Formula (I), D is a 9 or 10-membered bicyclic heteroarylene.

In another embodiment, for the Compounds of Formula (I), D is phenylene.

In still another embodiment, for the Compounds of Formula (I), D is naphthylene.

In another embodiment, for the Compounds of Formula (I), D is

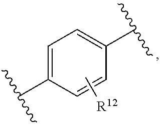

wherein R¹² is an optional ring substituent selected from F, -ODH₃, pyridyl, -ODH₂DH₂OH, -ODH₂DH₂OD(O)DH₃, cyclopropyl and thiophenyl

In yet another embodiment, for the Compounds of Formula (I), D is:

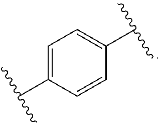 [SMILES: *c1ccc(*)cc1]

In another embodiment, for the Compounds of Formula (I), D is:

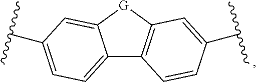

wherein G is —CF₂— or —Si(CH₃)₂—.

In one embodiment, for the Compounds of Formula (I), L is a bond.

In another embodiment, for the Compounds of Formula (I), L is —C≡C—.

In one embodiment, for the Compounds of Formula (I), C is a bond and D is 13 to 14-membered tricyclic heteroarylene, which can be optionally substituted as set forth above for the Compounds of Formula (I).

In one embodiment, for the Compounds of Formula (I), C and D are each:

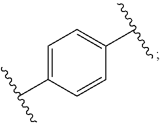 [SMILES: *c1ccc(*)cc1]

and
B and E are each independently:

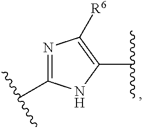

wherein each occurrence of R⁶ is independently H, F or Cl.

In another embodiment, for the Compounds of Formula (I), C and D are each:

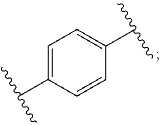 [SMILES: *c1ccc(*)cc1]

B and E are each independently:

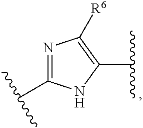

wherein each occurrence of R⁶ is independently H, F or Cl; and
L is a bond.

In one embodiment, for the Compounds of Formula (I), each occurrence of each occurrence of R⁴ is independently:

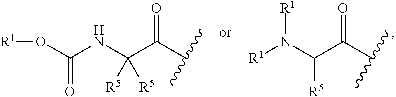

wherein each occurrence of R¹ is C₁-C₆ alkyl, and R⁵ is selected from C₁-C₆ alkyl, —CH₂—S—(C₁-C₆ alkyl), benzyl, —(CH₂)ₙ-aryl, —CH₂-heteroaryl, —(CH₂)ₙ-(3- to 7-membered cycloalkyl) and 4- to 7-membered heterocycloalkyl, wherein said C₁-C₆ alkyl group can be optionally substituted with —OH or —O—(C₁-C₆ alkyl), or two R⁵ groups that are attached to a common carbon atom, and the common carbon atom to which they are attached, can combine to form a 3 to 7-membered cycloalkyl group.

In another embodiment, for the Compounds of Formula (I), each occurrence of R⁴ is independently:

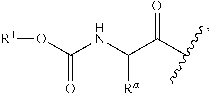

wherein R^{a} is selected from methyl, ethyl, propyl, isopropyl, cyclopropyl, tetrahydropyranyl, benzyl and phenyl and R¹ is selected from methyl, ethyl and isopropyl.

In another embodiment, for the Compounds of Formula (I), each occurrence of R⁴ is independently:

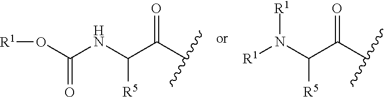

R¹ is methyl and R⁵ is selected from methyl, isopropyl, isobutyl, phenyl, cyclopropyl, cyclopentyl, cyclohexyl, —CH(OCH₃)CH₃, —CH(OH)CH₂CH₃, —CH(OH)CH(CH₃)₂, tetrahydropyranyl, oxepanyl, —CH₂-cyclopropyl, —CH₂—S—CH₃, and —CH₂-indolyl.

In another embodiment, for the Compounds of Formula (I), each occurrence of R⁴ is independently:

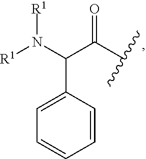

wherein each occurrence R¹ is methyl or ethyl.

In still another embodiment, for the Compounds of Formula (I), each occurrence of R⁴ is independently selected from:

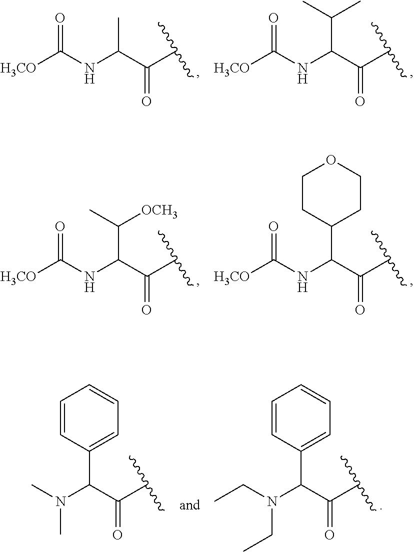

In another embodiment, for the Compounds of Formula (I), each occurrence of R⁴ is:

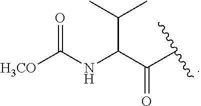 [SMILES: *C(=O)C(NC(=O)OC)C(C)C]

In one embodiment, for the Compounds of Formula (I), A and F are each independently selected from:

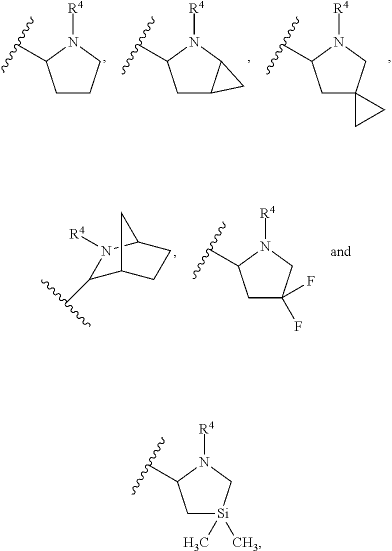

and each occurrence of R⁴ is independently selected from:

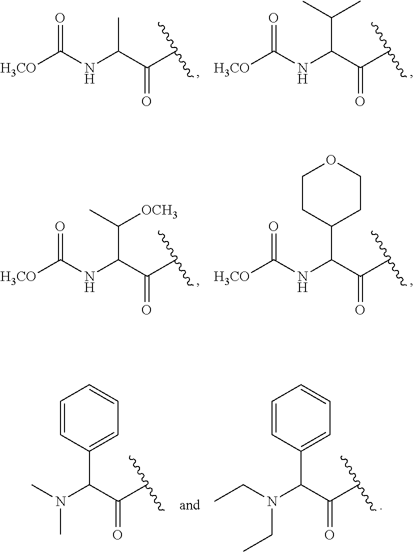

In one embodiment, for the Compounds of Formula (I), A and F are each independently selected from:

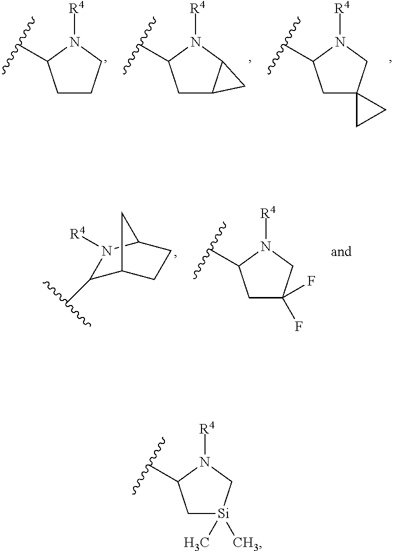

and each occurrence of R⁴ is:

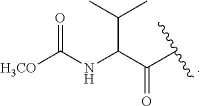 [SMILES: *C(=O)C(NC(=O)OC)C(C)C]

In one embodiment, the Compounds of Formula (I) have the formula (Ia):

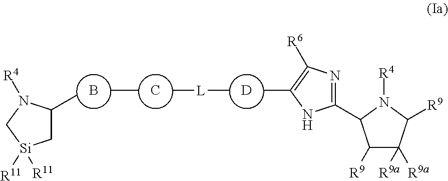

or a pharmaceutically acceptable salt thereof, wherein

B is imidazolyl or benzimidazolyl, each of which can be optionally substituted on a ring carbon atom with R⁶;

C is a bond or phenylene;

D is phenylene or 13 to 14-membered tricyclic heteroarylene, wherein said 13 to 14-membered tricyclic heteroarylene group can be optionally substituted on a ring carbon atom, ring nitrogen atom or ring silyl atom with up to 4 groups, each independently selected from C₁-C₆ alkyl and halo.

L is a bond or —C≡C—, such that when D is a 13 to 14-membered tricyclic heteroarylene group, then L and C are each a bond;

each occurrence of R¹ is C₁-C₆ alkyl;

each occurrence of R⁴ is independently:

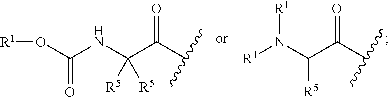

R⁵ is selected from C₁-C₆ alkyl, —CH₂—S—(C₁-C₆ alkyl), benzyl, —(CH₂)ₙ-aryl, —CH₂-heteroaryl, —(CH₂)ₙ-(3- to 7-membered cycloalkyl) and 4- to 7-membered heterocycloalkyl, wherein said C₁-C₆ alkyl group can be optionally substituted with —OH or —O—(C₁-C₆ alkyl), or two R⁵ groups that are attached to a common carbon atom, and the common carbon atom to which they are attached, can combine to form a 3 to 7-membered cycloalkyl group;

R⁶ is H, halo or 3 to 7-membered cycloalkyl;

each occurrence of R⁹ is: (i) H, or (ii) both R⁹ groups join to form a C₂-C₃ alkylene group, or (iii) one R⁹ group and one R^{9a} group and the ring carbon atoms to which they are attached join to form a 3 to 7-membered cycloalkyl group; and

each occurrence of R^{9a} is independently H or halo, or both R^{9a} groups and the common carbon atom to which they are attached, join to form a 3 to 7-membered cycloalkyl group.

In one embodiment, the Compounds of Formula (I) have the formula (Ib):

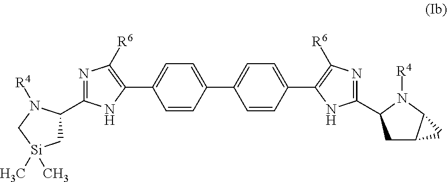

or a pharmaceutically acceptable salt thereof,
wherein:

each occurrence of R¹ is independently C₁-C₆ alkyl;

each occurrence of R⁴ is independently —C(O)CH(R⁷)C(O)OR¹ or —C(O)CH(R⁷)N(R¹)₂;

each occurrence of R⁷ is independently C₁-C₆ alkyl, phenyl or 4 to 7-membered heterocycloalkyl; and

each occurrence of R⁶ is H or halo.

In one embodiment, for the compounds of formula (Ib), each occurrence of R⁶ is H.

In another embodiment, for the compounds of formula (Ib), each occurrence of R⁶ is Cl.

In one embodiment, for the compounds of formula (Ib), each occurrence of R⁴ is —C(O)CH(R⁷)C(O)OR¹ and each occurrence of R⁷ is independently C₁-C₆ alkyl or 4 to 7-membered heterocycloalkyl.

In one embodiment, for the compounds of formula (Ib), each occurrence of R⁴ is —C(O)CH(R⁷)N(R¹)₂, and each occurrence of R⁷ is phenyl.

In one embodiment, for the compounds of formula (Ib), each occurrence of R⁴ is:

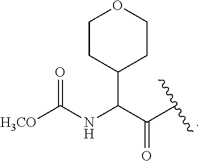 [SMILES: *C(=O)C(NC(=O)OC)C1CCOCC1]

In another embodiment, for the compounds of formula (Ib), each occurrence of R⁴ is:

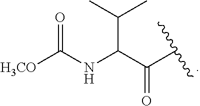 [SMILES: *C(=O)C(NC(=O)OC)C(C)C]

In one embodiment, for the compounds of formula (Ib), each occurrence of R⁴ is:

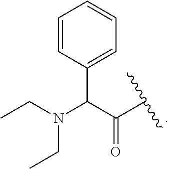 [SMILES: *C(=O)C(c1ccccc1)N(CC)CC]

In one embodiment, variables A, B, C, D, E, F and L of the Compounds of Formula (I) are selected independently from each other.

In another embodiment, a Compound of Formula (I) is in substantially purified form.
Other embodiments of the present invention include the following:
(a) A pharmaceutical composition comprising an effective amount of a Compound of Formula (I) or a pharmaceutically acceptable salt thereof, and a pharmaceutically acceptable carrier.
(b) The pharmaceutical composition of (a), further comprising a second therapeutic agent selected from the group consisting of HCV antiviral agents, immunomodulators, and anti-infective agents.
(c) The pharmaceutical composition of (b), wherein the HCV antiviral agent is an antiviral selected from the group consisting of HCV protease inhibitors and HCV NS5B polymerase inhibitors.
(d) A pharmaceutical combination that is (i) a Compound of Formula (I) and (ii) a second therapeutic agent selected from the group consisting of HCV antiviral agents, immunomodulators, and anti-infective agents; wherein the Compound of Formula (I) and the second therapeutic agent are each employed in an amount that renders the combination effective for inhibiting HCV replication, or for treating HCV infection and/or reducing the likelihood or severity of symptoms of HCV infection.
(e) The combination of (d), wherein the HCV antiviral agent is an antiviral selected from the group consisting of HCV protease inhibitors and HCV NS5B polymerase inhibitors.
(f) A method of inhibiting HCV replication in a subject in need thereof which comprises administering to the subject an effective amount of a Compound of Formula (I).
(g) A method of treating HCV infection and/or reducing the likelihood or severity of symptoms of HCV infection in a subject in need thereof which comprises administering to the subject an effective amount of a Compound of Formula (I).
(h) The method of (g), wherein the Compound of Formula (I) is administered in combination with an effective amount of at least one second therapeutic agent selected from the group consisting of HCV antiviral agents, immunomodulators, and anti-infective agents.
(i) The method of (h), wherein the HCV antiviral agent is an antiviral selected from the group consisting of HCV protease inhibitors and HCV NS5B polymerase inhibitors.
(j) A method of inhibiting HCV replication in a subject in need thereof which comprises administering to the subject the pharmaceutical composition of (a), (b) or (c) or the combination of (d) or (e).
(k) A method of treating HCV infection and/or reducing the likelihood or severity of symptoms of HCV infection in a subject in need thereof which comprises administering to the subject the pharmaceutical composition of (a), (b) or (c) or the combination of (d) or (e).

The present invention also includes a compound of the present invention for use (i) in, (ii) as a medicament for, or (iii) in the preparation of a medicament for: (a) inhibiting HCV replication or (b) treating HCV infection and/or reducing the likelihood or severity of symptoms of HCV infection. In these uses, the compounds of the present invention can optionally be employed in combination with one or more second therapeutic agents selected from HCV antiviral agents, anti-infective agents, and immunomodulators.

Additional embodiments of the invention include the pharmaceutical compositions, combinations and methods set forth in (a)-(k) above and the uses set forth in the preceding paragraph, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate.

It is further to be understood that the embodiments of compositions and methods provided as (a) through (k) above are understood to include all embodiments of the compounds, including such embodiments as result from combinations of embodiments.

Non-limiting examples of the Compounds of Formula (I) include compounds 1-237, as set forth in the Examples below, and pharmaceutically acceptable salts thereof.

### Methods For Making the Compounds of Formula (I)

The Compounds of Formula (I) may be prepared from known or readily prepared starting materials, following methods known to one skilled in the art of organic synthesis. Methods useful for making the Compounds of Formula (I) are set forth in the Examples below and generalized in Schemes 1-12 below. Alternative synthetic pathways and analogous structures will be apparent to those skilled in the art of organic synthesis. All stereoisomers and tautomeric forms of the compounds are contemplated.

Some commercially available starting materials and intermediates used for the synthesis of the Compounds of Formula (I) are available which contain intact fused tricyclic tricyclic ring systems. These starting materials and intermediates are available from commercial suppliers such as Sigma-Aldrich (St. Louis, Mo.) and Acros Organics Co. (Fair Lawn, N.J.). Such starting materials and intermediates compounds are used as received. When such fused tricyclic moieties are not commercially available, they can be prepared using methods well-known to those skilled in the art of organic synthesis. Such synthetic methods include, but are not limited to, those described in Kricka et al., J. Chem. Soc. Perkin Trans I, 859-863 (1973); Kricka et al., Chem. Rew., 74, 101-123, (1974); Kurfuerst et al., Coll. Czech. Chem. Comm., 54, 1705-1715, (1989); Saroja et al., J. Org. Chem. 69, 987-990, (2004); Fanta et al., Synth. 9-21, (1974), U.S. Patent Publication No. US2005038037; and International Publication No. WO2004039859.

Scheme 1 shows a method useful for making the phenyl imidazole compounds of formula A7 and A8, which are useful intermediates for making the Compounds of Formula (I).

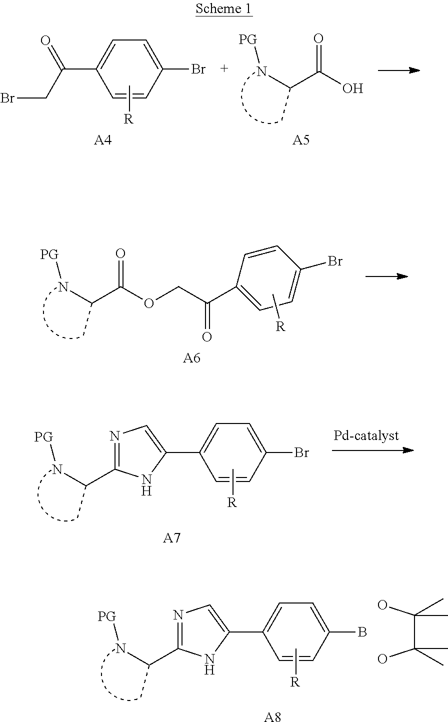

Coupling of bromoanoline A4 to a cyclic or acyclic N-protected α-amino acid A5 gives an amide of formula A6, which upon heating in ammonium acetate will cyclize to provide bromophenylimidazole A7. The bromide could be converted to a boronate A8 with a palladium catalyst.

Scheme 2 shows a method useful for making the halogenated compounds of formula B2, which are useful intermediates for making the Compounds of Formula (I).

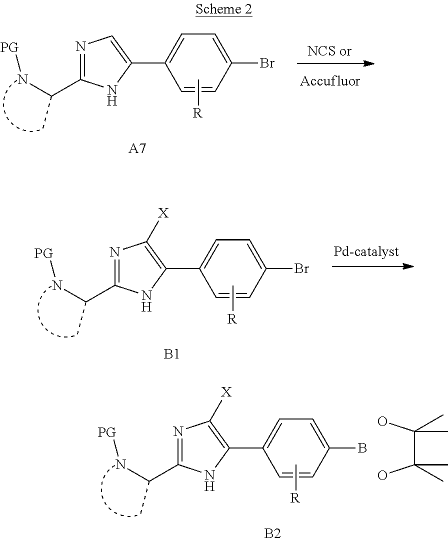

Treatment of A7 with a halogenating agent such as NCS or Accufluor should afford halogenated analog B1. The bromide B1 could be converted to a boronate B2 with a palladium catalyst.

Scheme 3 shows a method useful for making the boronic acid compounds of formula C4, which are useful intermediates for making the Compounds of Formula (I), where in “C” is a monocyclic 5 to 6-membered heteroaryl (examples: thiophene or pyridine).

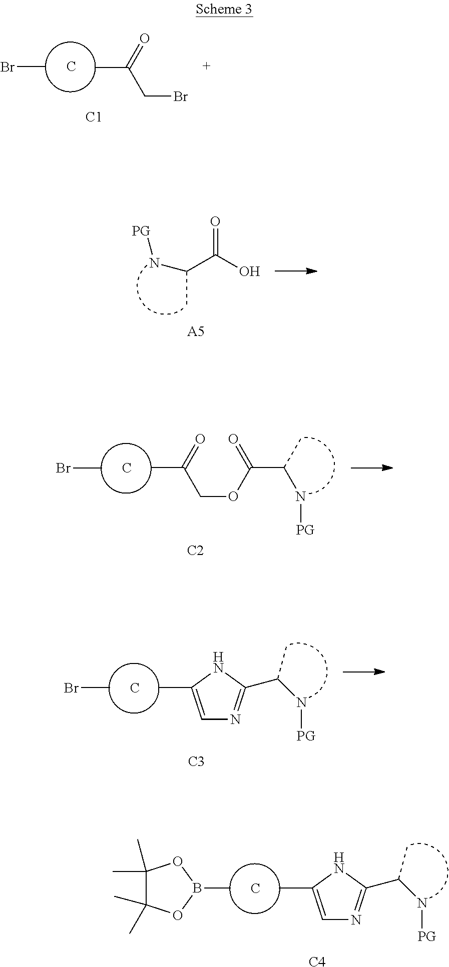

The Suzuki coupling partner C3 or C4 can be prepared from commercially available heteroaryl bromoacetyl compound of formula C1 (Scheme 3). When treated with an N-protected amino acid (PG-AA-OH) in the presence of an amine base, e.g., DIEA, a ketoester C2 is formed. If heated together with ammonium acetate, the ketoester is converted to the desired imidazole derivative C3. The bromide can then be converted to a boronate C4 with a palladium catalyzed reaction.

Scheme 4 shows methods useful for making the compounds of formula C1 and C3, which are useful intermediates for making the Compounds of Formula (I), wherein variable C is other than a bond and B is an imidazole ring.

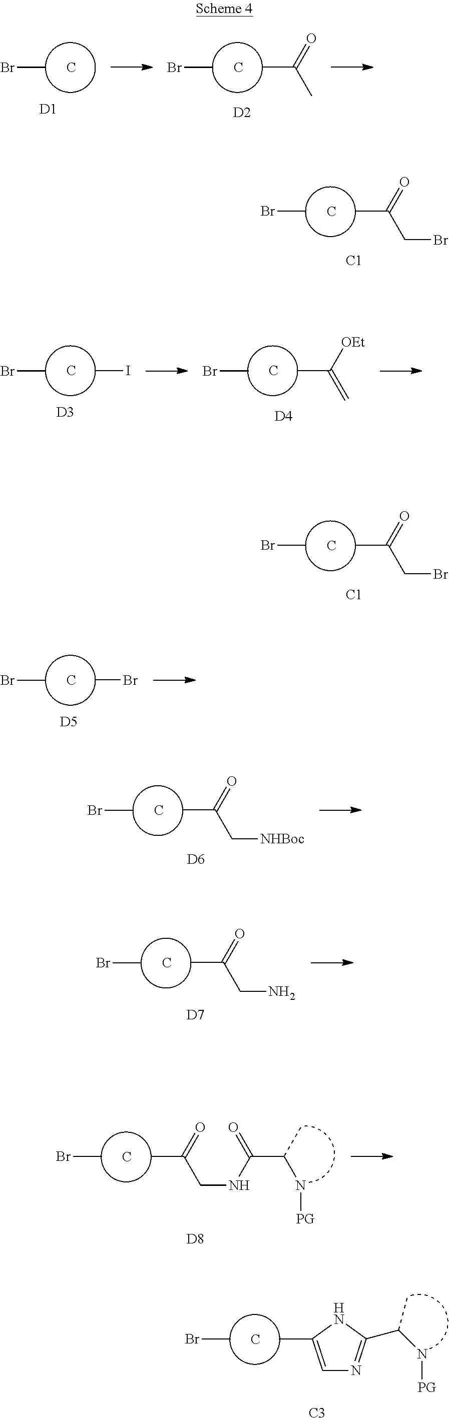

When heteroaryl bromoacetyl C1 is not commercially available, it can be prepared by performing Friedel-Crafts acylation on a heteroaryl bromide of formula D1 using well-known methods, (e.g., those described in Kricka et al., J. Chem. Soc. Perkin Trans I, 859-863 (1973), and Kricka et al., Chem. Rew., 74, 101-123, (1974)) to provide the acylated products of formula D2. A compound of formula D2 can then be brominated using bromine, for example, to provide the compounds of formula C1.

On the other hand, bromo-iodo substituted heteroaromatic rings D3 can undergo a Stille coupling with (α-ethoxyvinyl)tributylstannane in the presence of a palladium catalyst using the methods including, but not limited to those described in Choshi et al., J. Org. Chem., 62:2535-2543 (1997), and Scott et al., J. Am. Chem. Soc., 106:4630 (1984)), to provide the ethyl-vinyl ether intermediate D4. Treating D4 with N-bromosuccimide gives the desired bromoacetyl intermediate C1, which can then be elaborated to advanced intermediates C3 or C4 for Suzuki coupling.

Alternatively, a heteroaromatic dibromide of formula D5 can be lithiated using n-butyl lithium and then quenched with N-Boc-glycine Weinreb amide to provide a Boc-protected β-keto amino compound of formula D6. Removal of the Boc group using TFA, for example, provides an amine compound of formula D7, which can then be coupled with an N-protected amino acid using typical amide bond forming reagents such as HATU to provide a ketoamide compound of formula D8. Upon heated in the presence of ammonium acetate, compound D8 can be cyclized to the imidazole analog of formula C3.

Scheme 5 shows a method useful for making the boronic acid compounds of formula E4, which are useful intermediates for making the Compounds of Formula (I).

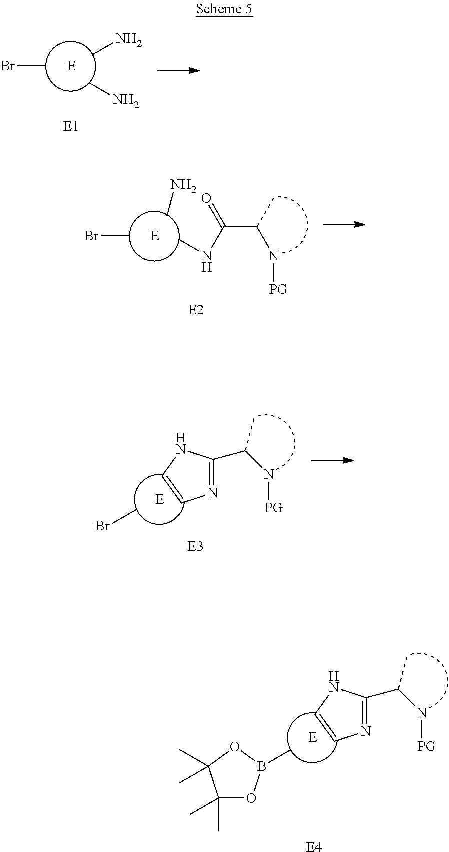

A heteroaromatic diamine E1 could be converted to a bicyclic imidazole E3 using the two step coupling-cyclization procedure described, for example, in Scheme 3. The corresponding boronate E4 can then easily be obtained from bromide E3 via well-known chemistry. Both E3 and E4 can be used as intermediate coupling partners in a Suzuki coupling process to provide the Compound of Formula (I).

Scheme 6 shows methods useful for making the Compounds of Formula (I) via a Suzuki Coupling process.

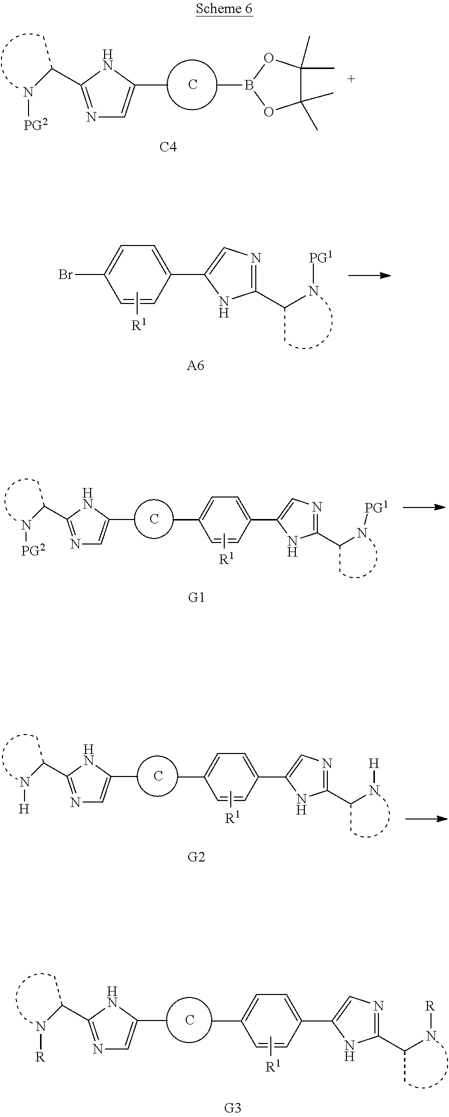

A Suzuki coupling between protected imidazole boronate C4 (or boronic acid, not shown) and the phenyl imidazole bromide A6 using, for example, the methods described in Angew Chem. Int. Ed. Engl., 40, 4544 (2001) provide the compounds of formula G1. Compounds of formula G1 can then be used to provide compounds of formula G2 by removal of the nitrogen protecting groups of G1. An appropriate cap of group R can be added to the deprotected amino groups of G2 using reactions including, but not limited to acylation (with an acyl chloride or amino acid coupling reagent such as HATU or HOBt/EDCI), sulfonylation (with a sulfonyl chloride) or alkylation (with alkyl halide or reductive amination) to provide the desired Compounds of Formula (I).

Scheme 7 shows methods useful for making the Compounds of Formula (I) via a Suzuki Coupling process where X and/or Y can be halogens or H.

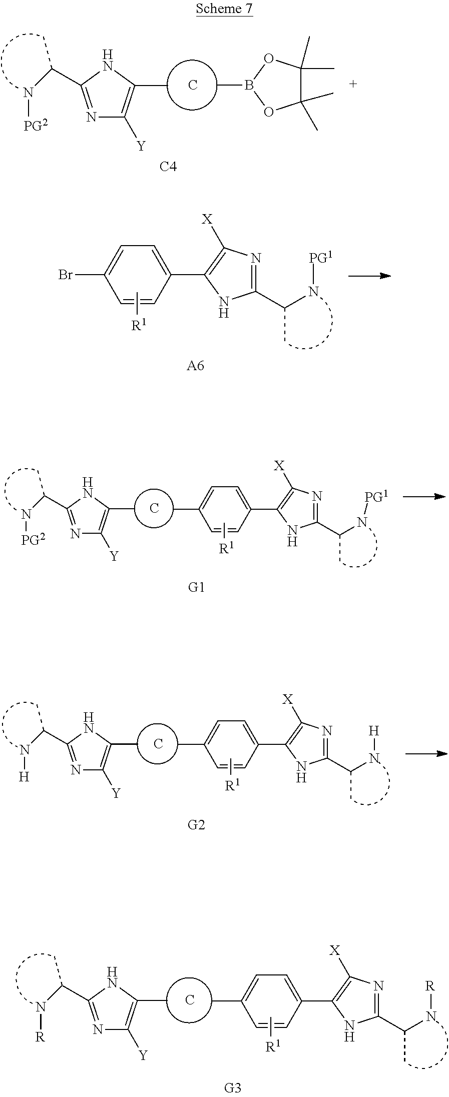

A Suzuki coupling between protected imidazole bOronate C4 (or boronic acid, not shown) and the phenyl imidazole bromide A6 using, for example, the methods described in Angew Chem. Int. Ed. Engl., 40, 4544 (2001) provide the compounds of formula G1. Compounds of formula G1 can then be used to provide compounds of formula G2 by removal of the nitrogen protecting groups of G1. An appropriate cap of group R can be added to the deprotected amino groups of G2 using reactions including, but not limited to acylation (with an acyl chloride or amino acid coupling reagent such as HATU or HOBt/EDCI), sulfonylation (with a sulfonyl chloride) or alkylation (with alkyl halide or reductive amination) to provide the desired Compounds of Formula (I).

Scheme 8 shows methods useful for making the Compounds of Formula (I) via a halogenation process.

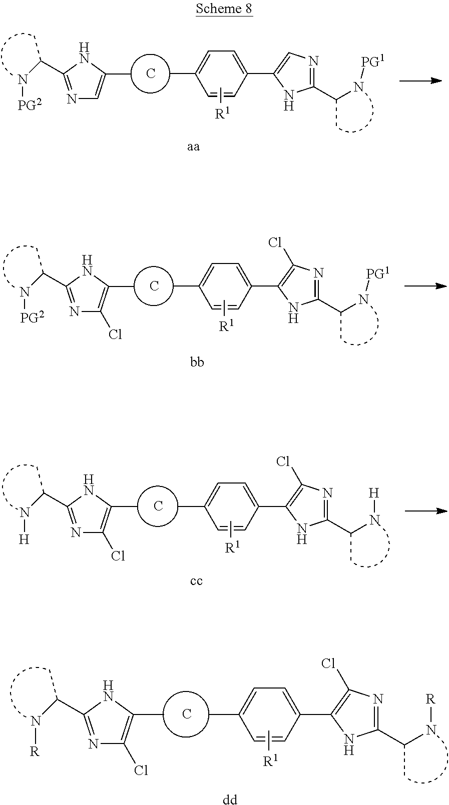

Halogenation of compounds of formula aa can then be used to provide compounds of formula bb. Removal of the nitrogen protecting groups of bb can afford compounds of formula cc. The appropriate cap of group R can be added to the deprotected amino groups of cc using reactions including, but not limited to acylation (with an acyl chloride or amino acid coupling reagent such as HATU or HOBt/EDCI), sulfonylation (with a sulfonyl chloride) or alkylation (with alkyl halide or reductive amination) to provide the desired Compounds of Formula (I).

Scheme 9 shows methods useful for making the Compounds of Formula (I) via a Sonongashira/Suzuki Coupling process.

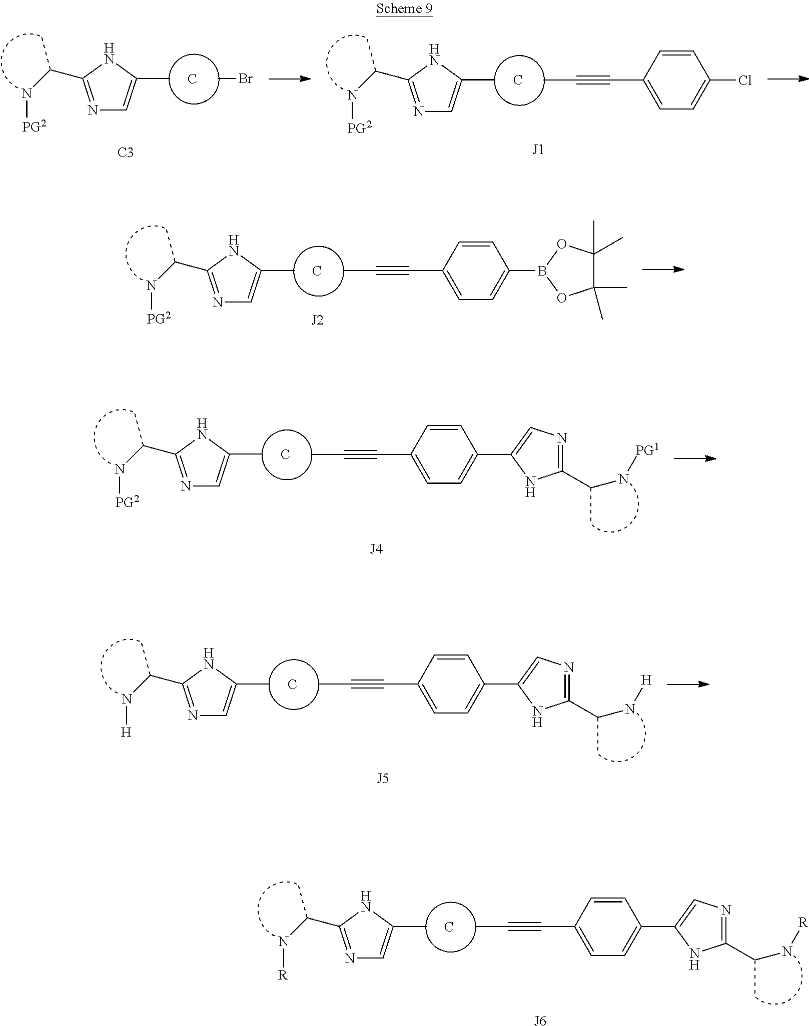

Similarly, a bromide of formula C3 and alkyne can be reaction under Sonogashira conditions, to provide coupled intermediates of formula J1. The compounds of formula J1 can then be further elaborated using, for example, the methods described in Scheme 6 above, to provide the Compounds of Formula (I), wherein C is a bond and B is a bicyclic heteroarylene group.

Scheme 10 shows methods useful for making the Compounds of Formula (I) via a Suzuki Coupling process.

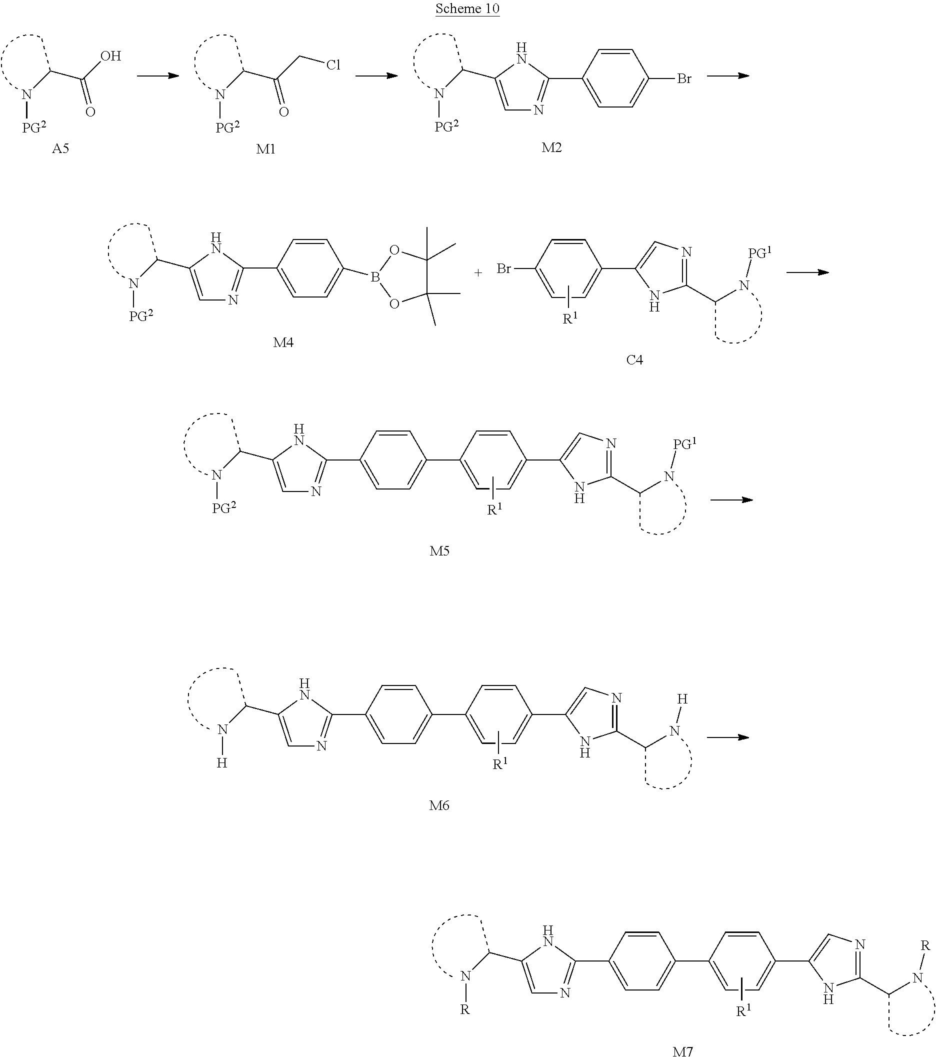

The carboxylic acid A5 can be homologated to provide products of formula M1. Treatment with substituted phenylamidine salts under basic conditions should provide products of formula M3. conditions similar to the methods described above to provide products of formula I1, which can be transformed to the final targets of formula I3, using methods well-known to those skilled in the art of organic synthesis, including those described in Scheme 6 above.

Scheme 11 shows methods useful for making the Compounds of Formula (I) via a Suzuki Coupling process.

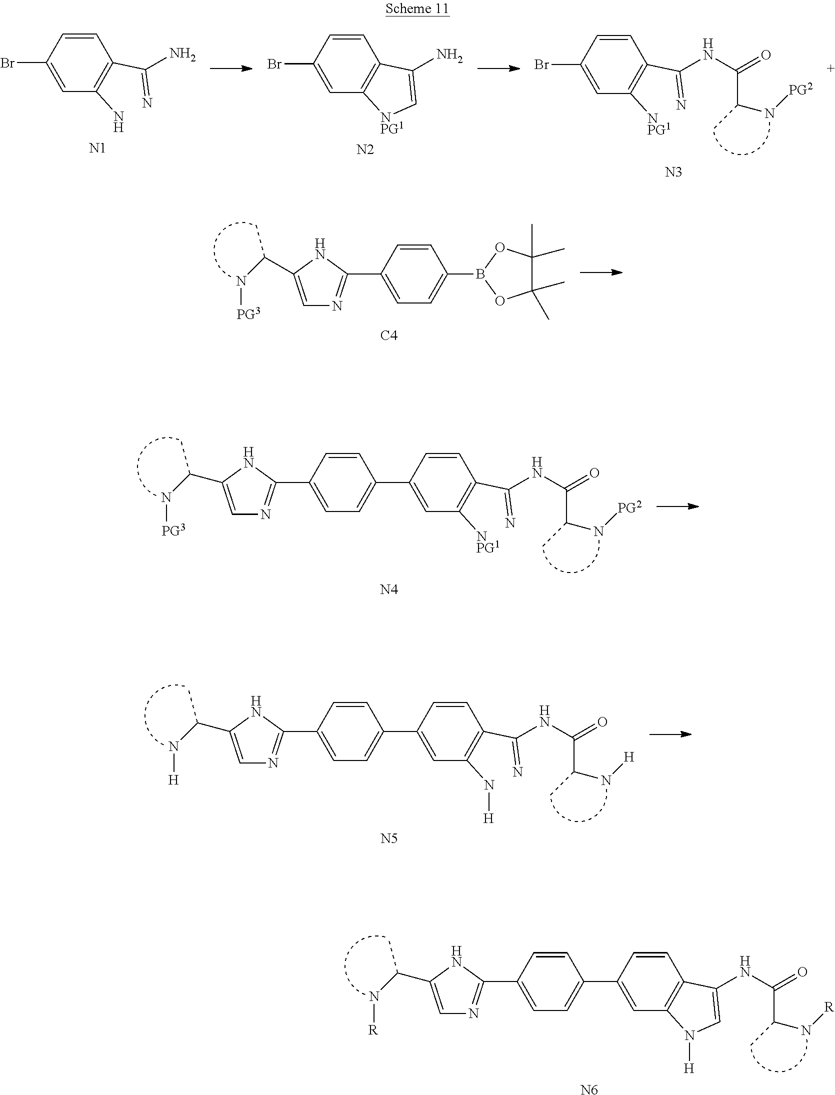

The bromoindazole N1 can be protected at N1 followed by aceylation at N3 to provide products of the formula N3. Treatment under Suzuki conditions with products of the formula C4 should provide compounds of the type N4 under basic conditions should provide products of formula M3. conditions similar to the methods described above to provide products of formula I1, which can be transformed to the final targets of formula I3, using methods well-known to those skilled in the art of organic synthesis, including those described in Scheme 6 above.

Scheme 12 shows alternative methods useful for making the Compounds of Formula (I) via a Suzuki Coupling process.

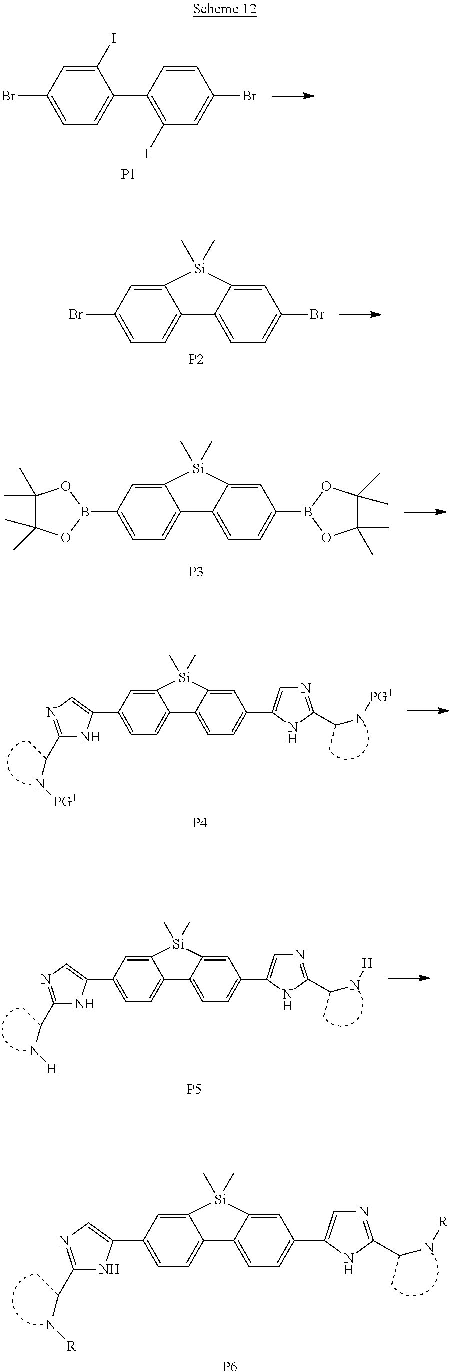

The dibromo phenyl adduct P1 (J. Am. Chem. Soc. 2005, 127, 7662) can be lithiated and trapped with dichlorodimethylsilane to provide products of formulas P2. The bromide P2 could be converted to a boronate P3 with a palladium catalyst. Treatment under Suzuki conditions under basic conditions should provide products of formula P4. Removal of the nitrogen protecting groups of P4 can afford compounds of formula P5. The appropriate cap of group R can be added to the deprotected amino groups of P5 using reactions including, but not limited to acylation (with an acyl chloride or amino acid coupling reagent such as HATU or HOBt/EDCI), sulfonylation (with a sulfonyl chloride) or alkylation (with alkyl halide or reductive amination) to provide the desired Compounds of Formula (I).

In some of the Silyl-Containing Heterocyclic Compounds contemplated in Schemes 1-12, the amino acids (such as, but not limited to proline, 4,4-difluoroproline, (S)-2-piperidine carboxylic acid, valine, alanine, norvaline, etc.) are incorporated as part of structures. Methods have been described in the general literature as well as in US Publication No. 2009/0068140 for the preparation of such amino acid-derived intermediates.

One skilled in the art of organic synthesis will also recognize that one route for the synthesis of the Compounds of Formula (I) may be more desirable depending on the choice of appendage substituents. Additionally, one skilled in the art will recognize that in some cases the order of reactions may differ from that presented herein to avoid functional group incompatibilities and can amend the synthetic route accordingly.

One skilled in the art of organic synthesis will recognize that the synthesis of the Compounds of Formula (I) may require the construction of an amide bond. Methods useful for making such amide bonds, include but are not limited to, the use of a reactive carboxy derivative (e.g., an acid halide, or ester at elevated temperatures) or the use of an acid with a coupling reagent (e.g., HOBt, EDCI, DCC, HATU, PyBrop) with an amine.

The preparation of various monocyclic and polycyclic heterocyclic ring systems contemplated in this invention have been described in the literature and in compendia such as “Comprehensive Heterocyclic Chemistry” editions I, II and III, published by Elsevier and edited by A. R. Katritzky & R J K Taylor. Manipulation of the required substitution patterns have also been described in the available chemical literature as summarized in compendia such as “Comprehensive Organic Chemistry” published by Elsevier and edited by D H R. Barton and W. D. Ollis; “Comprehensive Organic Functional Group Transformations” edited by edited by A. R. Katritzky & R J K Taylor and “Comprehensive Organic Transformation” published by Wily-CVH and edited by R. C. Larock.

The starting materials used and the intermediates prepared using the methods set forth in the Schemes above may be isolated and purified if desired using conventional techniques, including but not limited to filtration, distillation, crystallization, chromatography and alike. Such materials can be characterized using conventional means, including physical constants and spectral data.

### Uses of the Silyl-Containing Heterocyclic Compounds

The Silyl-Containing Heterocyclic Compounds are useful in human and veterinary medicine for treating or preventing a viral infection in a patient. In one embodiment, the Silyl-Containing Heterocyclic Compounds can be inhibitors of viral replication. In another embodiment, the Silyl-Containing Heterocyclic Compounds can be inhibitors of HCV replication. Accordingly, the Silyl-Containing Heterocyclic Compounds are useful for treating viral infections, such as HCV. In accordance with the invention, the Silyl-Containing Heterocyclic Compounds can be administered to a patient in need of treatment or prevention of a viral infection.

Accordingly, in one embodiment, the invention provides methods for treating a viral infection in a patient comprising administering to the patient an effective amount of at least one Silyl-Containing Heterocyclic Compound or a pharmaceutically acceptable salt thereof.

### Treatment or Prevention of a Flaviviridae Virus

The Silyl-Containing Heterocyclic Compounds can be useful for treating or preventing a viral infection caused by the Flaviviridae family of viruses.

Examples of Flaviviridae infections that can be treated or prevented using the present methods include but are not limited to, dengue fever, Japanese encephalitis, Kyasanur Forest disease, Murray Valley encephalitis, St. Louis encephalitis, Tick-borne encephalitis, West Nile encephalitis, yellow fever and Hepatitis C Virus (HCV) infection.

In one embodiment, the Flaviviridae infection being treated is hepatitis C virus infection.

### Treatment or Prevention of HCV Infection

The Silyl-Containing Heterocyclic Compounds are useful in the inhibition of HCV (e.g., HCV NS5A), the treatment of HCV infection and/or reduction of the likelihood or severity of symptoms of HCV infection and the inhibition of HCV viral replication and/or HCV viral production in a cell-based system. For example, the Silyl-Containing Heterocyclic Compounds are useful in treating infection by HCV after suspected past exposure to HCV by such means as blood transfusion, exchange of body fluids, bites, accidental needle stick, or exposure to patient blood during surgery or other medical procedures.

In one embodiment, the hepatitis C infection is acute hepatitis C. In another embodiment, the hepatitis C infection is chronic hepatitis C.

Accordingly, in one embodiment, the invention provides methods for treating HCV infection in a patient, the methods comprising administering to the patient an effective amount of at least one Silyl-Containing Heterocyclic Compound or a pharmaceutically acceptable salt thereof. In a specific embodiment, the amount administered is effective to treat or prevent infection by HCV in the patient. In another specific embodiment, the amount administered is effective to inhibit HCV viral replication and/or viral production in the patient.

The Silyl-Containing Heterocyclic Compounds are also useful in the preparation and execution of screening assays for antiviral compounds. For example the Silyl-Containing Heterocyclic Compounds are useful for identifying resistant HCV replicon cell lines harboring mutations within NS5A, which are excellent screening tools for more powerful antiviral compounds. Furthermore, the Silyl-Containing Heterocyclic Compounds are useful in establishing or determining the binding site of other antivirals to the HCV replicase.

The compositions and combinations of the present invention can be useful for treating a patient suffering from infection related to any HCV genotype. HCV types and subtypes may differ in their antigenicity, level of viremia, severity of disease produced, and response to interferon therapy as described in Holland et al., Pathology, 30(2):192-195 (1998). The nomenclature set forth in Simmonds et al., J Gen Virol, 74(Pt11):2391-2399 (1993) is widely used and classifies isolates into six major genotypes, 1 through 6, with two or more related subtypes, e.g., 1a and 1b. Additional genotypes 7-10 and 11 have been proposed, however the phylogenetic basis on which this classification is based has been questioned, and thus types 7, 8, 9 and 11 isolates have been reassigned as type 6, and type 10 isolates as type 3 (see Lamballerie et al., J Gen Virol, 78(Pt1):45-51 (1997)). The major genotypes have been defined as having sequence similarities of between 55 and 72% (mean 64.5%), and subtypes within types as having 75%-86% similarity (mean 80%) when sequenced in the NS-5 region (see Simmonds et al., J Gen Virol, 75(Pt 5):1053-1061 (1994)).

### Combination Therapy

In another embodiment, the present methods for treating or preventing HCV infection can further comprise the administration of one or more additional therapeutic agents which are not Silyl-Containing Heterocyclic Compounds.

In one embodiment, the additional therapeutic agent is an antiviral agent.

In another embodiment, the additional therapeutic agent is an immunomodulatory agent, such as an immunosuppressive agent.

Accordingly, in one embodiment, the present invention provides methods for treating a viral infection in a patient, the method comprising administering to the patient: (i) at least one Silyl-Containing Heterocyclic Compound, or a pharmaceutically acceptable salt thereof, and (ii) at least one additional therapeutic agent that is other than a Silyl-Containing Heterocyclic Compound, wherein the amounts administered are together effective to treat or prevent a viral infection.

When administering a combination therapy of the invention to a patient, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, a Silyl-Containing Heterocyclic Compound and an additional therapeutic agent may be present in fixed amounts (dosage amounts) in a single dosage unit (e.g., a capsule, a tablet and the like).

In one embodiment, the at least one Silyl-Containing Heterocyclic Compound is administered during a time when the additional therapeutic agent(s) exert their prophylactic or therapeutic effect, or vice versa.

In another embodiment, the at least one Silyl-Containing Heterocyclic Compound and the additional therapeutic agent(s) are administered in doses commonly employed when such agents are used as monotherapy for treating a viral infection.

In another embodiment, the at least one Silyl-Containing Heterocyclic Compound and the additional therapeutic agent(s) are administered in doses lower than the doses commonly employed when such agents are used as monotherapy for treating a viral infection.

In still another embodiment, the at least one Silyl-Containing Heterocyclic Compound and the additional therapeutic agent(s) act synergistically and are administered in doses lower than the doses commonly employed when such agents are used as monotherapy for treating a viral infection.

In one embodiment, the at least one Silyl-Containing Heterocyclic Compound and the additional therapeutic agent(s) are present in the same composition. In one embodiment, this composition is suitable for oral administration. In another embodiment, this composition is suitable for intravenous administration. In another embodiment, this composition is suitable for subcutaneous administration. In still another embodiment, this composition is suitable for parenteral administration.

Viral infections and virus-related disorders that can be treated or prevented using the combination therapy methods of the present invention include, but are not limited to, those listed above.

In one embodiment, the viral infection is HCV infection.

The at least one Silyl-Containing Heterocyclic Compound and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the administration of at least one Silyl-Containing Heterocyclic Compound and the additional therapeutic agent(s) may inhibit the resistance of a viral infection to these agents.

Non-limiting examples of additional therapeutic agents that may be useful in the present compositions and methods include an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, an antibody therapy (monoclonal or polyclonal), and any agent useful for treating an RNA-dependent polymerase-related disorder.

In one embodiment, the additional therapeutic agent is a viral protease inhibitor.

In another embodiment, the additional therapeutic agent is a viral replication inhibitor.

In another embodiment, the additional therapeutic agent is an HCV NS3 protease inhibitor.

In still another embodiment, the additional therapeutic agent is an HCV NS5B polymerase inhibitor.

In another embodiment, the additional therapeutic agent is a nucleoside inhibitor.

In another embodiment, the additional therapeutic agent is an interferon.

In yet another embodiment, the additional therapeutic agent is an HCV replicase inhibitor.

In another embodiment, the additional therapeutic agent is an antisense agent.

In another embodiment, the additional therapeutic agent is a therapeutic vaccine.

In a further embodiment, the additional therapeutic agent is a virion production inhibitor.

In another embodiment, the additional therapeutic agent is an antibody therapy.

In another embodiment, the additional therapeutic agent is an HCV NS2 inhibitor.

In still another embodiment, the additional therapeutic agent is an HCV NS4A inhibitor.

In another embodiment, the additional therapeutic agent is an HCV NS4B inhibitor.

In another embodiment, the additional therapeutic agent is an HCV NS5A inhibitor

In yet another embodiment, the additional therapeutic agent is an HCV NS3 helicase inhibitor.

In another embodiment, the additional therapeutic agent is an HCV IRES inhibitor.

In another embodiment, the additional therapeutic agent is an HCV p7 inhibitor.

In a further embodiment, the additional therapeutic agent is an HCV entry inhibitor.

In another embodiment, the additional therapeutic agent is an HCV assembly inhibitor.

In one embodiment, the additional therapeutic agents comprise a viral protease inhibitor and a viral polymerase inhibitor.

In still another embodiment, the additional therapeutic agents comprise a viral protease inhibitor and an immunomodulatory agent.

In yet another embodiment, the additional therapeutic agents comprise a polymerase inhibitor and an immunomodulatory agent.

In another embodiment, the additional therapeutic agents comprise a viral protease inhibitor and a nucleoside.

In another embodiment, the additional therapeutic agents comprise an immunomodulatory agent and a nucleoside.

In one embodiment, the additional therapeutic agents comprise an HCV protease inhibitor and an HCV polymerase inhibitor.

In another embodiment, the additional therapeutic agents comprise a nucleoside and an HCV NS5A inhibitor.

In another embodiment, the additional therapeutic agents comprise a viral protease inhibitor, an immunomodulatory agent and a nucleoside.

In a further embodiment, the additional therapeutic agents comprise a viral protease inhibitor, a viral polymerase inhibitor and an immunomodulatory agent.

In another embodiment, the additional therapeutic agent is ribavirin.

HCV polymerase inhibitors useful in the present compositions and methods include, but are not limited to, VP-19744 (Wyeth/ViroPharma), PSI-7851 (Pharmasset), RG7128 (Roche/Pharmasset), PSI-7977 (Pharmasset), PSI-938 (Pharmasset), PSI-879 (Pharmasset), PSI-661 (Pharmasset), PF-868554/filibuvir (Pfizer), VCH-759NX-759 (ViroChem Pharma/Vertex), HCV-371 (Wyeth/VirroPharma), HCV-796 (Wyeth/ViroPharma), IDX-184 (Idenix), IDX-375 (Idenix), NM-283 (Idenix/Novartis), GL-60667 (Genelabs), JTK-109 (Japan Tobacco), PSI-6130 (Pharmasset), R1479 (Roche), R-1626 (Roche), R-7128 (Roche), MK-0608 (Isis/Merck), INX-8014 (Inhibitex), INX-8018 (Inhibitex), INX-189 (Inhibitex), GS 9190 (Gilead), A-848837 (Abbott), ABT-333 (Abbott), ABT-072 (Abbott), A-837093 (Abbott), BI-207127 (Boehringer-Ingelheim), BILB-1941 (Boehringer-Ingelheim), MK-3281 (Merck), VCH-222/VX-222 (ViroChem/Vertex), VCH-916 (ViroChem), VCH-716(ViroChem), GSK-71185 (Glaxo SmithKline), ANA598 (Anadys), GSK-625433 (Glaxo SmithKline), XTL-2125 (XTL Biopharmaceuticals), and those disclosed in Ni et al., Current Opinion in Drug Discovery and Development, 7(4):446 (2004); Tan et al., Nature Reviews, 1:867 (2002); and Beaulieu et al., Current Opinion in Investigational Drugs, 5:838 (2004).

Other HCV polymerase inhibitors useful in the present compositions and methods include, but are not limited to, those disclosed in International Publication Nos. WO 08/082484, WO 08/082488, WO 08/083351, WO 08/136815, WO 09/032116, WO 09/032123, WO 09/032124 and WO 09/032125.

Interferons useful in the present compositions and methods include, but are not limited to, interferon alfa-2a, interferon alfa-2b, interferon alfacon-1 and PEG-interferon alpha conjugates. “PEG-interferon alpha conjugates” are interferon alpha molecules covalently attached to a PEG molecule. Illustrative PEG-interferon alpha conjugates include interferon alpha-2a (Roferon™, Hoffman La-Roche, Nutley, N.J.) in the form of pegylated interferon alpha-2a (e.g., as sold under the trade name Pegasys™), interferon alpha-2b (Intron™, from Schering-Plough Corporation) in the form of pegylated interferon alpha-2b (e.g., as sold under the trade name PEG-Intron™ from Schering-Plough Corporation), interferon alpha-2b-XL (e.g., as sold under the trade name PEG-Intron™), interferon alpha-2c (Berofor Alpha™, Boehringer Ingelheim, Ingelheim, Germany), PEG-interferon lambda (Bristol-Myers Squibb and ZymoGenetics), interferon alfa-2b alpha fusion polypeptides, interferon fused with the human blood protein albumin (Albuferon™, Human Genome Sciences), Omega Interferon (Intarcia), Locteron controlled release interferon (Biolex/OctoPlus), Biomed-510 (omega interferon), Peg-IL-29 (ZymoGenetics), Locteron CR (Octoplus), R-7025 (Roche), IFN-α-2b-XL (Flamel Technologies), belerofon (Nautilus) and consensus interferon as defined by determination of a consensus sequence of naturally occurring interferon alphas (Infergen™, Amgen, Thousand Oaks, Calif.).

Antibody therapy agents that may be useful in the present compositions and methods include, but are not limited to, antibodies specific to IL-10 (such as those disclosed in US Patent Publication No. US2005/0101770, humanized 12G8, a humanized monoclonal antibody against human IL-10, plasmids containing the nucleic acids encoding the humanized 12G8 light and heavy chains were deposited with the American Type Culture Collection (ATCC) as deposit numbers PTA-5923 and PTA-5922, respectively), and the like).

Examples of viral protease inhibitors useful in the present compositions and methods include, but are not limited to, an HCV protease inhibitor.

HCV protease inhibitors useful in the present compositions and methods include, but are not limited to, those disclosed in U.S. Pat. Nos. 7,494,988, 7,485,625, 7,449,447, 7,442,695, 7,425,576, 7,342,041, 7,253,160, 7,244,721, 7,205,330, 7,192,957, 7,186,747, 7,173,057, 7,169,760, 7,012,066, 6,914,122, 6,911,428, 6,894,072, 6,846,802, 6,838,475, 6,800,434, 6,767,991, 5,017,380, 4,933,443, 4,812,561 and 4,634,697; U.S. Patent Publication Nos. US20020068702, US20020160962, US20050119168, US20050176648, US20050209164, US20050249702 and US20070042968; and International Publication Nos. WO 03/006490, WO 03/087092, WO 04/092161 and WO 08/124148.

Additional HCV protease inhibitors useful in the present compositions and methods include, but are not limited to, VX-950 (Telaprevir, Vertex), VX-500 (Vertex), VX-813 (Vertex), VBY-376 (Virobay), BI-201335 (Boehringer Ingelheim), TMC-435 (Medivir/Tibotec), ABT-450 (Abbott/Enanta), TMC-435350 (Medivir), RG7227 (Danoprevir, InterMune/Roche), EA-058 (Abbott/Enanta), EA-063 (Abbott/Enanta), GS-9256 (Gilead), IDX-320 (Idenix), ACH-1625 (Achillion), ACH-2684 (Achillion), GS-9132 (Gilead/Achillion), ACH-1095 (Gilead/Achillon), IDX-136 (Idenix), IDX-316 (Idenix), ITMN-8356 (InterMune), ITMN-8347 (InterMune), ITMN-8096 (InterMune), ITMN-7587 (InterMune), BMS-650032 (Bristol-Myers Squibb), VX-985 (Vertex) and PHX1766 (Phenomix).

Further examples of HCV protease inhbitors useful in the present compositions and methods include, but are not limited to, those disclosed in Landro et al., Biochemistry, 36(31):9340-9348 (1997); Ingallinella et al., Biochemistry, 37(25):8906-8914 (1998); Llinás-Brunet et al., Bioorg Med Chem Lett, 8(13):1713-1718 (1998); Martin et al., Biochemistry, 37(33):11459-11468 (1998); Dimasi et al., J Virol, 71(10):7461-7469 (1997); Martin et al., Protein Eng, 10(5):607-614 (1997); Elzouki et al., J Hepat, 27(1):42-48 (1997); Bio World Today, 9(217):4 (Nov. 10, 1998); U.S. Patent Publication Nos. US2005/0249702 and US 2007/0274951; and International Publication Nos. WO 98/14181, WO 98/17679, WO 98/17679, WO 98/22496 and WO 99/07734 and WO 05/087731.

Further examples of HCV protease inhibitors useful in the present compositions and methods include, but are not limited to, the following compounds:

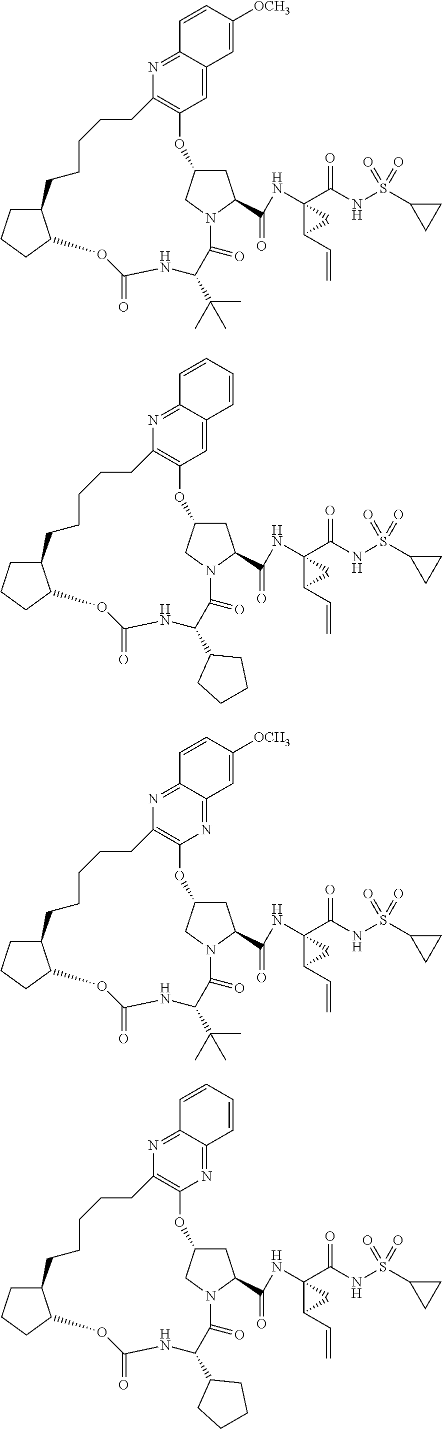

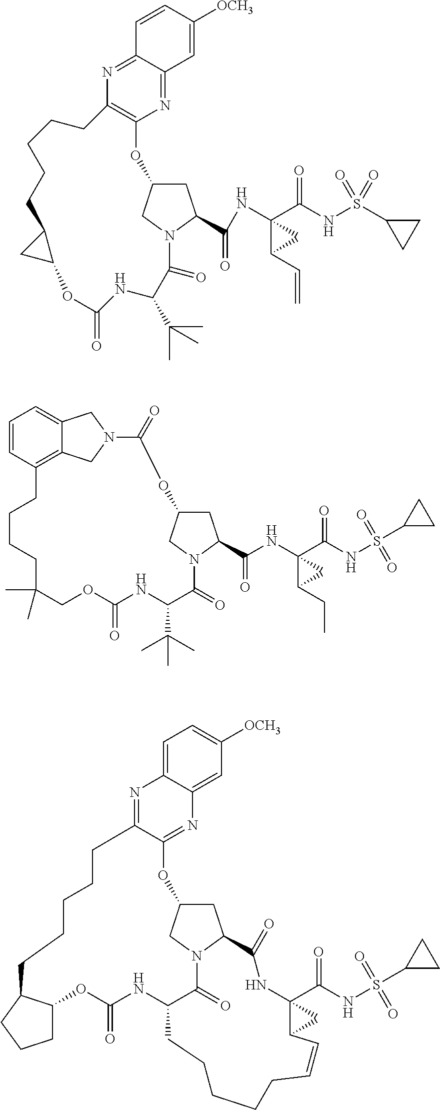 [SMILES: C=C[C@@H]1C[C@]1(NC(=O)[C@@H]1C[C@@H]2CN1C(=O)[C@H](C(C)(C)C)NC(=O)O[C@@H]1C[C@H]1CCCCCc1nc3ccc(OC)cc3nc1O2)C(=O)NS(=O)(=O)C1CC1 ; CC[C@@H]1C[C@]1(NC(=O)[C@@H]1C[C@@H]2CN1C(=O)[C@H](C(C)(C)C)NC(=O)OCC(C)(C)CCCCc1cccc3c1CN(C3)C(=O)O2)C(=O)NS(=O)(=O)C1CC1 ; COc1ccc2nc3c(nc2c1)O[C@@H]1C[C@H]2C(=O)N[C@]4(C(=O)NS(=O)(=O)C5CC5)C[C@H]4/C=C\CCCCC[C@H](NC(=O)O[C@@H]4CCC[C@H]4CCCCC3)C(=O)N2C1]

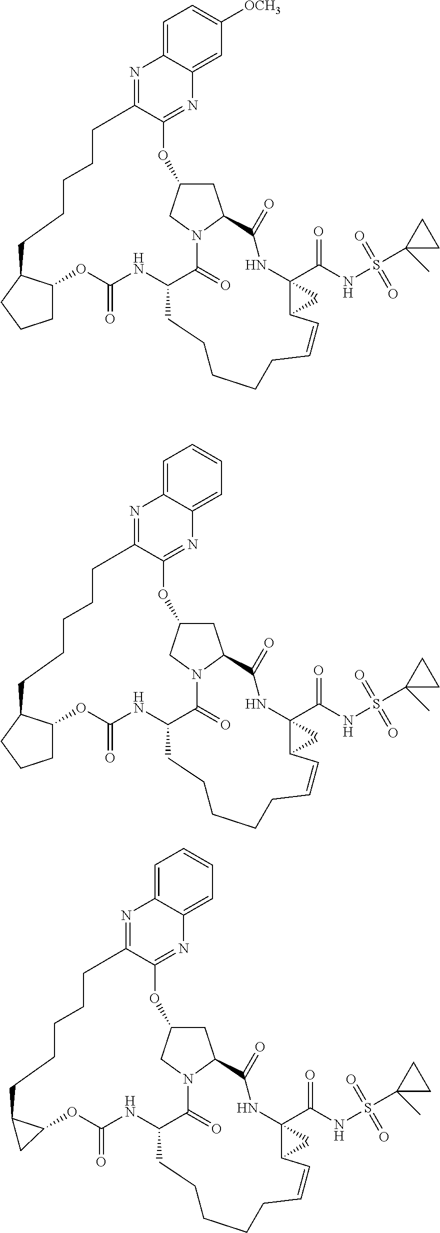

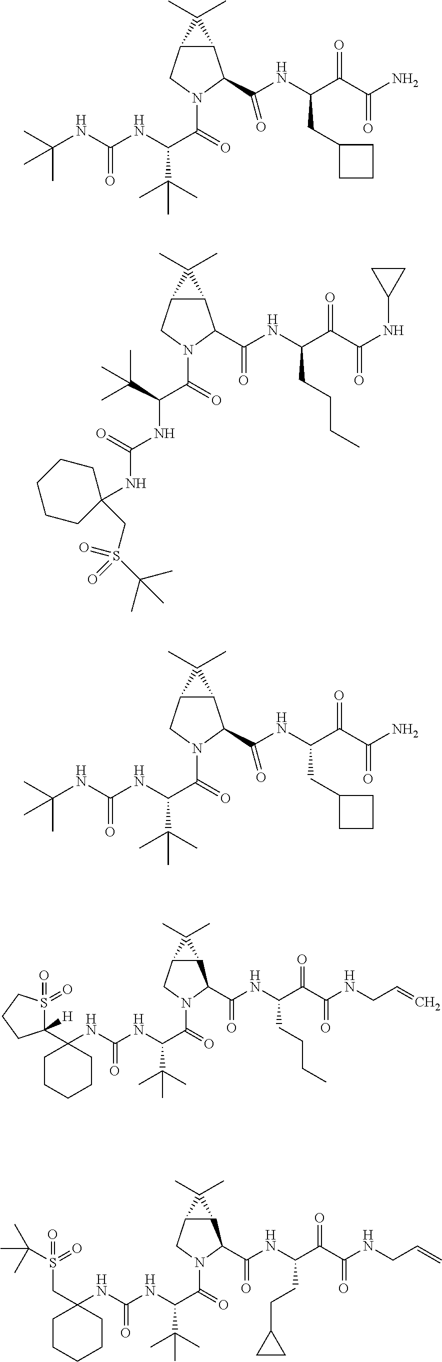

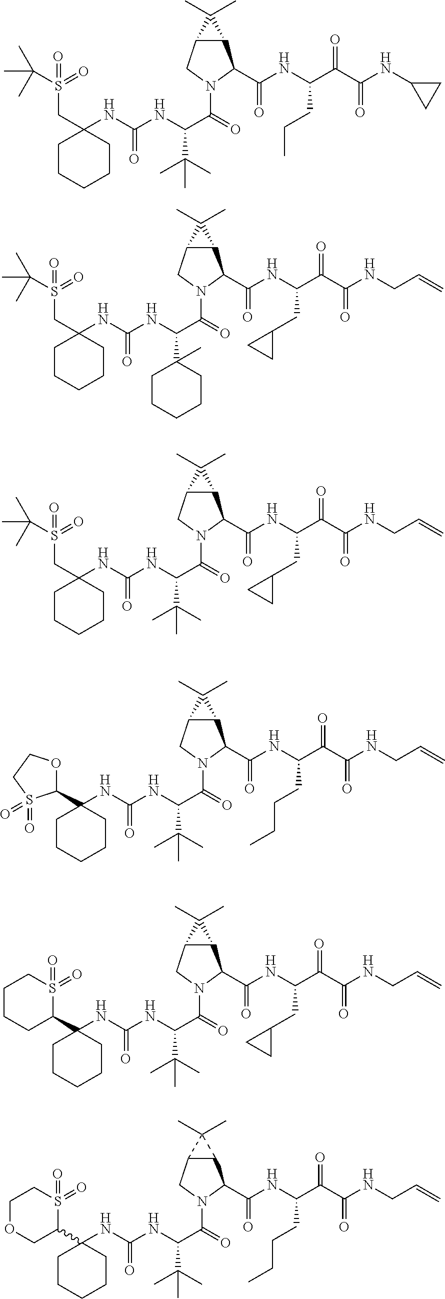

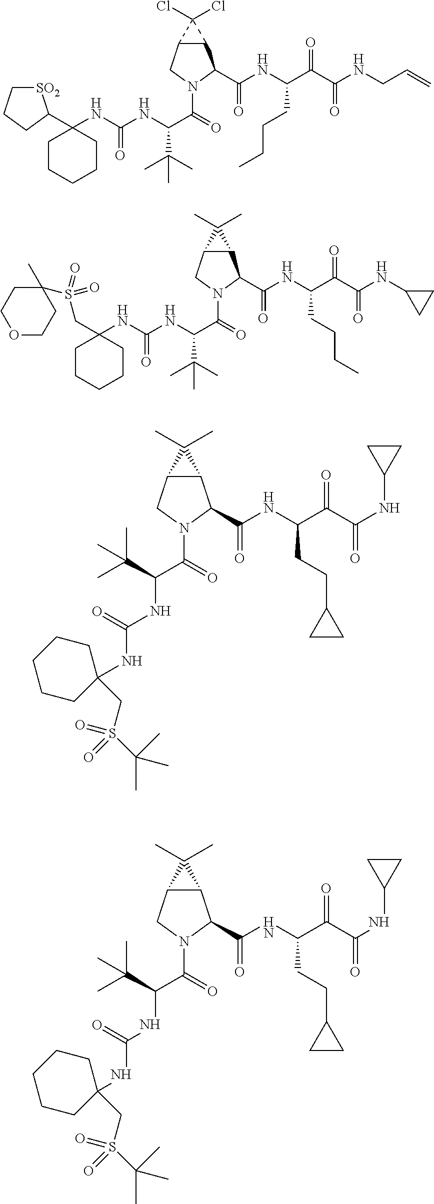

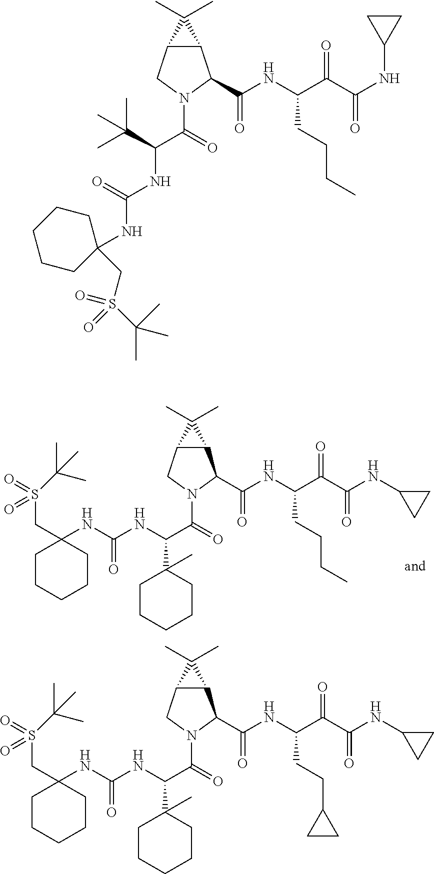

and pharmaceutically acceptable salts thereof

Viral replication inhibitors useful in the present compositions and methods include, but are not limited to, HCV replicase inhibitors, IRES inhibitors, NS4A inhibitors, NS3 helicase inhibitors, NS5A inhibitors, NS5B inhibitors, ribavirin, AZD-2836 (Astra Zeneca), viramidine, A-831 (Arrow Therapeutics), EDP-239 (Enanta), ACH-2928 (Achillion), GS-5885 (Gilead); an antisense agent or a therapeutic vaccine.

Viral entry inhibitors useful as second additional therapeutic agents in the present compositions and methods include, but are not limited to, PRO-206 (Progenies), REP-9C (REPICor), SP-30 (Samaritan Pharmaceuticals) and ITX-5061 (iTherx).

HCV NS4A inhibitors useful in the useful in the present compositions and methods include, but are not limited to, those disclosed in U.S. Pat. Nos. 7,476,686 and 7,273,885; U.S. Patent Publication No. US20090022688; and International Publication Nos. WO 2006/019831 and WO 2006/019832. Additional HCV NS4A inhibitors useful as second additional therapeutic agents in the present compositions and methods include, but are not limited to, AZD2836 (Astra Zeneca), ACH-1095 (Achillion) and ACH-806 (Achillion).

HCV NS5A inhibitors useful in the present compositions and methods include, but are not limited to, A-832 (Arrow Therapeutics), PPI-461 (Presidio), PPI-1301 (Presidio) and BMS-790052 (Bristol-Myers Squibb).

HCV replicase inhibitors useful in the present compositions and methods include, but are not limited to, those disclosed in U.S. Patent Publication No. US20090081636.

Therapeutic vaccines useful in the present compositions and methods include, but are not limited to, IC41 (Intercell Novartis), CSL123 (Chiron/CSL), GI 5005 (Globeimmune), TG-4040 (Transgene), GNI-103 (GENimmune), Hepavaxx C (ViRex Medical), ChronVac-C(Inovio/Tripep), PeviPRO™ (Pevion Biotect), HCV/MF59 (Chiron/Novartis), MBL-HCV1 (MassBiologics), GI-5005 (GlobeImmune), CT-011 (CureTech/Teva) and Civacir (NABI).

Examples of further additional therapeutic agents that may be useful in the present compositions and methods include, but are not limited to, Ritonavir (Abbott), TT033 (Benitec/Tacere Bio/Pfizer), Sirna-034 (Sirna Therapeutics), GNI-104 (GENimmune), GI-5005 (Globeimmune), IDX-102 (Idenix), Levovirin™ (ICN Pharmaceuticals, Costa Mesa, Calif.); Humax (Genmab), ITX-2155 (Ithrex/Novartis), PRO 206 (Progenics), HepaCide-I (NanoVirocides), MX3235 (Migenix), SCY-635 (Scynexis); KPE02003002 (Kemin Pharma), Lenocta (VioQuest Pharmaceuticals), IET—Interferon Enhancing Therapy (Transition Therapeutics), Zadaxin (SciClone Pharma), VP 50406™ (Viropharma, Incorporated, Exton, Pa.); Taribavirin (Valeant Pharmaceuticals); Nitazoxanide (Romark); Debio 025 (Debiopharm); GS-9450 (Gilead); PF-4878691 (Pfizer); ANA773 (Anadys); SCV-07 (SciClone Pharmaceuticals); NIM-881 (Novartis); ISIS 14803™ (ISIS Pharmaceuticals, Carlsbad, Calif.); Heptazyme™ (Ribozyme Pharmaceuticals, Boulder, Colo.); Thymosin™ (SciClone Pharmaceuticals, San Mateo, Calif.); Maxamine™ (Maxim Pharmaceuticals, San Diego, Calif.); NKB-122 (JenKen Bioscience Inc., North Carolina); Alinia (Romark Laboratories), INFORM-1 (a combination of R7128 and ITMN-191); and mycophenolate mofetil (Hoffman-LaRoche, Nutley, N.J.).

The doses and dosage regimen of the other agents used in the combination therapies of the present invention for the treatment or prevention of HCV infection can be determined by the attending clinician, taking into consideration the approved doses and dosage regimen in the package insert; the age, sex and general health of the patient; and the type and severity of the viral infection or related disease or disorder. When administered in combination, the Silyl-Containing Heterocyclic Compound(s) and the other agent(s) can be administered simultaneously (i.e., in the same composition or in separate compositions one right after the other) or sequentially. This particularly useful when the components of the combination are given on different dosing schedules, e.g., one component is administered once daily and another component is administered every six hours, or when the preferred pharmaceutical compositions are different, e.g., one is a tablet and one is a capsule. A kit comprising the separate dosage forms is therefore advantageous.

Generally, a total daily dosage of the at least one Silyl-Containing Heterocyclic Compound(s) alone, or when administered as combination therapy, can range from about 1 to about 2500 mg per day, although variations will necessarily occur depending on the target of therapy, the patient and the route of administration. In one embodiment, the dosage is from about 10 to about 1000 mg/day, administered in a single dose or in 2-4 divided doses. In another embodiment, the dosage is from about 1 to about 500 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In another embodiment, the dosage is from about 500 to about 1500 mg/day, administered in a single dose or in 2-4 divided doses. In still another embodiment, the dosage is from about 500 to about 1000 mg/day, administered in a single dose or in 2-4 divided doses. In yet another embodiment, the dosage is from about 100 to about 500 mg/day, administered in a single dose or in 2-4 divided doses.

In one embodiment, when the additional therapeutic agent is INTRON-A interferon alpha 2b (commercially available from Schering-Plough Corp.), this agent is administered by subcutaneous injection at 3MIU(12 mcg)/0.5 mL/TIW for 24 weeks or 48 weeks for first time treatment.

In another embodiment, when the additional therapeutic agent is PEG-INTRON interferon alpha 2b pegylated (commercially available from Schering-Plough Corp.), this agent is administered by subcutaneous injection at 1.5 mcg/kg/week, within a range of 40 to 150 mcg/week, for at least 24 weeks.

In another embodiment, when the additional therapeutic agent is ROFERON A interferon alpha 2a (commercially available from Hoffmann-La Roche), this agent is administered by subcutaneous or intramuscular injection at 3MIU(11.1 mcg/mL)/TIW for at least 48 to 52 weeks, or alternatively 6MIU/TIW for 12 weeks followed by 3MIU/TIW for 36 weeks.

In still another embodiment, when the additional therapeutic agent is PEGASUS interferon alpha 2a pegylated (commercially available from Hoffmann-La Roche), this agent is administered by subcutaneous injection at 180 mcg/1 mL or 180 mcg/0.5 mL, once a week for at least 24 weeks.

In yet another embodiment, when the additional therapeutic agent is INFERGEN interferon alphacon-1 (commercially available from Amgen), this agent is administered by subcutaneous injection at 9 mcg/TIW is 24 weeks for first time treatment and up to 15 mcg/TIW for 24 weeks for non-responsive or relapse treatment.

In a further embodiment, when the additional therapeutic agent is Ribavirin (commercially available as REBETOL ribavirin from Schering-Plough or COPEGUS ribavirin from Hoffmann-La Roche), this agent is administered at a daily dosage of from about 600 to about 1400 mg/day for at least 24 weeks.

In one embodiment, one or more compounds of the present invention are administered with one or more additional therapeutic agents selected from: an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a viral polymerase inhibitor a virion production inhibitor, a viral entry inhibitor, a viral assembly inhibitor, an antibody therapy (monoclonal or polyclonal), and any agent useful for treating an RNA-dependent polymerase-related disorder.

In another embodiment, one or more compounds of the present invention are administered with one or more additional therapeutic agents selected from an HCV protease inhibitor, an HCV polymerase inhibitor, an HCV replication inhibitor, a nucleoside, an interferon, a pegylated interferon and ribavirin. The combination therapies can include any combination of these additional therapeutic agents.

In another embodiment, one or more compounds of the present invention are administered with one additional therapeutic agent selected from an HCV protease inhibitor, an interferon, a pegylated interferon and ribavirin.

In still another embodiment, one or more compounds of the present invention are administered with two additional therapeutic agents selected from an HCV protease inhibitor, an HCV replication inhibitor, a nucleoside, an interferon, a pegylated interferon and ribavirin.

In another embodiment, one or more compounds of the present invention are administered with an HCV protease inhibitor and ribavirin. In another specific embodiment, one or more compounds of the present invention are administered with a pegylated interferon and ribavirin.

In another embodiment, one or more compounds of the present invention are administered with three additional therapeutic agents selected from an HCV protease inhibitor, an HCV replication inhibitor, a nucleoside, an interferon, a pegylated interferon and ribavirin.

In one embodiment, one or more compounds of the present invention are administered with one or more additional therapeutic agents selected from an HCV polymerase inhibitor, a viral protease inhibitor, an interferon, and a viral replication inhibitor. In another embodiment, one or more compounds of the present invention are administered with one or more additional therapeutic agents selected from an HCV polymerase inhibitor, a viral protease inhibitor, an interferon, and a viral replication inhibitor. In another embodiment, one or more compounds of the present invention are administered with one or more additional therapeutic agents selected from an HCV polymerase inhibitor, a viral protease inhibitor, an interferon, and ribavirin.

In one embodiment, one or more compounds of the present invention are administered with one additional therapeutic agent selected from an HCV polymerase inhibitor, a viral protease inhibitor, an interferon, and a viral replication inhibitor. In another embodiment, one or more compounds of the present invention are administered with ribavirin.

In one embodiment, one or more compounds of the present invention are administered with two additional therapeutic agents selected from an HCV polymerase inhibitor, a viral protease inhibitor, an interferon, and a viral replication inhibitor.

In another embodiment, one or more compounds of the present invention are administered with ribavirin, interferon and another therapeutic agent.

In another embodiment, one or more compounds of the present invention are administered with ribavirin, interferon and another therapeutic agent, wherein the additional therapeutic agent is selected from an HCV polymerase inhibitor, a viral protease inhibitor, and a viral replication inhibitor.

In still another embodiment, one or more compounds of the present invention are administered with ribavirin, interferon and a viral protease inhibitor.

In another embodiment, one or more compounds of the present invention are administered with ribavirin, interferon and an HCV protease inhibitor.

In another embodiment, one or more compounds of the present invention are administered with ribavirin, interferon and boceprevir or telaprevir.

In a further embodiment, one or more compounds of the present invention are administered with ribavirin, interferon and an HCV polymerase inhibitor.

In another embodiment, one or more compounds of the present invention are administered with pegylated-interferon alpha and ribavirin.

### Compositions and Administration

Due to their activity, the Silyl-Containing Heterocyclic Compounds are useful in veterinary and human medicine. As described above, the Silyl-Containing Heterocyclic Compounds are useful for treating or preventing HCV infection in a patient in need thereof.

When administered to a patient, the Silyl-Containing Heterocyclic Compounds can be administered as a component of a composition that comprises a pharmaceutically acceptable carrier or vehicle. The present invention provides pharmaceutical compositions comprising an effective amount of at least one Silyl-Containing Heterocyclic Compound and a pharmaceutically acceptable carrier. In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Solid form preparations include powders, tablets, dispersible granules, capsules, cachets and suppositories. Powders and tablets may be comprised of from about 0.5 to about 95 percent inventive composition. Tablets, powders, cachets and capsules can be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum, and the like. Sweetening and flavoring agents and preservatives may also be included where appropriate.

Liquid form preparations include solutions, suspensions and emulsions and may include water or water-propylene glycol solutions for parenteral injection.

Liquid form preparations may also include solutions for intranasal administration.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Additionally, the compositions of the present invention may be formulated in sustained release form to provide the rate controlled release of any one or more of the components or active ingredients to optimize therapeutic effects, i.e., antiviral activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the one or more Silyl-Containing Heterocyclic Compounds are administered orally.

In another embodiment, the one or more Silyl-Containing Heterocyclic Compounds are administered intravenously.

In one embodiment, a pharmaceutical preparation comprising at least one Silyl-Containing Heterocyclic Compound is in unit dosage form. In such form, the preparation is subdivided into unit doses containing effective amounts of the active components.

Compositions can be prepared according to conventional mixing, granulating or coating methods, respectively, and the present compositions can contain, in one embodiment, from about 0.1% to about 99% of the Silyl-Containing Heterocyclic Compound(s) by weight or volume. In various embodiments, the present compositions can contain, in one embodiment, from about 1% to about 70% or from about 5% to about 60% of the Silyl-Containing Heterocyclic Compound(s) by weight or volume.

The quantity of Silyl-Containing Heterocyclic Compound in a unit dose of preparation may be varied or adjusted from about 1 mg to about 2500 mg. In various embodiment, the quantity is from about 10 mg to about 1000 mg, 1 mg to about 500 mg, 1 mg to about 100 mg, and 1 mg to about 100 mg.

For convenience, the total daily dosage may be divided and administered in portions during the day if desired. In one embodiment, the daily dosage is administered in one portion. In another embodiment, the total daily dosage is administered in two divided doses over a 24 hour period. In another embodiment, the total daily dosage is administered in three divided doses over a 24 hour period. In still another embodiment, the total daily dosage is administered in four divided doses over a 24 hour period.

The amount and frequency of administration of the Silyl-Containing Heterocyclic Compounds will be regulated according to the judgment of the attending clinician considering such factors as age, condition and size of the patient as well as severity of the symptoms being treated. Generally, a total daily dosage of the Silyl-Containing Heterocyclic Compounds range from about 0.1 to about 2000 mg per day, although variations will necessarily occur depending on the target of therapy, the patient and the route of administration. In one embodiment, the dosage is from about 1 to about 200 mg/day, administered in a single dose or in 2-4 divided doses. In another embodiment, the dosage is from about 10 to about 2000 mg/day, administered in a single dose or in 2-4 divided doses. In another embodiment, the dosage is from about 100 to about 2000 mg/day, administered in a single dose or in 2-4 divided doses. In still another embodiment, the dosage is from about 500 to about 2000 mg/day, administered in a single dose or in 2-4 divided doses.

The compositions of the invention can further comprise one or more additional therapeutic agents, selected from those listed above herein. Accordingly, in one embodiment, the present invention provides compositions comprising: (i) at least one Silyl-Containing Heterocyclic Compound or a pharmaceutically acceptable salt thereof; (ii) one or more additional therapeutic agents that are not a Silyl-Containing Heterocyclic Compound; and (iii) a pharmaceutically acceptable carrier, wherein the amounts in the composition are together effective to treat HCV infection.

In one embodiment, the present invention provides compositions comprising a Compound of Formula (I) and a pharmaceutically acceptable carrier.

In another embodiment, the present invention provides compositions comprising a Compound of Formula (I), a pharmaceutically acceptable carrier, and a second therapeutic agent selected from the group consisting of HCV antiviral agents, immunomodulators, and anti-infective agents.

In another embodiment, the present invention provides compositions comprising a Compound of Formula (I), a pharmaceutically acceptable carrier, and wto additional therapeutic agents, each of which are independently selected from the group consisting of HCV antiviral agents, immunomodulators, and anti-infective agents.

### Kits

In one aspect, the present invention provides a kit comprising a therapeutically effective amount of at least one Silyl-Containing Heterocyclic Compound, or a pharmaceutically acceptable salt, solvate, ester or prodrug of said compound and a pharmaceutically acceptable carrier, vehicle or diluent.

In another aspect the present invention provides a kit comprising an amount of at least one Silyl-Containing Heterocyclic Compound, or a pharmaceutically acceptable salt, solvate, ester or prodrug of said compound and an amount of at least one additional therapeutic agent listed above, wherein the amounts of the two or more active ingredients result in a desired therapeutic effect. In one embodiment, the one or more Silyl-Containing Heterocyclic Compounds and the one or more additional therapeutic agents are provided in the same container. In one embodiment, the one or more Silyl-Containing Heterocyclic Compounds and the one or more additional therapeutic agents are provided in separate containers.

### EXAMPLES

### General Methods

Solvents, reagents, and intermediates that are commercially available were used as received. Reagents and intermediates that are not commercially available were prepared in the manner as described below. ¹H NMR spectra were obtained on a Bruker Avance 500 (500 MHz) and are reported as ppm downfield from Me₄Si with number of protons, multiplicities, and coupling constants in Hertz indicated parenthetically. Where LC/MS data are presented, analyses was performed using an Applied Biosystems API-100 mass spectrometer and Shimadzu SCL-10A LC column: Altech platinum C18, 3 micron, 33 mm×7 mm ID; gradient flow: 0 minutes—10% CH₃CN, 5 minutes—95% CH₃CN, 5-7 minutes—95% CH₃CN, 7 minutes—stop. The retention time and observed parent ion are given. Flash column chromatography was performed using pre-packed normal phase silica from Biotage, Inc. or bulk silica from Fisher Scientific. Unless otherwise indicated, column chromatography was performed using a gradient elution of hexanes/ethyl acetate, from 100% hexanes to 100% ethyl acetate.

### Example 1

### Preparation of Intermediate Compound Int-1a

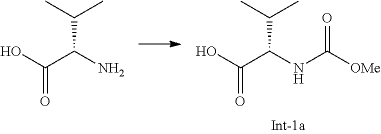

To a solution of L-valine (10.0 g, 85.3 mmol) in 1M aqueous NaOH solution (86 mL) at room temperature was added solid sodium carbonate (4.60 g, 43.4 mmol). The reaction mixture was cooled to 0° C. (ice bath) and then methyl chloroformate (7.20 mL, 93.6 mmol) was added dropwise over 20 minutes. The reaction mixture was then allowed to warm to room temperature, and allowed to stir at room temperature for an additional 4 hours. The reaction mixture was then diluted with diethyl ether (100 mL), the resulting solution was cooled to at 0° C., and then concentrated hydrochloric acid (18 mL, 216 mmol) was added slowly. The reaction was extracted with EtOAc (3×100 mL) and the combined organics were dried over MgSO₄, filtered and concentrated in vacuo to provide Compound Int-1a (13.5 g, 90%), which was used without further purification.

The following intermediates can be prepared by the reaction of L-valine with isopropyl chloroformate (Aldrich Inc.), 2-methoxyethyl chloroformate (Aldrich) or with 1-methylcyclopropyl hydroxysuccinimide respectively, using the method described above:

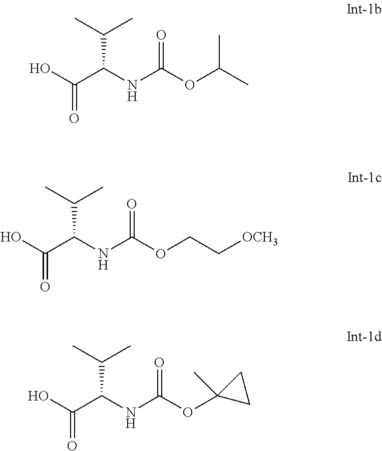

### Example 2

### Preparation of Intermediate Compound Int-2a

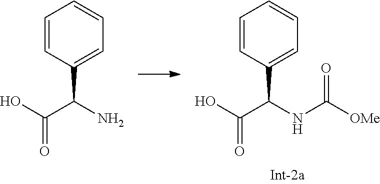 [SMILES: N[C@@H](C(=O)O)c1ccccc1 ; COC(=O)N[C@@H](C(=O)O)c1ccccc1]

To a solution of D-phenylglycine (10.0 g, 66.1 mmol) and NaOH (21.2 g, 265 mmol) in water (60 mL) at 0° C. was added methyl chloroformate (10.2 mL, 133 mmol) dropwise over 20 minutes. The resulting mixture was allowed to stir at 0° C. for 1 hour, then was acidified using concentrated hydrochloric acid (25 mL, 300 mmol). The acidic solution was extracted with EtOAc (3×100 mL) and the combined organics were dried over MgSO₄, filtered and concentrated in vacuo to provide Compound Int-2a (12.6 g, 91%), which was used without further purification.

The following intermediates can be prepared by the reaction of glycine, L-Alanine and 4-F phenylglycine, respectively with methyl chloroformate (Aldrich Inc.) using the method described above:

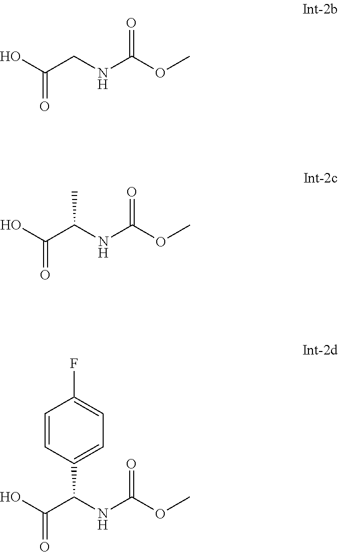

### Example 3

### Preparation of Intermediate Compound Int-3a

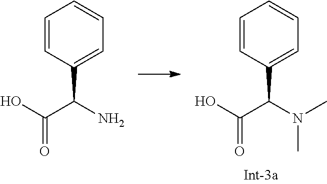

A solution of D-phenylglycine (20.0 g, 132 mmol), 37% aqueous formaldehyde (66 mL, 814 mmol) and 5% Pd on carbon (8.0 g, mmol) in a mixture of methanol (80 mL) and 1 N HCl (60 mL) was placed on a hydrogenation shaker and shook under an atmosphere of 35-40 psi hydrogen for 4 hours. The reaction was then flushed with nitrogen, filtered through a celite pad and concentrated in vacuo to provide Compound Int-3a (29.7 g, quant.) as a white solid, which was used without further purification.

Using this method, and substituting ethanol for methanol, compound Int-3b was prepared.

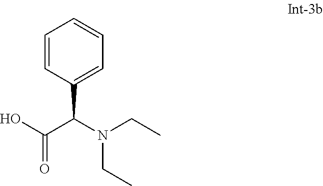

### Example 4

### Preparation of Intermediate Compound Int-4e

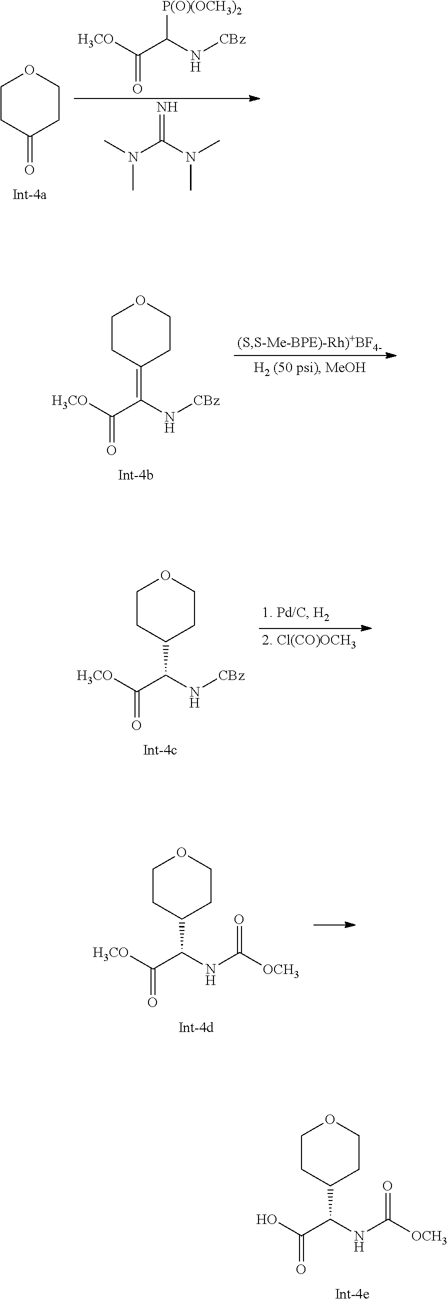

### Step A—Synthesis of Intermediate Compound Int-4b

To a solution of methyl 2-(benzyloxycarbonylamino)-2-(dimethoxyphosphoryl) acetate (10.0 g, 30.2 mmol, made as described in Hamada et al., Organic Letters; English; 20: 4664-4667 (2009)) in THF (100 mL) at −20° C. was added tetramethylguanidine (4.20 mL, 33.2 mmol). The reaction mixture was allowed to stir at −20° C. for 1 hour then a solution of compound Int-4a (3.1 mL, 33.2 mmol) in THF (5 mL) was added and the reaction mixture was warmed to room temperature and allowed to stir for about 15 hours. EtOAc (200 mL) was added and the organic mixture was washed with water (3×50 mL) and brine (50 mL). The organic layers were combined and dried with Na₂SO₄, filtered and concentrated in vacuo. The crude product was purified using flash chromatography on an ISCO 330 g Redi-Sep column using 0-35% EtOAc/hexanes as the eluent to provide Compound Int-4b as a white solid (615 mg, 45%). ¹H NMR (CDCl₃) δ 7.40-7.30 (m, 5H), 6.00 (br s, 1H), 5.12 (s, 2H), 3.80-3.65 (m, 7H), 2.92 (m, 2H), 2.52-2.48 (m, 2H).

### Step B—Synthesis of Intermediate Compound Int-4c

To a solution of Int-4b (2.43 g, 7.96 mmol) in methanol (160 mL) previously purged with N₂ was added (−)-1,2-Bis((2S,5S)-2,5-dimethylphospholano)ethane (cyclooctadiene)rhodium(I) tetrafluoroborate (487 mg, 0.880 mmol) under N₂. The mixture was shaken in a Parr shaker apparatus for 18 hours at 50 psi of H₂. After evacuating the hydrogen, the suspension was filtered and the filtrate was concentrated to provide Compound Int-4c as a white solid (1.30 g, 53%). ¹H NMR (CDCl₃) δ 7.40-7.30 (m, 5H), 5.32 (br s, 1H), 5.12 (s, 2H), 4.40-4.30 (m, 1H), 4.00-3.95 (m, 2H), 3.75 (s, 3H), 3.40-3.25 (m, 2H), 2.10-1.95 (m, 1H), 1.50-1.45 (m, 4H).

### Step C—Synthesis of Intermediate Compound Int-4d

To a suspension of 50% palladium on carbon (10% wet, 200 mg) in absolute ethanol (20 mL) under nitrogen was added Int-4c (1.06 g, 3.45 mmol). With stirring, the solution was placed in vacuo for 30 seconds and then was opened to a hydrogen gas balloon for 2 hours. After evacuating the hydrogen, the suspension was filtered through a Celite pad and the pad washed with ethanol (2×20 mL). The filtrate was concentrated to provide a colorless oil (585 mg, 98%). ¹H NMR (CDCl₃) δ 4.06-3.96 (m, 2H), 3.73 (s, 3H), 3.48-3.28 (m, 3H), 1.92-1.78 (m, 1H), 1.61-1.47 (m, 6H).

To a solution of the colorless oil (585 mg, 3.37 mmol) and triethylamine (0.710 mL, 5.09 mmol) in CH₂Cl₂ (6 mL) was added methyl chloroformate (0.290 mL, 3.76 mmol). The reaction mixture was allowed to stir at room temperature for about 15 hours. Water (15 mL) was added and the aqueous mixture was extracted with CH₂Cl₂ (3×20 mL) The combined organic layers were dried over Na₂SO₄, filtered and concentrated in vacuo. The crude product was purified using flash chromatography on an ISCO 24 g Redi-Sep column using 0-3% MeOH/CH₂Cl₂ as the eluent to provide Compound Int-4d as a colorless oil (600 mg, 77%). ¹H NMR (CDCl₃) δ 5.27-5.18 (m, 1H), 4.38-4.28 (m, 1H), 4.06-3.96 (m, 2H), 3.75 (s, 3H), 3.69 (s, 3H), 3.39-3.30 (m, 2H), 2.09-1.94 (m, 1H), 1.59-1.48 (m, 4H).

### Step D—Synthesis of Intermediate Compound Int-4e

To a solution of compound Int-4d (600 mg, 2.59 mmol) in THF (5 mL) was added lithium hydroxide monohydrate (218 mg, 5.19 mmol) in water (5 mL). The reaction mixture was allowed to stir at room temperature for 2 hours then concentrated to half volume. The aqueous mixture was then acidified with 6N HCl and extracted with EtOAc (7×50 mL). The combined organic layers were dried over Na₂SO₄, filtered and concentrated to provide Compound Int-4e as an off-white solid (485 mg, 86%). ¹H NMR (CD₃OD) δ 4.09-4.07 (m, 1H), 3.96-3.92 (m, 2H), 3.65 (s, 3H), 3.40-3.34 (m, 2H), 2.10-1.99 (m, 1H), 1.56-1.47 (m, 4H).

### Example 5

### Preparation of Intermediate Compound Int-5f

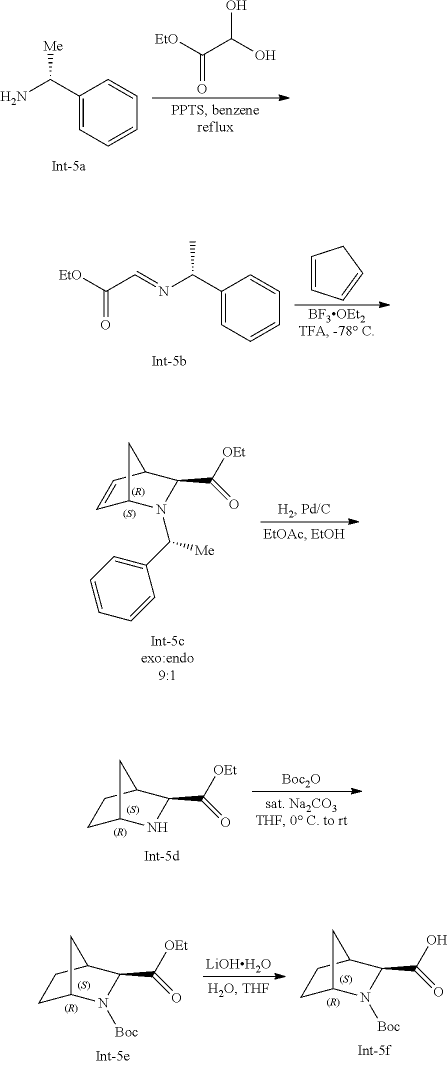

### Step A—Synthesis of Intermediate Compound Int-5b

A stirred mixture of Int-5a (50.0 g, 0.412 mol), ethyl glyoxylate (81.5 mL, 50% in toluene, 0.412 mol) and PPTS (0.50 g, 2.00 mmol) in benzene (600 mL) was heated to reflux in a Dean-Stark apparatus until no further water (=8 mL) azeotroped from the reaction (˜4 h). The resulting mixture was concentrated in vacuo. The crude residue Int-5b was used without purification: ¹H NMR (300 MHz, CDCl₃) δ 7.72 (s, 1H), 7.36-7.24 (m, 5H), 4.61 (q, J=6.9 Hz, 1H), 4.35 (q, J=7.2 Hz, 2H), 1.62 (d, J=6.6 Hz, 3H), 1.34 (t, J=7.2 Hz, 3H).

### Step B—Synthesis of Intermediate Compound Int-5c

To a stirred solution of crude Int-5b in methylene chloride (600 mL) at −78° C. were added the following in 10 minute intervals: TFA (31.0 mL, 0.416 mol), boron trifluoride etherate (51.3 mL, 0.416 mol) and freshly distilled cyclopentadiene (32.7 g, 0.494 mol). After less than 2 minutes the reaction forms a thick brown mass. After 6 hours at −78° C. the reaction was allowed to slowly warm to room temperature for about 15 hours, at which time the reaction had formed a dark brown solution. The reaction was quenched with saturated aqueous Na₂CO₃ (˜900 mL) and allowed to stir for 30 minutes. The resultant solids were removed by filtration through Celite®. The aqueous filtrate was extracted with methylene chloride (3×100 mL). The combined extracts were washed with saturated aqueous NaCl (2×75 mL), dried over Na₂SO₄, filtered and concentrated in vacuo. The crude product was purified using flash column chromatography (silica; 8×18 cm) using 10% to 25% ethyl acetate/hexanes as the eluent to provide endo Int-5c (10.9 g, 9%) as a brown oil: ¹H NMR (300 MHz, CDCl₃) δ 7.34-7.19 (m, 5H), 6.00-5.95 (m, 1H), 4.18 (q, J=7.1 Hz, 3H), 3.47 (s, 1H), 3.03 (s, 1H), 2.97 (q, J=6.5 Hz, 1H), 2.41 (s, 1H), 1.86 (d, J=8.2 Hz, 1H), 1.26 (t, J=6.6 Hz, 3H), 1.17 (t, J=6.6 Hz, 3H). Exo Int-6c (84.3 g, 74%) was collected as a brown oil: ¹H NMR (300 MHz, CDCl₃) δ 7.34-7.19 (m, 5H), 6.36-6.33 (m, 1H), 6.22-6.18 (m, 1H), 4.37 (s, 1H), 3.87 (q, J=6.8 Hz, 2H), 3.10 (q, J=6.5 Hz, 1H), 2.96 (s, 1H), 2.27 (s, 1H), 2.20 (d, J=8.4 Hz, 1H), 1.48 (d, J=6.5 Hz, 3H), 1.01 (d, J=7.0 Hz, 3H), 1.00 (m, 1H).

### Step C—Synthesis of Intermediate Compound Int-5d

A mixture of exo-Int-5c (15.8 g, 0.582 mol) and 10% Pd/C (4.07 g, 50% wet) in a 1:2 mixture of EtOH/EtOAc (150 mL) was shaken in a Parr hydrogenation apparatus under an atmosphere of H₂ (50 psi). After 23 hours the mixture was filtered through Celite® and the filtrate concentrated in vacuo. ¹H NMR analysis of the resulting residue (10.8 g) showed some aromatic resonances present. Repetition of the hydrogenation procedure using 10% Pd/C (2.0 g) afforded Int-5d (10.0 g, quant.) as a brown oil: ¹H NMR (300 MHz, CDCl₃) δ 4.18 (q, J=7.2 Hz, 3H), 3.54 (s, 1H), 3.32 (s, 1H), 2.62 (s, 1H), 2.23 (s, 1H), 1.64-1.39 (m, 5H), 1.31-1.20 (m, 4H).

### Step D—Synthesis of Intermediate Compound Int-5e

To a stirred mixture of Int-5d (36.6 g, 0.236 mol) and saturated aqueous Na₂CO₃ (300 mL) in THF (600 mL) at 0° C. was added di-tert-butyl dicarbonate (59.0 g, 0.270 mol). The reaction mixture was allowed to slowly warm to room temperature over 6 hours. After 68 hours the reaction mixture was diluted with EtOAc (250 mL) and water (250 mL). The aqueous layer was extracted with EtOAc (2×200 mL) and the combined extracts were washed with saturated aqueous NaCl (2×75 mL), dried over Na₂SO₄, filtered and concentrated in vacuo. The resulting residue was purified using flash column chromatography (silica; 16×10 cm) using 10-20% ethyl acetate/hexanes as the eluent to provide Compound Int-5e (49.0 g, 84%) as a pale yellow oil: ¹H NMR (300 MHz, CDCl₃) δ 4.35 (s, 0.6H), 4.22-4.10 (m, 2.4H), 3.81 (s, 0.45H), 3.71 (s, 0.55H), 2.66 (s, 1H), 1.96-1.90 (m, 1H), 1.76-1.50 (m, 3H), 1.55-1.45 (m, 5H), 1.39 (s, 5H), 1.30-1.23 (m, 4H).

### Step E—Synthesis of Intermediate Compound Int-5f

To a stirred mixture of Int-5e (49.0 g, 0.182 mmol) in 1:1 THF/water (600 mL) was added LiOH.H₂O (15.3 g, 0.364 mol). The reaction mixture was warmed to 60° C. for 47 hours, cooled to room temperature and concentrated in vacuo to remove excess THF. The resulting residue was diluted with CH₂Cl₂ (200 mL) then acidified with 2N HCl until pH ˜4. The aqueous layer was extracted with CH₂Cl₂ (4×100 mL) and the combined extracts were washed with saturated aqueous NaCl (25 mL), dried over Na₂SO₄, filtered and concentrated in vacuo to provide Compound Int-5f (41.2 g, 93%) as an off white solid: ¹H NMR (400 MHz, DMSO-d₆) δ 12.44 (s, 1H), 4.13 (s, 0.56H), 4.06 (s, 0.47H), 3.61 (d, J=4.0 Hz, 1H), 2.59 (s, 1H), 1.75-1.45 (m, 5H), 1.39 (s, 4H), 1.32 (s, 5H), 1.23 (t, J=8.4 Hz, 1H); Optical Rotation: [α]^{D}₂₅ −169.0° (c=1.1, CHCl₃).

### Example 6

### Preparation of Intermediate Compound Int-6e

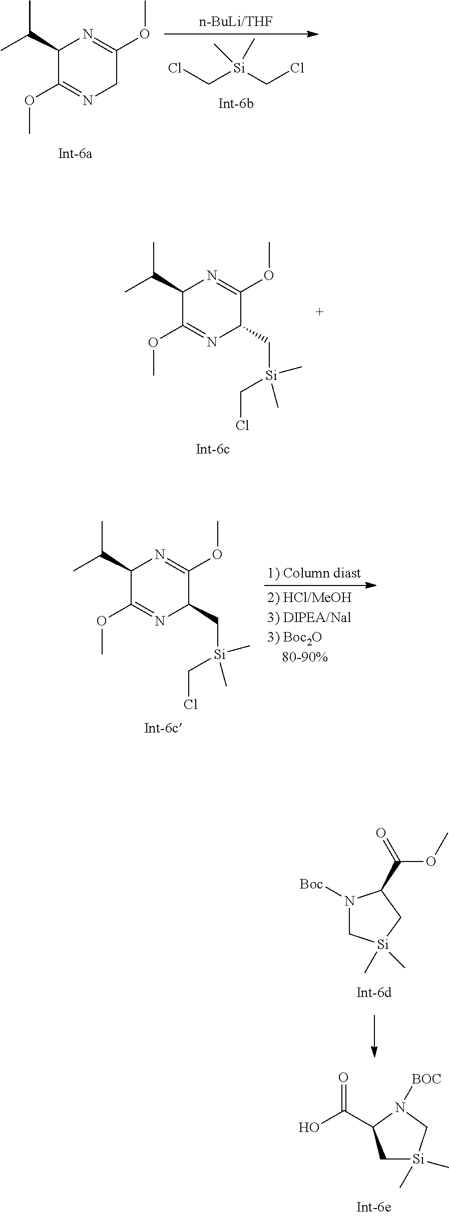

### Step A—Synthesis of Intermediate Compound Int-6c

A 5 L-3 necked round bottomed flask, equipped with a mechanical stirrer, temperature probe, addition funnel and N₂ inlet, was charged with the Schollkopf chiral auxiliary-(Int-6a, 200 g, 1.09 mol, 1.0 eq), bis(chloromethyl)dimethylsilane (Int-6b, 256 g, 1.63 mol, 1.5 eq), and THF (2 L, Aldrich anhydrous). The flask was cooled in a dry ice/2-propanol bath until the internal temperature reached −75° C. n-Butyl lithium (Aldrich 2.5 M in hexanes, 478 mL, 1.19 mol, 1.09 eq) was added via a dropping funnel over 1 hour while maintaining the internal reaction temperature between −67° C. and −76° C. The resulting orange-red solution was allowed to gradually warm to room temperature for about 15 hours. The reaction mixture was then re-cooled to 0° C. and quenched with 500 mL of water. Diethyl ether (2 L) was added and the layers were separated. The aqueous layer was extracted with 1 L of diethyl ether. The combined organic layers was washed with water and brine, dried with MgSO₄, filtered, and concentrated in vacuo to provide 480 g of an orange oil. This material was left in vacuo for about 15 hours to provide 420 g of oil (mixture of Int-6c and Int-6c′). The crude product was split into two batches and purified via silica gel chromatography on a 1.6 Kg flash column. The column was eluted with gradient of 0-4% Et₂O in hexanes. The product fractions were concentrated in vacuo at a bath temperature at or below 40° C. to provide 190 grams of Compound Int-6c (60% yield).

### Step B—Synthesis of Intermediate Compound Int-6d

A 5 L, 3-necked round bottomed flask equipped with a mechanical stirrer, addition funnel, temperature probe, external water bath and N₂ inlet was charged with compound Int-6c (196 g, 0.643 mol, 1.0 eq) and methanol (1.5 L). Aqueous HCl (500 mL of 10% by volume) was added at room temperature over 30 minutes, with a mild exotherm observed. The temperature increased to 37° C. then dropped back down. The reaction mixture was allowed to stir at room temperature for 3 hours and was monitored by TLC and LCMS. The reaction mixture was then concentrated in vacuo to an oil. Additional methanol (3×200 mL) was added and the reaction mixture was concentrated in vacuo again. The resulting crude product was dried under house vacuum for about 15 hours. The crude product was then dissolved in CH₂Cl₂ (750 mL) and Et₂O (1250 mL) and sodium iodide (96.4 g, 0.643 mol, 1.0 eq) was added. Diisopropylethylamine (336 mL, 1.929 mol, 3.0 eq) was added slowly over 25 minutes with efficient stirring, causing the temperature to increase to 35° C. then decrease again. The reaction mixture was allowed to stir at room temperature for 2 hours, at which time the MS of an aliquot indicated consumption of the starting material. The reaction mixture was allowed to stir for an additional 2 hours and then Boc-anhydride (281 g, 1.286 mol, 2.0 eq) was added. The reaction mixture was then allowed to stir at room temperature/. After two days, the reaction mixture was diluted with EtOAc (2 L) and water (1 L), and he layers were separated. The aqueous phase was extracted with 500 mL of EtOAc. The combined organic layers were washed with water (500 mL), and brine (500 mL), dried with MgSO₄, filtered, and concentrated in vacuo to a yellow oil (380 g). The crude product was split into two 180 g portions for convenience and each portion was purified via flash silica gel chromatography. Column conditions for a 180 g portion of crude product are as follows. The 180 gram sample of crude product was loaded onto a 191 g SiO₂ cartridge and purified on a 1.5 Kg SiO₂ column. The column was eluted using a 0%-20% EtOAc/hexanes gradient as the mobile phase to provide 52 grams of pure Int-6d and additional fractions of Int-6d that contained a small amount of a Boc-valine impurity. The impure fractions from the two columns were recombined and re-purified. After chromatography, compound Int-6d was obtained as an oil which solidified to a white solid on standing (128 g, 65% yield over the three steps.)

### Step C—Synthesis of Intermediate Compound Int-6e

A solution of Int-6d (8.5 g, 31.1 mmol) in methanol (100 mL) and 1.0 M aqueous KOH solution (48 mL, 48 mmol) was allowed to stir at room temperature for about 15 hours, neutralized with 48 mL of 1.0 M aqueous HCl solution to pH ˜5, and concentrated in vacuo to an oil. The resulting resulting residue was extracted with dichloromethane (2×100 mL) and the combined organic layers were concentrated in vacuo to provide Compound Int-6e as a gel (7.74 g, 96%). Chiral purity was determined using a Chiralcell AD-H column, SFC mode, CO₂/MeOH 90/10.

### Example 7

### Preparation of Intermediate Compound Int-7g

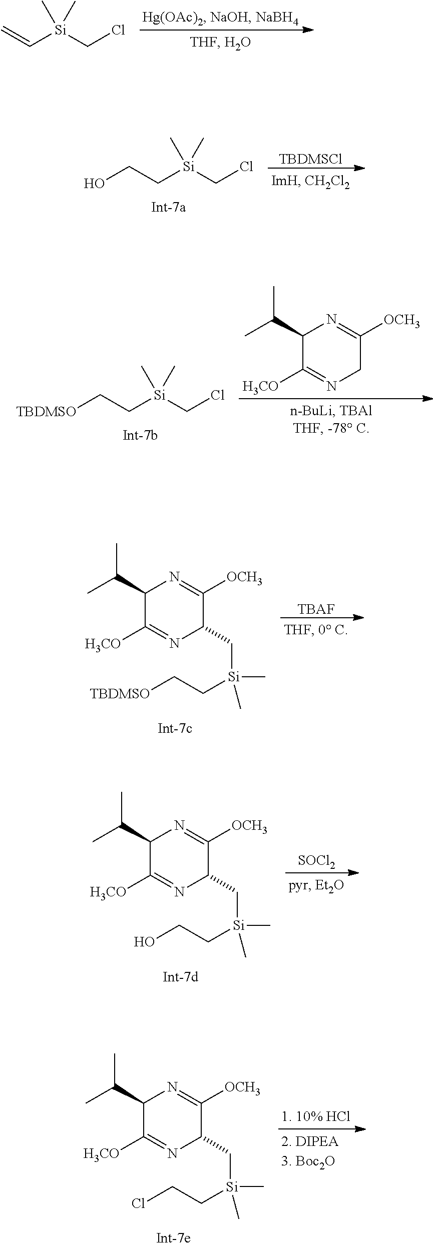

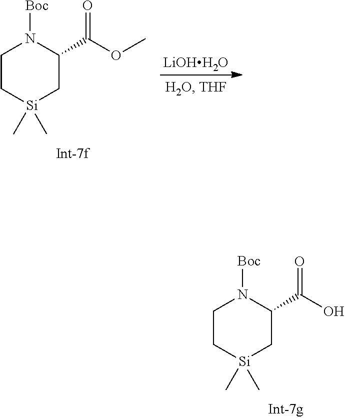

### Step A—Synthesis of Intermediate Compound Int-7a

Mercuric acetate (14.3 g, 44.8 mmol) was dissolved in water (45 mL), and THF (45 mL) was added. To this yellow solution at room temperature was added (chloromethyl)-dimethylvinylsilane (5.65 g, 41.9 mmol) which became homogeneous in 30 seconds. The resulting solution was allowed to stir for 5 minutes, then aqueous NaOH (3M, 45 mL) was added, followed by a solution (45 mL) of NaBH₄ (0.5M) in 3M NaOH. Diethyl ether (160 mL) was added and the mixture stirred at room temperature for and additional 1 hr. The mixture was then saturated with NaCl and the layers separated. The organic layer was washed with brine (100 mL), dried with Na₂SO₄, and concentrated in vacuo to provide Compound Int-7a as a colorless oil (5.72 g, 89%). ¹H NMR (CDCl₃) δ 3.84-3.75 (m, 2H), 2.81 (s, 2H), 1.34-1.31 (m, 1H), 1.10-1.05 (m, 2H), 0.148 (s, 6H).

### Step B—Synthesis of Intermediate Compound Int-7b

To a solution of Int-7a (5.72 g, 37.4 mmol) in CH₂Cl₂ (50 mL) was added imidazole (3.82 g, 56.1 mmol). The mixture was allowed to stir at 0° C. and tert-butyldimethylsilyl chloride (8.46 g, 56.1 mmol) was slowly added over 10 minutes and the reaction mixture was warmed to room temperature and allowed to stir for about 15 hours. Water (50 mL) was added and the layers separated. The aqueous layer was extracted with CH₂Cl₂ (3×30 mL) and the combined organic layers were dried over Na₂SO₄, filtered and concentrated in vacuo at 80° C. to remove residual tert-butyldimethylsilyl chloride and afford the desired product Int-7b as a colorless oil (9.82 g, 98%). ¹H NMR (CDCl₃) δ 3.75 (t, J=7.4 Hz, 2H), 2.78 (s, 2H), 0.99 (t, J=7.4 Hz, 2H), 0.87 (s, 9H), 0.011 (s, 6H), 0.02 (s, 6H).

### Step C—Synthesis of Intermediate Compound Int-7c

To a solution of (R)-2-isopropyl-3,6-dimethoxy-2,5-dihydropyrazine (6.16 g, 33.4 mmol) in THF (60 mL) was added TBAI (617 mg, 1.67 mmol). The mixture was cooled to −78° C. and a solution of n-BuLi (14.7 mL, 2.5M in hexanes, 36.75 mmol) was slowly added over 10 minutes. The reaction mixture was allowed to stir at −78° C. for 30 minutes, then Int-7b in THF (20 mL) was slowly added over 10 minutes. The reaction was allowed to stir at −78° C. for 2 hours then allowed to warmed to room temperature and allowed to stir for about 15 hours. The reaction was quenched by addition of MeOH (5 mL), concentrated in vacuo, water added (50 mL) followed by diethyl ether (50 mL) and the layers were separated. The organic layer was washed with water (2×50 mL) then dried over Na₂SO₄, filtered and concentrated in vacuo to provide the crude product. Further purification by column chromatography on a 330 g ISCO Redi-Sep silica gel column using a eluent of CH₂Cl₂ with a gradient of 0-10% EtOAc/hexanes afforded the desired product Int-7c as a light amber oil (8.65 g, 63%). ¹H NMR (CDCl₃) δ 4.07-3.99 (m, 1H), 3.94-3.89 (m, 1H), 3.79-3.71 (m, 2H), 3.68-3.63 (m, 611), 2.32-2.17 (m, 1H), 1.25-1.21 (m, 1H), 1.06-0.95 (m, 5H), 0.88 (s, 10H), 0.74-0.68 (m, 1H), 0.69-0.66 (m, 2H), 0.12-0.02 (m, 12H).

### Step D—Synthesis of Intermediate Compound Int-7d

To a THF solution (60 mL) of Int-7c (8.65 g, 20.8 mmol) cooled to 0° C. was slowly added a solution of tetrabutylammonium fluoride (31.3 mL, 1.0M in THF, 31.0 mmol) over 5 minutes. The reaction mixture was allowed to warm to room temperature for about 15 hours with stirring. The reaction was then concentrated in vacuo, and the crude product chromatographed on a 120 g ISCO Redi-Sep silica gel column using a CH₂Cl₂ with gradient of 0-3% MeOH/CH₂Cl₂ as the eluent to provide Compound Int-7d as a colorless oil (4.69 g, 99%). ¹H NMR (CDCl₃) b 4.15-4.05 (m, 1H), 3.98-3.91 (m, 1H), 3.84-3.73 (m, 2H), 3.69 (s, 6H), 2.39-2.32 (m, 1H), 2.30-2.18 (m, 1H), 1.37-1.29 (m, 1H), 1.10-1.01 (m, 5H), 0.93-0.85 (m, 2H), 0.74-0.68 (m, 2H), 0.14-0.08 (m, 6H).

### Step F—Synthesis of Intermediate Compound Int-7e

To a Et₂O (30 mL) solution of compound Int-7d (2.12 g, 267 mmol) was added pyridine (720 μL, 8.82 mmol). The mixture was cooled to 0° C. and thionyl chloride (575 μL, 7.90 mmol) in Et₂O (2 mL) was slowly added over 5 minutes. The reaction mixture was allowed to warm to room temperature for about 15 hours with stirring. The reaction mixture was filtered and the filtrate concentrated in vacuo to provide the crude product. Further purification by column chromatography using a 80 g ISCO Redi-Sep silica gel column with CH₂Cl₂ and a gradient of 0-3% MeOH as the eluent provided compound Int-7e as an amber oil (417 mg, 16%). ¹H NMR (CDCl₃) δ 4.22-3.62 (m, 7H), 2.50-2.13 (m, 4H), 1.58-1.41 (m, 1H), 1.32-0.65 (m, 9H), 0.24-0.04 (m, 6H).

### Step G—Synthesis of Intermediate Compound Int-7f

To a solution of Int-7e (417 mg, 1.40 mmol) in MeOH (10 mL) was added a 10% aqueous HCl solution (10 mL). The resulting mixture was allowed to stir at room temperature for about 15 hours and concentrated in vacuo. The resulting resulting residue was coevaporated with MeOH (3×30 mL) and then dissolved in CH₂Cl₂ (3 mL) and Et₂O (6 mL). To this solution was added diisopropylethylamine (750 μL, 4.30 mmol) and the reaction allowed to stir at room temperature After 7 hours di-tert-butyl dicarbonate (703 mg, 3.22 mmol) was added and the reaction was allowed to stir for about 15 hours at room temperature and then concentrated in vacuo. The crude product was further purified using column chromatographed using a 12 g ISCO Redi-Sep silica gel column with CH₂Cl₂ and gradient of 0-50% EtOAc/hexanes mixture as the eluent to provide Compound Int-7f as an amber oil (94 mg, 23%). ¹H NMR (CDCl₃) δ 4.22-4.01 (m, 1H), 4.10-3.94 (m, 1H), 3.85-3.70 (m, 3H), 2.32-2.09 (m, 1H), 1.44 (s, 7H), 1.24-0.88 (m, 6H), 0.16-0.05 (m, 6H).

### Step H—Synthesis of Intermediate Compound Int-7g

To a solution of compound Int-7f (218 mg, 0.758 mmol) in THF (3 mL) was added lithium hydroxide monohydrate (64 mg, 1.52 mmol) in water (3 mL). The reaction mixture was allowed to stir at room temperature for about 15 hours then concentrated in vacuo to half volume. The aqueous mixture was then acidified with 1N HCl to pH 4 and extracted with EtOAc (5×30 mL). The combined organic layers were dried over Na₂SO₄, filtered and concentrated in vacuo to provide Compound Int-7g as an off-white solid (157 mg, 87%). ¹H NMR (CDCl₃) 81.44 (s, 8H), 1.34-0.78 (m, 9H), 0.17-0.03 (m, 6H).

### Example 8

### Preparation of Intermediate Compound Int-8f

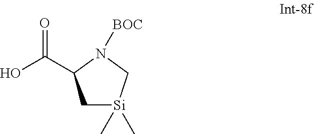 [SMILES: CC(C)(C)OC(=O)N1C[Si](C)(C)C[C@H]1C(=O)O]

### Step A—Synthesis of Intermediate Compound Int-8b

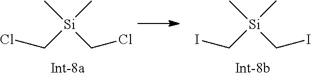

Bis(chloromethyl)dimethylsilane (Int-8a, 50 g, 0.32 mol), sodium iodide (181 g, 1.21 mol), and dried acetone (1 liter) were added to a 2-liter round-bottomed flask. The resulting suspension was refluxed with stirring for 3.5 hours before cooled to room temperature. After filtration, the filtrate was concentrated in vacuo and the residue obtained was treated with ethyl acetate (500 mL). The suspension was filtered again and the residue obtained was concentrated in vacuo to provide Int-8b as an oil (90.5 g, 84%). This material was pure enough for the next reaction.

### Step B—Synthesis of Intermediate Compound Int-8d

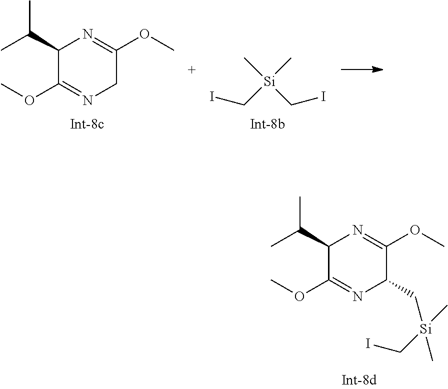

(R)-2,5-Dihydro-3,6-dimethoxy-2-isopropylpyrazine (Int-8c, 25 g, 135.7 mmol) and dried THF (500 mL) were added to a dried 1-liter flask which was cooled to −78° C. and maintained under nitrogen atmosphere. A solution of 2.5 M n-BuLi in hexane (54 mL, 135 mmol) was added slowly via a syringe. The resulting solution was allowed to stir at the cold temperature for 30 minutes before addition of Int-8b (90.5 g, 266.2 mmol) via a syringe. The reaction mixture was continued to stir for 4 hours and warmed to room temperature gradually over a period of 1 hour. After addition of water (100 mL) and diethyl ether (1.0 liter), the solution was washed with water (2×200 mL) and dried over sodium sulfate. The solution was concentrated in vacuo and the residue obtained was purified using a 330 g ISCO silica column on Combi-Flash with 0-1% ether in hexanes as an eluent to provide Int-8d as an oil (18.5 g, 35%).

### Step C—Synthesis of Intermediate Compound Int-8e

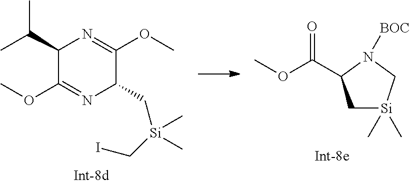

The intermediate material Int-8d (18.5 g, 46.7 mmol) was dissolved in methanol (105 mL) in a 500 mL flask. 35 mL of 10% aqueous HCl solution was added slowly. The resulting mixture was allowed to stir at room temperature for 5 hours and concentrated to dryness. The residue obtained was co-evaporated 4 times with methanol (120 mL) and then dissolved in dichloromethane (80 mL) and diethyl ether (120 mL). To this solution was added N,N-diisopropylethylamine (18 mL, 135 mmol). The reaction mixture was allowed to stir at room temperature for 7 hours prior to addition of di-tert-butyl dicarbonate (23.5 g, 108 mmol). The solution was continued to stir at room temperature for about 15 hours and concentrated in vacuo. The residue obtained was taken up with ethyl acetate (300 mL), washed with water (200 mL), dried over sodium sulfate, and concentrated again. The crude product was purified using a 330 g ISCO silica column with 0-20% ethyl acetate in hexanes to provide 5 as a colorless oil (8.5 g, 67%).

### Step D—Synthesis of Intermediate Compound Int-8f

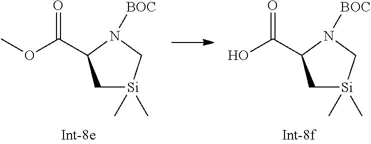

A solution of Int-8e (8.5 g, 31.1 mmol) in methanol (100 mL) and 1.0 M aqueous KOH solution (48 mL, 48 mmol) was allowed to stir at room temperature for about 15 hours, neutralized with 48 mL of 1.0 M aqueous HCl solution to PH ˜5, and concentrated to dryness. The residue obtained was extracted with dichloromethane (2×100 mL) The combined organic solutions were concentrated in vacuo to provide 6 as a gel (7.74 g, 96%).

### Example 9

### Preparation of Intermediate Compound Int-9c

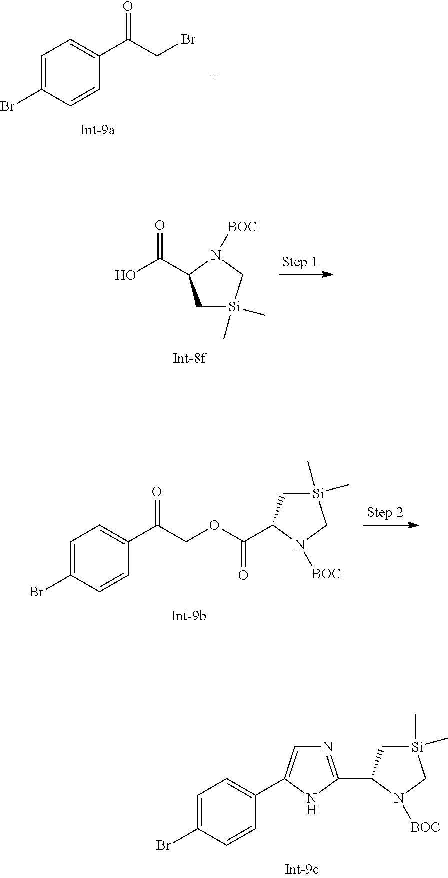 [SMILES: O=C(CBr)c1ccc(Br)cc1 ; CC(C)(C)OC(=O)N1C[Si](C)(C)C[C@H]1C(=O)O ; CC(C)(C)OC(=O)N1C[Si](C)(C)C[C@H]1C(=O)OCC(=O)c1ccc(Br)cc1 ; CC(C)(C)OC(=O)N1C[Si](C)(C)C[C@H]1c1ncc(-c2ccc(Br)cc2)[nH]1]

### Step 1—Synthesis of Intermediate Int-9b

The starting materials Int-9a (9.0 g, 32.4 mmol) and Int-8f (7.74 g, 29.85 mmol) were dissolved in DMF (50 mL). Triethylamine (10 mL, 71.83 mmol) was added slowly at room temperature. The mixture was allowed to stir at this temperature for about 15 hours, diluted with ethyl acetate (500 mL), washed with brine (3×100 mL), dried over sodium sulfate, and concentrated in vacuo. The residue obtained was purified using a 220 g ISCO silica column with 0-20% ethyl acetate in hexanes as an eluent to provide Int-9b as a gel (12.3 g, 83%).

### Step 2—Synthesis of Intermediate Int-9c

A mixture of Int-9b (12.3 g, 26.96 mmol), ammonium acetate (18.0 g, 233.68 mmol), and xylenes (50 mL) in a 350 mL pressure vessel was allowed to stir at 120° C. for two hours. After cooling to room temperature, the suspension was concentrated in vacuo. The residue obtained was dissolved in ethyl acetate (300 mL), washed with water (100 mL) and saturated sodium carbonate solution (100 mL), dried over sodium sulfate, and concentrated in vacuo. The residue obtained was then purified using a 330 g ISCO silica column with 10-50% ethyl acetate in hexanes as an eluent to provide Int-9c as a pale solid (8.5 g, 72%).

### Example 10

### Preparation of Intermediate Compound Int-10a

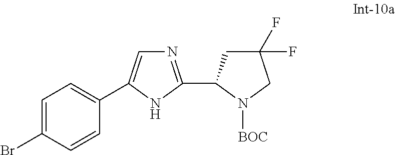 [SMILES: CC(C)(C)OC(=O)N1CC(F)(F)C[C@H]1c1ncc(-c2ccc(Br)cc2)[nH]1]

Intermediate Int-10a was prepared from the commercially available N-Boc-4,4-difluoro-L-proline (Aldrich) using the method described in Example 9.

### Example 11

### Preparation of Intermediate Compound Int-11a

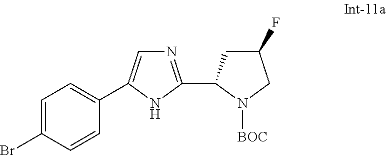 [SMILES: CC(C)(C)OC(=O)N1C[C@H](F)C[C@H]1c1ncc(-c2ccc(Br)cc2)[nH]1]

Intermediate Int-11a was prepared from the commercially available N-Boc-trans-fluoro-L-proline (Alfa) using the method described in Example 9.

### Example 12

### Preparation of Intermediate Compound Int-12a

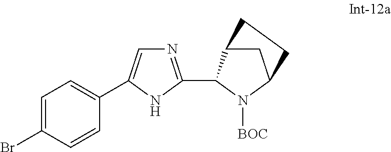 [SMILES: CC(C)(C)OC(=O)N1[C@@H]2CC[C@@H](C2)[C@H]1c1ncc(-c2ccc(Br)cc2)[nH]1]

Intermediate Int-12a was prepared from the commercially available (1R,3S,4S)—N-Boc-2-azabicyclo[2.2.1]-heptane-3-carboxylic acid (Aldrich) using the method described in Example 9.

### Example 13

### Preparation of Intermediate Compound Int-13a

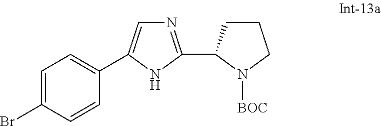 [SMILES: CC(C)(C)OC(=O)N1CCC[C@H]1c1ncc(-c2ccc(Br)cc2)[nH]1]

Intermediate Int-13a was prepared from commercially available N-Boc-L-proline (Aldrich) using the method described in Example 9.

### Example 14

### Preparation of Intermediate Compound Int-14a

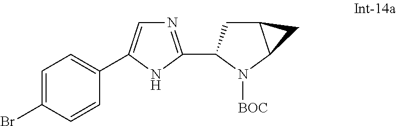 [SMILES: CC(C)(C)OC(=O)N1[C@@H]2C[C@@H]2C[C@H]1c1ncc(-c2ccc(Br)cc2)[nH]1]

Intermediate Int-14a was prepared from commercially available (1S,3S,5R)-2-(tert-butoxycarbonyl)-2-azabicyclo[3.1.0]hexane-3-carboxylic acid (Wuxi Apptech Co.), using the method described in Example 9.

### Example 15

### Preparation of Intermediate Compound Int-15a

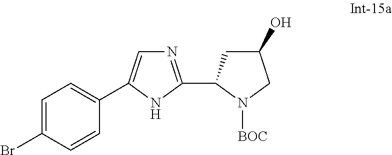 [SMILES: CC(C)(C)OC(=O)N1C[C@H](O)C[C@H]1c1ncc(-c2ccc(Br)cc2)[nH]1]

Intermediate Int-15a was prepared from BOC—HYP—OH, which is commercially available from Aldrich, using the method described in Example 9.

### Example 16

### Preparation of Intermediate Compound Int-16a

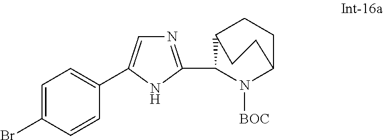 [SMILES: CC(C)(C)OC(=O)N1C2CCC(CC2)[C@H]1c1ncc(-c2ccc(Br)cc2)[nH]1]

Intermediate Int-16a was prepared from 2(S)-azabicyclo[2.2.2]-octane-2,3-dicarboxylic acid 2-tert-butyl ester, which is commercially available from Wuxi Apptech Co., using the method described in Example 9.

### Example 17

### Preparation of Intermediate Compound Int-17a

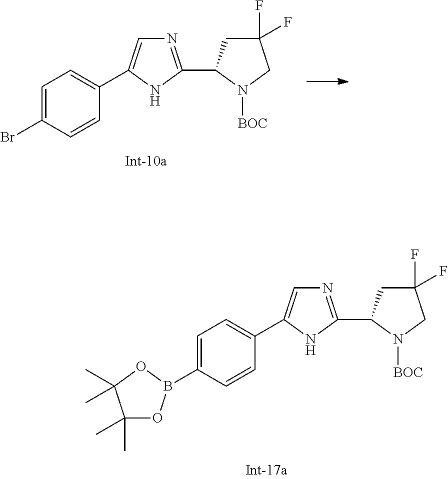 [SMILES: CC(C)(C)OC(=O)N1CC(F)(F)C[C@H]1c1ncc(-c2ccc(Br)cc2)[nH]1 ; CC(C)(C)OC(=O)N1CC(F)(F)C[C@H]1c1ncc(-c2ccc(B3OC(C)(C)C(C)(C)O3)cc2)[nH]1]

Int-10a (5.7 g, 13.31 mmol), bis(pinacolaton)diboron (6.8 g, 26.78 mmol), tetrakis(triphenylphosphine) palladium (0) (0.76 g, 0.66 mmol) and potassium acetate (2.0 g, 20.37 mmol) were taken up in dioxane (15 mL). The resulting suspension was degassed and stirred at 80° C. for about 15 hours. After cooling to room temperature, the mixture was filtered and the filtrate was concentrated in vacuo. The resulting residue was purified using a 220 g ISCO silica column on Combi-Flash Rf with elution of 0-4% methanol in dichloromethane to provide Int-17a as a wax (5.4 g, 85%).

### Example 18

### Preparation of Intermediate Compound Int-18a

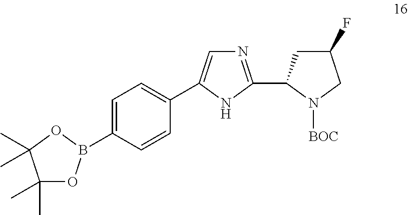 [SMILES: CC(C)(C)OC(=O)N1C[C@H](F)C[C@H]1c1ncc(-c2ccc(B3OC(C)(C)C(C)(C)O3)cc2)[nH]1]

Intermediate Int-18a was prepared from intermediate bromide Int-11a using the method described in Example 17.

### Example 19

### Preparation of Intermediate Compound Int-19a

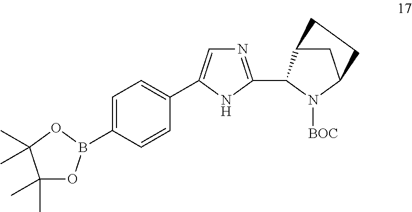 [SMILES: CC(C)(C)OC(=O)N1[C@@H]2CC[C@@H](C2)[C@H]1c1ncc(-c2ccc(B3OC(C)(C)C(C)(C)O3)cc2)[nH]1]

Intermediate Int-19a was prepared from intermediate bromide Int-12a using the method described in Example 17.

### Example 20

### Preparation of Intermediate Compound Int-20a

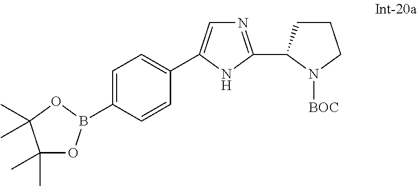 [SMILES: CC(C)(C)OC(=O)N1CCC[C@H]1c1ncc(-c2ccc(B3OC(C)(C)C(C)(C)O3)cc2)[nH]1]

Intermediate Int-20a was prepared from intermediate bromide Int-13a using the method described in Example 17.

### Example 21

### Preparation of Intermediate Compound Int-21a

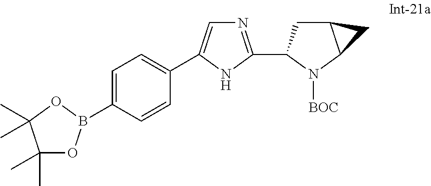 [SMILES: CC(C)(C)OC(=O)N1[C@@H]2C[C@@H]2C[C@H]1c1ncc(-c2ccc(B3OC(C)(C)C(C)(C)O3)cc2)[nH]1]

Intermediate Int-21a was prepared from intermediate bromide Int-14a using the method described in Example 17.

### Example 22

### Preparation of Intermediate Compound Int-22a

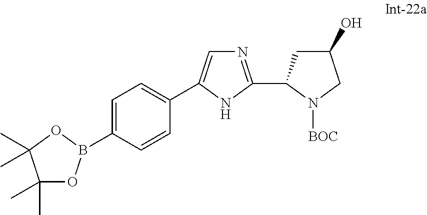 [SMILES: CC(C)(C)OC(=O)N1C[C@H](O)C[C@H]1c1ncc(-c2ccc(B3OC(C)(C)C(C)(C)O3)cc2)[nH]1]

Intermediate Int-22a was prepared from intermediate bromide Int-15a using the method described in Example 17.

### Example 23

### Preparation of Intermediate Compound Int-23a

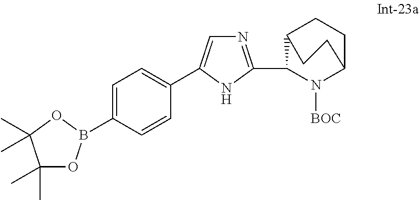 [SMILES: CC(C)(C)OC(=O)N1C2CCC(CC2)[C@H]1c1ncc(-c2ccc(B3OC(C)(C)C(C)(C)O3)cc2)[nH]1]

Intermediate Int-23a was prepared from intermediate bromide Int-16a using the method described in Example 17.

### Example 24

### Preparation of Intermediate Compound Int-24a

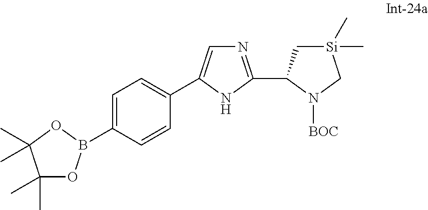

Intermediate Int-24a was prepared from intermediate bromide Int-9c using the method described in Example 17.

### Example 25

### Preparation of Intermediate Compound Int-25c

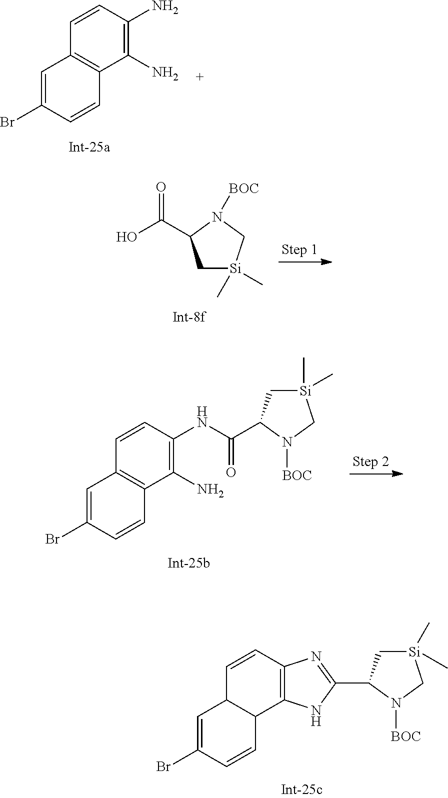 [SMILES: Nc1ccc2cc(Br)ccc2c1N ; CC(C)(C)OC(=O)N1C[Si](C)(C)C[C@H]1C(=O)O ; CC(C)(C)OC(=O)N1C[Si](C)(C)C[C@H]1C(=O)Nc1ccc2cc(Br)ccc2c1N ; CC(C)(C)OC(=O)N1C[Si](C)(C)C[C@H]1c1nc2c([nH]1)C1C=CC(Br)=CC1C=C2]

### Step 1—Synthesis of Intermediate Int-25a

A solution of compound Int-25a (2.7 g, 11.4 mmol), compound Int-8f (2.2 g, 7.77 mmol), Hunig's base (2 mL, 15 mmol), and HATU (3.0 g, 7.89 mmol) was cooled to 0° C. and allowed to stir at this temperature for The resulting 6.5 hours. The reaction mixture was then diluted with water (150 mL) and filtered. The collected solid was purified using a 330 g ISCO silica column on Combi-Flash Rf with elution of 0-5% methanol in dichloromethane to provide compound Int-25b as a foam (3.55 g, 96%).

### Step 2—Synthesis of Intermediate Int-25c

A mixture of compound Int-25b (2.0 g, 4.18 mmol) and acetic acid (20 mL) was allowed to stir at 60° C. for 5 hours and then cooled to room temperature. After evaporation of acetic acid in vacuo, the residue obtained was purified using a 120 g ISCO silica column on Combi-Flas RF with elution of 0-5% methanol in dichloromethane to provide compound Int-25c as a solid (1.56 g, 81%).

Intermediate compounds Int-25d to Int-25g were prepared using the method above and substituting the appropriate reactants and/or reagents.

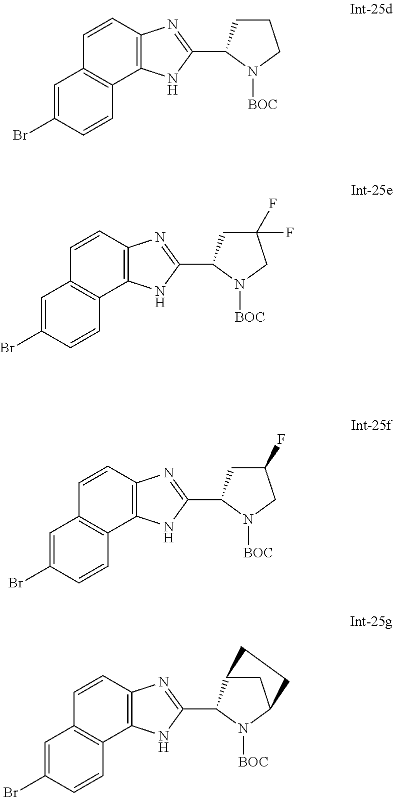 [SMILES: CC(C)(C)OC(=O)N1CCC[C@H]1c1nc2ccc3cc(Br)ccc3c2[nH]1 ; CC(C)(C)OC(=O)N1CC(F)(F)C[C@H]1c1nc2ccc3cc(Br)ccc3c2[nH]1 ; CC(C)(C)OC(=O)N1C[C@H](F)C[C@H]1c1nc2ccc3cc(Br)ccc3c2[nH]1 ; CC(C)(C)OC(=O)N1[C@@H]2CC[C@@H](C2)[C@H]1c1nc2ccc3cc(Br)ccc3c2[nH]1]

### Example 26

### Preparation of Intermediate Compound Int-26a

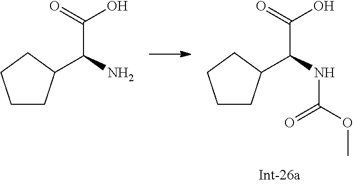

To a round bottom flask charged with a stir bar was added L-cyclopentylglycine (2.5 g, 17 mmol) followed by 1N NaOH (17.5 mL) The mixture was stirred for 10 min at rt whereupon sodium carbonate (0.93 g, 8.7 mmol) was added and the mixture was cooled to 0° C. Methyl chloroformate (1.5 mL, 19 mmol) was added dropwise and the mixture was allowed to warm to room temperature and allowed to stir for 12 hours, then Et₂O (50 mL) was added. The organic layer was decanted off and this procedure was repeated an additional two times. The remaining aqueous layer was cooled to 0° C. whereupon conc. HCl was added dropwise to pH=2 whereupon the mixture was diluted with CH₂Cl₂ (100 mL) The layers were separated and the aqueous layer was extracted with CH₂Cl₂ (2×100 mL). The organic layers were combined, washed with brine (1×50 mL), dried (Na₂SO₄), filtered, and concentrated under reduced pressure. The resulting clear oil was placed under high vacuum to provide 3.18 g (85%) of compound Int-26a a white solid.

### Example 27

### Preparation of Intermediate Compound Int-27a

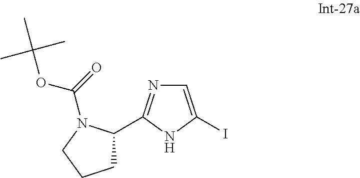 [SMILES: CC(C)(C)OC(=O)N1CCC[C@H]1c1ncc(I)[nH]1]

Intermediate compound Int-27a was made using the method described in International Publication No. WO 2010065668.

### Example 28

### Preparation of Compounds 1-3

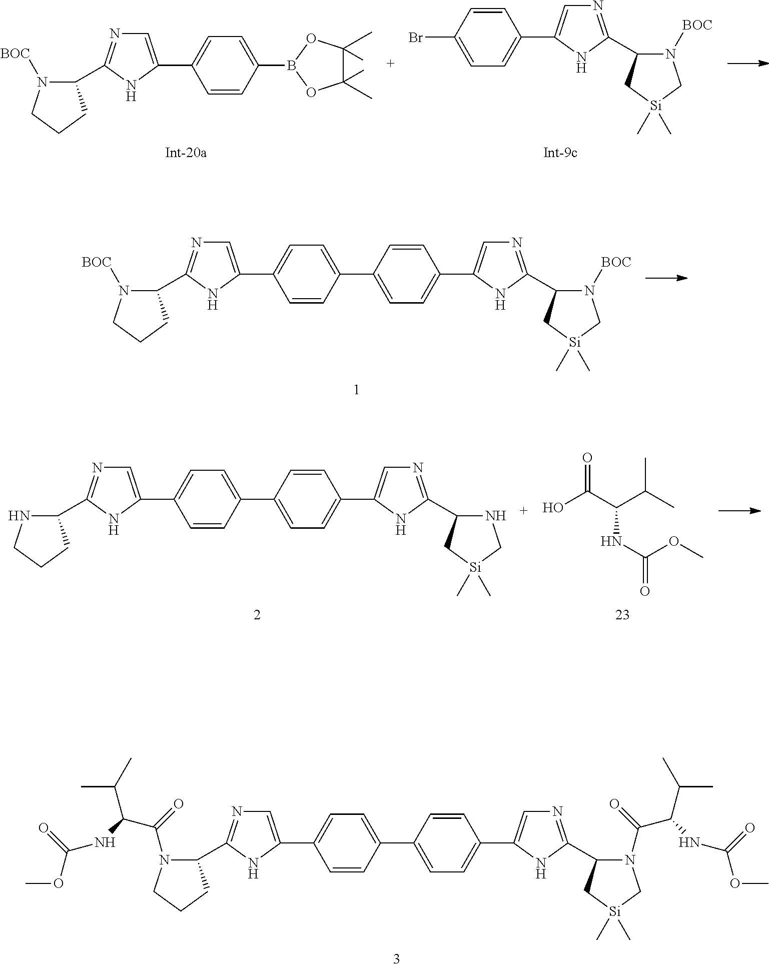

### Step A—Synthesis of Compound 1

Compound Int-20a (450 mg, 1.02 mmol), compound Int-9c (300 mg, 0.687 mmol), PdCl₂dppf dichloromethane complex (60 mg, 0.073 mmol), a solution of sodium carbonate (1.5M, 1.0 mL, 1.5 mmol), and 1,4-dioxane (8 mL) were added to a 200 ml flask. The resulting mixture was degassed and refluxed under nitrogen atmosphere for 6 hours. After cooled to room temperature and concentrated, the resulting residue was purified using a 120 g ISCO silica column on Combi-Flash with 0-4% methanol in dichloromethane as the eluent to provide 1 as a white solid (270 mg, 59%). LCMS anal. calcd. for: C₃₇H₄₈N₆O₄Si 668.4. Found: 669.3 (M+H)⁺.

### Step B—Synthesis of Compound 2

Compound 1 (270 mg, 0.404 mmol) was dissolved in dichloromethane (3 mL) and trifluoroacetic acid (3 mL). The resulting solution was stirred at room temperature for 4 hours and then concentrated under vacuum to provide compound 2 as a white solid (190 mg), which was used for the next reaction without purification.

### Step C—Synthesis of Compound 3

Compound 2 (190 mg, ˜0.4 mmol), Int-1a (160 mg, 0.913 mmol), Hunig's base (0.4 mL, 3.0 mmol), HATU (350 mg, 0.92 mmol), and DMF (3 mL) were added to a 100 ml flask at 0° C. The resulting solution was stirred at room temperature for 2 hours. The purification of the reaction solution by Gilson reverse phase chromatography (0-90% acetonitrile in water with 0.1% TFA as an eluent) provided 3 as a white solid (100 mg, 32%). LCMS anal. calcd. for: C₄₁H₅₄N₈O₆Si 782.4. Found: 783.3 (M+H)⁺.

Compounds 4-9, depicted in the table below, were made using the methods described in the Example above and substituting the appropriate reactants and/or reagents.

| | | | | | 1a | |
|---|---|---|---|---|---|---|
| | | | 1a | 1b | Y93H | 2b |
| | | Mass | EC90 | EC90 | EC90 | EC90 |
| No. | Structure | Obsvd | nM | nM | nM | nM |
| 2 | 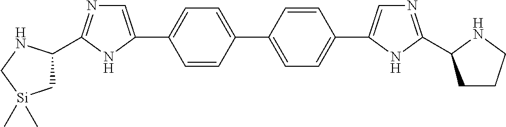 [SMILES: C[Si]1(C)CN[C@H](c2ncc(-c3ccc(-c4ccc(-c5cnc([C@@H]6CCCN6)[nH]5)cc4)cc3)[nH]2)C1] | 469.2 | >100 | >1000 | — | — |
| 3 | 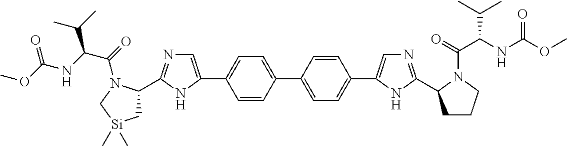 | 783.3 | 0.16 | 0.005 | 398 | — |
| 4 | 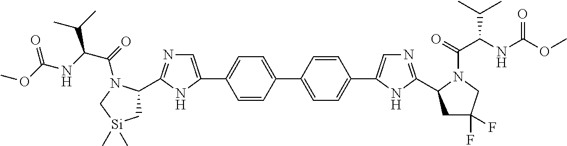 | 819.4 | 0.10 | 0.007 | 272 | — |
| 5 | 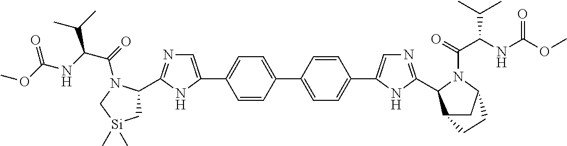 | 809.4 | 0.21 | 0.004 | 203 | — |
| 6 | 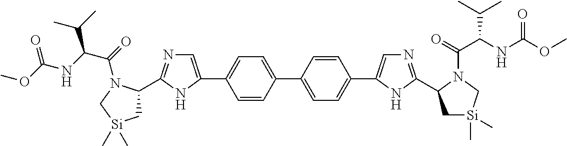 | 827.4 | 0.98 | 0.015 | >1000 | — |
| 7 | 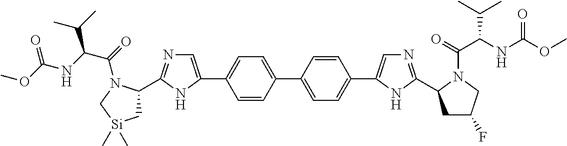 | 801.4 | 0.77 | 0.009 | 229 | — |
| 8 | 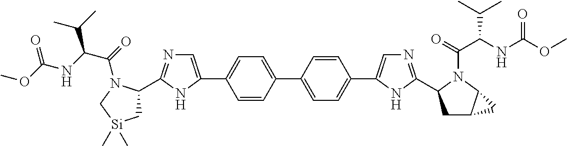 | 794.7 | 0.029 | 0.002 | >100 | 249 |
| 9 | 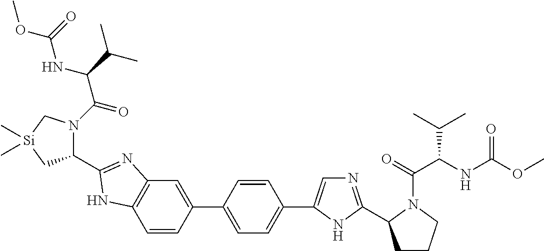 | 757.4 | >1 | <1 | >1000 | — |

### Example 29

### Preparation of Compounds 10-12

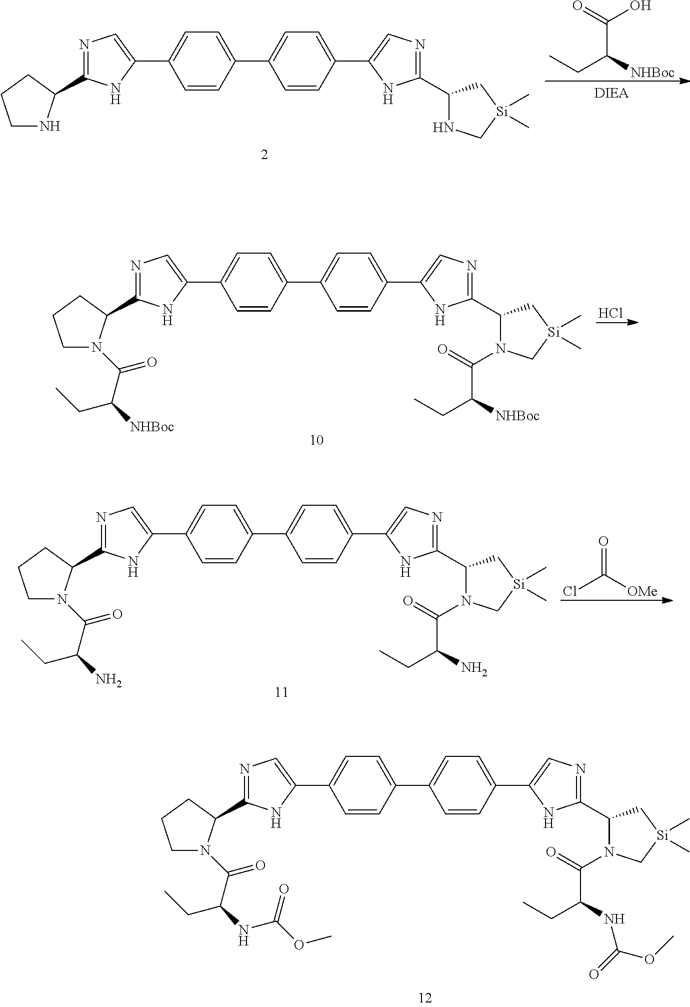

Compound 1 (32 mg, 0.068 mmol), (S)-2-(tert-butoxycarbonylamino)butanoic acid (35 mg, 0.171 mmol) and DIEA (59.6 μl, 0.341 mmol) were taken up in a mixture of acetonitrile (350 μl) and THF (350 μl). The reaction was shaken for 1 minute, followed by addition of 1-propanephosphonic acid cyclic anhydride (61 μl, 0.205 mmol, 40% in EtOAc) and shaking at 25° C. for 18 hours. MS confirmed the formation of compound 10, then 0.3 mL of 4N HCl in dioxane was added into reaction mixture. The reaction was allowed to stir for 3 hours, then was concentrated in vacuo to provide compound 11. Compound 11 was then dissolved in 0.5 mL THF & 0.5 mL of acetonitrile followed by addition of methyl chloroformate (25.8 mg, 0.273 mmol) and the reaction was shaked for 18 hours. 4N HCl 0.2 mL was added into mixture, which was then allowed to stir for 3 hours at room temperature, then concentrated in vacuo. The resulting residue was dissolved in 1 mL DMSO, filtered and further purified using reverse-phase LC to provide compound 12 (8.8 mg, 0.01165 mmol, 17.1% overall yield).

Compounds 13-23, depicted in the table below, were made using the methods described in the Example above and substituting the appropriate reactants and/or reagents.

| | | | | | 1a | |
|---|---|---|---|---|---|---|
| | | | 1a | 1b | Y93H | 2b |
| | | Mass | EC90 | EC90 | EC90 | EC90 |
| No. | Structure | Obsvd | nM | nM | nM | nM |
| 12 | 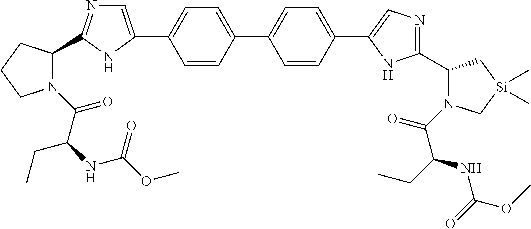 [SMILES: CC[C@H](NC(=O)OC)C(=O)N1CCC[C@H]1c1ncc(-c2ccc(-c3ccc(-c4cnc([C@@H]5C[Si](C)(C)CN5C(=O)[C@H](CC)NC(=O)OC)[nH]4)cc3)cc2)[nH]1] | 755.9 | 0.187 | 18.15 | 693.8 | 534.6 |
| 13 | 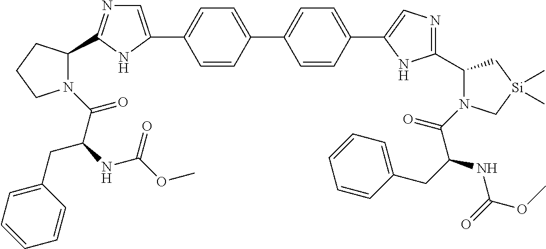 [SMILES: COC(=O)N[C@@H](Cc1ccccc1)C(=O)N1CCC[C@H]1c1ncc(-c2ccc(-c3ccc(-c4cnc([C@@H]5C[Si](C)(C)CN5C(=O)[C@H](Cc5ccccc5)NC(=O)OC)[nH]4)cc3)cc2)[nH]1] | 880.0 | 29.83 | 923.3 | 1000 | 1000 |
| 14 | 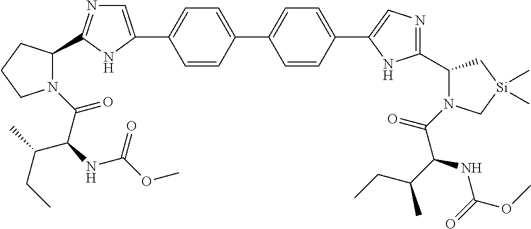 [SMILES: CC[C@H](C)[C@H](NC(=O)OC)C(=O)N1CCC[C@H]1c1ncc(-c2ccc(-c3ccc(-c4cnc([C@@H]5C[Si](C)(C)CN5C(=O)[C@@H](NC(=O)OC)[C@@H](C)CC)[nH]4)cc3)cc2)[nH]1] | 812.0 | 3.22 | 23.8 | 1000 | 1000 |
| 15 | 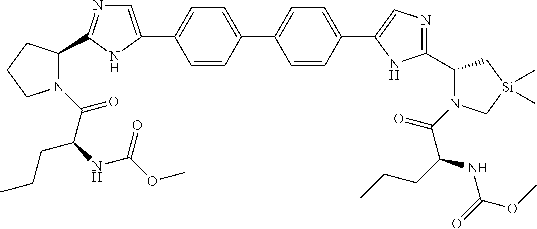 [SMILES: CCC[C@H](NC(=O)OC)C(=O)N1CCC[C@H]1c1ncc(-c2ccc(-c3ccc(-c4cnc([C@@H]5C[Si](C)(C)CN5C(=O)[C@H](CCC)NC(=O)OC)[nH]4)cc3)cc2)[nH]1] | 783.9 | 2.426 | 45.8 | 541.7 | 515.3 |
| 16 | 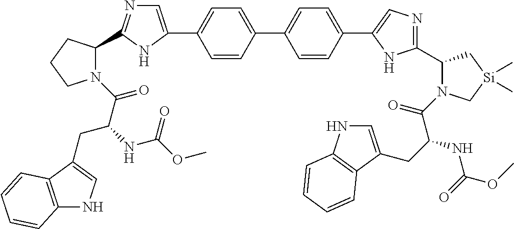 [SMILES: COC(=O)N[C@H](Cc1c[nH]c2ccccc12)C(=O)N1CCC[C@H]1c1ncc(-c2ccc(-c3ccc(-c4cnc([C@@H]5C[Si](C)(C)CN5C(=O)[C@@H](Cc5c[nH]c6ccccc56)NC(=O)OC)[nH]4)cc3)cc2)[nH]1] | 958.0 | 1000 | 1000 | 500 | 1000 |
| 17 | 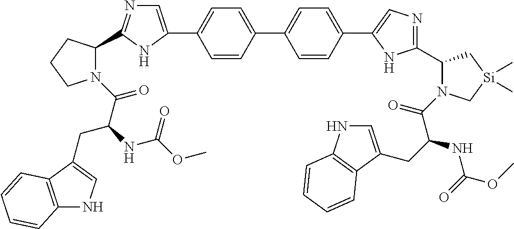 [SMILES: COC(=O)N[C@@H](Cc1c[nH]c2ccccc12)C(=O)N1CCC[C@H]1c1ncc(-c2ccc(-c3ccc(-c4cnc([C@@H]5C[Si](C)(C)CN5C(=O)[C@H](Cc5c[nH]c6ccccc56)NC(=O)OC)[nH]4)cc3)cc2)[nH]1] | 958.1 | 9.361 | 1000 | 1000 | 1000 |
| 18 | 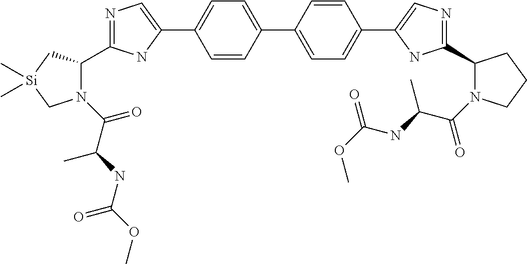 [SMILES: COC(=O)N[C@@H](C)C(=O)N1CCC[C@@H]1c1ncc(-c2ccc(-c3ccc(-c4cnc([C@H]5C[Si](C)(C)CN5C(=O)[C@H](C)NC(=O)OC)[nH]4)cc3)cc2)[nH]1] | 727.8 | 987.6 | 1000 | 1000 | 1000 |
| 19 | 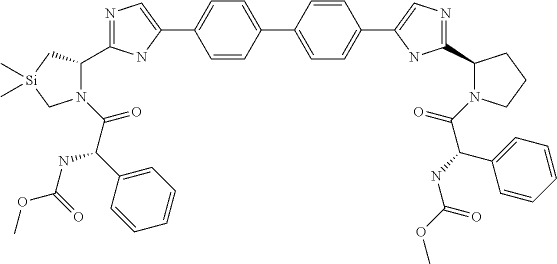 [SMILES: COC(=O)N[C@H](C(=O)N1CCC[C@@H]1c1ncc(-c2ccc(-c3ccc(-c4cnc([C@H]5C[Si](C)(C)CN5C(=O)[C@@H](NC(=O)OC)c5ccccc5)[nH]4)cc3)cc2)[nH]1)c1ccccc1] | 852.0 | 25.54 | 11.04 | 1000 | 999.1 |
| 20 | 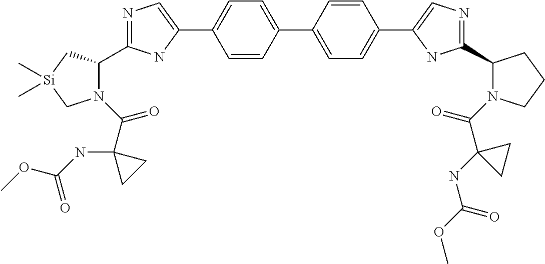 [SMILES: COC(=O)NC1(C(=O)N2CCC[C@@H]2c2ncc(-c3ccc(-c4ccc(-c5cnc([C@H]6C[Si](C)(C)CN6C(=O)C6(NC(=O)OC)CC6)[nH]5)cc4)cc3)[nH]2)CC1] | 751.9 | 1000 | 1000 | 1000 | 1000 |
| 21 | 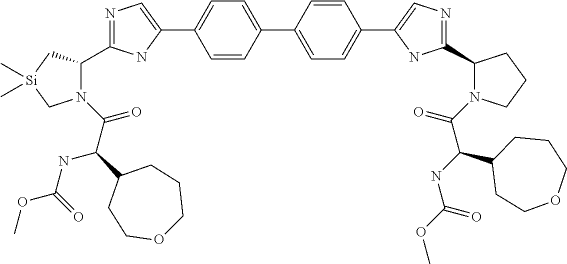 [SMILES: COC(=O)N[C@@H](C(=O)N1CCC[C@@H]1c1ncc(-c2ccc(-c3ccc(-c4cnc([C@H]5C[Si](C)(C)CN5C(=O)[C@H](NC(=O)OC)C5CCCOCC5)[nH]4)cc3)cc2)[nH]1)C1CCCOCC1] | 896.1 | 4.423 | 18.7 | 1000 | 204.5 |
| 22 | 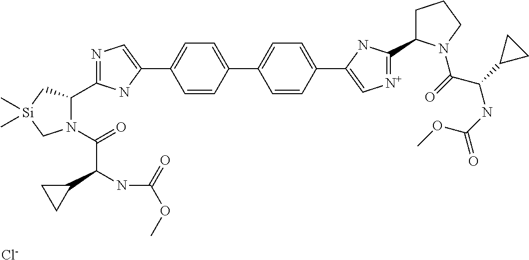 [SMILES: COC(=O)N[C@H](C(=O)N1C[Si](C)(C)C[C@@H]1c1ncc(-c2ccc(-c3ccc(-c4c[nH+]c([C@H]5CCCN5C(=O)[C@@H](NC(=O)OC)C5CC5)[nH]4)cc3)cc2)[nH]1)C1CC1 ; [Cl-]] | 780.0 | 1000 | 1000 | 1000 | 1000 |
| 23 | 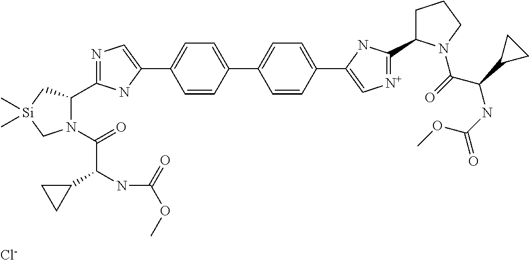 [SMILES: COC(=O)N[C@@H](C(=O)N1C[Si](C)(C)C[C@@H]1c1ncc(-c2ccc(-c3ccc(-c4c[nH+]c([C@H]5CCCN5C(=O)[C@H](NC(=O)OC)C5CC5)[nH]4)cc3)cc2)[nH]1)C1CC1 ; [Cl-]] | 780.0 | 1.163 | — | 1000 | 549.7 |

### Example 30

### Preparation of Compound 24

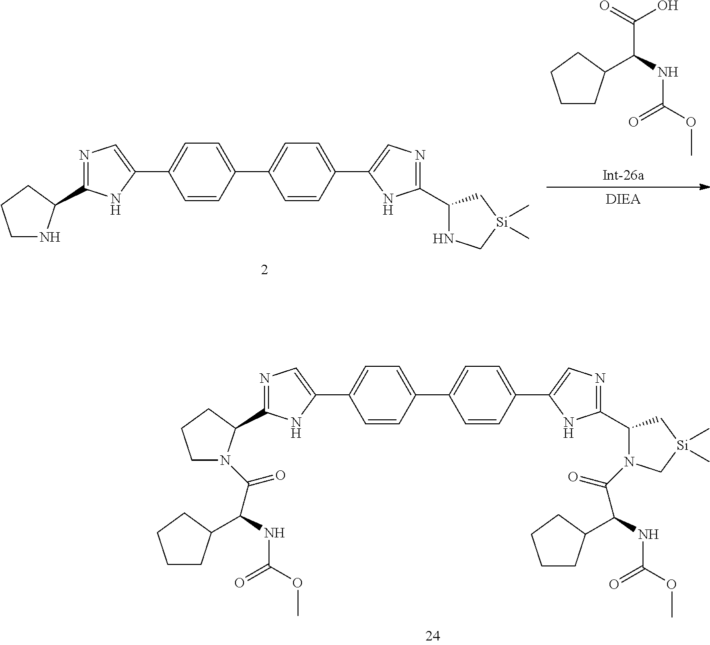 [SMILES: C[Si]1(C)CN[C@H](c2ncc(-c3ccc(-c4ccc(-c5cnc([C@@H]6CCCN6)[nH]5)cc4)cc3)[nH]2)C1 ; COC(=O)N[C@H](C(=O)O)C1CCCC1 ; COC(=O)N[C@H](C(=O)N1CCC[C@H]1c1ncc(-c2ccc(-c3ccc(-c4cnc([C@@H]5C[Si](C)(C)CN5C(=O)[C@@H](NC(=O)OC)C5CCCC5)[nH]4)cc3)cc2)[nH]1)C1CCCC1]

Compound 2 (32 mg, 0.068 mmol), along with (S)-2-cyclopentyl-2-(methoxycarbonylamino)acetic acid Int-26a (31 mg, 0.153 mmol), DIEA (44.3 μl, 0.254 mmol), acetonitrile (350 THF (350 μl) was shaken for 1 min, followed by addition of 1-propanephosphonic acid cyclic anhydride (45.3 μl, 0.152 mmol, 40% in EtOAc) and shaking at 25° C. for 18 hours. MS confirmed the formation of product. Solvent removed under vacuum. The resulting residue was dissolved in 1 mL DMSO, filtered and further purified using reverse-phase LC to provide compound 24 (9 mg, 0.01077 mmol, 15.8% yield).

Compounds 25-39, depicted in the table below, were made using the methods described in the Example above and substituting the appropriate reactants and/or reagents.

| | | | | | 1a | |
|---|---|---|---|---|---|---|
| | | | 1a | 1b | Y93H | 2b |
| | | Mass | EC90 | EC90 | EC90 | EC90 |
| No. | Structure | Obsvd | nM | nM | nM | nM |
| 24 | 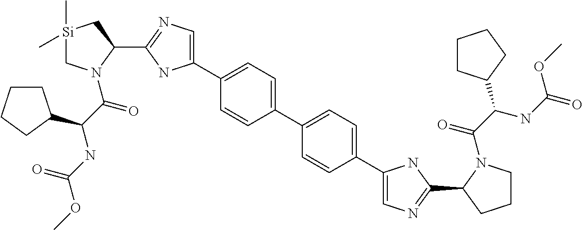 [SMILES: COC(=O)N[C@H](C(=O)N1CCC[C@H]1c1ncc(-c2ccc(-c3ccc(-c4cnc([C@@H]5C[Si](C)(C)CN5C(=O)[C@@H](NC(=O)OC)C5CCCC5)[nH]4)cc3)cc2)[nH]1)C1CCCC1] | 836.0 | 2.912 | 17.3 | 1000 | 381.9 |
| 25 | 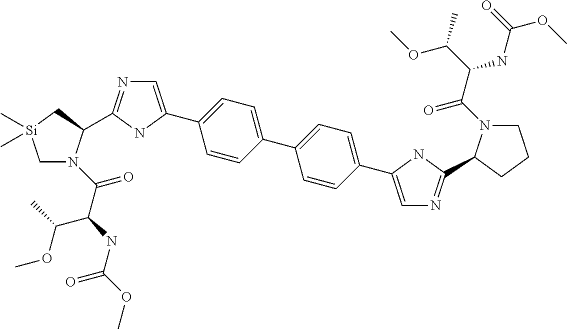 | 815.89 | 0.7202 | 4.772 | 1000 | 135.6 |
| 26 | 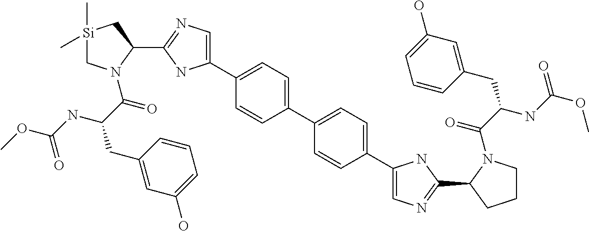 | 912.0 | 11.52 | 387.3 | 1000 | 1000 |
| 27 | 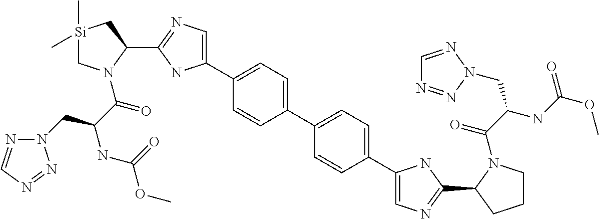 [SMILES: COC(=O)N[C@@H](Cn1ncnn1)C(=O)N1CCC[C@H]1c1ncc(-c2ccc(-c3ccc(-c4cnc([C@@H]5C[Si](C)(C)CN5C(=O)[C@H](Cn5ncnn5)NC(=O)OC)[nH]4)cc3)cc2)[nH]1] | 863.9 | 6.114 | 2.167 | 1000 | 138.2 |
| 28 | 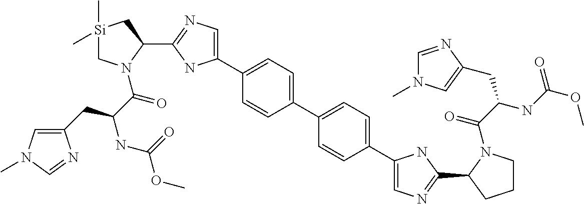 | 888.0 | 11.92 | 282.5 | 1000 | 1000 |
| 29 | 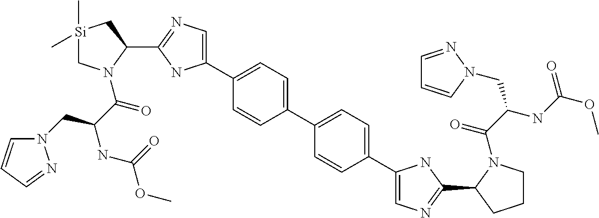 [SMILES: COC(=O)N[C@@H](Cn1cccn1)C(=O)N1CCC[C@H]1c1ncc(-c2ccc(-c3ccc(-c4cnc([C@@H]5C[Si](C)(C)CN5C(=O)[C@H](Cn5cccn5)NC(=O)OC)[nH]4)cc3)cc2)[nH]1] | 860.0 | 5.319 | 138.7 | 1000 | 860.7 |
| 30 | 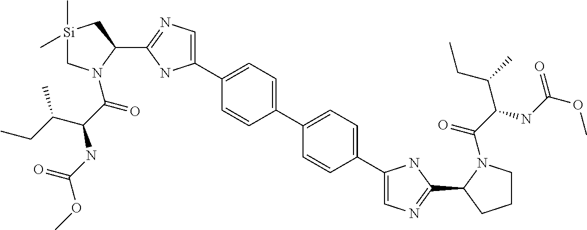 [SMILES: CC[C@H](C)[C@H](NC(=O)OC)C(=O)N1CCC[C@H]1c1ncc(-c2ccc(-c3ccc(-c4cnc([C@@H]5C[Si](C)(C)CN5C(=O)[C@@H](NC(=O)OC)[C@@H](C)CC)[nH]4)cc3)cc2)[nH]1] | 811.9 | 1.335 | 7.626 | 610.2 | 248.1 |
| 31 | 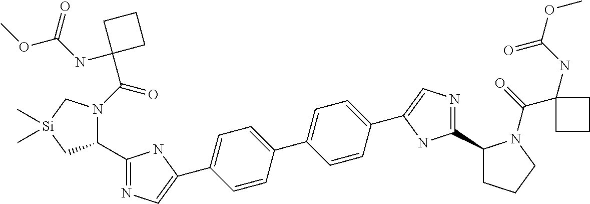 [SMILES: COC(=O)NC1(C(=O)N2CCC[C@H]2c2ncc(-c3ccc(-c4ccc(-c5cnc([C@@H]6C[Si](C)(C)CN6C(=O)C6(NC(=O)OC)CCC6)[nH]5)cc4)cc3)[nH]2)CCC1] | 779.6 | — | 2.2 | >1000 | >1000 |
| 32 | 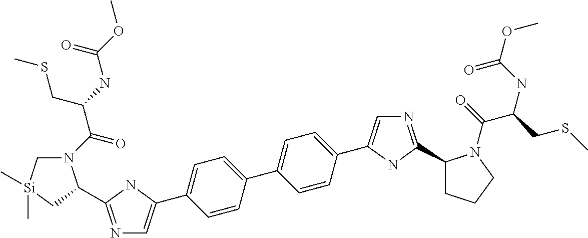 | 819.49 | — | 0.03 | >1000 | 144 |
| 33 | 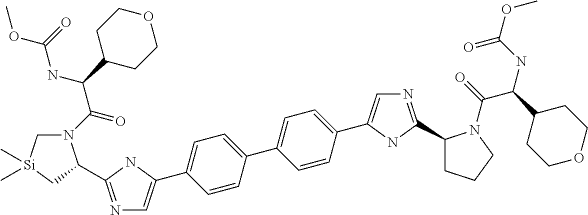 [SMILES: COC(=O)N[C@H](C(=O)N1CCC[C@H]1c1ncc(-c2ccc(-c3ccc(-c4cnc([C@@H]5C[Si](C)(C)CN5C(=O)[C@@H](NC(=O)OC)C5CCOCC5)[nH]4)cc3)cc2)[nH]1)C1CCOCC1] | 867.60 | — | 0.09 | 43 | 7.6 |
| 34 | 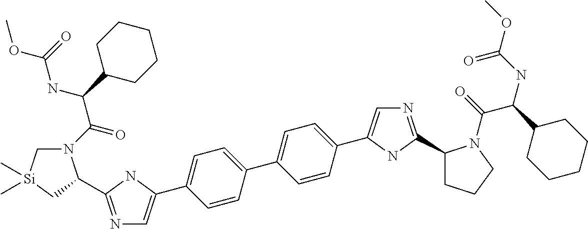 [SMILES: COC(=O)N[C@H](C(=O)N1CCC[C@H]1c1ncc(-c2ccc(-c3ccc(-c4cnc([C@@H]5C[Si](C)(C)CN5C(=O)[C@@H](NC(=O)OC)C5CCCCC5)[nH]4)cc3)cc2)[nH]1)C1CCCCC1] | 863.71 | 2.5 | 0.04 | >1000 | 648 |
| 35 | 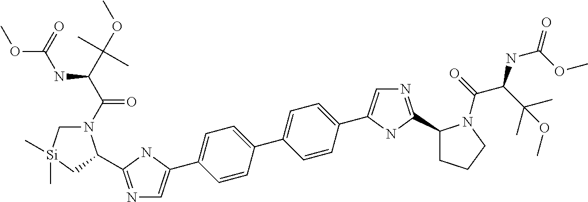 | 843.61 | — | 0.09 | >1000 | >1000 |
| 36 | 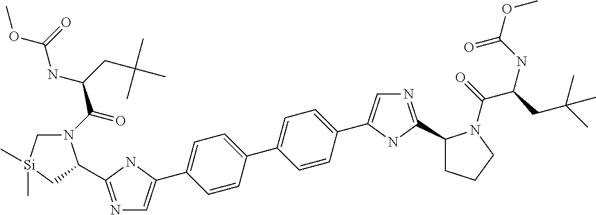 [SMILES: COC(=O)N[C@@H](CC(C)(C)C)C(=O)N1CCC[C@H]1c1ncc(-c2ccc(-c3ccc(-c4cnc([C@@H]5C[Si](C)(C)CN5C(=O)[C@H](CC(C)(C)C)NC(=O)OC)[nH]4)cc3)cc2)[nH]1] | 839.7 | — | 0.03 | 617 | 434 |
| 37 | 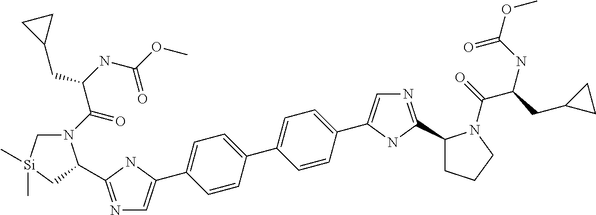 [SMILES: COC(=O)N[C@@H](CC1CC1)C(=O)N1CCC[C@H]1c1ncc(-c2ccc(-c3ccc(-c4cnc([C@@H]5C[Si](C)(C)CN5C(=O)[C@H](CC5CC5)NC(=O)OC)[nH]4)cc3)cc2)[nH]1] | 807.6 | — | 0.03 | 425 | 888 |
| 38 | 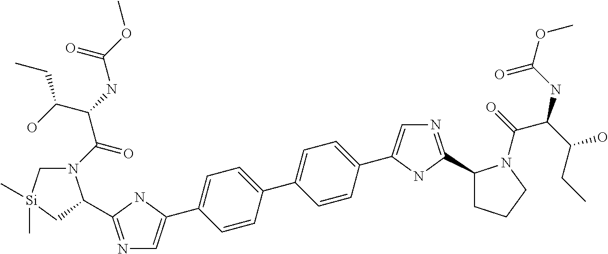 [SMILES: CC[C@@H](O)[C@H](NC(=O)OC)C(=O)N1CCC[C@H]1c1ncc(-c2ccc(-c3ccc(-c4cnc([C@@H]5C[Si](C)(C)CN5C(=O)[C@@H](NC(=O)OC)[C@H](O)CC)[nH]4)cc3)cc2)[nH]1] | 815.6 | — | 0.21 | >1000 | >1000 |
| 39 | 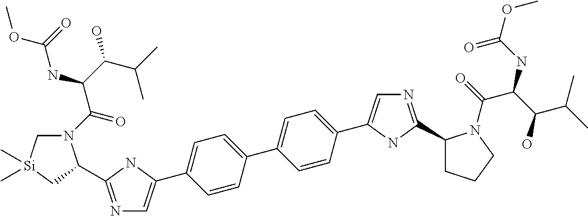 [SMILES: COC(=O)N[C@H](C(=O)N1CCC[C@H]1c1ncc(-c2ccc(-c3ccc(-c4cnc([C@@H]5C[Si](C)(C)CN5C(=O)[C@@H](NC(=O)OC)[C@H](O)C(C)C)[nH]4)cc3)cc2)[nH]1)[C@H](O)C(C)C] | 843.6 | — | 0.03 | >1000 | 510 |

### Example 31

### Preparation of Compounds 40 and 41

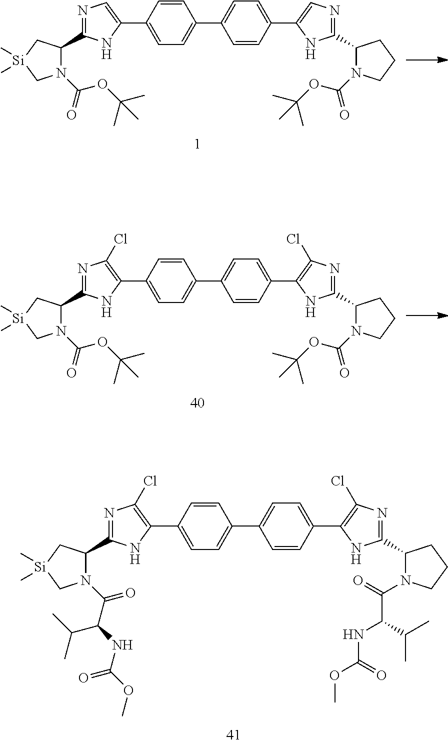 [SMILES: CC(C)(C)OC(=O)N1CCC[C@H]1c1ncc(-c2ccc(-c3ccc(-c4cnc([C@@H]5C[Si](C)(C)CN5C(=O)OC(C)(C)C)[nH]4)cc3)cc2)[nH]1 ; CC(C)(C)OC(=O)N1CCC[C@H]1c1nc(Cl)c(-c2ccc(-c3ccc(-c4[nH]c([C@@H]5C[Si](C)(C)CN5C(=O)OC(C)(C)C)nc4Cl)cc3)cc2)[nH]1 ; COC(=O)N[C@H](C(=O)N1CCC[C@H]1c1nc(Cl)c(-c2ccc(-c3ccc(-c4[nH]c([C@@H]5C[Si](C)(C)CN5C(=O)[C@@H](NC(=O)OC)C(C)C)nc4Cl)cc3)cc2)[nH]1)C(C)C]

### Step A—Preparation Compound 40

A solution of (R)-tert-butyl 5-(5-(4′-(2-((S)-1-(tert-butoxycarbonyl)pyrrolidin-2-yl)-1H-imidazol-5-yl)biphenyl-4-yl)-1H-imidazol-2-yl)-3,3-dimethyl-1,3-azasilolidine-1-carboxylate 1 (152 mg, 0.227 mmol) and NCS (67 mg, 0.502 mmol) in DMF (1.0 mL) was stirred at 50° C. for 2 hours. The reaction was concentrated in vacuo and the resulting residue was purified using silica gel chromatography (12 g column, 0% to 5% MeOH/DCM (12 minutes) to provide (R)-tert-butyl-5-(5-(4′-(2-((S)-1-(tert-butoxycarbonyl)pyrrolidin-2-yl)-4-chloro-1H-imidazol-5-yl)biphenyl-4-yl)-4-chloro-1H-imidazol-2-yl)-3,3-dimethyl-1,3-azasilolidine-1-carboxylate 40 (99 mg, 0.134 mmol, 59.1% yield).

### Step B—Preparation of Compound 41

A solution of (R)-tert-butyl 5-(5-(4′-(2-((S)-1-(tert-butoxycarbonyl)pyrrolidin-2-yl)-4-chloro-1H-imidazol-5-yl)biphenyl-4-yl)-4-chloro-1H-imidazol-2-yl)-3,3-dimethyl-1,3-azasilolidine-1-carboxylate 40 (99 mg, 0.134 mmol) in hydrochloric acid (1 mL, 4.00 mmol) in dioxane and methanol (0.5 mL) was allowed to stir for 3 hours at room temperature. The reaction was concentrated in vacuo and the resulting solid was used with purification. To a solution of the crude material prepared above (82 mg, 0.153 mmol), (S)-2-(methoxycarbonylamino)-3-methylbutanoic acid (66.8 mg, 0.381 mmol) and DIEA (0.266 mL, 1.525 mmol) in acetonitrile (0.5 mL) and THF (0.500 mL) was added 2,4,6-tripropyl-1,3,5,2,4,6-trioxatriphosphorinane-2,4,6-trioxide (0.227 mL, 0.381 mmol). The reaction was stirred at room temperature for 90 minutes and then quenched with HCl (0.2 mL, 0.800 mmol) and allowed to stir for 2 hours at room temperature. The reaction was concentrated in vacuo and the resulting residue was purified using reverse phase chromatography (C18 Luna 21×100 mm, 10:90 to 100:00 CH₃CN/H₂O (10 min)) to provide Compound 41 (38 mg, 0.044 mmol, 28.9% yield).

| | | | | | 1a | |
|---|---|---|---|---|---|---|
| | | | 1a | 1b | Y93H | 2b |
| | | Mass | EC90 | EC90 | EC90 | EC90 |
| No. | Structure | Obsvd | nM | nM | nM | nM |
| 41 | 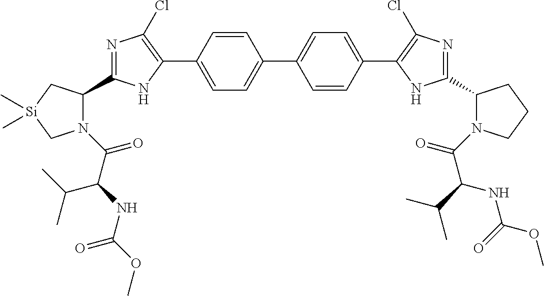 | 851.2 | 0.26 | 217 | 189 | 100 |

### Example 32

### Preparation of Compound 42

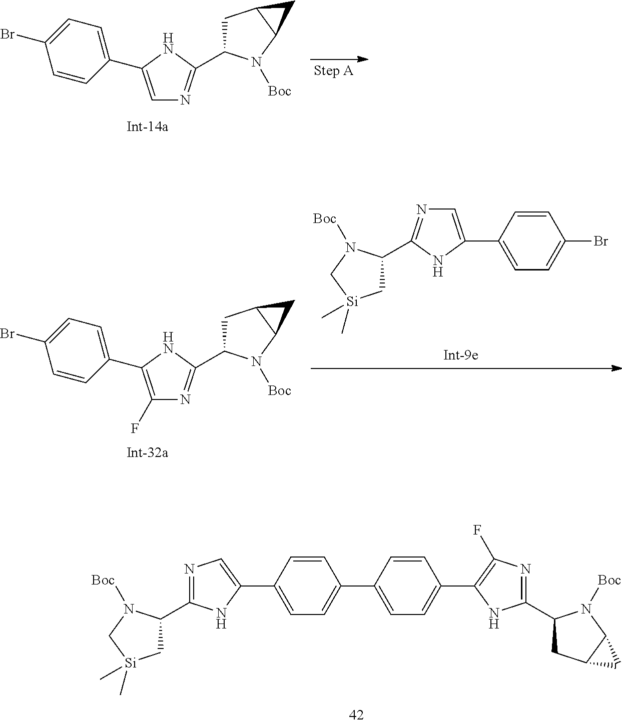

### Step A—Preparation of Compound Int-32a

Compound Int-14a (100 mg, 0.25 mmol) and AccuFlour (127 mg, 0.20 mmol, 0.8 eq) were added to a microwave tube (0.5-2 mL) and suspended in DMF (1 mL). The mixture was subjected microwave reactor (100° C., 30 min) and cooled to room temperature. The same reaction was repeated 5 times and the combined solution was added to ice-water. The mixture was extracted with ethyl acetate. The combined organic fractions were washed with brine, dried (Na₂SO₄), filtered and the solvent was evaporated in vacuo. The resulting residue was purified using ISCO 120 g gold column (Hex-40% EtOAc/Hex) to provide Int-32a (38 mg, 0.090 mmol, 7.28% yield) and Compound A (rec.SM) (150 mg, 0.371 mmol, 30.0% yield). LC-MS=422.1.

### Step B—Preparation of Compound 42

A mixture of Compound Int-9c (0.209 g, 0.48 mmol), bis(pinacolato)diboron (0.134 g, 0.528 mmol), KOAc (0.066 g, 0.480 mmol), and Pd(dppf)Cl₂ (0.035 g, 0.048 mmol) in 1,4-Dioxane (3 mL) was degassed (by N₂ flush) and heated to 100° C. for 2 h. After cooled to room temperature, the crude mixture was treated with Int-32a (100 mg, 0.237 mmol), Pd(dppf)Cl₂ (10.24 mg, 0.014 mmol) and 1N K₂CO₃ (0.5 mL, 0.500 mmol) and the mixture was degassed and stirred at 100° C. for 2 h. The mixture was cooled to room temperature, diluted in EtOAc, and filtered through celite pad. The filtrate was concentrated in vacuo and the resulting residue was purified using ISCO 40 g gold column (Hex to 70% EtOAc/Hex) to provide Compound 42 (100 mg, 0.143 mmol, 60.4% yield). LC-MS=700.3.

| | | | | | 1a | |
|---|---|---|---|---|---|---|
| | | | 1a | 1b | Y93H | 2b |
| | | Mass | EC90 | EC90 | EC90 | EC90 |
| No. | Structure | Obsvd | nM | nM | nM | nM |
| 42 | 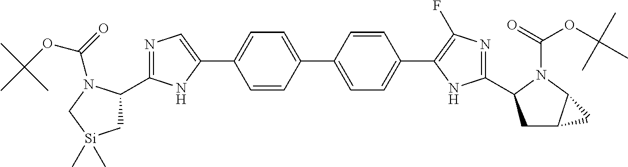 [SMILES: CC(C)(C)OC(=O)N1C[Si](C)(C)C[C@H]1c1ncc(-c2ccc(-c3ccc(-c4[nH]c([C@@H]5C[C@H]6C[C@H]6N5C(=O)OC(C)(C)C)nc4F)cc3)cc2)[nH]1] | 699.3 | >1 | — | >1000 | >1000 |

### Example 33

### Preparation of Compounds 43 and 44

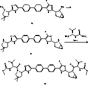 [SMILES: CC(C)(C)OC(=O)N1C[Si](C)(C)C[C@H]1c1ncc(-c2ccc(-c3ccc(-c4[nH]c([C@@H]5C[C@H]6C[C@H]6N5C(=O)OC(C)(C)C)nc4F)cc3)cc2)[nH]1 ; C[Si]1(C)CN[C@H](c2ncc(-c3ccc(-c4ccc(-c5[nH]c([C@@H]6C[C@H]7C[C@H]7N6)nc5F)cc4)cc3)[nH]2)C1 ; COC(=O)N[C@H](C(=O)O)C(C)C ; COC(=O)N[C@H](C(=O)N1C[Si](C)(C)C[C@H]1c1ncc(-c2ccc(-c3ccc(-c4[nH]c([C@@H]5C[C@H]6C[C@H]6N5C(=O)[C@@H](NC(=O)OC)C(C)C)nc4F)cc3)cc2)[nH]1)C(C)C]

### Step A—Preparation of Compound 43

Trifluoroacetic acid (0.9 mL, 11.68 mmol) was added to a stirred, cooled 0° C. mixture of Compound 42 (58 mg, 0.083 mmol) in CH₂Cl₂ (2 mL) and the mixture was stirred at room temperature for 90 min. The mixture was concentrated in vacuo and the resulting residue was dissolved in MeOH followed by treatment with 2N HCl in ether. The mixture was concentrated to dryness providing 43 (41.4 mg, 0.083 mmol, 100% yield) which was used without further purification.

### Step B—Preparation of Compound 44

Compound 43 (47.4 mg, 0.083 mmol) was dissolved in DMF (1 mL) and treated with Compound Int-1a (29.1 mg, 0.166 mmol). To this were added N,N-diisopropylethylamine (0.101 mL, 0.581 mmol) and HATU (63.1 mg, 0.166 mmol) at −15° C. The mixture was allowed to stir for 10 minutes and allowed to warm to 0° C. After 3 hours at 0° C., the reaction was quenched by 0.3 ml of water and the mixture was filtered/purified using reverse-phase HPLC eluting with Acetonitrile/Water+0.1% TFA, to provide Compound 44 (45 mg, 0.043 mmol, 52.1% yield) as a yellow solid. LC-MS=813.3.

| | | | | | 1a | |
|---|---|---|---|---|---|---|
| | | | 1a | 1b | Y93H | 2b |
| | | Mass | EC90 | EC90 | EC90 | EC90 |
| No. | Structure | Obsvd | nM | nM | nM | nM |
| 44 | 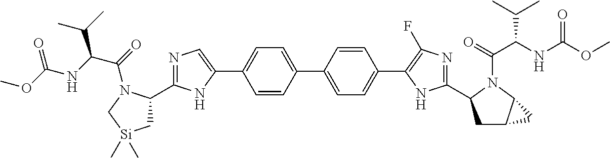 | 813.3 | 0.17 | — | 285 | 168 |

### Example 34

### Preparation of Compound 45

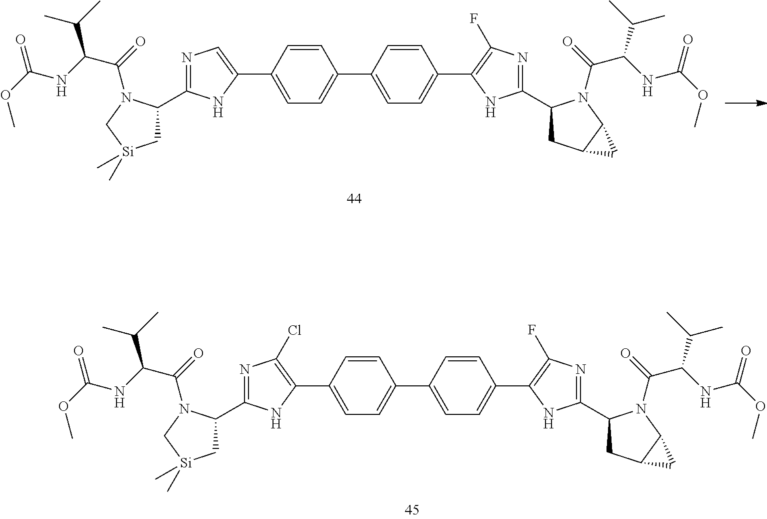 [SMILES: COC(=O)N[C@H](C(=O)N1C[Si](C)(C)C[C@H]1c1ncc(-c2ccc(-c3ccc(-c4[nH]c([C@@H]5C[C@H]6C[C@H]6N5C(=O)[C@@H](NC(=O)OC)C(C)C)nc4F)cc3)cc2)[nH]1)C(C)C ; COC(=O)N[C@H](C(=O)N1C[Si](C)(C)C[C@H]1c1nc(Cl)c(-c2ccc(-c3ccc(-c4[nH]c([C@@H]5C[C@H]6C[C@H]6N5C(=O)[C@@H](NC(=O)OC)C(C)C)nc4F)cc3)cc2)[nH]1)C(C)C]

N-Chlorosuccinimide (5.36 mg, 0.040 mmol) was added to a stirred mixture of Compound 44 (38 mg, 0.037 mmol) and N,N-diisopropylethylamine (6.36 μl, 0.037 mmol) in DMF (0.7 mL) and the mixture was stirred at 50° C. for Overnight. The mixture was added to a water and the organic layers were extracted by EtOAc. The combined organic solution was washed with brine, dried (Na₂SO₄), and concentrated in vacuo. The resulting residue was purified using PTLC (EA/Hex=1/2) to provide Compound 45 (18.3 mg, 0.017 mmol, 46.6% yield). LC-MS=847.2.

| | | | | | 1a | |
|---|---|---|---|---|---|---|
| | | | 1a | 1b | Y93H | 2b |
| | | Mass | EC90 | EC90 | EC90 | EC90 |
| No. | Structure | Obsvd | nM | nM | nM | nM |
| 45 | 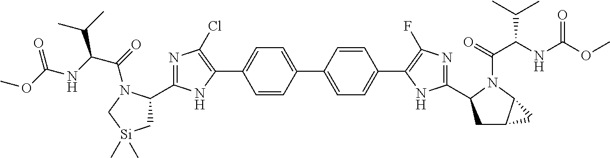 | 847.2 | 0.13 | 0.017 | 144 | 66 |

### Example 35

### Preparation of Compounds 46-48

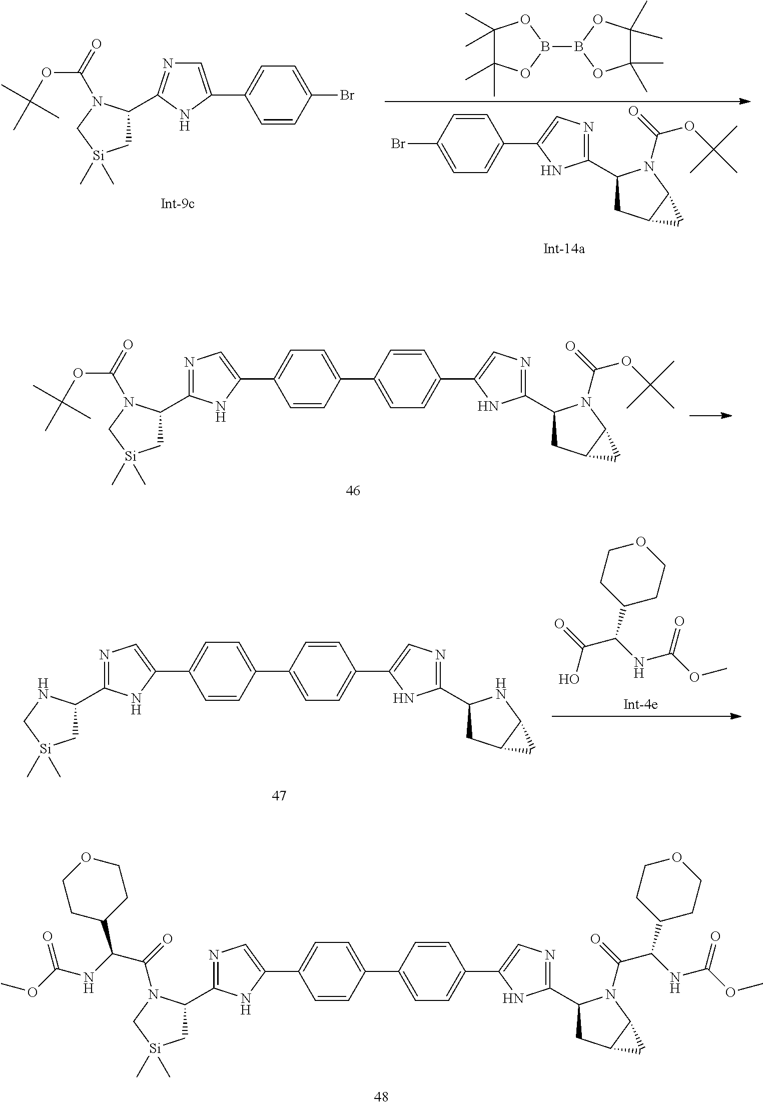

### Step A—Preparation of Compound 46

To a 100 mL round bottom flask charged with a stir bar was added intermediate bromide Int-9c (1.0 g, 2.3 mmol), bis(pinacolato)diboron (0.64 g, 2.5 mmol), KOAc (0.68 g, 6.9 mmol), and PdCl₂dppf-CH₂Cl₂ (0.37 g, 0.46 mmol). Dioxane (˜15 mL) was added to the flask and a N₂ line was inserted. Using the N₂ line, the reaction mixture was degassed under house vacuum and filled with N₂ five times. The tube was heated to 90° C. and stirred under N₂ for 4 h whereupon analysis by LC-MS deemed the reaction to be complete. This crude reaction was taken on without further purification to Step 2.

To the crude boronate from above was added intermediate Int-14a (0.53 g, 1.3 mmol), PdCl₂dppf.CH₂Cl₂ (0.19 g, 0.24 mmol), and 1M K₂CO₃ (˜3.5 mL). The flask was flushed with N₂, capped, and heated to 95° C. The mixture was allowed to stir for 12 hours at 95° C. whereupon the reaction was deemed to be complete by LC-MS. The mixture was cooled to room temperature and was diluted with EtOAc (100 mL) and water (20 mL). The layers were separated and the aqueous layer was extracted with EtOAc (3×75 mL). The organic layers were combined and were washed with brine (1×50 mL). The organic layer was dried (Na₂SO₄), filtered, and concentrated in vacuo to provide a crude maroon semisolid was placed under high vacuum. The crude material was purified using ISCO (120 g silica Gold column) using a gradient of 100% hexanes to 100% EtOAc to provide 420 mg (35%) of compound 46 as an off-white solid. LC-MS=681.3.

### Step B—Preparation of Compound 47

To intermediate 46 (0.10 g, 0.14 mmol) in CH₂Cl₂ (3 mL) under N₂ was added TFA (1 mL) in one portion and the resultant solution was stirred under N₂ at rt 1.5 hr. The mixture was concentrated to dryness and was treated with 4.0 M HCl/Dioxane (3 mL) for 30 min. The mixture was concentrated in vacuo and was placed under vacuum to provide 90 mg (99%) of compound 47 as the tetrahydrochloride salt. LC-MS=493.2.

### Step C—Preparation of Compound 48

To a solution of intermediate 47 (71 mg, 0.14 mmol) in DMF (1.5 mL) at −15° C. (acetone/ice bath) was added S-2-(methoxycarbonylamino)-2-(tetrahydro-2H-pyran-4-yl)acetic acid Int-4e (66 mg, 0.30 mmol) followed by HATU (0.115 g, 0.30 mmol). The mixture was stirred or 15 minutes whereupon DIEA (0.13 mL, 0.72 mmol) was added dropwise. This mixture was then stirred at −15° C. for 90 minutes whereupon the mixture quenched by adding 3 mL H₂O and 15 mL EtOAc and the layers were separated. The aqueous layer was extracted with EtOAc (2×7 mL) and the organic layers were combined. The organic layer was washed with H₂O (3×3 mL), brine (3×3 mL), and dried over anhydrous Na₂SO₄, filtered, and concentrated to provide ˜0.2 g of brown residue which was kept under vacuum for ˜1 hr. The crude material was purified using reverse-phase HPLC (Gilson) using a C18 column with a gradient: 0% ACN to 90% ACN/10% water (both with 0.1% TFA) to provide 85 mg (61%) of compound 48 as a light yellow dihydrochloride salt after treatment with HCl. LC-MS=879.3.

| | | | | | 1a | |
|---|---|---|---|---|---|---|
| | | | 1a | 1b | Y93H | 2b |
| | | Mass | EC90 | EC90 | EC90 | EC90 |
| No. | Structure | Obsvd | nM | nM | nM | nM |
| 46 | 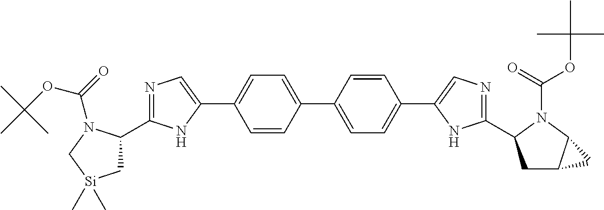 [SMILES: CC(C)(C)OC(=O)N1C[Si](C)(C)C[C@H]1c1ncc(-c2ccc(-c3ccc(-c4cnc([C@@H]5C[C@H]6C[C@H]6N5C(=O)OC(C)(C)C)[nH]4)cc3)cc2)[nH]1] | 681.3 | >1000 | — | >1000 | 422 |
| 48 | 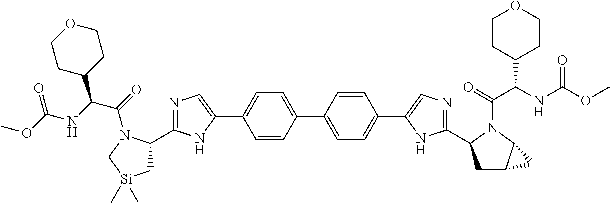 | 879.3 | 0.18 | — | 144 | 9 |

### Example 36

### Preparation of Compounds 49-51

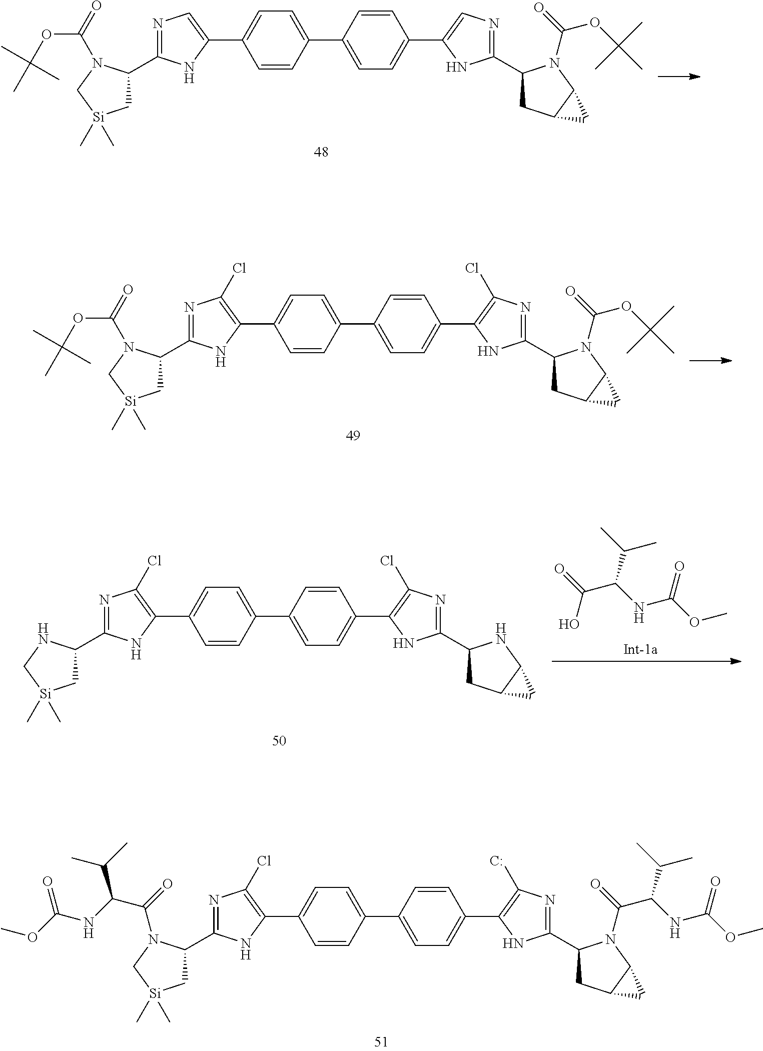

### Step A—Preparation of Compound 49

To a flask charged with bis Boc adduct 48 (0.22 g, 0.33 mmol) in DMF (3 mL) at rt was added NCS (96 mg, 0.72 mmol) in a single portion. The mixture was heated to 50° C. and was stirred overnight for 14 h. The mixture was cooled to room temperature, concentrated in vacuo, and placed under high vacuum to provide a brown semisolid. The crude material was purified using ISCO using a 40 g column and a gradient of 100% hexanes to 100% EtOAc to provide 215 mg (87%) of compound 49 as an off-white solid. LC-MS=751.2

### Step B—Preparation of Compound 50

To a mixture of bis Boc adduct 49 (71 mg, 0.10 mmol) in MeOH (0.5 mL) at rt was added 4N HCl in dioxane (˜0.25 mL) to provide a yellow, homogenous solution. The mixture was allowed to stir for 3 h at rt, concentrated in vacuo, and was placed under high vacuum to provide 66.5 mg (99%) of 50 as a light yellow solid. LC-MS=551.0. This material was taken onto the next step without further purification.

### Step C—Preparation of Compound 51

To a solution of the HCl salt 50 obtained from (66 mg, 0.095 mmol) in DMF (1 mL) at −10° C. (ice/acetone) was added S-ValMoc Int-1a (35 mg, 0.20 mmol), HATU (76 mg, 0.20 mmol), followed by dropwise addition of DIEA (0.10 mL, 0.57 mmol) to provide an orange, homogenous solution. The resulting solution was allowed to stir for 1.5 hours at −10° C. whereupon the mixture was diluted with water (1.5 mL) and EtOAc (4 mL). The mixture was allowed to warm to room temperature and the layers were separated. The aqueous layer was extracted with EtOAc (3×4 mL) and the organic layers were combined. The organic layer was washed with brine (1×3 mL), dried (Na₂SO₄) filtered, and concentrated in vacuo. The crude material was purified using reverse-phase HPLC (Gilson) using a C18 column with a gradient: 10% ACN to 90% ACN/10% water (w/ 0.1% TFA) to provide 35 mg (39%) of compound 51 as a light yellow dihydrochloride salt after treatment with HCl. LC-MS=865.3.

Compounds 52-53, depicted in the table below, were made using the methods described in the Example above and substituting the appropriate reactants and/or reagents.

| | | | | | 1a | |
|---|---|---|---|---|---|---|
| | | | 1a | 1b | Y93H | 2b |
| | | Mass | EC90 | EC90 | EC90 | EC90 |
| No. | Structure | Obsvd | nM | nM | nM | nM |
| 49 | 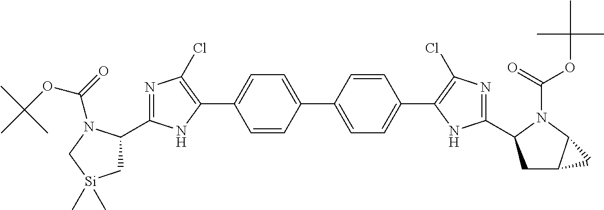 [SMILES: CC(C)(C)OC(=O)N1C[Si](C)(C)C[C@H]1c1nc(Cl)c(-c2ccc(-c3ccc(-c4[nH]c([C@@H]5C[C@H]6C[C@H]6N5C(=O)OC(C)(C)C)nc4Cl)cc3)cc2)[nH]1] | 749.2 | >1000 | — | >1000 | >1000 |
| 51 | 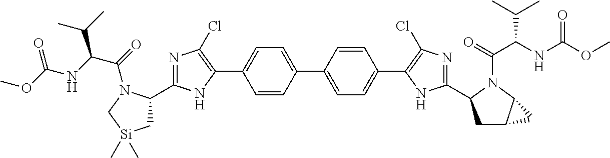 | 863.3 | 0.008 | — | 13.7 | 2.9 |
| 52 | 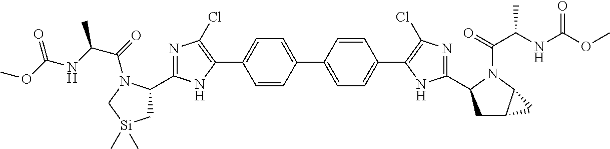 | 807.3 | 0.021 | 0.007 | 9.5 | 57 |
| 53 | 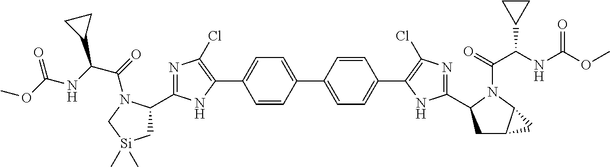 | 859.3 | 0.010 | 0.010 | 26.8 | 11.3 |

### Example 37

### Preparation of Compound 54

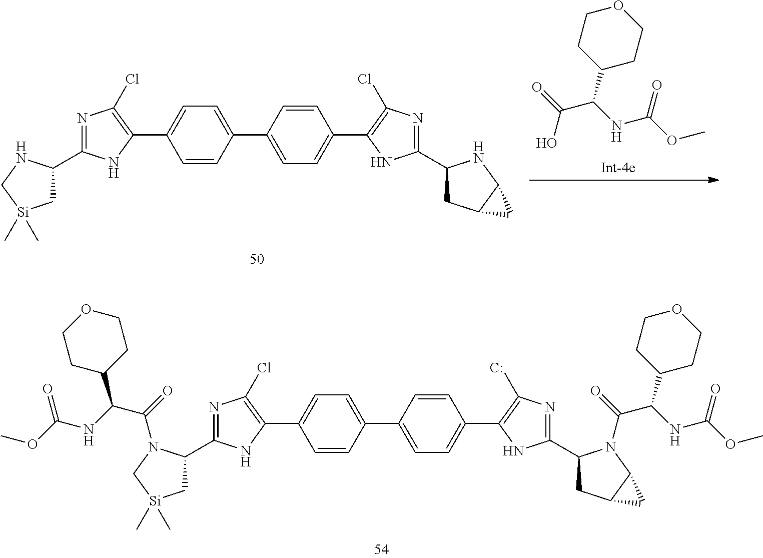

Using the method described in Example 35, Step C, compound 50 (61 mg, 0.087 mmol) was reacted Int-4e(40 mg, 0.18 mmol) to provide 80 mg (90%) of compound 54 as the dihydrochloride salt after treatment with HCl. LC-MS: 947.3.

| | | | | | 1a | |
|---|---|---|---|---|---|---|
| | | | 1a | 1b | Y93H | 2b |
| | | Mass | EC90 | EC90 | EC90 | EC90 |
| No. | Structure | Obsvd | nM | nM | nM | nM |
| 54 | 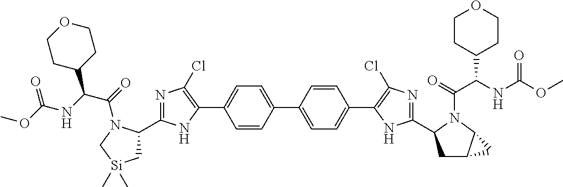 | 947.3 | 0.023 | — | 2.5 | 0.48 |

### Example 38

### Preparation of Compound 55

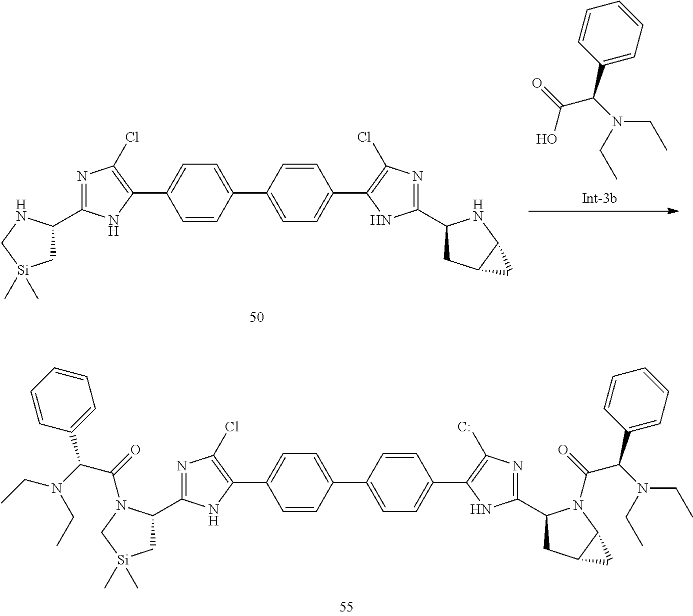

Using the method described in Example 35, Step C, compound 50 (66 mg, 0.095 mmol) was treated with (R)-2-(diethylamino)-2-phenylacetic acid Int-3b (41 mg, 0.20 mmol) to provide 55 mg (54%) of compound 55 as the dihydrochloride salt after HCl treatment. LC-MS: 927.4.

| | | | | | 1a | |
|---|---|---|---|---|---|---|
| | | | 1a | 1b | Y93H | 2b |
| | | Mass | EC90 | EC90 | EC90 | EC90 |
| No. | Structure | Obsvd | nM | nM | nM | nM |
| 55 | 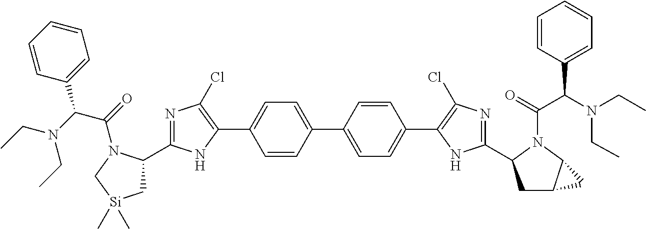 [SMILES: CCN(CC)[C@@H](C(=O)N1C[Si](C)(C)C[C@H]1c1nc(Cl)c(-c2ccc(-c3ccc(-c4[nH]c([C@@H]5C[C@H]6C[C@H]6N5C(=O)[C@@H](c5ccccc5)N(CC)CC)nc4Cl)cc3)cc2)[nH]1)c1ccccc1] | 927.4 | 0.047 | 0.019 | 16.4 | 0.16 |

### Example 39

### Preparation of Compounds 56-58

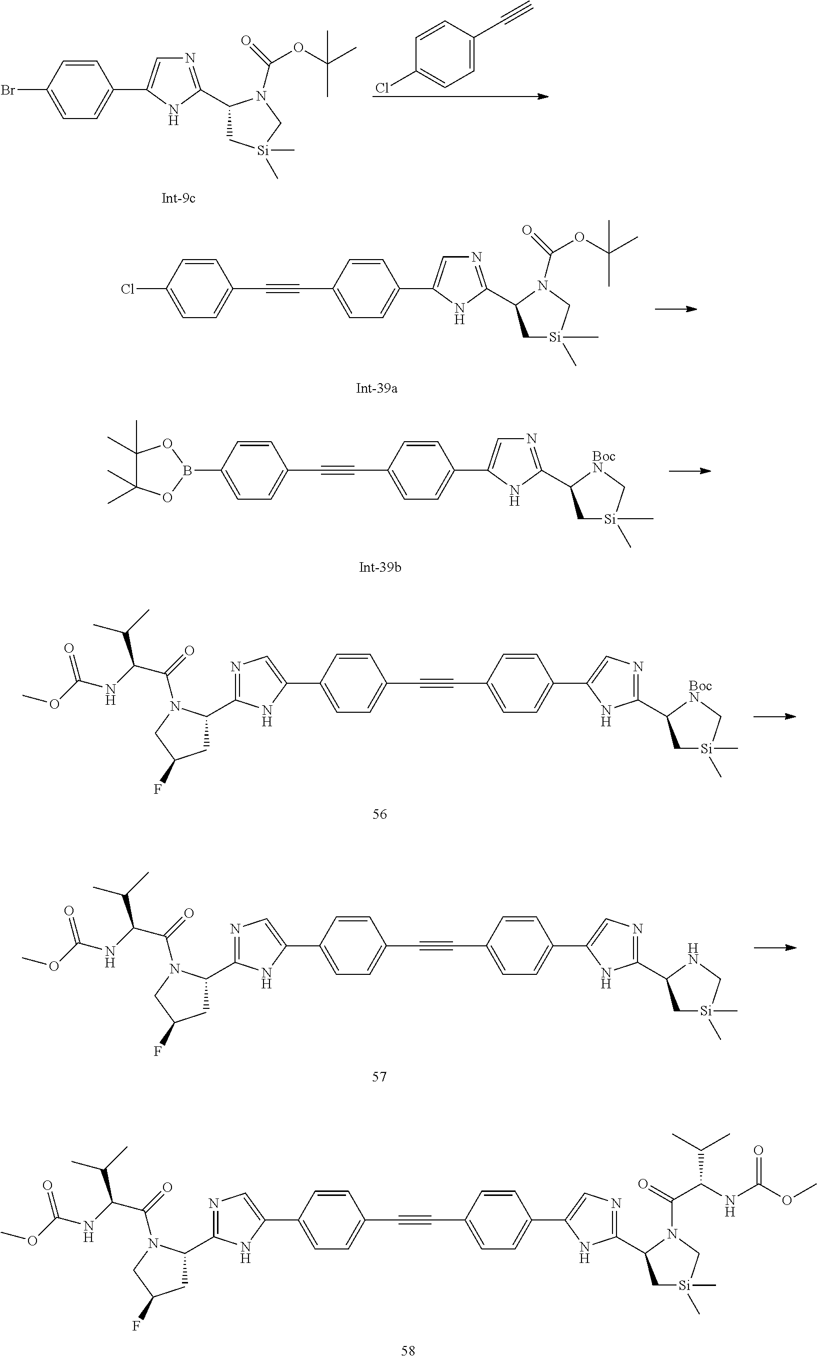

### Step A—Preparation of Compound Int-39a

To a 40 mL flask was added (S)-tert-butyl 5-(5-(4-bromophenyl)-1H-imidazol-2-yl)-3,3-dimethyl-1,3-azasilolidine-1-carboxylate Int-9c (300 mg, 0.687 mmol), bis(triphenylphosphino)palladium(II)dichloride (48.2 mg, 0.069 mmol), and copper(I)iodide (131 mg, 0.687 mmol). The flask was degassed, filled with nitrogen, and caped with a cap with septan. DMF (6874 μl) was added via syringe and the flask was stirred at 100° C. for 6 hours under nitrogen. 1-Chloro-4-ethynylbenzene (469 mg, 3.44 mmol) was dissolved in 2 mL DMF. 0.25 mL was added via syringe into the reaction mixture at 100° C. every 30 minutes. After cooling down, the solution was diluted with 10 mL EtOAc, filtered, and concentrated in vacuo. The product was purified using SiO₂ chromatography (40 g, Hexane/EtOAc 0% to 80%) to provide Int-39a (310 mg, 92%).

### Step B—Preparation of Compound Int-39b

Compound Int-39a (300 mg, 0.610 mmol), Pd₂(dba)₃ (112 mg, 0.122 mmol), X-Phos (116 mg, 0.244 mmol), and KOAc (359 mg, 3.66 mmol) are added into a 50 mL flask. After the flask was flashed with nitrogen, 1,4-dioxane (6 mL) was added. The mixture was stirred at 110° C. for 3 hours. After cooling down, EtOAc (15 mL) was added and the solution was filtered and concentrated in vacuo. The product was purified using SiO₂ chromatography (24 g, Hexane/EtOAc 0% to 40%) to provide Int-39b (340 mg, 96%)

### Step C—Preparation of Compound 56

Compound Int-39b (340 mg, 0.583 mmol), methyl (S)-1-((2S,4R)-2-(5-bromo-1H-imidazol-2-yl)-4-fluoropyrrolidin-1-yl)-3-methyl-1-oxobutan-2-ylcarbamate (251 mg, 0.641 mmol), and PdCl₂(dppf) (85 mg, 0.117 mmol) are added into a 40 mL flask. After the flask was flashed with nitrogen, 1,4-dioxane (5.8 mL) and 1M K₂CO₃ (2.9 mL, 2.91 mmol) were added. The mixture was stirred at 90° C. for 16 hours. After cooling down, the aqueous layer was separated and extracted with 5 mL EtOAc. The organic layers were combined and dried over anhydrous Na₂SO₄. The solution was filtered and concentrated in vacuo. The product was purified using SiO₂ chromatography (24 g, solvent A: DCM; solvent B: 10% MeOH in EtOAc. % of B in A: 0% to 80%) to provide 56 (239 mg, 53.4%).

### Step D—Preparation of Compound 57

Compound 56 (230 mg, 0.299 mmol) and MeOH (2995 μl) were placed in a 40 mL flask. HCl (7487 μl, 29.9 mmol) was added. The solution was stirred at 25° C. for 1 hour. The solution was concentrated in vacuo and dried under vacuum for overnight to provide 57 (230 mg, 99%).

### Step E—Preparation of Compound 58

Compound 57 (20 mg, 0.030 mmol), (5)-2-(methoxycarbonylamino)-3-methylbutanoic acid Int-1a (5.77 mg, 0.033 mmol), HATU (12.53 mg, 0.033 mmol), and DMF (1000 μl) were added into a 40 mL flask. The reaction mixture was cooled to 0° C. and Diisopropylethylamine (26.7 μl, 0.150 mmol) were added. The solution was stirred at 0° C. for 1 hour. Water (0.1 mL) and TFA (0.1 mL) were added at 0° C. The solution was then stirred at room temperature for 30 minutes. The solution was purified using C18 column (15.5 g, CH₃CN/water 10% to 70%, with 0.05% TFA) to provide compound 58 (13 mg, 41.2%).

Compound 59, depicted in the table below, was made using the methods described in the Example above and substituting the appropriate reactants and/or reagents.

| | | | | | 1a | |
|---|---|---|---|---|---|---|
| | | | 1a | 1b | Y93H | 2b |
| | | Mass | EC90 | EC90 | EC90 | EC90 |
| No. | Structure | Obsvd | nM | nM | nM | nM |
| 56 | 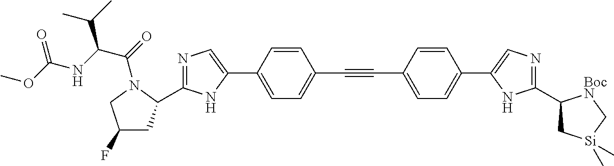 | 768.4 | 681 | — | >1000 | — |
| 57 | 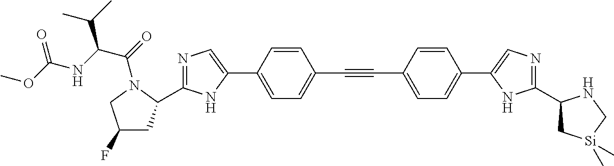 | 668.4 | 780 | — | >1000 | — |
| 58 | 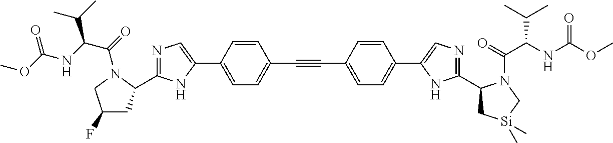 | 825.4 | >1 | 0.03 | >100 | >100 |
| 59 | 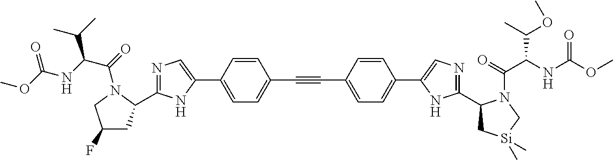 | 841.4 | >1 | 0.02 | >100 | >100 |

### Example 40

### Preparation of Compounds 60-63

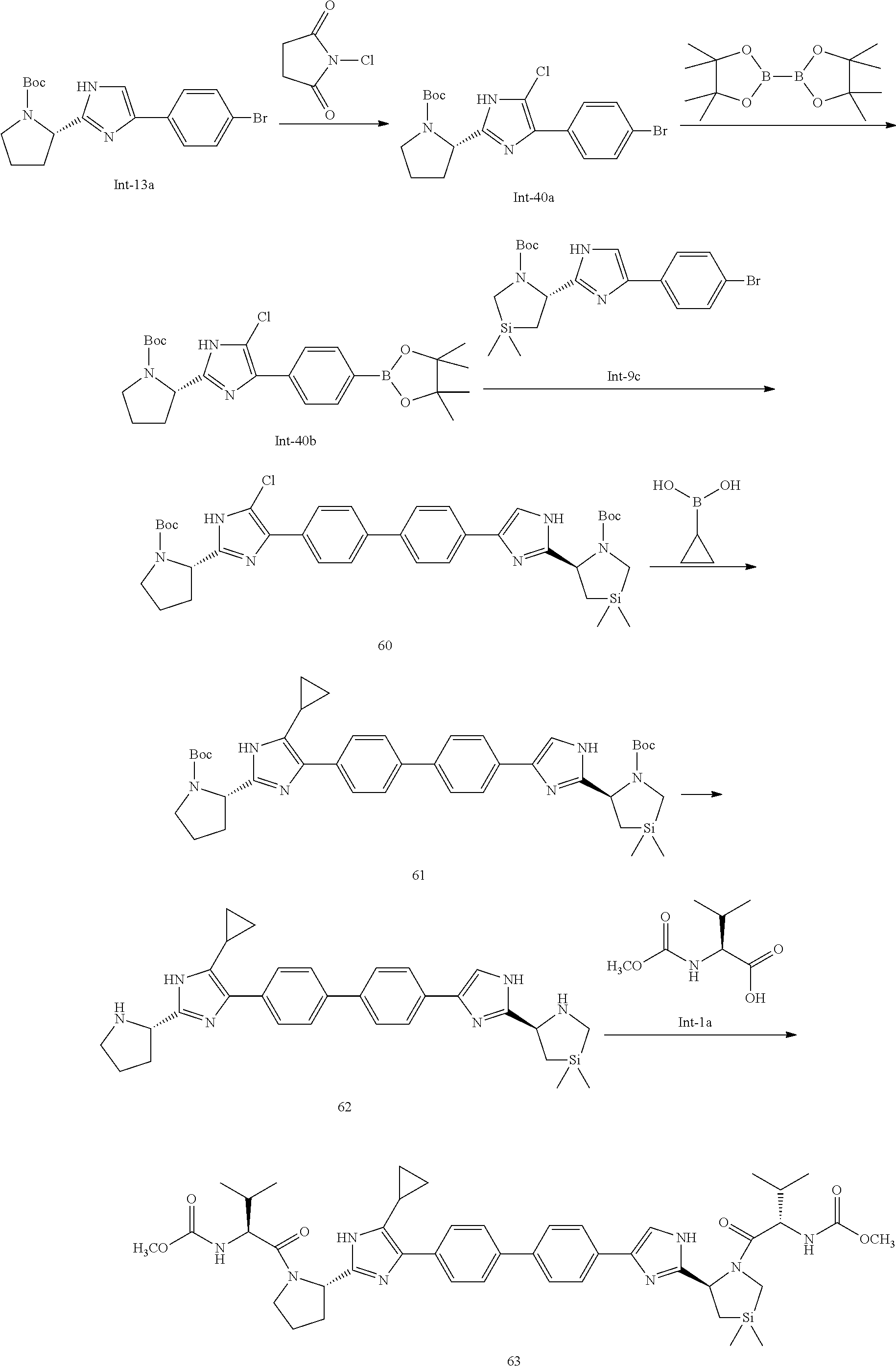

### Step A—Preparation of Compound Int-40a

A solution of compound Int-13a (3 g, 7.65 mmol) and NCS (1.23 g, 9.18 mmol) in MeOH (50 mL) was stirred at 50° C. overnight. The reaction was concentrated in vacuo and the resulting residue was purified using silica gel chromatography (40 g column, 0% to 10% EtOAc/Hex) to provide compound Int-40a (3 g, 7.03 mmol, 92% yield).

### Step B—Preparation of Compound 60

Compound Int-40a (150 mg, 0.35 mmol), KOAc (103 mg, 1.06 mmol), bis(pinacolato)diboron (0.11 g, 0.42 mmol) and PdCl₂(dppf)₂ (57.4 mg, 0.07 mmol) and were added into a microwave tube. After the flask was flashed with N₂, dioxane (3 mL) was added. The mixture was stirred at 90° C. for 3 hours and was cooled to room temperature to provide compound Int-40b, which was not isolated. To this reaction mixture was added compound Int-9c (154 mg, 0.35 mmol), PdCl₂(dppf)₂ (58 mg, 0.07 mmol) and K₂CO₃ (1 N aq., 1.06 mL). The tube was sealed and degassed and reheated to 90° C. overnight. After cooling, EtOAc (100 mL) was added and it was washed with brine (100 mL). The organic layer was separated, dried (Na₂SO₄), and concentrated in vacuo. The crude material was purified on a ISCO column (24 g) eluted with CH₂Cl₂ and EtOAc/MeOH/NH₃.H₂O(100:10:1) 0% to 100 to provide 175 mg (71%) of compound 60.

### Step C—Preparation of Compound 61

Compound 60 (72 mg, 0.1 mmol), cyclopropyl boronic acid (88 mg, 1.0 mmol), Pd₂dba₃ (10.6 mg, 10.2 μmol), X-Phos (9.8 mg, 0.02 mmol) and K₂CO₃ (1 N aq., 307 μl) were added to a 20 mL of microwave tube. The tube was sealed and degassed. The reaction was stirred at 110° C. for 5 hr. The crude material was purified using GRACE Davison column system eluted with CH₂Cl₂ with EtOAc/MeOH/NH₃.H₂O (100:10:1) 0% to 100% afford 33 mg (46%) of compound 61.

### Step D—Preparation of Compound 62

Compound 61 (31 mg, 0.044 mmol) was dissolved in dioxane (2 mL) and 4.0 N HCl in dioxane (0.5 mL) was added and the mixture was stirred at rt for 1.5 hr. The solvent was removed and the crude product 62 was dried under vacuum. This material was used in the next reaction without further purification.

### Step E—Preparation of Compound 63

To a solution of compound 62 (15 mg, 0.026 mmol) in DMF (1.5 mL) at 0° C. was added HATU (20.6 mg, 0.054 mmol) and (S)-2-(methoxycarbonylamino)-3-methylbutanoic acid Int-1a (9.5 mg, 0.054 mmol) followed by addition of DIEA (0.027 mL, 0.16 mmol). The reaction was stirred at 0° C. for 1.5 hr. Water was added and the mixture was diluted with EtOAc. The organic layer was washed with brine, dried (Na₂SO₄), and concentrated in vacuo. The crude material was purified using Reverse Phase HPLC C18 column eluted with H₂O and AcCN with TFA (0.1%) 0% to 90% to provide 11 mg (41%) of compound 63.

Compounds 64-67, depicted in the table below, were made using the methods described in the Example above and substituting the appropriate reactants and/or reagents.

| | | | | | 1a | |
|---|---|---|---|---|---|---|
| | | | 1a | 1b | Y93H | 2b |
| | | Mass | EC90 | EC90 | EC90 | EC90 |
| No. | Structure | Obsvd | nM | nM | nM | nM |
| 60 | 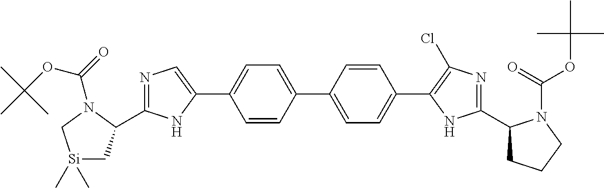 [SMILES: CC(C)(C)OC(=O)N1C[Si](C)(C)C[C@H]1c1ncc(-c2ccc(-c3ccc(-c4[nH]c([C@@H]5CCCN5C(=O)OC(C)(C)C)nc4Cl)cc3)cc2)[nH]1] | 703.2 | — | — | — | — |
| 61 | 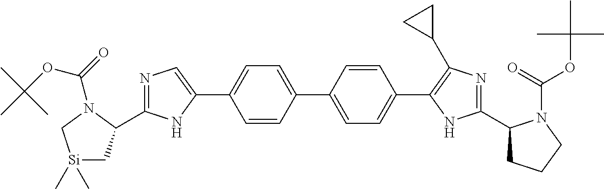 [SMILES: CC(C)(C)OC(=O)N1C[Si](C)(C)C[C@H]1c1ncc(-c2ccc(-c3ccc(-c4[nH]c([C@@H]5CCCN5C(=O)OC(C)(C)C)nc4C4CC4)cc3)cc2)[nH]1] | 709.4 | 81 | — | 655 | — |
| 63 | 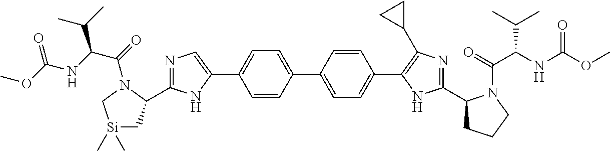 | 823.3 | 0.69 | — | 882 | 451 |
| 64 | 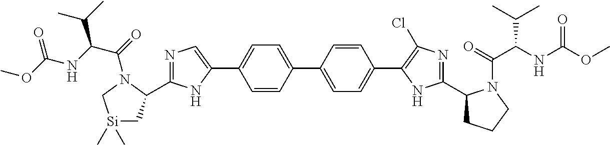 | 818.3 | 0.17 | — | 344 | 99 |
| 65 | 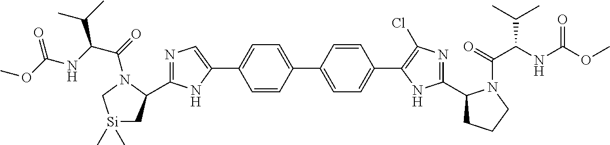 | 817.2 | — | — | — | — |
| 66 | 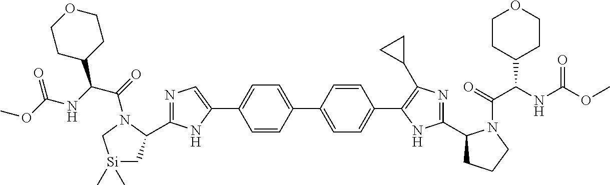 | 907.3 | 0.07 | — | 88 | 1.6 |
| 67 | 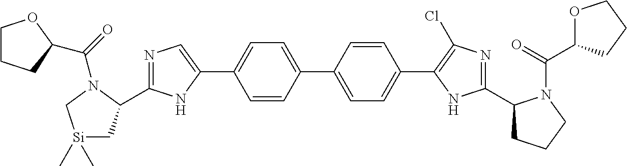 [SMILES: C[Si]1(C)C[C@@H](c2ncc(-c3ccc(-c4ccc(-c5[nH]c([C@@H]6CCCN6C(=O)[C@H]6CCCO6)nc5Cl)cc4)cc3)[nH]2)N(C(=O)[C@H]2CCCO2)C1] | 699.2 | 211 | — | 510 | >1000 |

### Example 41

### Preparation of Compounds 68 and 69

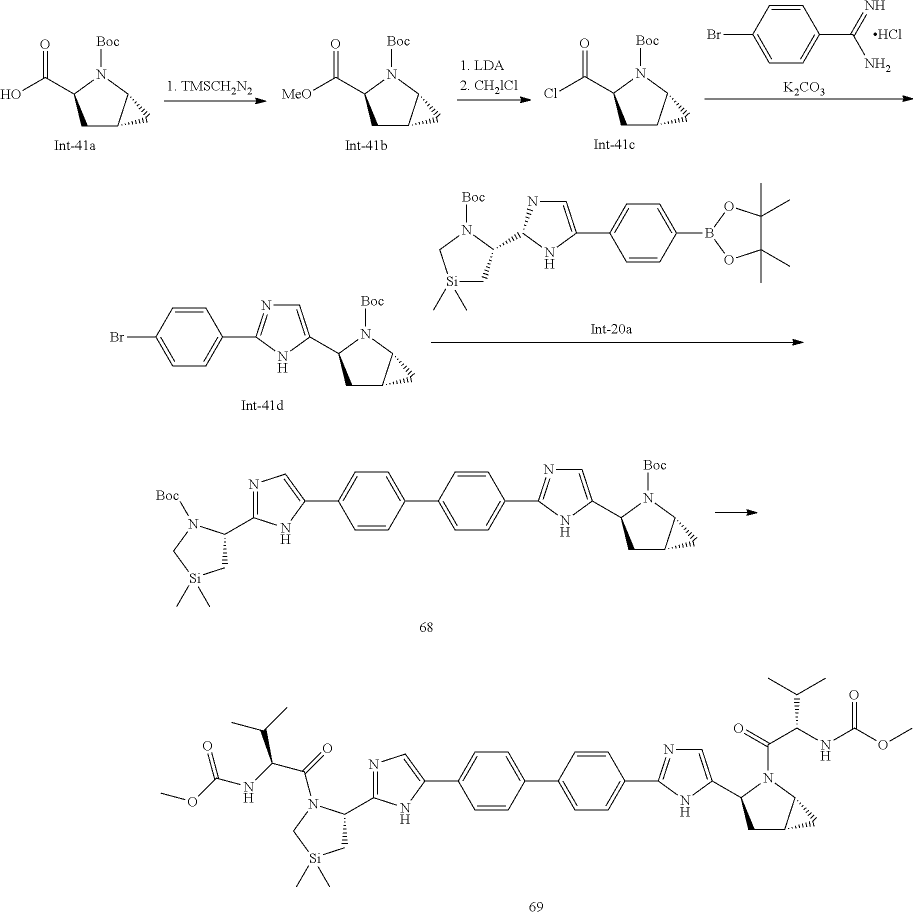

### Step A—Preparation of Compound Int-41b

To a solution of (1R,3S,5R)-2-(tert-butoxycarbonyl)-2-azabicyclo[3.1.0]hexane-3-carboxylic acid Int-41a (2 g, 8.80 mmol) in MeOH (10 mL) and ether (10 mL), trimethylsilyldiazomethane (8.80 mL, 17.60 mmol) (2 M solution in toluene) was added drop wise at ice water bath. Then the resulting solution was recovered room temperature and allowed to stir for 14 h at rt. Evaporated of the solvents and the product, (1R,3S,5R)-2-tert-butyl 3-methyl 2-azabicyclo[3.1.0]hexane-2,3-dicarboxylate Int-41b (2.1 g, 8.80 mmol, 100% yield) was used crude as such in next step.

### Step B—Preparation of Compound Int-41c

To a solution of (1R,3S,5R)-2-tert-butyl 3-methyl 2-azabicyclo[3.1.0]hexane-2,3-dicarboxylate Int-41b (0.5 g, 2.072 mmol) and chloroiodomethane (0.602 mL, 8.29 mmol) in THF (5 mL), LDA (5.18 mL, 10.36 mmol) (2 M solution in THF/heptane) was added dropwise at −75° C. (dry ice-IPA bath). This was allowed to stir for 10 minutes at −75° C. To the reaction mixture was added acetic acid (2 mL) in THF (10 mL) drop wise. This was allowed to stir for additional 10 minutes at −75° C. Diluted with EtOAc (100 mL), washed with brine, NaHCO₃ solution, water. The EtOAc layer was dried over MgSO₄ and evaporated to residue which was purified using column chromatography. (EtOAc-Hexane) to provide (1R,3S,5R)-tert-butyl 3-(2-chloroacetyl)-2-azabicyclo[3.1.0]hexane-2-carboxylate Int-41c (230 mg, 0.886 mmol, 42.7% yield).

### Step C—Preparation of Compound Int-41d

A solution of 4-bromobenzimidamide hydrochloride (163 mg, 0.693 mmol) and K₂CO₃ (192 mg, 1.386 mmol) in THF (1 mL)/Water (0.2 mL) was heated at 65° C. for 5 min. (1R,3S,5R)-tert-butyl 3-(2-chloroacetyl)-2-azabicyclo[3.1.0]hexane-2-carboxylate Int-41c (90 mg, 0.347 mmol) in THF (1 mL) was added drop wise. The reaction mixture was allowed to stir for 14 hours at 65° C. Diluted with EtOAc (100 mL) and washed with water, brine. Organic layer was dried over MgSO₄ and evaporated to residue. This was purified using column chromatography (Silicagel 12 g, EtOAc/Hexane) system to provide (1R,3S,5R)-tert-butyl 3-(2-(4-bromophenyl)-1H-imidazol-5-yl)-2-azabicyclo[3.1.0]hexane-2-carboxylate Int-41d (20 mg, 0.049 mmol, 14.28% yield).

### Step D—Preparation of Compound 68

Using the method described in Example 35, Step A, compound Int-41d (25 mg, 0.062 mmol) was reacted with compound Int-20a (0.11 g, 0.23 mmol) to provide compound 68 (8 mg, 0.012 mmol, 19%).

### Step E—Synthesis of Compound 69

Using the methods described in Example 40, Steps D and E, compound 68 (6 mg, 0.008 mmol) was converted to compound 69 (4 mg. 0.004 mmol, 44%).

Compounds 70-75, depicted in the table below, were made using the methods described in the Example above and substituting the appropriate reactants and/or reagents.

| | | | | | 1a | |
|---|---|---|---|---|---|---|
| | | | 1a | 1b | Y93H | 2b |
| | | Mass | EC90 | EC90 | EC90 | EC90 |
| No. | Structure | Obsvd | nM | nM | nM | nM |
| 68 | 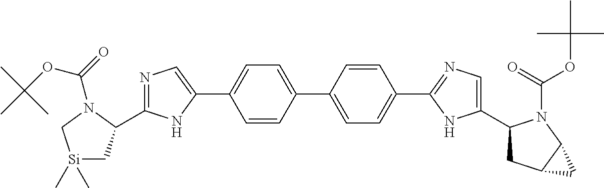 [SMILES: CC(C)(C)OC(=O)N1C[Si](C)(C)C[C@H]1c1ncc(-c2ccc(-c3ccc(-c4ncc([C@@H]5C[C@H]6C[C@H]6N5C(=O)OC(C)(C)C)[nH]4)cc3)cc2)[nH]1] | 681.2 | >1000 | — | >1000 | — |
| 69 | 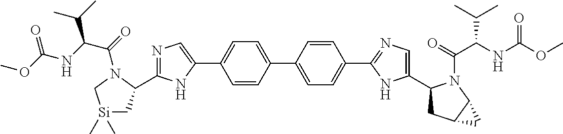 | 795.4 | 0.84 | 0.019 | >100 | 371 |
| 70 | 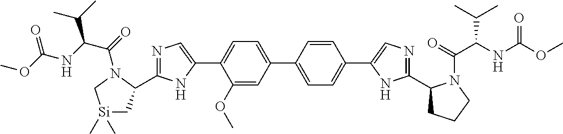 | 813.3 | >1 | 0.006 | >1000 | — |
| 71 | 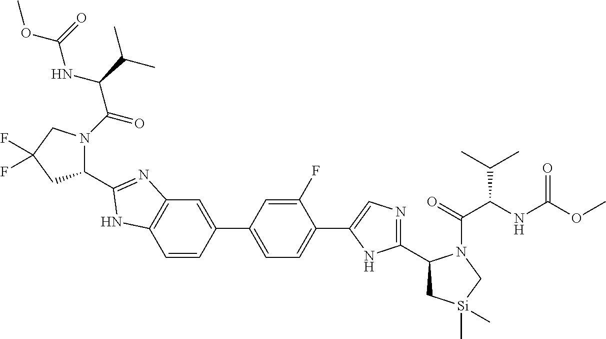 [SMILES: COC(=O)N[C@H](C(=O)N1C[Si](C)(C)C[C@H]1c1ncc(-c2ccc(-c3ccc4[nH]c([C@@H]5CC(F)(F)CN5C(=O)[C@@H](NC(=O)OC)C(C)C)nc4c3)cc2F)[nH]1)C(C)C] | 811.3 | >1 | 0.005 | >1000 | — |
| 72 | 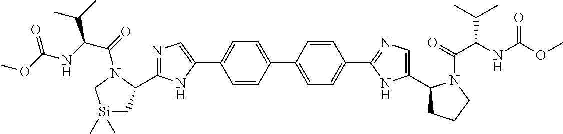 | 783.3 | 0.092 | 0.005 | 330 | — |
| 73 | 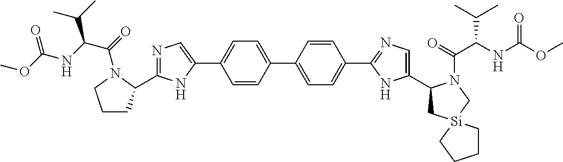 | 809.5 | 0.38 | 0.006 | 973 | — |
| 74 | 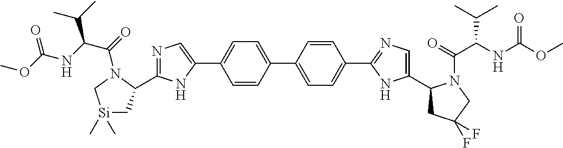 | 819.5 | 0.27 | — | 644 | — |
| 75 | 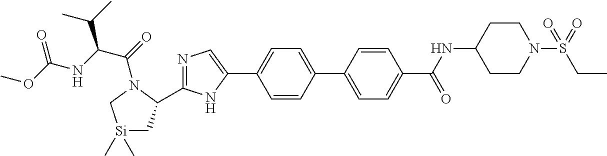 [SMILES: CCS(=O)(=O)N1CCC(NC(=O)c2ccc(-c3ccc(-c4cnc([C@@H]5C[Si](C)(C)CN5C(=O)[C@@H](NC(=O)OC)C(C)C)[nH]4)cc3)cc2)CC1] | 709.3 | 159 | 0.028 | >1000 | >1000 |

### Example 42

### Preparation of Compounds 76-79

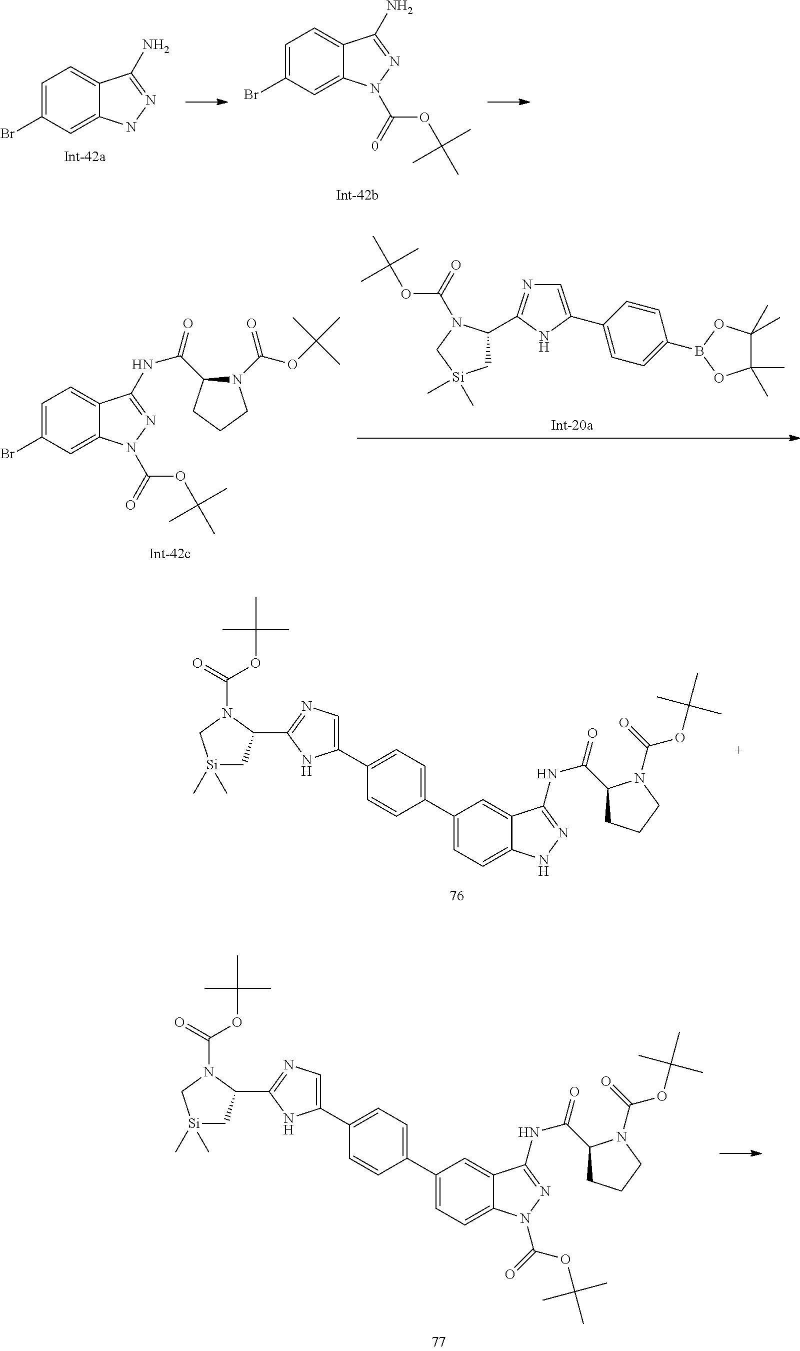

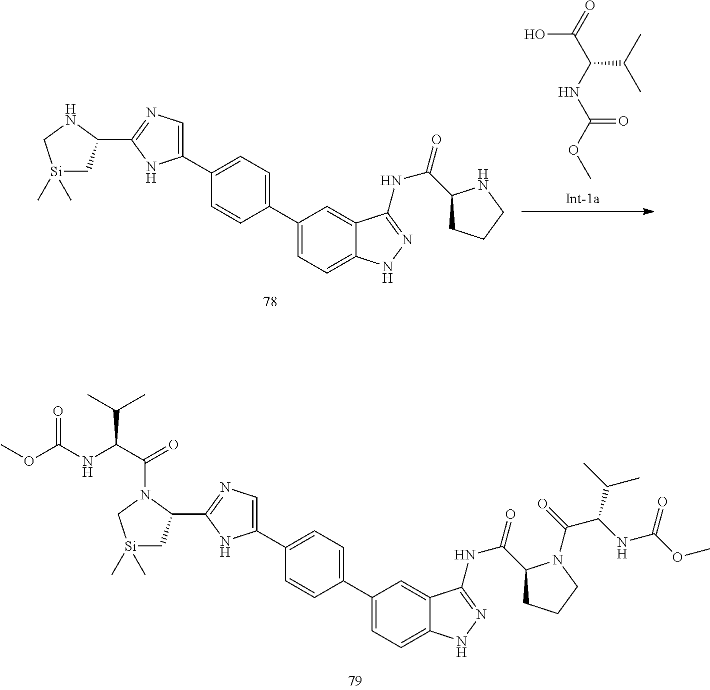

### Step A—Synthesis of Compound Int-42b

To a solution of 6-bromo-1H-indazol-3-amine Int-42a (5 g, 24 mmol) in THF (100 mL) at rt was added Boc₂O (5.2 g, 23.7 mmol) followed by DMAP (0.10 g, 0.82 mmol). The resulting mixture was allowed to stir for 72 h. A small portion of Boc₂O (0.60 g, 2.8 mmol) was added and the mixture was allowed to stir for 2 h. The reaction mixture was concentrated in vacuo to provide a yellowish semisolid. The material was taken up in CH₂Cl₂ (˜20 mL) and was filtered to removed a light yellow solid which was set aside. The filtrate was loaded directly onto a 220 g silica gel column and a gradient of 100% hexanes to 100% EtOAc was run over ˜50 minutes to provide 5.3 g (72%) of Int-42b as a yellow solid.

### Step B—Synthesis of Compound Int-42c

To a solution of Boc-L-Pro-OH (3.3 g, 15 mmol) in DCE (20 mL) was added ethyl 2-ethoxyquinoline-1(2H)-carboxylate (EEDQ, 3.7 g, 15 mmol) and the solution was allowed to stir for 10 min. A solution of tert-butyl-3-amino-6-bromo-1H-indazole-1-carboxylate Int-42b (2.4 g, 7.6 mmol) in DCE (20 mL) was added to provide a homogenous solution. The mixture was affixed with a reflux condenser and was heated to 70° C. and was stirred overnight. The mixture was cooled to room temperature and was partitioned between sat. aq. NaHCO₃ (10 mL) and EtOAc (50 mL). The layers were separated and aqueous layer was extracted with EtOAc (2×50 mL). The organic layers were combined, washed with brine (1×10 mL), dried (Na₂SO₄), filtered, and was concentrated in vacuo to provide a yellow/orange semisolid. The crude material was dissolved in CH₂Cl₂ (5 mL) and was loaded onto a 120 g silica gel column. A gradient of 100% hexanes to 100% EtOAc was run over 45 minutes to provide 3.4 g (86%) of compound Int-42c as a yellow crystalline solid. LC-MS=509.1.

### Step C—Synthesis of Compounds 76 and 77

To a 250 mL round bottom flash charged with a stir bar was added bromoindazole adduct Int-42c (1.0 g, 1.96 mmol) and compound Int-20a (1.2 g, 2.75 mmol) followed by dry dioxane (13 mL) PdCl₂(dppf).CH₂Cl₂ (0.32 g, 0.39 mmol) was added followed by 1M K₂CO₃ (5.0 mL) to provide a yellow, biphasic mixture. The mixture was affixed with a reflux condenser and was degassed under house vacuum and filled with N2. This protocol was repeated six times and the mixture was heated to 95° C. The mixture was allowed to stir for 12 hours at 95° C. whereupon the mixture was cooled to room temperature. The mixture was partitioned between EtOAc (10 mL) and water (2 mL) and the layers were separated. The aqueous mixture was extracted with EtOAc (2×10 mL) and the layers were separated. The organic layer was washed with brine (1×4 mL), dried (Na₂SO₄), filtered, and concentrated in vacuo. The crude material was purified using flash chromatography using an ISCO (80 g column) using a gradient of 100% CH₂Cl₂ to 10% CH₂Cl₂/MeOH to provide two fractions as follows:

Compound 76: 86 mg (18%): LC-MS=686.3.

Compound 77: 205 mg (38%): LC-MS=787.4.

### Step D—Synthesis of Compound 78

To a solution of compound 77 (0.21 g, 0.26 mmol) in MeOH (2 mL) was added 4N HCl (˜1 mL) at rt. The resulting orange, homogenous mixture was stirred at rt for 2 hours and was concentrated in vacuo with heating to provide a light orange solid. This material was placed under high vacuum overnight afford 160 mg (99%) of compound 78 as a maize solid which was used in the next transformation without further purification or characterization. LC-MS=486.2.

### Step E—Synthesis of Compound 79

To a solution of compound 78 (0.16 g, 0.26 mmol) in DMF (2 mL) at −10° C. (ice/acetone) was added (S)-2-(methoxycarbonylamino)-3-methylbutanoic acid Int-1a (96 mg, 0.55 mmol), HATU (0.21 g, 0.55 mmol), followed by dropwise addition of DIEA (0.27 mL, 1.57 mmol) to provide an orange, homogenous solution. The resulting solution was allowed to stir for 1 hour at −10° C. whereupon the mixture was diluted with water (1.5 mL) and EtOAc (4 mL). The mixture was allowed to warm to room temperature and the layers were separated. The aqueous layer was extracted with EtOAc (3×4 mL) and the organic layers were combined. The organic layer was washed with brine (1×3 mL), dried (Na₂SO₄), filtered, and concentrated in vacuo. The resulting orange/brown semisolid was placed under high vacuum. The crude material was purified using reverse-phase HPLC (Gilson) using a C18 column with a gradient: 0% ACN to 90% ACN/10% water (both with 0.1% TFA) to provide 132 mg (57%) of compound 79 as a light yellow dihydrochloride salt after treatment with HCl. LC-MS=800.4.

| | | | | | 1a | |
|---|---|---|---|---|---|---|
| | | | 1a | 1b | Y93H | 2b |
| | | Mass | EC90 | EC90 | EC90 | EC90 |
| No. | Structure | Obsvd | nM | nM | nM | nM |
| 76 | 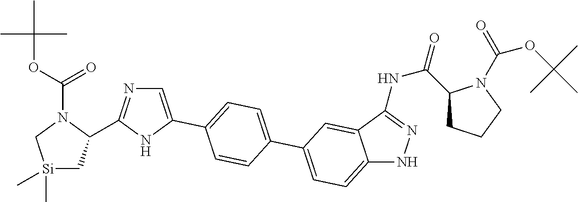 [SMILES: CC(C)(C)OC(=O)N1CCC[C@H]1C(=O)Nc1n[nH]c2ccc(-c3ccc(-c4cnc([C@@H]5C[Si](C)(C)CN5C(=O)OC(C)(C)C)[nH]4)cc3)cc12] | 686.3 | >1000 | — | >10000 | — |
| 77 | 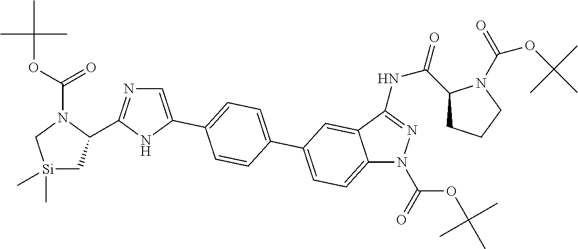 [SMILES: CC(C)(C)OC(=O)N1CCC[C@H]1C(=O)Nc1nn(C(=O)OC(C)(C)C)c2ccc(-c3ccc(-c4cnc([C@@H]5C[Si](C)(C)CN5C(=O)OC(C)(C)C)[nH]4)cc3)cc12] | 787.4 | >1000 | — | >10000 | — |
| 79 | 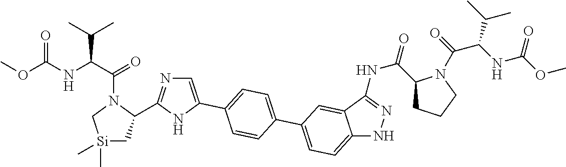 | 800.4 | >1 | >1 | >100 | >100 |

### Example 43

### Preparation of Compounds 80-82

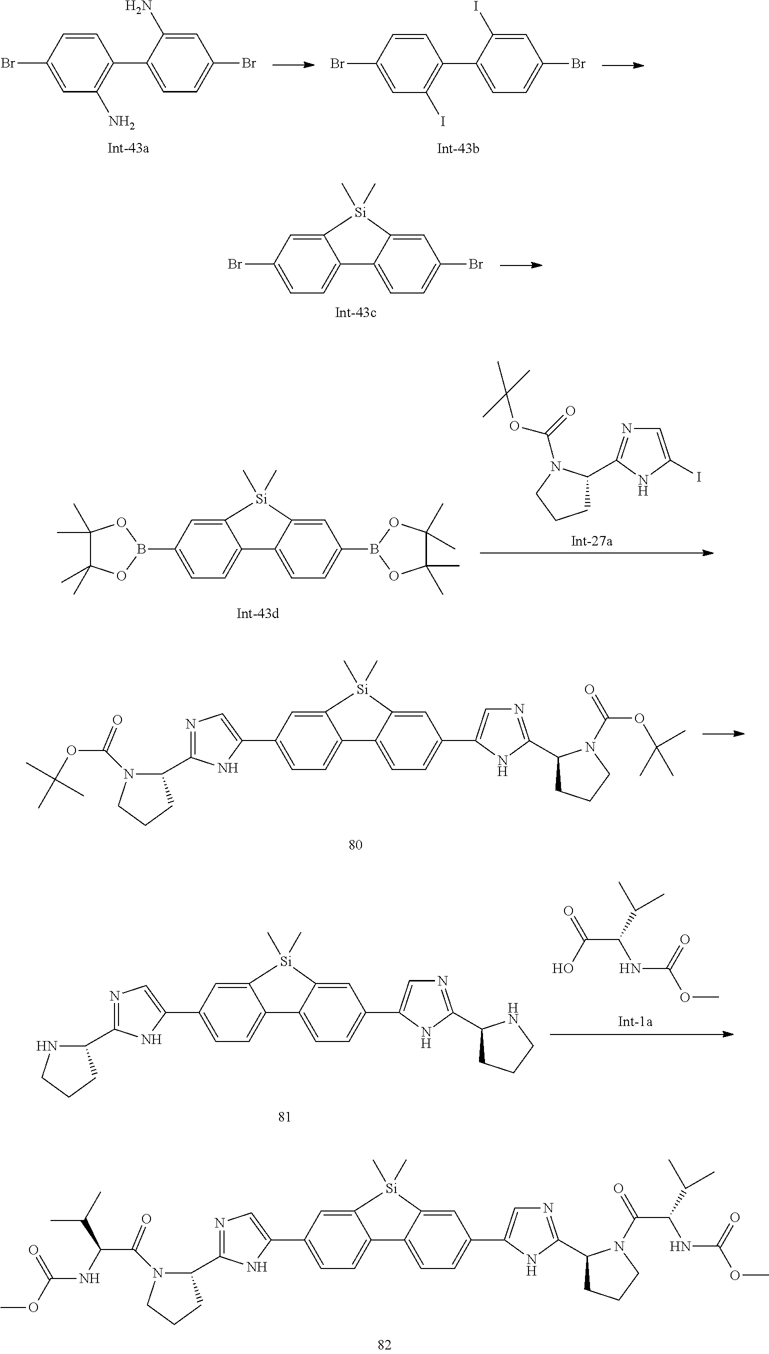

### Step A—Preparation of Compound Int-43b

To a stirred suspension of 2,2′-diamino-4,4′-dibromobiphenyl Int-43a (11.2 g, 33 mmol) (prepared from J. Am. Chem. Soc. 2005, 127, 7662) in conc. HCl (50 mL)/water (50 mL) at 0° C. was added a 50% soln of NaNO₂ in water (30 mL, 73 mmol) over 30 minutes while maintaining the temperature <5° C. The mixture was allowed to stir for an additional 30 minutes whereupon a soln of KI (54 g, 0.33 mol) in water (120 mL) was added dropwise over 1 h. The resulting mixture was heated to 60° C. and was allowed to stir for 5 h. The mixture was cooled to room temperature and was filtered. The resultant solid was washed with EtOAc (˜500 mL) and the resultant filtrate was washed with brine (2×50 mL), dried (Na₂SO₄), filtered, and concentrated in vacuo. The crude product was purified using flash chromatography using 100% hexanes to provide 5.1 g (27%) of compound Int-43b as a colorless oil.

### Step B—Preparation of Compound Int-43c

To a stirred solution of compound Int-43b (2.8 g, 4.9 mmol) in THF (90 mL) at −78° C. was added n-BuLi (2.5 M in hexanes, 7.8 mL, 19.5 mmol) dropwise over ˜2.5 h. Once the addition was complete, the mixture was allowed to stir for one hour at −78° C. whereupon Me₂SiCl₂ (0.65 mL, 5.4 mmol) was added rapidly. The mixture was allowed to stir for an additional 35 minutes at −78° C., the cooling bath was removed, and the mixture was stirred overnight at rt. Water (10 mL) was added followed by dilution with hexanes (100 mL) and the layers were separated. The organic layer was washed with brine (1×25 mL), dried (Na₂SO₄), filtered, and concentrated in vacuo. The crude product was purified using flash chromatography eluting with hexanes to provide 0.82 g (45%) of compound Int-43c as a white solid.

### Step C—Preparation of Compound Int-43d

To a stirred solution of compound Int-43c (0.82 g, 2.2 mmol) in THF (20 mL) at −78° C. was added t-BuLi (1.7 M in pentane, 5.3 mL, 9.1 mmol) dropwise over ˜40 minutes. The mixture was stirred an additional 35 minutes at −78° C. whereupon 2-isopropoxy-4,4,5,5-tetramethyl[1,3,2]dioxaborolane (1.1 mL, 5.5 mmol) was added over 5 min. The mixture was allowed to warm to room temperature and stir overnight. Water (25 mL) was added followed by dilution with hexanes (100 mL) and the layers were separated. The aqueous layer was extracted with Et₂O (3×25 mL) and the organic layers were combined. The organic layer was washed with brine (2×10 mL), dried (Na₂SO₄), filtered, and concentrated in vacuo. The crude product was purified using flash chromatography using a gradient of 100% hexanes to 75% hexanes/25% EtOAc to provide 0.18 g (45%) of compound Int-43d as a white solid.

### Step D—Preparation of Compound 80

To a 20 ml tube equipped with a stir bar was added compound Int-43d (0.18 g, 0.38 mmol), (S)-tert-butyl 2-(5-iodo-1H-imidazol-2-yl)pyrrolidine-1-carboxylate Int-27a (0.34 g, 0.94 mmol), Pd₂dba₃ (34 mg, 0.038 mmol), AmPhos (40 mg, 0.15 mmol), K₂CO₃ (0.26 g, 1.9 mmol) followed by water (2 mL) and DME (12 mL). The mixture was degassed under vacuum and filled with Argon and this protocol was repeated several times. The tube was heated to 85° C., allowed to stir for 12 hours, and was cooled to room temperature. The mixture was diluted with EtOAc (5 mL) and was filtered thru a pad of Celite washing the pad with EtOAc (100 mL). The resulting filtrate was washed with brine (2×5 mL), dried (Na₂SO₄), filtered, and concentrated in vacuo. The crude product was purified using flash chromatography using a gradient of 100% CH₂Cl₂ to 95% CH₂Cl₂/MeOH to provide 0.19 g (75%) of compound 80 as a light yellow solid.

### Step E—Preparation of Compound 81

To a solution of compound 80 (0.16 g, 0.23 mmol) in CH₂Cl₂ (4 mL) at 0° C. was added TFA (1 mL). The mixture was allowed to stir for 2 hours and was concentrated in vacuo. The resultant resulting residue was dissolved in MeOH (1 mL), treated with 4N HCl in dioxane (0.6 mL), and was concentrated in vacuo to provide compound 81, which was used without further purification.

### Step F—Preparation of Compound 82

To a solution of compound 81 (0.14 g, 0.23 mmol) in DMF (6 mL) at 0° C. was added (S)-2-((methoxycarbonyl)amino)-3-methylbutanoic acid Int-1a (90 mg, 0.51 mmol), DIEA (0.40 mL, 2.3 mmol), and HATU (0.20 g, 0.53 mmol). The mixture was allowed to stir for 10 minutes at 0° C., water (50 mL) was added, and the resultant solid was filtered off and dried under vacuum. The crude solid was purified using flash chromatography using a gradient of 100% hexanes to 25% hexanes/75% EtOAc to provide 89 mg (49%) of compound 82 as a yellow solid. LC-MS=795.7.

Compounds 83 and 84, depicted in the table below, were made using the methods described in the Example above and substituting the appropriate reactants and/or reagents.

| | | | | | 1a | |
|---|---|---|---|---|---|---|
| | | | 1a | 1b | Y93H | 2b |
| | | Mass | EC90 | EC90 | EC90 | EC90 |
| No. | Structure | Obsvd | nM | nM | nM | nM |
| 80 | 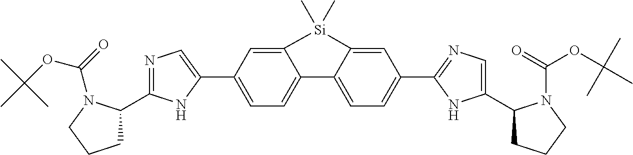 [SMILES: CC(C)(C)OC(=O)N1CCC[C@H]1c1cnc(-c2ccc3c(c2)[Si](C)(C)c2cc(-c4cnc([C@@H]5CCCN5C(=O)OC(C)(C)C)[nH]4)ccc2-3)[nH]1] | 680.9 | >1 | 467 | — | — |
| 82 | 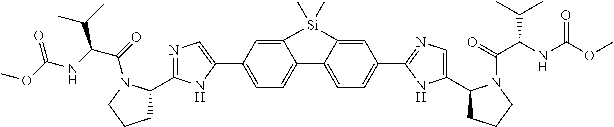 | 795.7 | 0.076 | 0.043 | 811 | — |
| 83 | 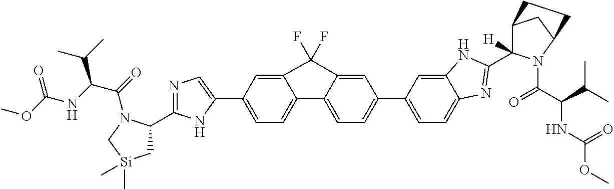 | — | — | — | — | — |
| 84 | 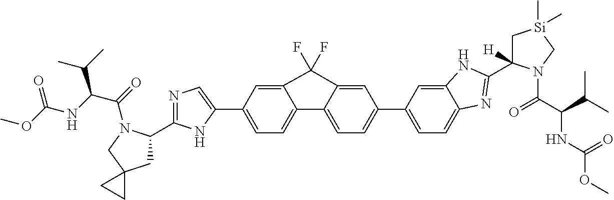 | — | — | — | — | — |

NA = Not Available

Compounds 85-210, depicted in the table below, can be made using the methods described in the Examples above.

| No. | Structure |
|---|---|
| 85 | 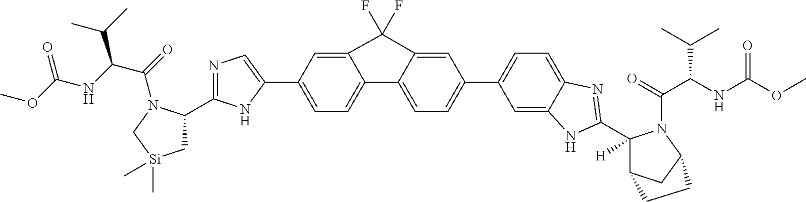 |
| 86 | 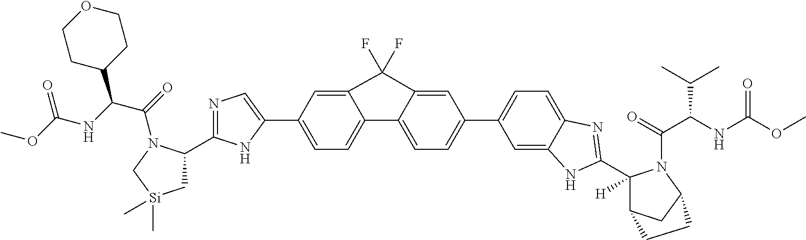 |
| 87 | 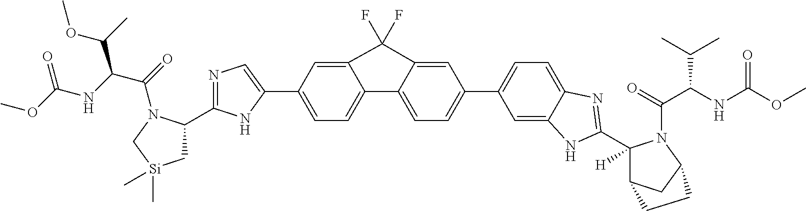 |
| 88 | 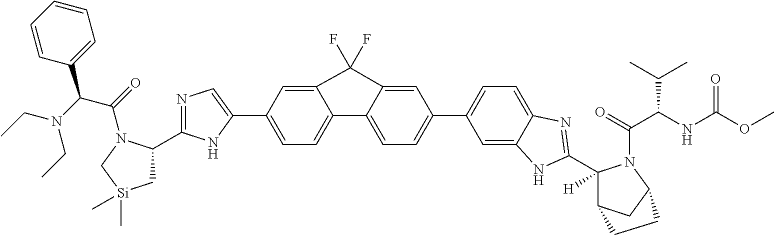 [SMILES: CCN(CC)[C@H](C(=O)N1C[Si](C)(C)C[C@H]1c1ncc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)[nH]1)c1ccccc1] |
| 89 | 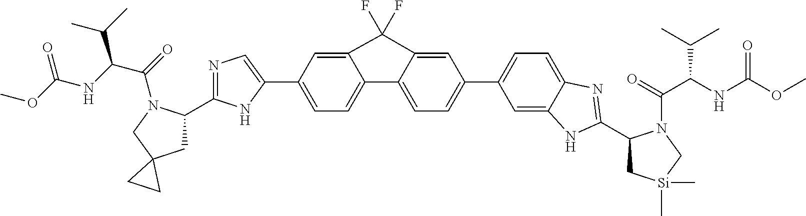 |
| 90 | 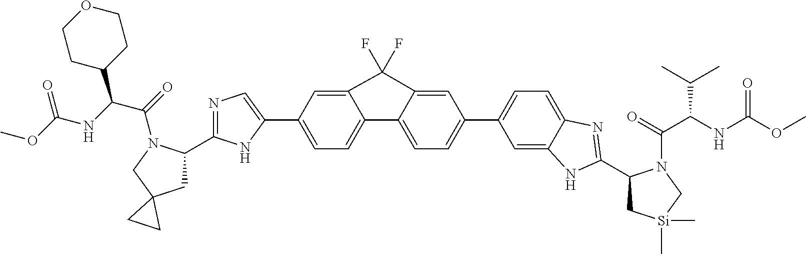 |
| 97 | 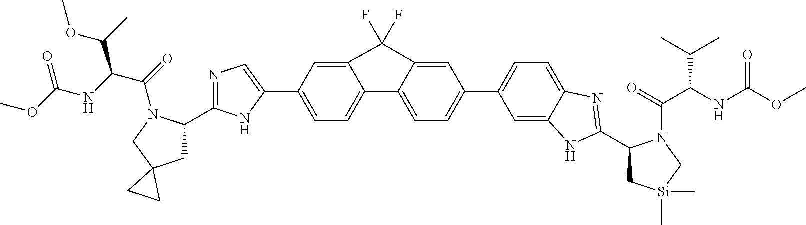 |
| 92 | 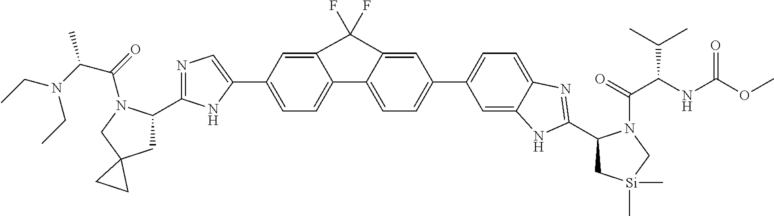 [SMILES: CCN(CC)[C@H](C)C(=O)N1CC2(CC2)C[C@H]1c1ncc(-c2ccc3c(c2)C(F)(F)c2cc(-c4ccc5nc([C@@H]6C[Si](C)(C)CN6C(=O)[C@@H](NC(=O)OC)C(C)C)[nH]c5c4)ccc2-3)[nH]1] |
| 93 | 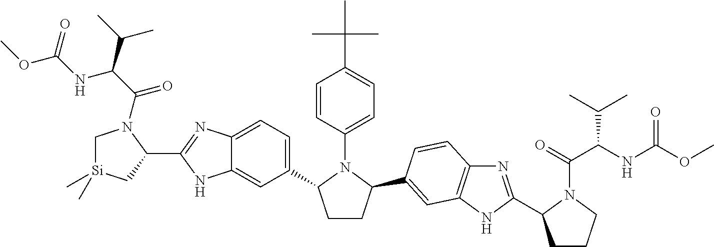 |
| 94 | 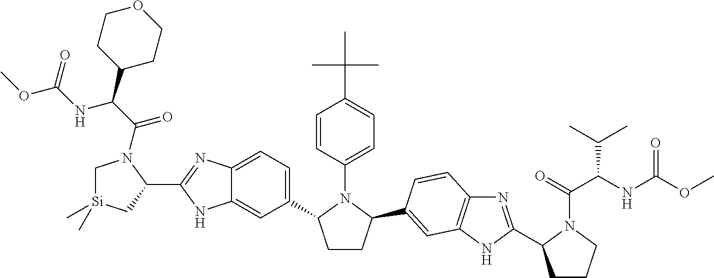 |
| 95 | 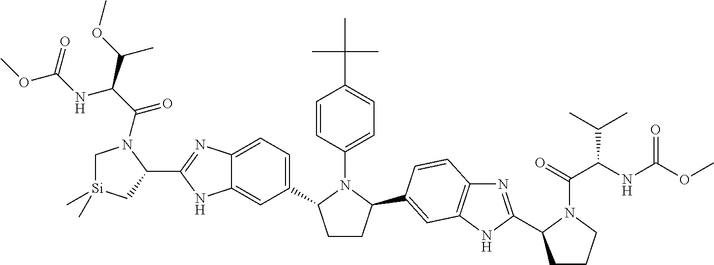 |
| 96 | 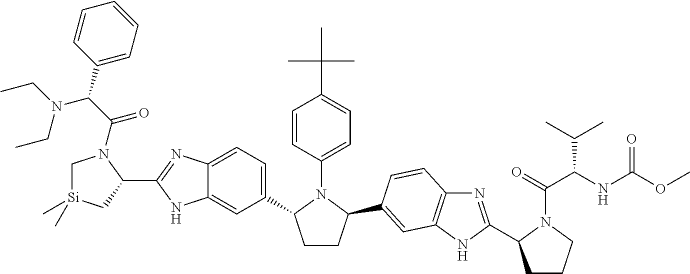 [SMILES: CCN(CC)[C@@H](C(=O)N1C[Si](C)(C)C[C@H]1c1nc2ccc([C@H]3CC[C@H](c4ccc5nc([C@@H]6CCCN6C(=O)[C@@H](NC(=O)OC)C(C)C)[nH]c5c4)N3c3ccc(C(C)(C)C)cc3)cc2[nH]1)c1ccccc1] |
| 97 | 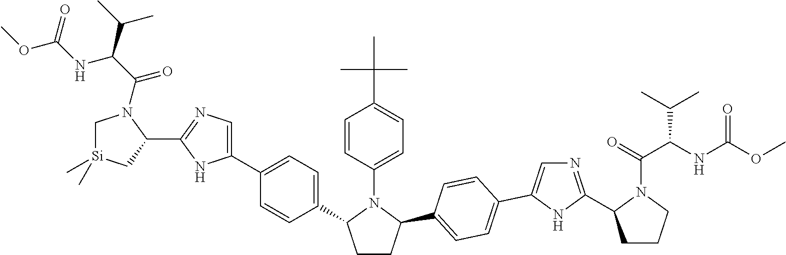 |
| 98 | 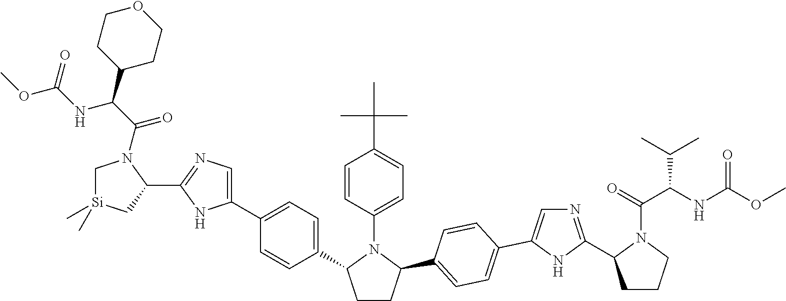 |
| 99 | 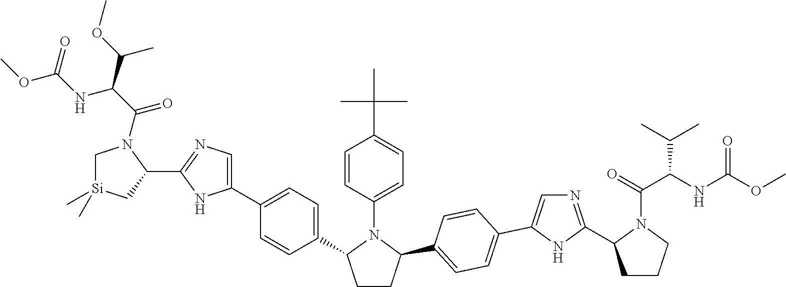 |
| 100 | 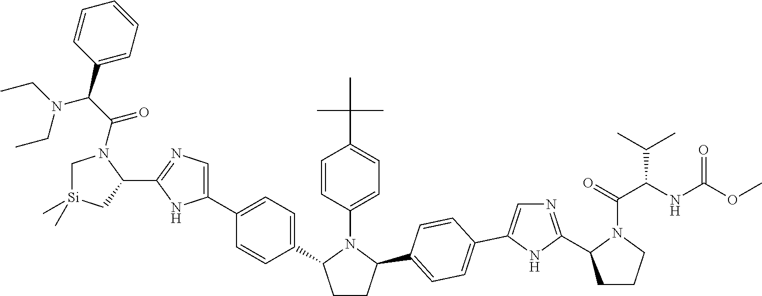 [SMILES: CCN(CC)[C@H](C(=O)N1C[Si](C)(C)C[C@H]1c1ncc(-c2ccc([C@H]3CC[C@H](c4ccc(-c5cnc([C@@H]6CCCN6C(=O)[C@@H](NC(=O)OC)C(C)C)[nH]5)cc4)N3c3ccc(C(C)(C)C)cc3)cc2)[nH]1)c1ccccc1] |
| 101 | 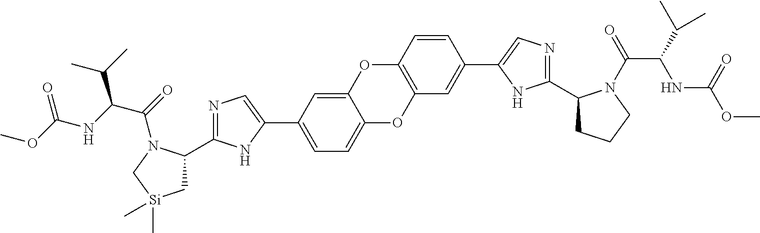 |
| 102 | 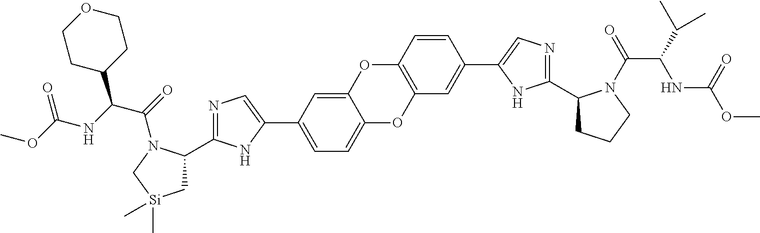 |
| 103 | 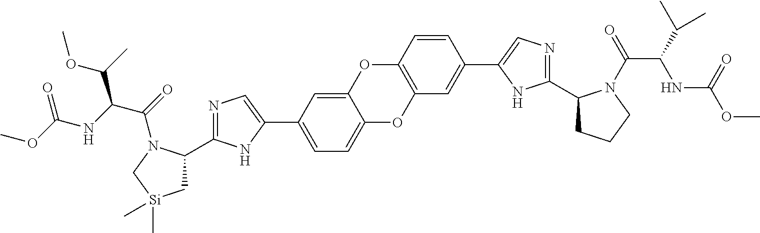 |
| 104 | 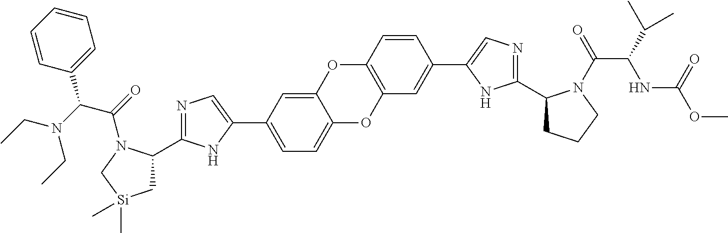 [SMILES: CCN(CC)[C@@H](C(=O)N1C[Si](C)(C)C[C@H]1c1ncc(-c2ccc3c(c2)Oc2ccc(-c4cnc([C@@H]5CCCN5C(=O)[C@@H](NC(=O)OC)C(C)C)[nH]4)cc2O3)[nH]1)c1ccccc1] |
| 105 | 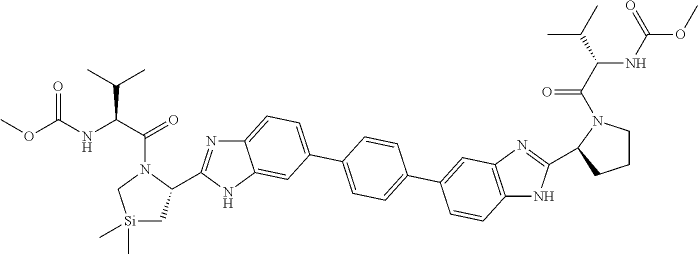 |
| 106 | 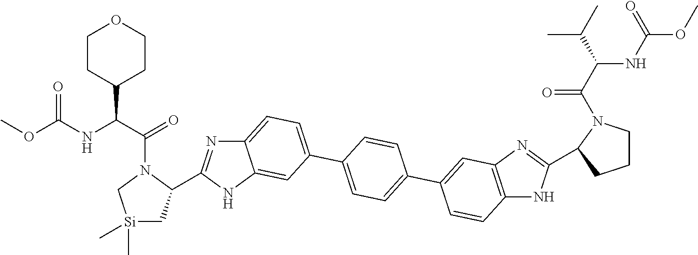 |
| 107 | 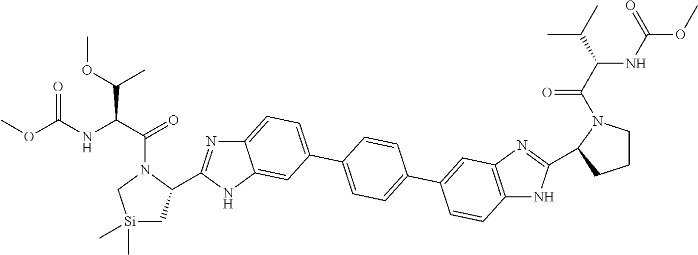 |
| 108 | 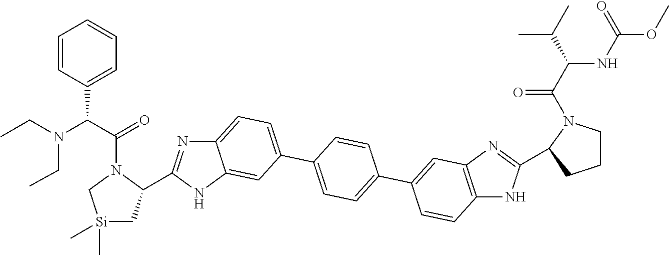 [SMILES: CCN(CC)[C@@H](C(=O)N1C[Si](C)(C)C[C@H]1c1nc2ccc(-c3ccc(-c4ccc5[nH]c([C@@H]6CCCN6C(=O)[C@@H](NC(=O)OC)C(C)C)nc5c4)cc3)cc2[nH]1)c1ccccc1] |
| 109 | 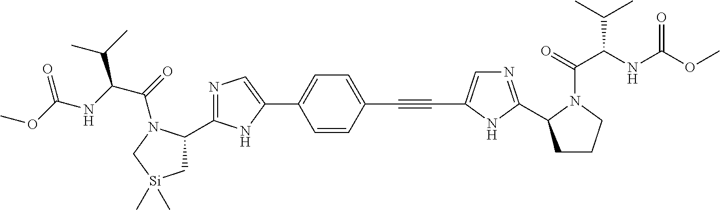 |
| 110 | 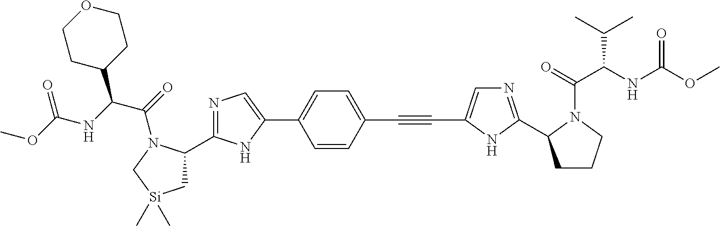 |
| 111 | 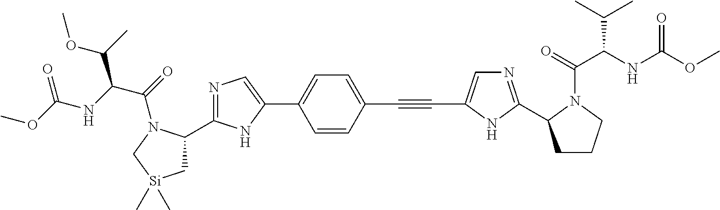 |
| 112 | 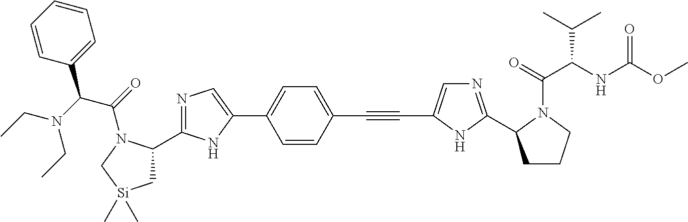 [SMILES: CCN(CC)[C@H](C(=O)N1C[Si](C)(C)C[C@H]1c1ncc(-c2ccc(C#Cc3cnc([C@@H]4CCCN4C(=O)[C@@H](NC(=O)OC)C(C)C)[nH]3)cc2)[nH]1)c1ccccc1] |
| 113 | 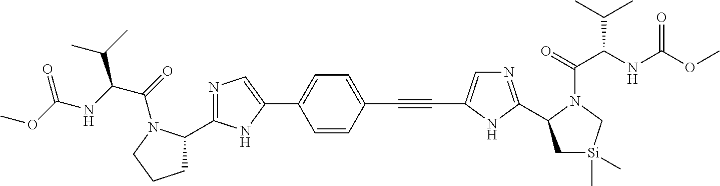 |
| 114 | 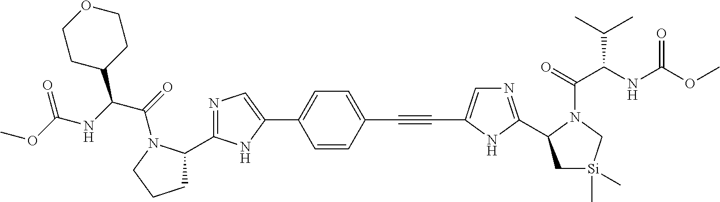 |
| 115 | 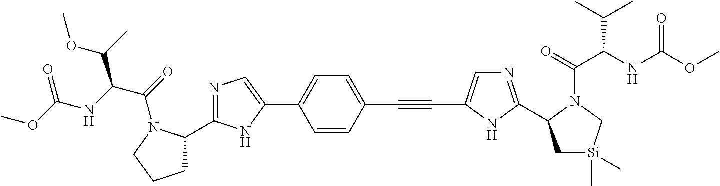 |
| 116 | 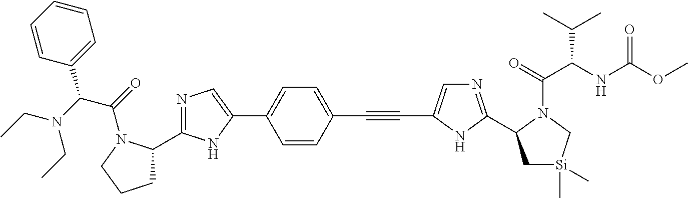 [SMILES: CCN(CC)[C@@H](C(=O)N1CCC[C@H]1c1ncc(-c2ccc(C#Cc3cnc([C@@H]4C[Si](C)(C)CN4C(=O)[C@@H](NC(=O)OC)C(C)C)[nH]3)cc2)[nH]1)c1ccccc1] |
| 117 | 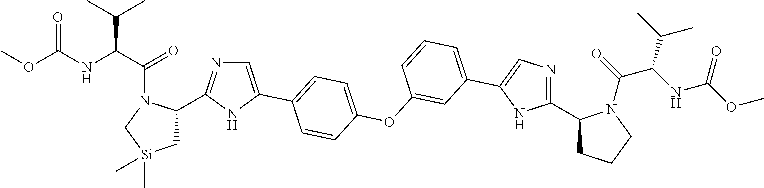 |
| 118 | 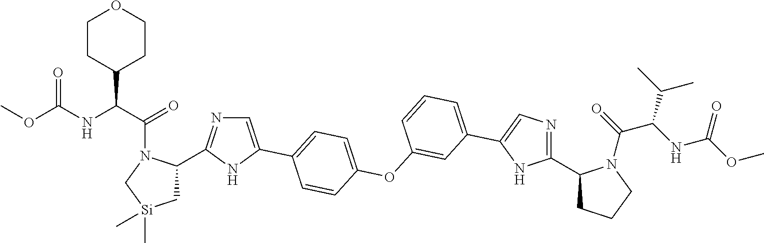 |
| 119 | 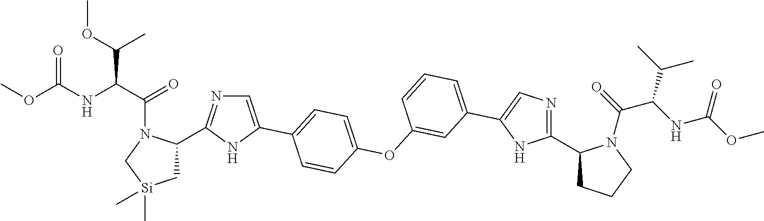 |
| 120 | 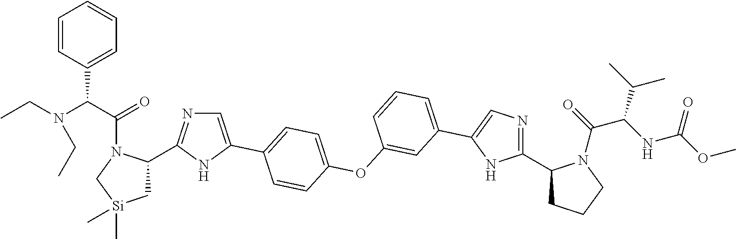 [SMILES: CCN(CC)[C@@H](C(=O)N1C[Si](C)(C)C[C@H]1c1ncc(-c2ccc(Oc3cccc(-c4cnc([C@@H]5CCCN5C(=O)[C@@H](NC(=O)OC)C(C)C)[nH]4)c3)cc2)[nH]1)c1ccccc1] |
| 121 | 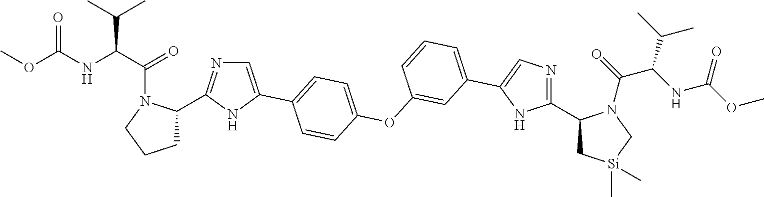 |
| 122 | 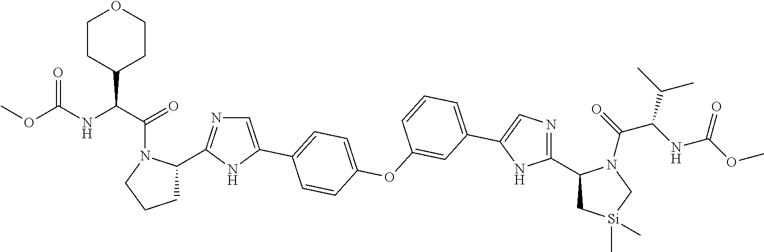 |
| 123 | 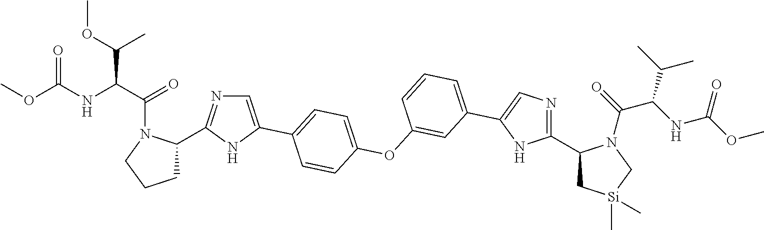 |
| 124 | 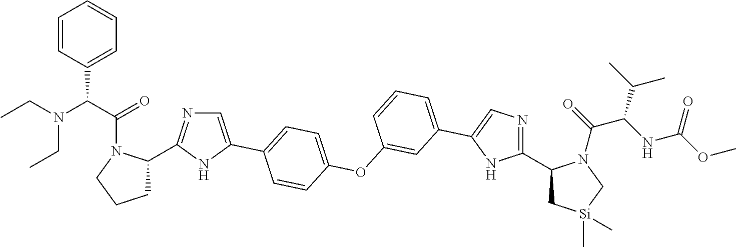 [SMILES: CCN(CC)[C@@H](C(=O)N1CCC[C@H]1c1ncc(-c2ccc(Oc3cccc(-c4cnc([C@@H]5C[Si](C)(C)CN5C(=O)[C@@H](NC(=O)OC)C(C)C)[nH]4)c3)cc2)[nH]1)c1ccccc1] |
| 125 | 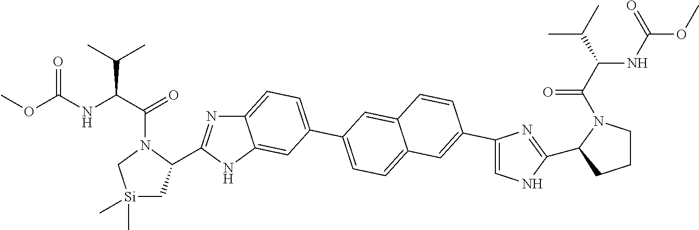 |
| 126 | 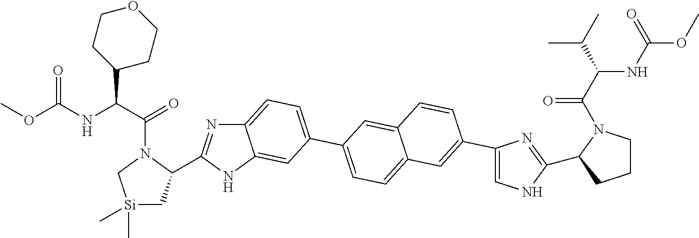 |
| 127 | 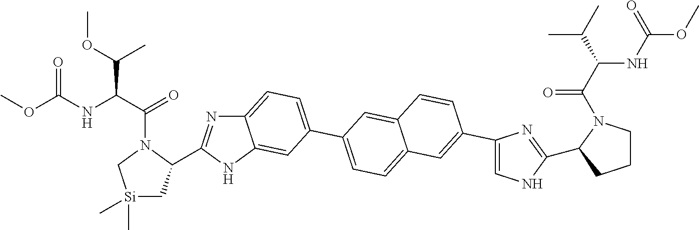 |
| 128 | 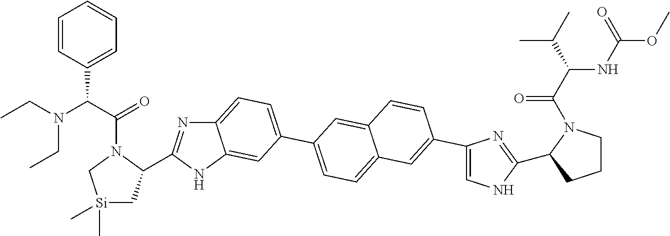 [SMILES: CCN(CC)[C@@H](C(=O)N1C[Si](C)(C)C[C@H]1c1nc2ccc(-c3ccc4cc(-c5c[nH]c([C@@H]6CCCN6C(=O)[C@@H](NC(=O)OC)C(C)C)n5)ccc4c3)cc2[nH]1)c1ccccc1] |
| 129 | 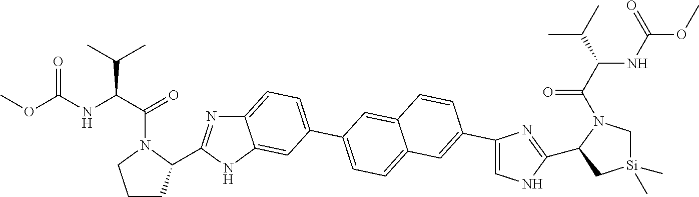 |
| 130 | 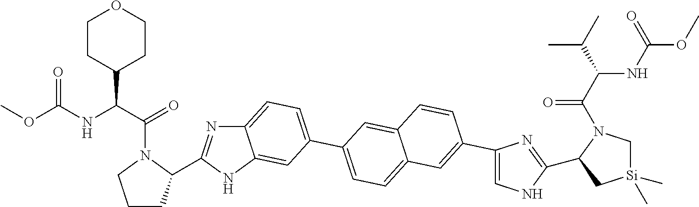 |
| 131 | 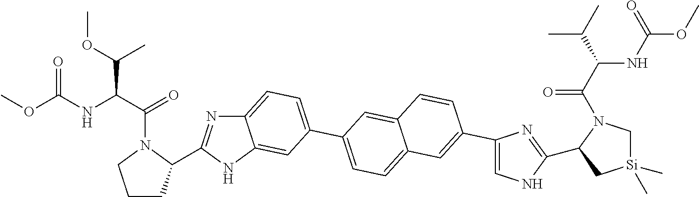 |
| 132 | 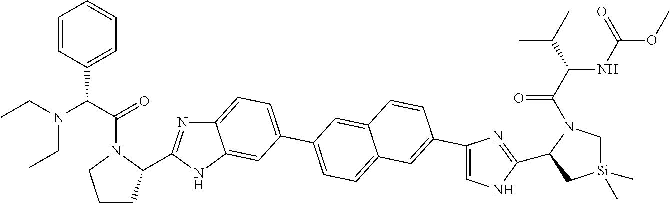 [SMILES: CCN(CC)[C@@H](C(=O)N1CCC[C@H]1c1nc2ccc(-c3ccc4cc(-c5c[nH]c([C@@H]6C[Si](C)(C)CN6C(=O)[C@@H](NC(=O)OC)C(C)C)n5)ccc4c3)cc2[nH]1)c1ccccc1] |
| 133 | 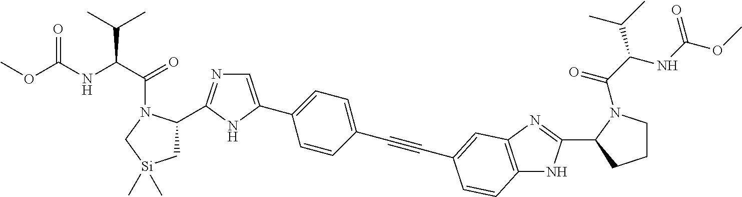 |
| 134 | 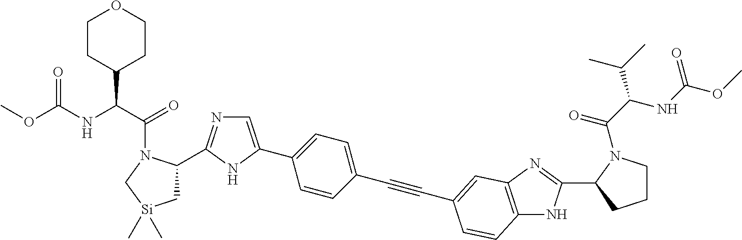 |
| 135 | 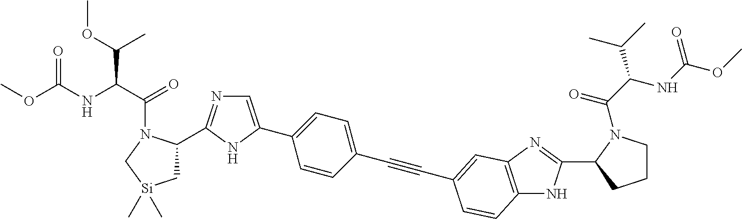 |
| 136 | 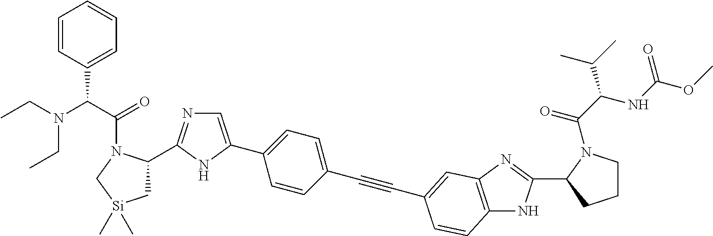 [SMILES: CCN(CC)[C@@H](C(=O)N1C[Si](C)(C)C[C@H]1c1ncc(-c2ccc(C#Cc3ccc4[nH]c([C@@H]5CCCN5C(=O)[C@@H](NC(=O)OC)C(C)C)nc4c3)cc2)[nH]1)c1ccccc1] |
| 137 | 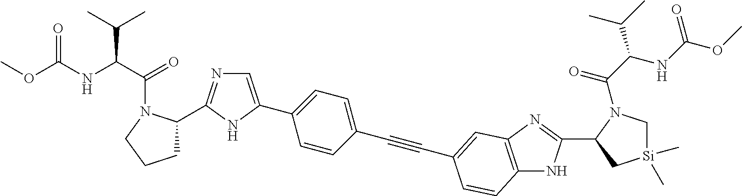 |
| 138 | 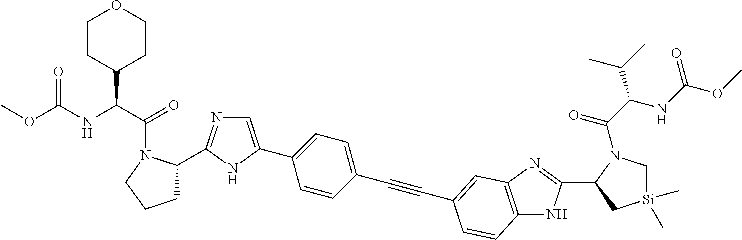 |
| 139 | 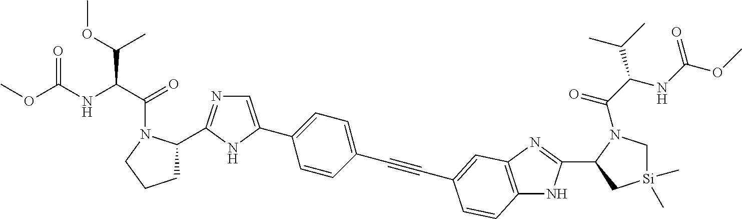 |
| 140 | 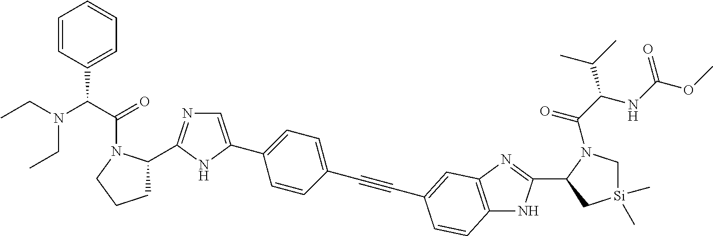 [SMILES: CCN(CC)[C@@H](C(=O)N1CCC[C@H]1c1ncc(-c2ccc(C#Cc3ccc4[nH]c([C@@H]5C[Si](C)(C)CN5C(=O)[C@@H](NC(=O)OC)C(C)C)nc4c3)cc2)[nH]1)c1ccccc1] |
| 141 | 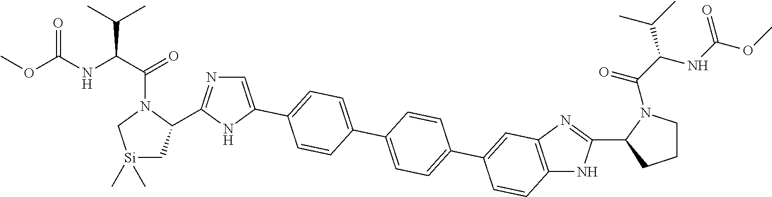 |
| 142 | 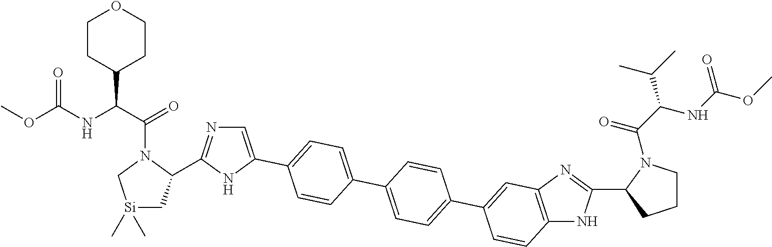 |
| 143 | 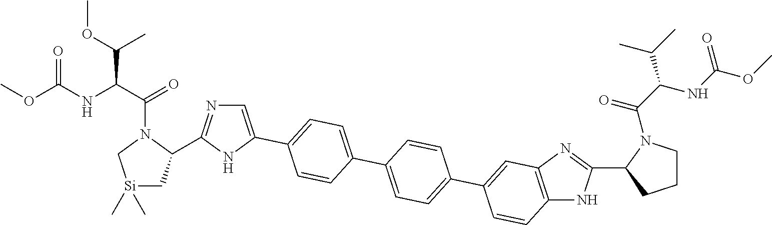 |
| 144 | 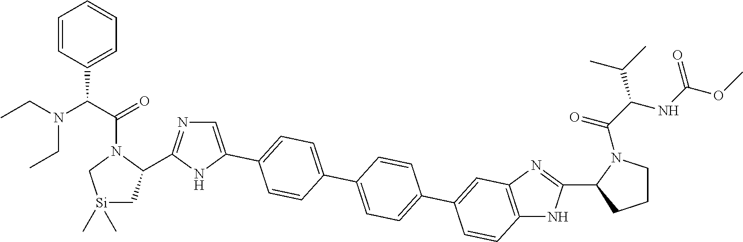 [SMILES: CCN(CC)[C@@H](C(=O)N1C[Si](C)(C)C[C@H]1c1ncc(-c2ccc(-c3ccc(-c4ccc5[nH]c([C@@H]6CCCN6C(=O)[C@@H](NC(=O)OC)C(C)C)nc5c4)cc3)cc2)[nH]1)c1ccccc1] |
| 145 | 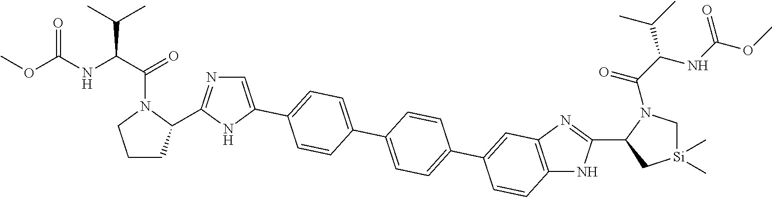 |
| 146 | 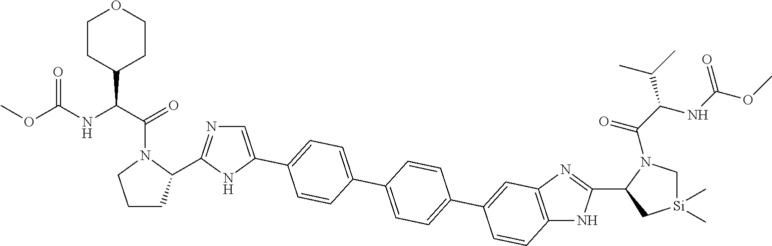 |
| 147 | 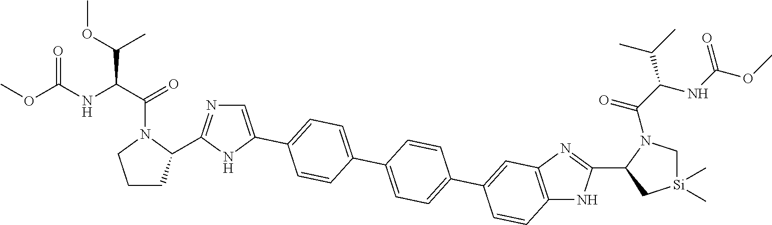 |
| 148 | 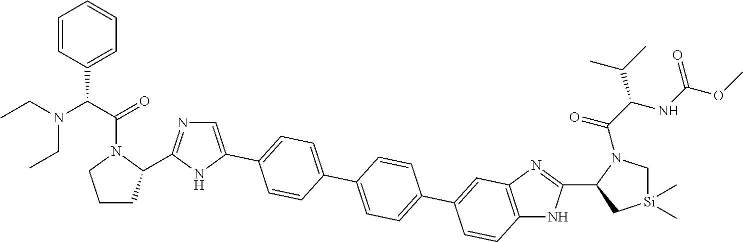 [SMILES: CCN(CC)[C@@H](C(=O)N1CCC[C@H]1c1ncc(-c2ccc(-c3ccc(-c4ccc5[nH]c([C@@H]6C[Si](C)(C)CN6C(=O)[C@@H](NC(=O)OC)C(C)C)nc5c4)cc3)cc2)[nH]1)c1ccccc1] |
| 149 | 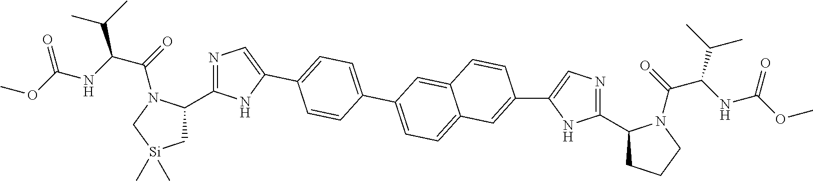 |
| 150 | 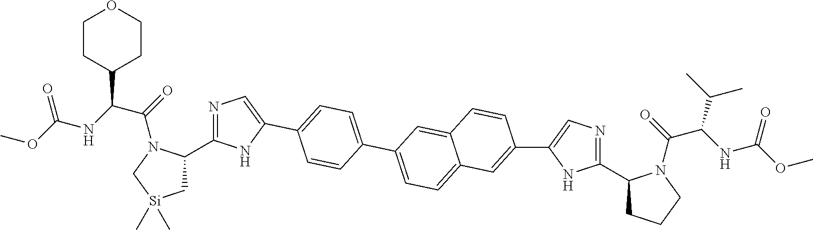 |
| 151 | 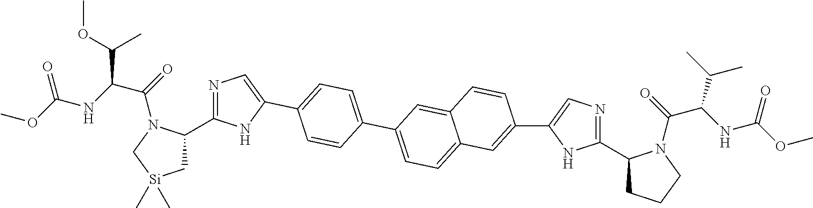 |
| 152 | 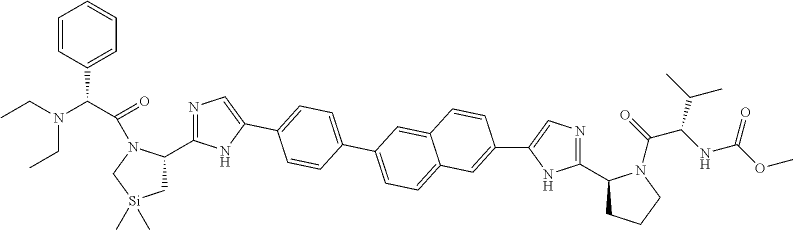 [SMILES: CCN(CC)[C@@H](C(=O)N1C[Si](C)(C)C[C@H]1c1ncc(-c2ccc(-c3ccc4cc(-c5cnc([C@@H]6CCCN6C(=O)[C@@H](NC(=O)OC)C(C)C)[nH]5)ccc4c3)cc2)[nH]1)c1ccccc1] |
| 153 | 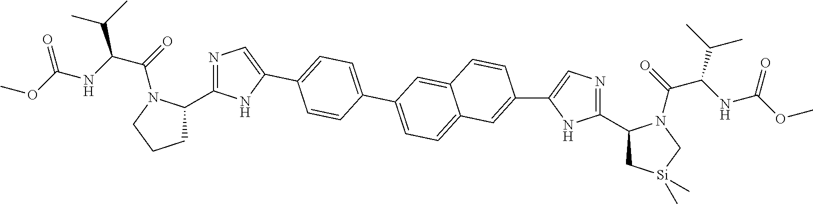 |
| 154 | 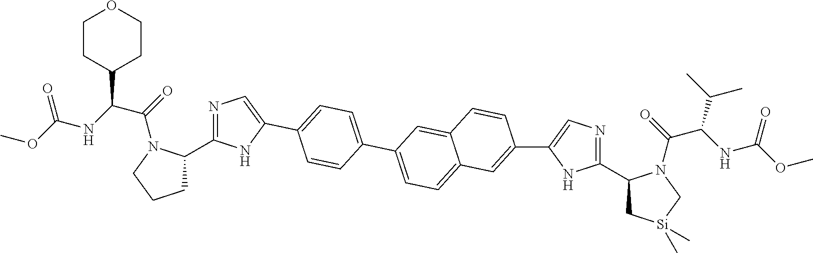 |
| 155 | 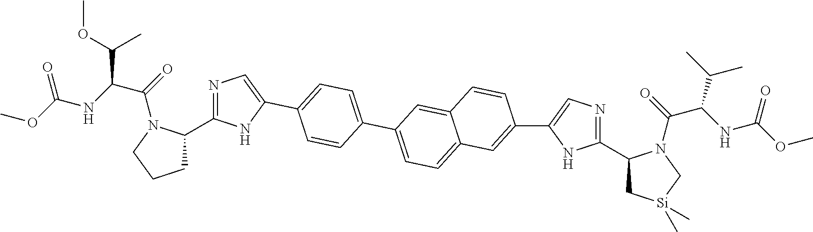 |
| 156 | 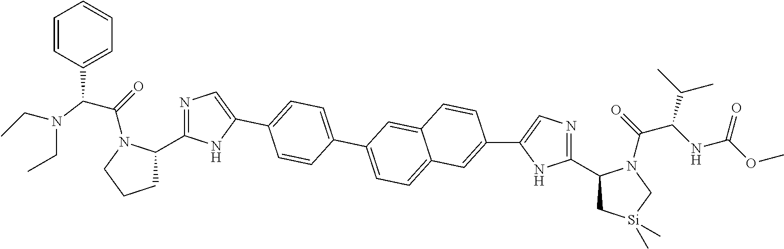 [SMILES: CCN(CC)[C@@H](C(=O)N1CCC[C@H]1c1ncc(-c2ccc(-c3ccc4cc(-c5cnc([C@@H]6C[Si](C)(C)CN6C(=O)[C@@H](NC(=O)OC)C(C)C)[nH]5)ccc4c3)cc2)[nH]1)c1ccccc1] |
| 157 | 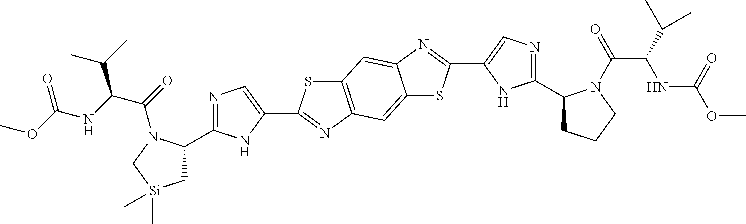 |
| 158 | 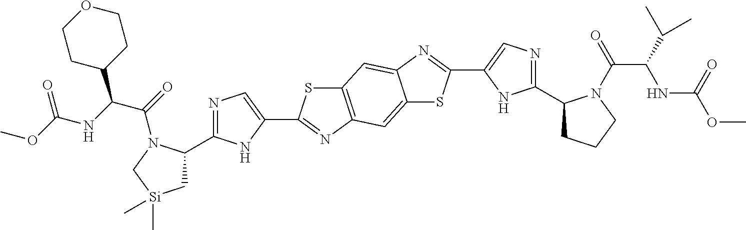 |
| 159 | 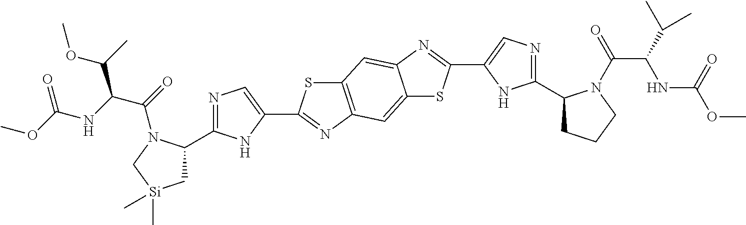 |
| 160 | 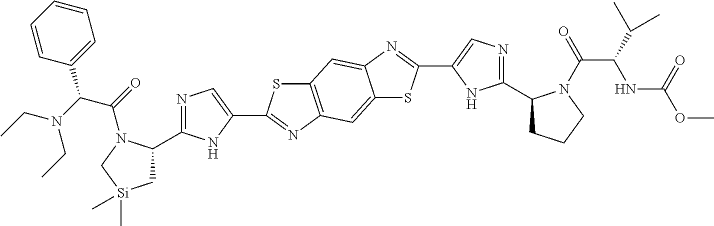 [SMILES: CCN(CC)[C@@H](C(=O)N1C[Si](C)(C)C[C@H]1c1ncc(-c2nc3cc4sc(-c5cnc([C@@H]6CCCN6C(=O)[C@@H](NC(=O)OC)C(C)C)[nH]5)nc4cc3s2)[nH]1)c1ccccc1] |
| 161 | 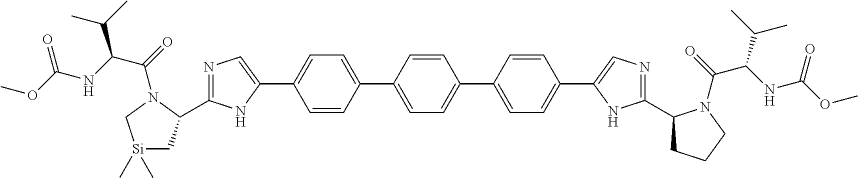 |
| 162 | 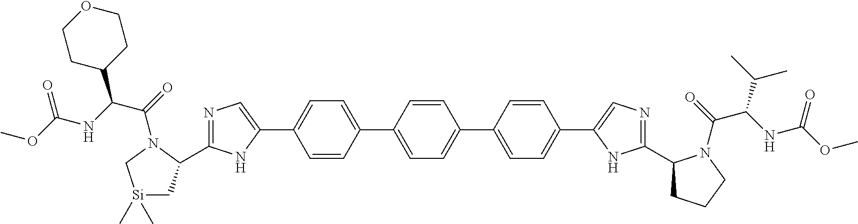 |
| 163 | 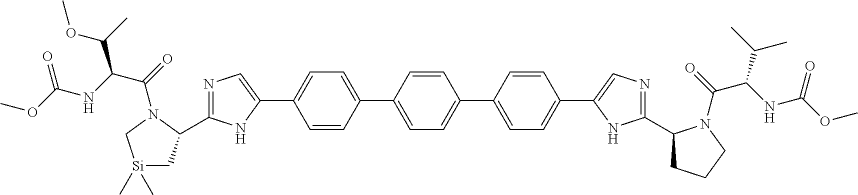 |
| 164 | 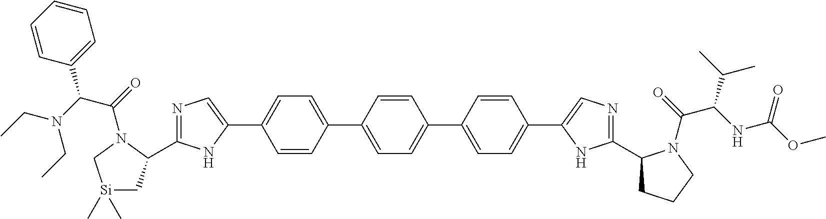 [SMILES: CCN(CC)[C@@H](C(=O)N1C[Si](C)(C)C[C@H]1c1ncc(-c2ccc(-c3ccc(-c4ccc(-c5cnc([C@@H]6CCCN6C(=O)[C@@H](NC(=O)OC)C(C)C)[nH]5)cc4)cc3)cc2)[nH]1)c1ccccc1] |
| 168 | 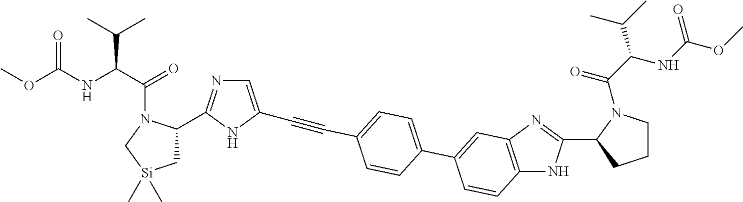 |
| 166 | 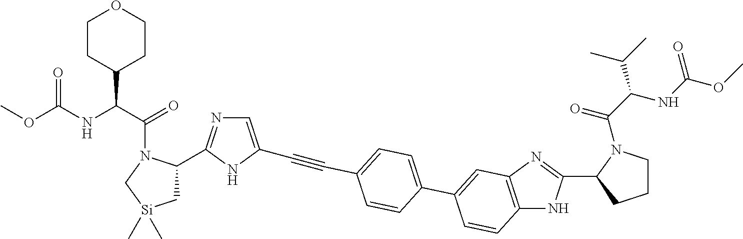 |
| 167 | 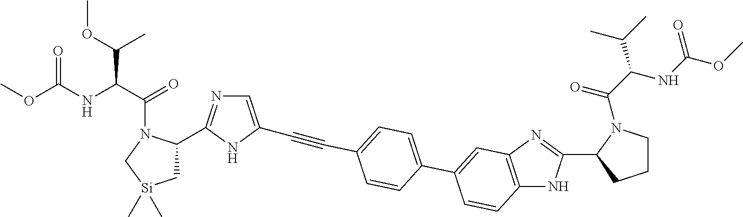 |
| 168 | 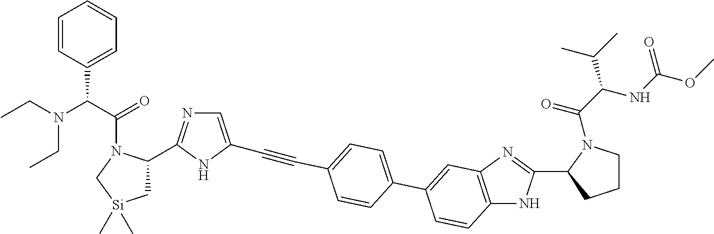 [SMILES: CCN(CC)[C@@H](C(=O)N1C[Si](C)(C)C[C@H]1c1ncc(C#Cc2ccc(-c3ccc4[nH]c([C@@H]5CCCN5C(=O)[C@@H](NC(=O)OC)C(C)C)nc4c3)cc2)[nH]1)c1ccccc1] |
| 169 | 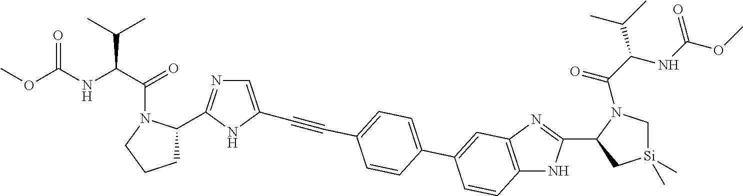 |
| 170 | 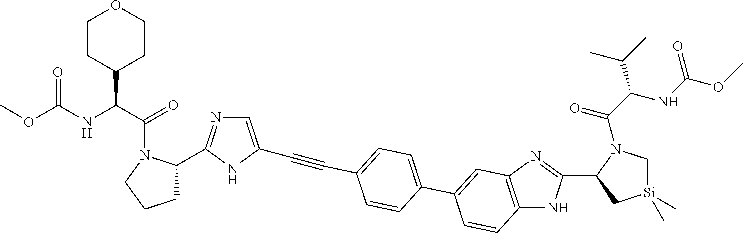 |
| 171 | 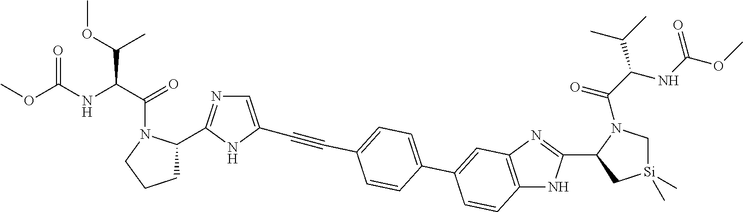 |
| 172 | 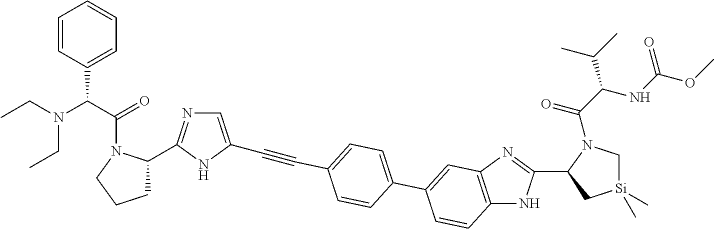 [SMILES: CCN(CC)[C@@H](C(=O)N1CCC[C@H]1c1ncc(C#Cc2ccc(-c3ccc4[nH]c([C@@H]5C[Si](C)(C)CN5C(=O)[C@@H](NC(=O)OC)C(C)C)nc4c3)cc2)[nH]1)c1ccccc1] |
| 173 | 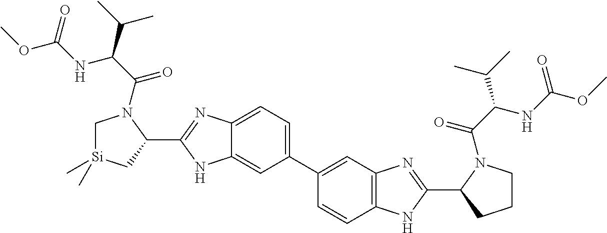 |
| 174 | 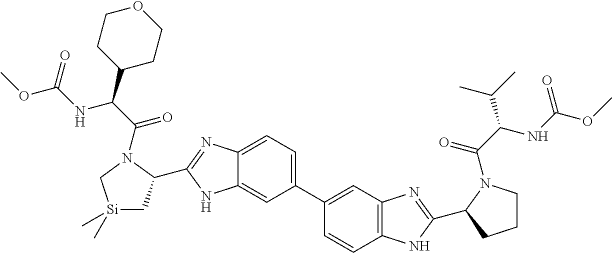 |
| 175 | 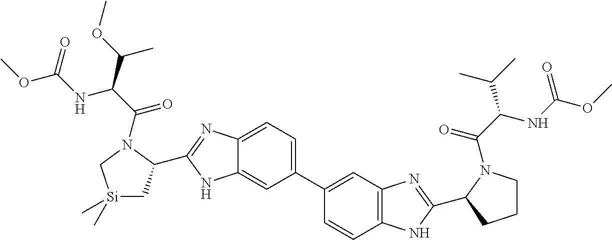 |
| 176 | 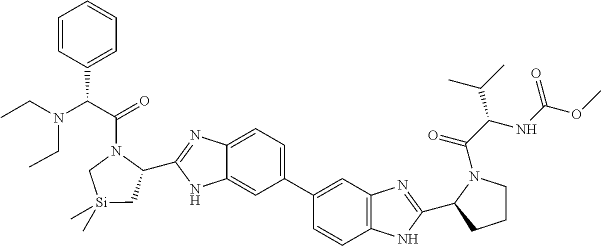 [SMILES: CCN(CC)[C@@H](C(=O)N1C[Si](C)(C)C[C@H]1c1nc2ccc(-c3ccc4[nH]c([C@@H]5CCCN5C(=O)[C@@H](NC(=O)OC)C(C)C)nc4c3)cc2[nH]1)c1ccccc1] |
| 177 | 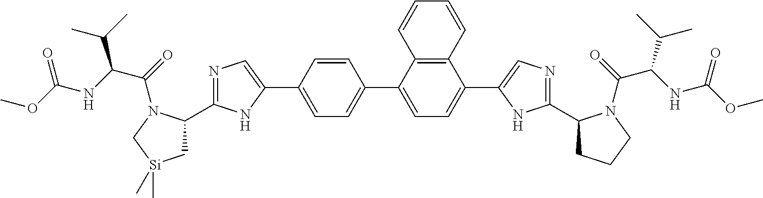 |
| 178 | 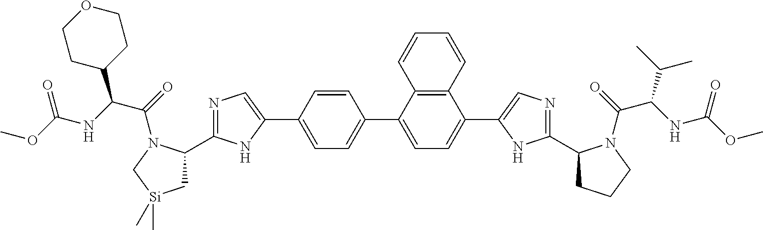 |
| 179 | 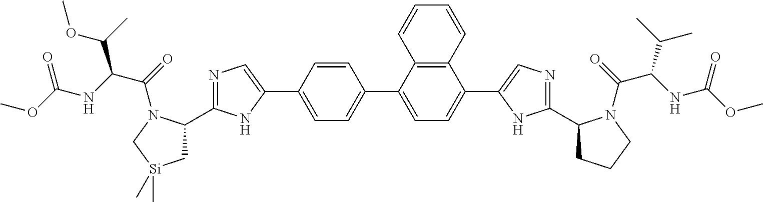 |
| 180 | 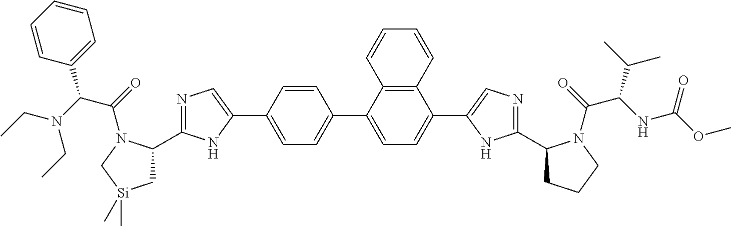 [SMILES: CCN(CC)[C@@H](C(=O)N1C[Si](C)(C)C[C@H]1c1ncc(-c2ccc(-c3ccc(-c4cnc([C@@H]5CCCN5C(=O)[C@@H](NC(=O)OC)C(C)C)[nH]4)c4ccccc34)cc2)[nH]1)c1ccccc1] |
| 181 | 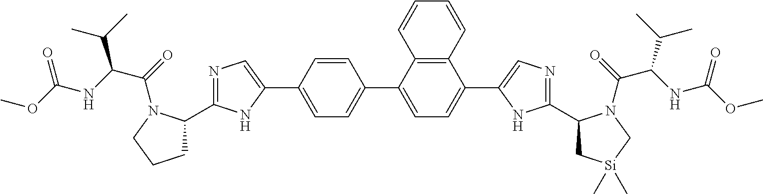 |
| 182 | 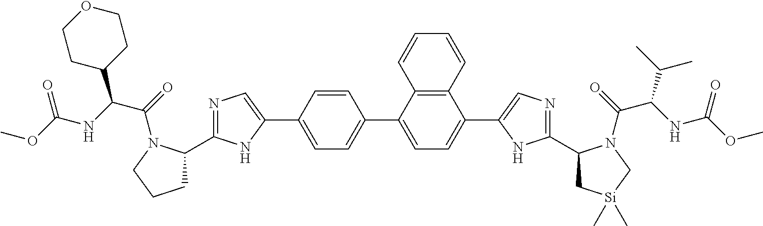 |
| 183 | 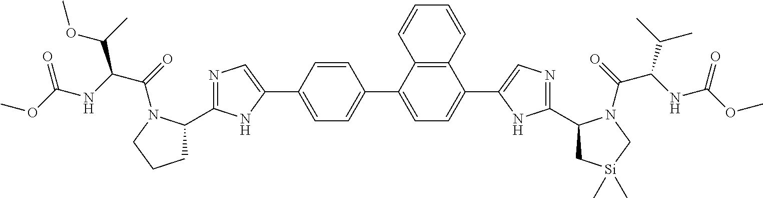 |
| 184 | 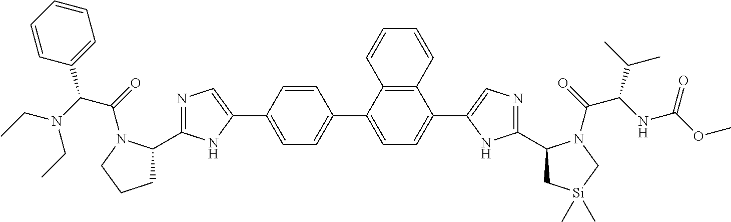 [SMILES: CCN(CC)[C@@H](C(=O)N1CCC[C@H]1c1ncc(-c2ccc(-c3ccc(-c4cnc([C@@H]5C[Si](C)(C)CN5C(=O)[C@@H](NC(=O)OC)C(C)C)[nH]4)c4ccccc34)cc2)[nH]1)c1ccccc1] |
| 185 | 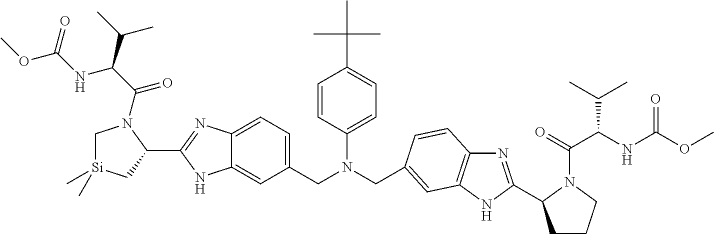 |
| 186 | 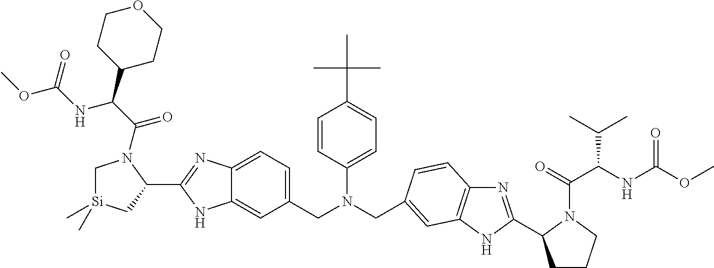 |
| 187 | 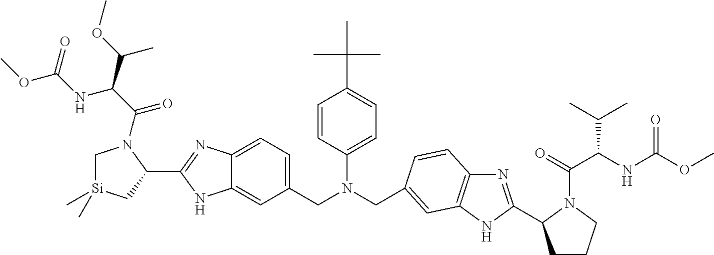 |
| 188 | 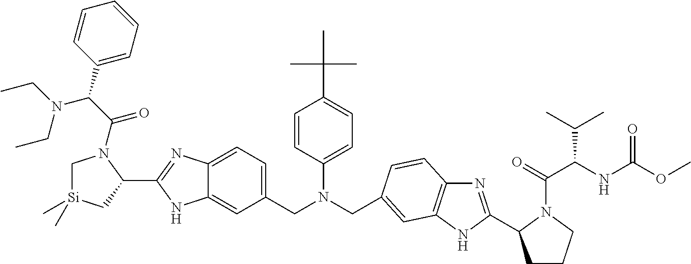 [SMILES: CCN(CC)[C@@H](C(=O)N1C[Si](C)(C)C[C@H]1c1nc2ccc(CN(Cc3ccc4nc([C@@H]5CCCN5C(=O)[C@@H](NC(=O)OC)C(C)C)[nH]c4c3)c3ccc(C(C)(C)C)cc3)cc2[nH]1)c1ccccc1] |
| 189 | 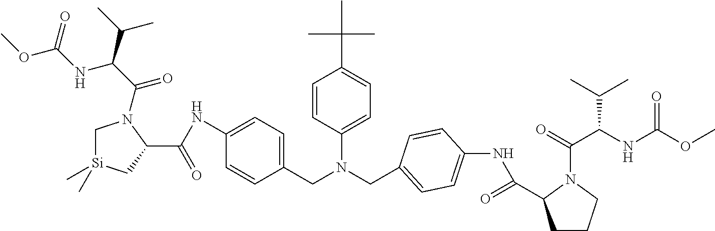 |
| 190 | 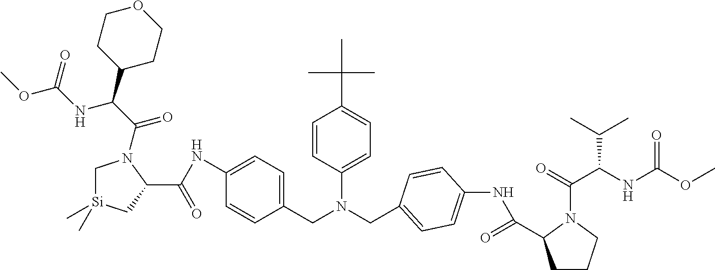 |
| 191 | 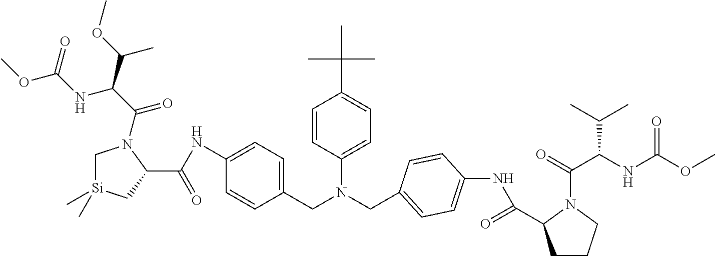 |
| 192 | 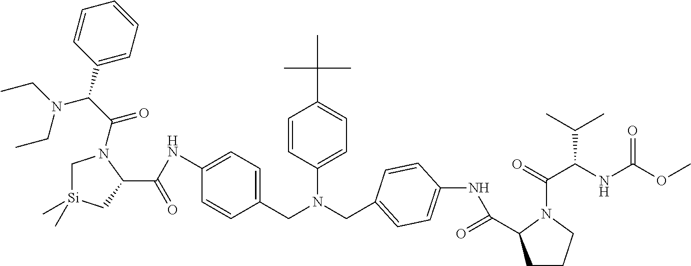 [SMILES: CCN(CC)[C@@H](C(=O)N1C[Si](C)(C)C[C@H]1C(=O)Nc1ccc(CN(Cc2ccc(NC(=O)[C@@H]3CCCN3C(=O)[C@@H](NC(=O)OC)C(C)C)cc2)c2ccc(C(C)(C)C)cc2)cc1)c1ccccc1] |
| 193 | 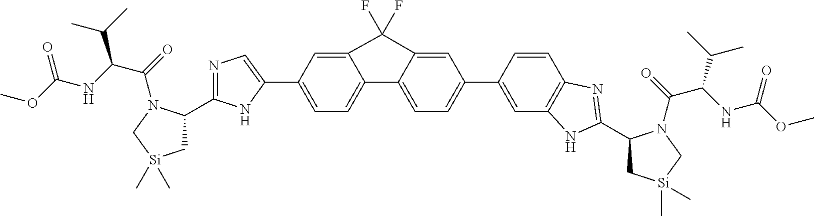 |
| 194 | 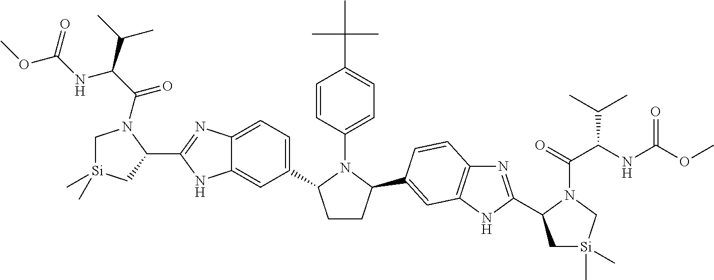 |
| 195 | 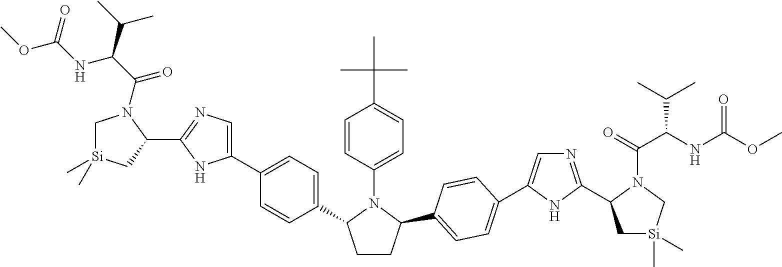 |
| 196 | 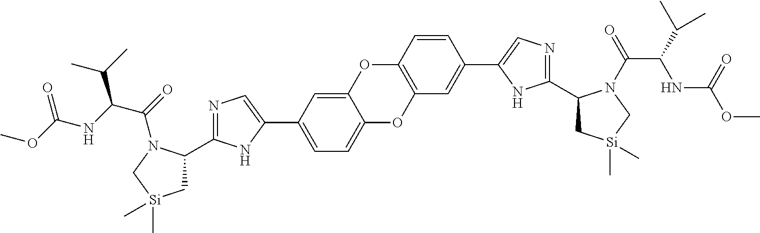 |
| 197 | 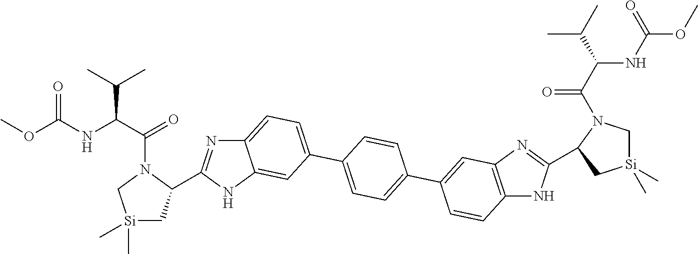 |
| 198 | 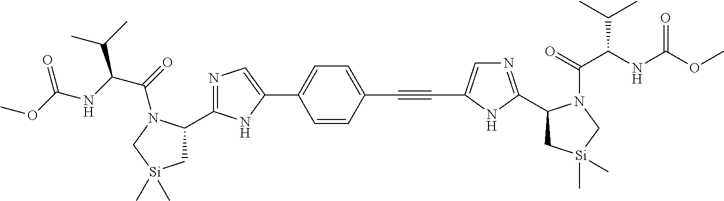 |
| 199 | 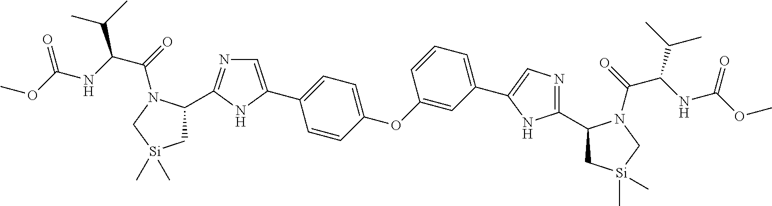 |
| 200 | 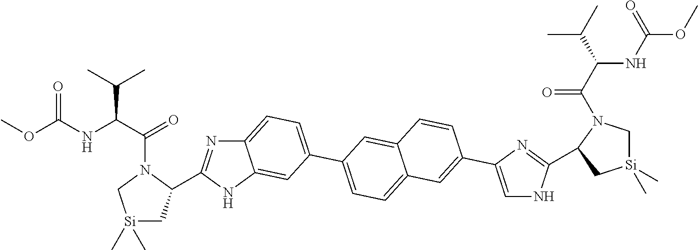 |
| 201 | 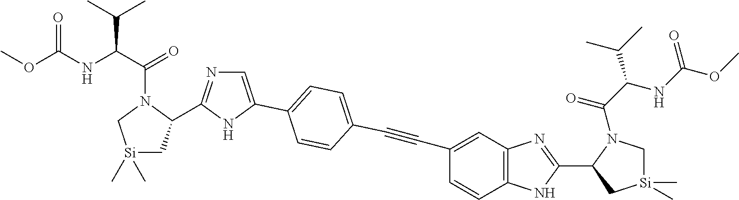 |
| 202 | 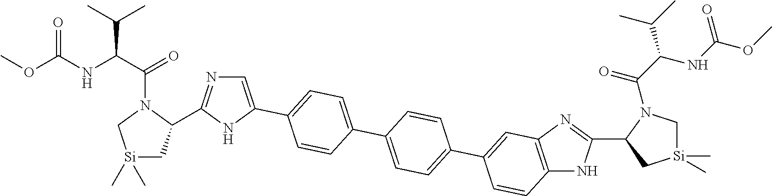 |
| 203 | 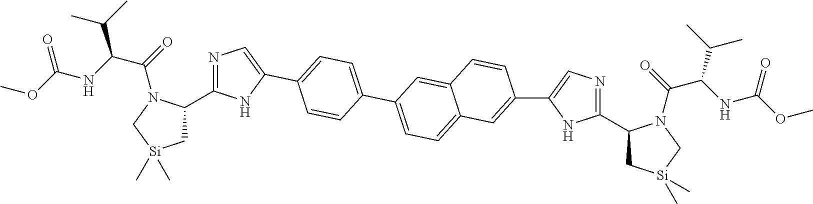 |
| 204 | 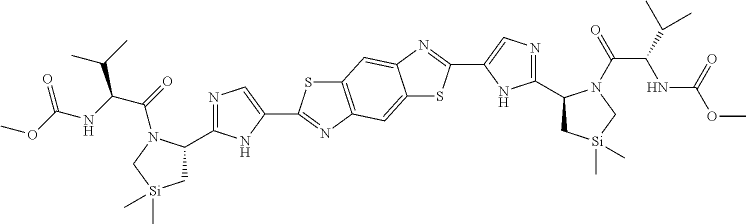 |
| 205 | 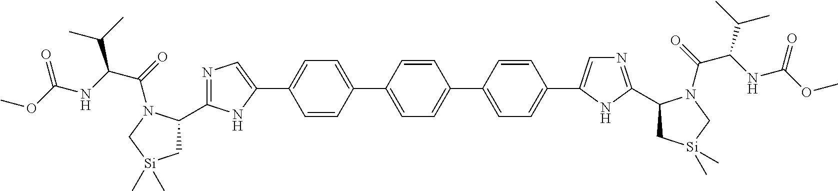 |
| 206 | 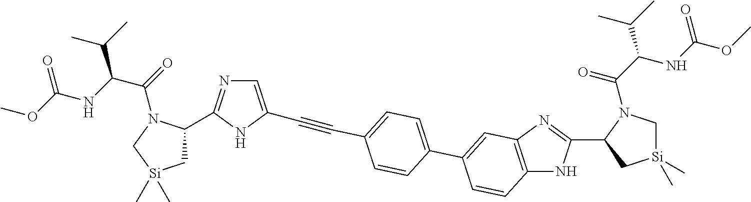 |
| 207 | 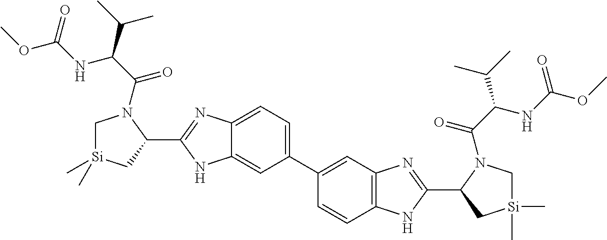 |
| 208 | 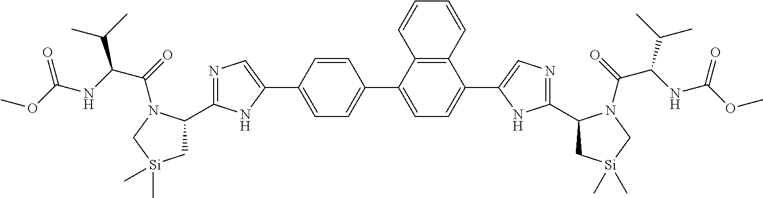 |
| 209 | 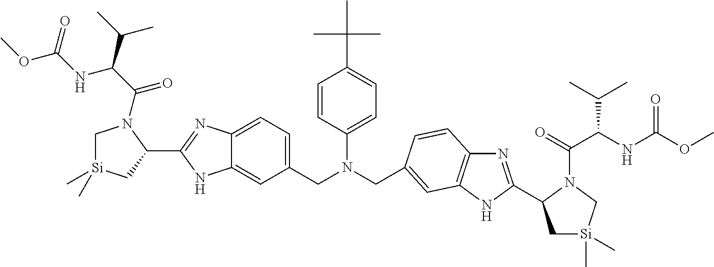 |
| 210 | 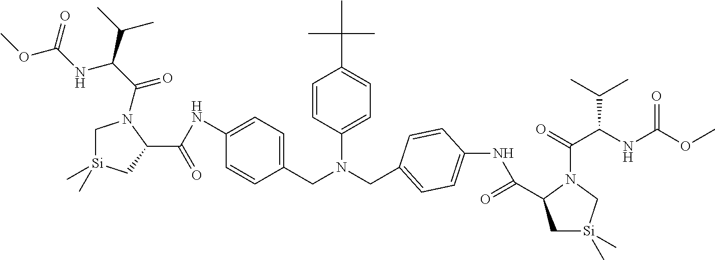 |

### Example 44

### Cell-Based HCV Replicon Assay

Measurement of inhibition by compounds of the present invention was performed using the HCV replicon system. Several different replicons encoding different HCV genotypes or mutations were used. In addition, potency measurements were made using different formats of the replicon assay, including different ways of measurements and different plating formats. See Jan M. Vrolijk et al., A replicons-based bioassay for the measurement of interferons in patients with chronic hepatitis C, 110 J. VIROLOGICAL METHODS 201 (2003); Steven S. Carroll et al., Inhibition of Hepatitis C Virus RNA Replication by 2′-Modified Nucleoside Analogs, 278(14) J. BIOLOGICAL CHEMISTRY 11979 (2003). However, the underlying principles are common to all of these determinations, and are outlined below.

### TaqMan®-Based Assay Protocol:

Compounds of the present invention were assayed for cell-based anti-HCV activity using the following protocol. Replicon cells were seeded at 5000 cells/well in 96-well collagen I-coated Nunc plates in the presence of the test compound. Various concentrations of test compound, typically in 10 serial 2-fold dilutions, were added to the assay mixture, with the starting concentration ranging from 250 μM to 1 μM. The final concentration of DMSO was 0.5%, fetal bovine serum was 5%, in the assay media. Cells were harvested on day 3 by the addition of 1× cell lysis buffer (Ambion cat #8721). The replicon RNA level was measured using real time PCR (TaqMan® assay). The amplicon was located in 5B. The PCR primers were: 5B.2F, ATGGACAGGCGCCCTGA (SEQ. ID NO. 1); 5B.2R, TTGATGGGCAGCTTGGTTTC (SEQ. ID NO. 2); the probe sequence was FAM-labeled CACGCCATGCGCTGCGG (SEQ. ID NO. 3). GAPDH RNA was used as endogenous control and was amplified in the same reaction as NS5B (multiplex PCR) using primers and VIC-labeled probe recommended by the manufacturer (PE Applied Biosystem). The real-time room temperature-PCR reactions were run on ABI PRISM 7900HT Sequence Detection System using the following program: 48° C. for 30 minutes, 95° C. for 10 minutes, 40 cycles of 95° C. for 15 sec, 60° C. for 1 minute. The ACT values (CT_{5B}-CT_{GAPDH}) were plotted against the concentration of test compound and fitted to the sigmoid dose-response model using XLfit4 (MDL). EC₅₀ was defined as the concentration of inhibitor necessary to achieve ΔCT=1 over the projected baseline; EC₉₀ the concentration necessary to achieve ΔCT=3.2 over the baseline. Alternatively, to quantitate the absolute amount of replicon RNA, a standard curve was established by including serially diluted T7 transcripts of replicon RNA in the Taqman assay. All TaqMan® reagents were from PE Applied Biosystems. Such an assay procedure was described in detail in e.g. Malcolm et al., Antimicrobial Agents and Chemotherapy 50: 1013-1020 (2006).

HCV replicon EC₅₀ assay data for various replicons and mutants was calculated for selected compounds of the present invention using this method and is provided in the tables above herein. This data indicates that the compounds of the present invention are highly active versus a wide variety of HCV NS5A replicons and mutants.

The study of the HCV life cycle has been difficult due to the lack of a cell-culture system to support the HCV virus. To date, compounds in different structural classes acting on different sites within the HCV polyprotein have demonstrated efficacy in various species, including humans, in reducing HCV viral titers. Furthermore, the subgenomic replicon assay is highly correlated with efficacy in non-humans and humans infected with HCV. See K. del Carmen et al., Annals of Hepatology, 2004, 3:54.

It is accepted that the HCV replicon system described above is useful for the development and the evaluation of antiviral drugs. See Pietschmann, T. & Bartenschlager, R., Current Opinion in Drug Discovery Research 2001, 4:657-664).

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

1. A compound having the formula: 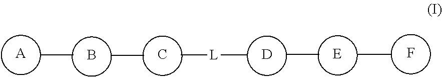
or a pharmaceutically acceptable salt thereof,
wherein:
A and F are each independently R¹² or 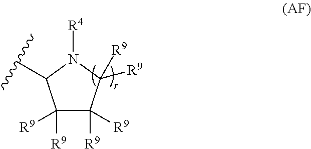
wherein each occurrence of (AF) can be independently and optionally fused to a benzene ring and wherein any two R⁹ groups that are attached to the same (AF) group, together with the ring carbon atom(s) to which they are attached, can join to form a 3 to 7-membered cycloalkyl group, such that when the group corresponding to variable D does not contains the group —Si(R¹¹)₂— as a ring member, then at least one of A and F is R¹²;
B and E are each independently imidazolyl or benzimidazolyl, wherein said imidazolyl group and said benzimidazolyl group can be optionally and independently substituted on a ring carbon atoms with R⁶;
C is selected from a bond, phenylene, naphthylene and 5 or 6-membered monocyclic heteroarylene, wherein said phenylene group, said naphthylene group and said 5 or 6-membered monocyclic heteroarylene group can be optionally and independently substituted on one or more ring carbon atoms with R¹⁰;
D is selected from phenylene, naphthylene, 5 or 6-membered monocyclic heteroarylene, 9 or 10-membered bicyclic heteroarylene and 13 to 14-membered tricyclic heteroarylene, wherein said 5-membered monocyclic heteroarylene group, said 9 or 10-membered bicyclic heteroarylene group and said 13 to 14-membered tricyclic heteroarylene group can be optionally and independently substituted on one or more ring carbon atoms with R¹⁰, and wherein said 9 or 10-membered bicyclic heteroarylene group and said 13 to 14-membered tricyclic heteroarylene group can optionally contain the group —Si(R¹¹)₂— as a ring member;
L is selected from a bond, C₁-C₃ alkylene, —CH═CH— and —C≡C—, such that when D is 13 to 14-membered tricyclic heteroarylene, then L is a bond;
each occurrence of R¹ is independently selected from C₁-C₆ alkyl, C₁-C₆ haloalkyl, 3- to 7-membered cycloalkyl, 4- to 7-membered heterocycloalkyl, aryl and 5 or 6-membered monocyclic heteroaryl, wherein said 3- to 7-membered cycloalkyl group, said 4- to 7-membered heterocycloalkyl group, said aryl group or said 5 or 6-membered monocyclic heteroaryl group can be optionally substituted with up to three groups, which can be the same or different, and are selected from C₁-C₆ alkyl, 3- to 7-membered cycloalkyl, 4- to 7-membered heterocycloalkyl, aryl, heteroaryl, halo, C₁-C₆haloalkyl, —Si(R¹¹)₃, —CN, —OR², —N(R²)₂, —C(O)R³, —C(O)OR², —C(O)N(R²)₂, —NHC(O)R³, —NHC(O)NHR², —NHC(O)OR², —OC(O)R³, —SR² and —S(O)₂R³;
each occurrence of R² is independently selected from H, C₁-C₆ alkyl, C₁-C₆haloalkyl, —C₁-C₆alkylene-OC(O)(C₁-C₆alkyl), C₁-C₆hydroxyalkyl, 3 to 7-membered cycloalkyl, 4 to 7-membered heterocycloalkyl, aryl and 5 or 6-membered monocyclic heteroaryl wherein said 3- to 7-membered cycloalkyl group, said 4- to 7-membered heterocycloalkyl group, said aryl group or said 5 or 6-membered monocyclic heteroaryl group can be optionally and independently substituted with up to three groups, each independently selected from —OH, halo, C₁-C₆ alkyl, C₁-C₆ haloalkyl, —NH(C₁-C₆ alkyl) and —N(C₁-C₆ alkyl)₂;
each occurrence of R³ is independently selected from C₁-C₆ alkyl, C₁-C₆ haloalkyl, 3 to 7-membered cycloalkyl, 4 to 7-membered heterocycloalkyl, aryl, and 5 or 6-membered monocyclic heteroaryl;
each occurrence of R⁴ is independently selected from H, —C₁-C₆ alkyl, C₁-C₆haloalkyl, —C(O)R¹, —C(O)OR¹, —C(O)C(R⁵)₂NHC(O)OR¹ and —C(O)—C(R⁵)₂—N(R¹)₂;
each occurrence of R⁵ is independently selected from H, C₁-C₆ alkyl, C₁-C₆haloalkyl, -alkylene-O—(C₁-C₆ alkyl), C₁-C₆ silylalkyl, —(CH₂)ₙ-aryl, —(CH₂)ₙ-(3- to 7-membered cycloalkyl), —(CH₂)ₙ-(4- to 7-membered heterocycloalkyl group) and —(CH₂)ₙ-(5 or 6-membered monocyclic heteroaryl), wherein said 3- to 7-membered cycloalkyl group, said 4- to 7-membered heterocycloalkyl group, said aryl group or said 5 or 6-membered monocyclic heteroaryl group can be optionally and independently substituted with up to three R⁷ groups, or two R⁵ groups that are attached to the same carbon atom, together with the common carbon atom to which they are attached, can join to form a 3-6 membered cycloalkyl group;
each occurrence of R⁶ is independently selected from H, halo, C₁-C₆ alkyl and 3 to 7-membered cycloalkyl;
each occurrence of R⁷ is independently selected from H, C₁-C₆ alkyl, halo, —C₁-C₆ haloalkyl, C₁-C₆ hydroxyalkyl, —OH, —C(O)NH—(C₁-C₆ alkyl), —C(O)N(C₁-C₆ alkyl)₂, —O—(C₁-C₆ alkyl), —NH₂, —NH(C₁-C₆ alkyl), —N(C₁-C₆ alkyl)₂ and —NHC(O)—(C₁-C₆ alkyl) and —Si(R¹¹)₃;
each occurrence of R⁹ is independently selected from H, halo and C₁-C₆ alkyl;
each occurrence of R¹⁰ is independently selected from H, C₁-C₆ alkyl, C₁-C₆haloalkyl, 3 to 7-membered cycloalkyl, 4 to 7-membered heterocycloalkyl, aryl, 5 or 6-membered monocyclic heteroaryl, halo, —CN, —OR³, —N(R²)₂, —C(O)R³, —C(O)OR², —C(O)N(R²)₂, —NHC(O)R³, —NHC(O)NHR², —NHC(O)OR², —OC(O)R³, —SR², —S(O)₂R³ and Si(R¹¹)₃, wherein any two R¹⁰ groups that are attached to the same ring, together with the ring carbon atom(s) to which they are attached, can optionally join to form a 5 to 7-membered cycloalkyl group or 4- to 7-membered heterocycloalkyl group;
each occurrence of R¹¹ is independently selected from C₁-C₆ alkyl, 3- to 7-membered cycloalkyl, 4- to 7-membered heterocycloalkyl, aryl, heteroaryl, C₁-C₆ haloalkyl, —CN and —OR², wherein two R¹¹ groups that are attached to the same silicon atom, together with the common silicon atom to which they are attached, can optionally join to form a 4- to 7-membered spirocyclic silicon-containing heterocycloalkyl ring;
each occurrence of R¹² is independently a monocyclic 5 to 7-membered silylheterocycloalkyl ring or a bicyclic 7 to 11-membered bicyclic silylheterocycloalkyl ring wherein said silylheterocycloalkyl rings contains as heteroatom ring members:
(i) one —Si(R¹¹)₂— group; and
(ii) one —N(R⁴)— group;
wherein an R¹² group can be optionally and independently substituted on one or more ring carbon atoms with R¹⁰;
each occurrence of n is independently 0, 1, 2 or 3; and
each occurrence of r is independently 0 or 1.

2. The compound of claim 1, wherein A and F are each independently selected from:

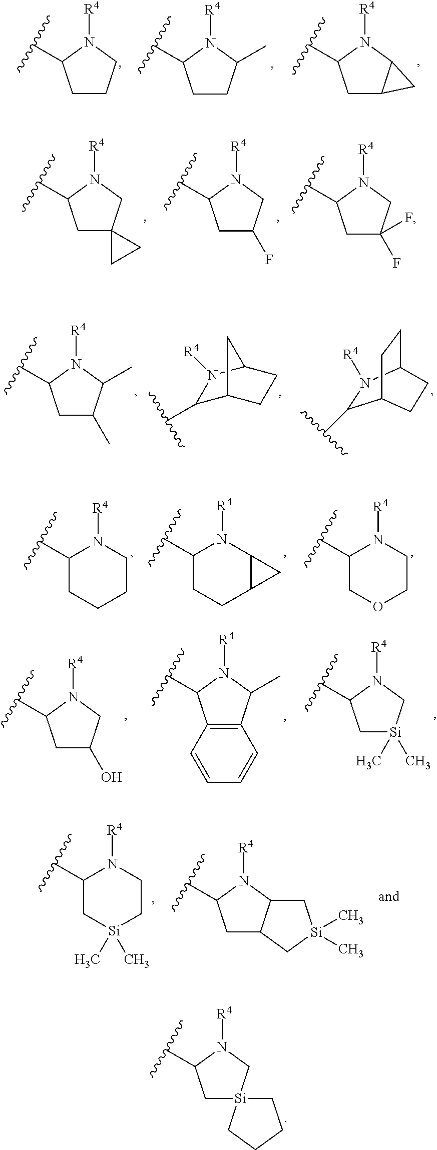

3. The compound of claim 1, wherein A and F are each independently selected from:

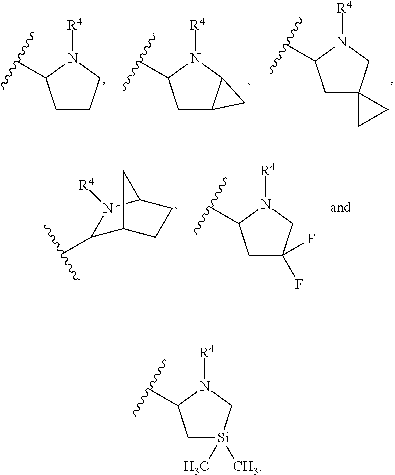

4. The compound of claim 1, wherein each occurrence of R⁴ is independently: 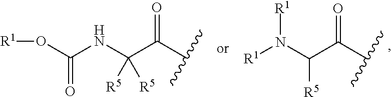
wherein each occurrence of R¹ is C₁-C₆ alkyl, and R⁵ is selected from C₁-C₆ alkyl, —CH₂—S—(C₁-C₆ alkyl), benzyl, —(CH₂)ₙ-aryl, —CH₂-heteroaryl, —(CH₂)ₙ-(3- to 7-membered cycloalkyl) and 4- to 7-membered heterocycloalkyl, wherein said C₁-C₆ alkyl group can be optionally substituted with —OH or —O—(C₁-C₆ alkyl), or two R⁵ groups that are attached to a common carbon atom, and the common carbon atom to which they are attached, can combine to form a 3 to 7-membered cycloalkyl group.

5. The compound of claim 4, wherein each occurrence of R⁴ is independently: 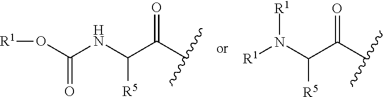
R¹ is methyl and R⁵ is selected from methyl, isopropyl, isobutyl, phenyl, cyclopropyl, cyclopentyl, cyclohexyl, —CH(OCH₃)CH₃, —CH(OH)CH₂CH₃, —CH(OH)CH(CH₃)₂, tetrahydropyranyl, oxepanyl, —CH₂-cyclopropyl, —CH₂—S—CH₃, and —CH₂-indolyl.

6. The compound of claim 4, wherein each occurrence of R⁴ is:

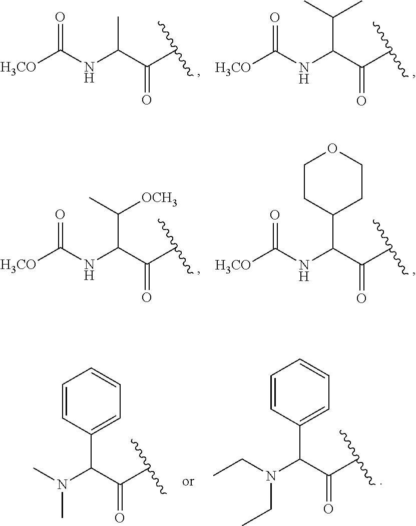

7. The compound of claim 1, wherein B and E are each independently 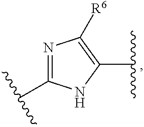
wherein R⁶ is H, F, Cl or cyclopropyl.

8. The compound of claim 1, wherein one of B and E is 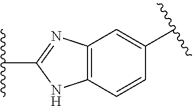 [SMILES: *c1ccc2[nH]c(*)nc2c1]
and the other of B and E is: 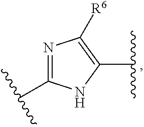
wherein R⁶ is H, F, Cl or cyclopropyl.

9. The compound of claim 1, wherein L is a bond or —C≡C—.

10. The compound of claim 1, wherein C is a bond or phenylene.

11. The compound of claim 1, wherein D is phenylene or 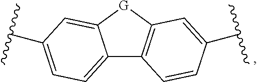
wherein G is —CF₂— or —Si(CH₃)₂—.

12. The compound of claim 1, having the formula: 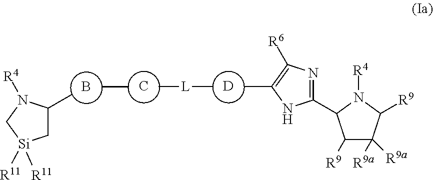
or a pharmaceutically acceptable salt thereof, wherein
B is imidazolyl or benzimidazolyl, each of which can be optionally substituted on a ring carbon atom with R⁶;
C is a bond or phenylene;
D is phenylene or 13 to 14-membered tricyclic heteroarylene, wherein said 13 to 14-membered tricyclic heteroarylene group can be optionally substituted on a ring carbon atom, ring nitrogen atom or ring silyl atom with up to 4 groups, each independently selected from C₁-C₆ alkyl and halo.
L is a bond or —C≡C—, such that when D is a 13 to 14-membered tricyclic heteroarylene group, then L and C are each a bond;
each occurrence of R¹ is C₁-C₆ alkyl;
each occurrence of R⁴ is independently: 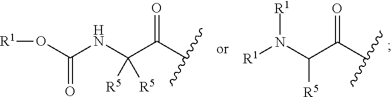
R⁵ is selected from C₁-C₆ alkyl, —CH₂—S—(C₁-C₆ alkyl), benzyl, —(CH₂)ₙ-aryl, —CH₂-heteroaryl, —(CH₂)ₙ-(3- to 7-membered cycloalkyl) and 4- to 7-membered heterocycloalkyl, wherein said C₁-C₆ alkyl group can be optionally substituted with —OH or —O—(C₁-C₆ alkyl), or two R⁵ groups that are attached to a common carbon atom, and the common carbon atom to which they are attached, can combine to form a 3 to 7-membered cycloalkyl group;
R⁶ is H, halo or 3 to 7-membered cycloalkyl;
each occurrence of R⁹ is: (i) H, or (ii) both R⁹ groups join to form a C₂-C₃ alkylene group, or (iii) one R⁹ group and one R^{9a} group and the ring carbon atoms to which they are attached join to form a 3 to 7-membered cycloalkyl group; and
each occurrence of R^{9a} is independently H or halo, or both R^{9a} groups and the common carbon atom to which they are attached, join to form a 3 to 7-membered cycloalkyl group.

13. The compound of claim 1, having the formula: 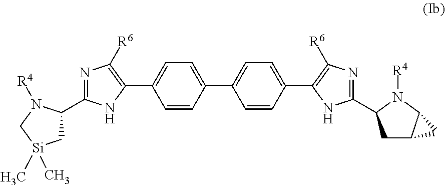
or a pharmaceutically acceptable salt thereof,
wherein:
each occurrence of R¹ is independently C₁-C₆ alkyl;
each occurrence of R⁴ is independently —C(O)CH(R⁷)C(O)OR¹ or —C(O)CH(R⁷)N(R¹)₂;
each occurrence of R⁷ is independently C₁-C₆ alkyl, phenyl or 4 to 7-membered heterocycloalkyl; and
each occurrence of R⁶ is H or halo.

14. The compound of claim 13, wherein each occurrence of R⁴ is independently each occurrence of R⁴ is: 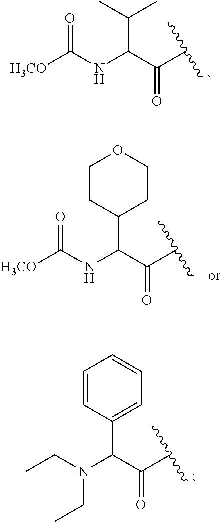 [SMILES: *C(=O)C(NC(=O)OC)C(C)C ; *C(=O)C(NC(=O)OC)C1CCOCC1 ; *C(=O)C(c1ccccc1)N(CC)CC]
and
each occurrence of R⁶ is H or C1.

15. The compound of claim 1 that is a compound numbered from 1 to 210 in the above specification, or a pharmaceutically acceptable salt thereof.

16. (canceled)

17. A pharmaceutical composition comprising an effective amount of a compound of claim 1, or a pharmaceutically acceptable salt thereof, and a pharmaceutically acceptable carrier.

18. The pharmaceutical composition of claim 17, further comprising a second therapeutic agent selected from the group consisting of HCV antiviral agents, immunomodulators, and anti-infective agents.

19. The pharmaceutical composition of claim 18, further comprising a third therapeutic agent selected from the group consisting of HCV protease inhibitors, HCV NS5A inhibitors and nucleoside and non-nucleoside HCV NS5B polymerase inhibitors.

20. (canceled)

21. A method of treating a patient infected with HCV comprising the step of administering to said patient a compound of claim 1, or a pharmaceutically acceptable salt thereof, in an amount effective to prevent and/or treat infection by HCV in said patient.

22. The method of claim 21, further comprising the step of administering an HCV protease inhibitor to said patient.

23. (canceled)
